(19) United States
(12) Patent Application Publication
(10) Pub. No.: US 2012/0252054 A1
(43) Pub. Date: Oct. 4, 2012

(54) SYSTEM FOR TRANSFORMATION OF THE CHLOROPLAST GENOME OF SCENEDESMUS SP. AND DUNALIELLA SP.

(75) Inventors: Kyle M. Botsch, San Diego, CA (US); Amy C. Curran, San Diego, CA (US); Wendy Levine, Santee, CA (US); Bryan O'Neill, Carlsbad, CA (US); Shawn Joseph Szyjka, San Diego, CA (US); Michael Mendez, San Diego, CA (US)

(73) Assignee: SAPPHIRE ENERGY INC, SAN DIEGO, CA (US)

(21) Appl. No.: 13/496,149

(22) PCT Filed: Sep. 14, 2010
(86) PCT No.: PCT/US10/48828

§ 371 Date: Jun. 21, 2012

Related U.S. Application Data
(60) Provisional application No. 61/242,735, filed on Sep. 15, 2009.

(51) Int. Cl. C12N 1/13; C12N 15/79; C12Q 1/02

(52) U.S. Cl. 435/29; 435/257.2; 435/471

(57) ABSTRACT

The present disclosure relates to methods of transforming various species of algae, for example, algae from the genus Scenedesmus and the genus Dunaliella, vectors and nucleic acid constructs useful in conducting such transformations, and recombinant algae, for example, Scenedesmus and Dunaliella produced using the vectors and methods disclosed herein.

### CROSS REFERENCE TO RELATED APPLICATION

This application claims the benefit of U.S. Provisional Application No. 61/242,735, filed Sep. 15, 2009, the entire contents of which are incorporated by reference for all purposes.

### INCORPORATION BY REFERENCE

All publications, patents, patent applications, public databases, public database entries, and other references cited in this application, are herein, incorporated by reference in their entirety as if each individual publication, patent, patent application, public database, public database entry, or other reference was specifically and individually indicated to be incorporated by reference.

### BACKGROUND

Algae are unicellular organisms, producing oxygen by photosynthesis. One group, the microalgae, are useful for biotechnology applications for many reasons, including their high growth rate and tolerance to varying environmental conditions. The use of microalgae in a variety of industrial processes for commercially important products is known and/or has been suggested. For example, microalgae have uses in the production of nutritional supplements, pharmaceuticals, natural dyes, a food source for fish and crustaceans, biological control of agricultural pests, production of oxygen and removal of nitrogen, phosphorus and toxic substances in sewage treatment, and pollution controls, such as biodegradation of plastics or uptake of carbon dioxide.

Microalgae, like other organisms, contain lipids and fatty acids as membrane components, storage products, metabolites and sources of energy. Some algal strains, diatoms, and cyanobacteria have been found to contain proportionally high levels of lipids (over 30%). Microalgal strains with high oil or lipid content are of great interest in the search for a sustainable feedstock for the production of biofuels.

Some wild-type algae are suitable for use in various industrial applications. However, it is recognized that by modification of algae to improve particular characteristics useful for the aforementioned applications, the relevant processes are more likely to be commercially viable. To this end, algal strains can be developed which have improved characteristics over wild-type strains. Such developments have been made by traditional techniques of screening and mutation and selection. Further, recombinant DNA technologies have been widely suggested for algae. Such approaches may increase the economic validity of production of commercially valuable products.

One area in which algae have received increasing attention is the production of fuel products. Fuel products, such as oil, petrochemicals, and other substances useful for the production of petrochemicals are increasingly in demand. Much of today's fuel products are generated from fossil fuels, which are not considered renewable energy sources, as they are the result of organic material being covered by successive layers of sediment over the course of millions of years. There is also a growing desire to lessen dependence on imported crude oil. Public awareness regarding pollution and environmental hazards has also increased. As a result, there has been a growing interest and need for alternative methods to produce fuel products. Thus, there exists a pressing need for alternative methods to develop fuel products that are renewable, sustainable, and less harmful to the environment. One potential source of alternative production of fuel and fuel precursors is genetically modified organisms, such as bacteria and plants, including algae. To date, algae have yet to be successfully developed as a commercially viable platform for biofuel production, due mainly to the high cost of harvesting and processing of algae for recovery of the biofuel. Thus, a need exists to develop host organisms such as algae (for example, Scenedesmus sp., Chlamydomonas sp., and Dunaliella sp.) and bacteria for which such costs are reduced. One way of genetically modifying an organism is to transform the organism with a nucleic acid that encodes for a protein, wherein expression of the protein results, for example, in the increased production of a product, or in the production of a product that the organism does not usually make.

### SUMMARY

1. An isolated Scenedesmus sp. comprising a chloroplast genome that has been transformed with an exogenous nucleotide sequence, wherein the exogenous nucleotide sequence comprises a nucleic acid sequence encoding at least one protein. 2. The isolated Scenedesmus sp. of claim 1, wherein the protein is involved in the isoprenoid biosynthesis pathway. 3. The isolated Scenedesmus sp. of claim 2, wherein the protein a synthase. 4. The isolated Scenedesmus sp. of claim 3, wherein the synthase is a farnesyl-diphosphate (FPP) synthase. 5. The isolated Scenedesmus sp. of claim 4, wherein the FPP synthase is from G. gallus. 6. The isolated Scenedesmus sp. of claim 3, wherein the synthase is a fusicoccadiene synthase. 7. The isolated Scenedesmus sp. of claim 6, wherein the fusicoccadiene synthase is from P. amygdali. 8. The isolated Scenedesmus sp. of claim 3, wherein the synthase is a bisabolene synthase. 9. The isolated Scenedesmus sp. of claim 8, wherein the bisabolene synthase is from A. grandis. 10. The isolated Scenedesmus sp. of claim 1, wherein the exogenous nucleotide sequence is at least 0.5 kb, at least 1.0 kb, at least 2 kb, at least 3 kb, at least 5 kb, at least 8 kb, at least 11 kb, or at least 19 kb. 11. The isolated Scenedesmus sp. of claim 1, wherein the nucleic acid sequence encodes for two proteins, three proteins, or four proteins. 12. The isolated Scenedesmus sp. of claim 1, wherein the exogenous nucleotide further comprises a second nucleic acid sequence encoding a selectable marker. 13. The isolated Scenedesmus sp. of claim 12, wherein the marker is chloramphenicol acetyltransferase (CAT), erythromycin esterase, or cytosine deaminase. 14. The isolated Scenedesmus sp. of claim 1, wherein the Scenedesmus sp. is S. dimorphus. 15. The isolated Scenedesmus sp. of claim 1, wherein the Scenedesmus sp. is S. obliquus. 16. The isolated Scenedesmus sp. of claim 1, wherein the nucleic acid sequence encodes for a biomass-degrading enzyme. 17. The isolated Scenedesmus sp. of claim 16, wherein the biomass-degrading enzyme is a galactanase, a xylanase, a protease, a carbohydrase, a lipase, a reductase, an oxidase, a transglutaminase, or a phytase. 18. The isolated Scenedesmus sp. of claim 16, wherein the biomass degrading enzyme is an endoxylanase, an exo-β-glucanase, an endo-β-glucanase, a β-glucosidase, an endoxylanase, or a lignase. 19. The isolated Scenedesmus sp. of claim 1, wherein the nucleic acid sequence encodes for an esterase, 20. The isolated Scenedesmus sp. of claim 19, wherein the esterase is an erythromycin esterase, 23. The isolated Scenedesmus sp. of claim 1, wherein the nucleic acid sequence encodes for a deaminase. 22. The isolated Scenedesmus sp. of claim 1, wherein the nucleic acid sequence encodes for a betaine aldehyde dehydrogenase. 23. The isolated Scenedesmus sp. of any of claims 1 to 22, wherein the nucleic acid sequence is codon optimized for expression in the chloroplast genome of the Scenedesmus sp. 24. An isolated Scenedesmus sp. comprising a chloroplast genome transformed with an exogenous nucleotide sequence wherein the transformed Scenedesmus sp. has an isoprenoid content that is different than an untransformed Scenedesmus sp. that is the same species as the isolated Scenedesmus sp., and wherein the exogenous nucleotide sequence comprises a nucleic acid encoding for an enzyme involved in isoprenoid biosynthesis. 25. The isolated Scenedesmus sp. of claim 24, wherein the nucleic acid does not encode for an ent-kaurene synthase. 26. The isolated Scenedesmus sp. of claim 24, wherein the nucleic acid is codon optimized for expression in the chloroplast genome of the Scenedesmus sp.

27. An isolated Scenedesmus sp. comprising a chloroplast genome transformed with an exogenous nucleotide sequence wherein the transformed Scenedesmus sp. has an increased accumulation of fatty acid based lipids and/or a change in the types of lipids, as compared to an untransformed Scenedesmus sp. that is the same species as the isolated Scenedesmus sp., and wherein the exogenous nucleotide comprises a nucleic acid sequence encoding for an enzyme involved in fatty acid synthesis. 28. The isolated Scenedesmus sp. of claim 27, wherein the nucleic acid is codon optimized for expression in the chloroplast genome of the Scenedesmus sp

29. A method of transforming a chloroplast genome of a Scenedesmus sp. with a vector, wherein the vector comprises: i) a first nucleotide sequence of a Scenedesmus sp. chloroplast genome; ii) a second nucleotide sequence of a Scenedesmus sp. chloroplast genome; iii) a third nucleotide sequence comprising an exogenous nucleotide sequence, wherein the exogenous nucleotide sequence comprises a nucleic acid encoding a protein of interest, wherein the third nucleotide sequence is located between the first and second nucleotide sequences, and wherein the vector is used to transform the chloroplast genome of the Scenedesmus sp.; and iv) a promoter configured for expression of the protein of interest. 30. The method of claim 29, wherein the third nucleotide sequence further comprises a second nucleic acid sequence encoding a second protein of interest. 31. The method of claim 29, wherein the promoter is a psbD or a tufA promoter. 32. The method of claim 29, wherein the Scenedesmus sp. is S. dimorphus, 33. The method of claim 29, wherein the Scenedesmus sp, is S. obliquus. 34. The method of claim 29, wherein the first nucleotide sequence is at least 500 bp, at least 1000 bp, or at least 1,500 bp in length, and the first nucleotide sequence is homologous to a first portion of the genome of the Scenedesmus sp., and the second nucleotide sequence is at least 500 bp, at least 1000 bp, or at least 1,500 bp in length, and the second nucleotide sequence is homologous to a second portion of the genome of the Scenedesmus sp. 35. The method of claim 29, wherein the third nucleotide sequence is at least 0.5 kb, at least 1.0 kb, at least 2 kb, at least 3 kb, at least 5 kb, at least 8 kb, at least 11 kb, or at least 19 kb in size. 36. The method of claim 29, wherein the nucleic acid is codon optimized for expression in the chloroplast genome of the Scenedesmus sp. 37. A transformed chloroplast genome of a Scenedesmus sp., transformed by the method of claim 29.

38. A method of transforming a chloroplast genome of a Scenedesmus sp. with at least one exogenous nucleotide sequence, comprising: i) obtaining the exogenous nucleotide sequence, wherein the exogenous nucleotide sequence comprises a nucleic acid sequence encoding a protein; ii) binding the exogenous nucleotide sequence onto a particle; and iii) shooting the exogenous nucleotide sequence into the Scenedesmus sp. by particle bombardment, wherein the chloroplast genome is transformed with the exogenous nucleotide sequence. 39. The method of claim 38, wherein the exogenous nucleotide sequence is at least 0.5 kb, at least 1.0 kb, at least 2 kb, at least 3 kb, at least 5 kb, at least 8 kb, at least 11 kb, or at least 19 kb in size. 40. The method of claim 38, wherein the nucleic acid is codon optimized for expression in the chloroplast genome of the Scenedesmus sp. 41. The method of claim 38, wherein the particle is a gold particle or a tungsten particle. 42. The method of claim 41, wherein the gold particle is about 550 nm to about 1000 nm in diameter. 43. The method of claim 38, wherein the particle bombardment is carried out by a biolistic device. 44. The method of claim 43, wherein the biolistic device has a helium pressure of about 300 psi to about 500 psi. 45. The method of claim 43, wherein the biolistic device has a helium pressure of at least 300 psi, at least 350 psi, at least 400 psi, at least 425 psi, at least 450 psi, at least 500 psi, or at least 500 psi. 46. The method of claim 38, wherein the exogenous nucleotide sequence bound to the particle is shot at a distance of about 2 to about 4 cm from the Scenedesmus sp. 47. The method of claim 43, wherein the biolistic device is a Helicos Gene Gun or an Accell Gene Gun. 48. The method of claim 38, wherein the nucleic acid encodes for a protein involved in isoprenoid biosynthesis. 49. The method of claim 38, wherein the nucleic acid encodes for a protein involved in fatty acid biosynthesis. 50. The method of claim 38, wherein the Scenedesmus sp. is S. dimorphus. 51. The method of claim 38, wherein the Scenedesmus sp. is S. obliquus. 52. A transformed chloroplast genome of a Scenedesmus sp., transformed by the method of claim 38.

53. A method for obtaining a region of a chloroplast genome of a green algae, wherein the region is useful in the transformation of the green algae, comprising: 1) obtaining genomic DNA of the green algae; 2) obtaining a degenerate forward primer, wherein the forward primer is directed towards a psbB gene of the green algae; 3) obtaining a degenerate reverse primer, wherein the reverse primer is directed towards a psbH gene of the green algae; and 4) using the primers of step 2) and step 3) to amplify the region of the chloroplast genome of the green algae, wherein the nucleotide sequence of the amplified region is obtained. 54. The method of claim 53, wherein the amplified region is amplified by PCR. 55. The method of claim 53, wherein the sequenced region is cloned into a vector. 56. The method of claim 53, wherein the degenerate forward is primer 4099 (SEQ ID NO: 129) or forward primer 4100 (SEQ ID NO: 130), and wherein the degenerate reverse primer is primer 4101 (SEQ ID NO: 131) or reverse primer 4102 (SEQ ID NO: 132). 57. The method of claim 53, wherein the forward primer is primer 4099 (SEQ ID NO: 12.9) and the reverse primer is primer 4102 (SEQ ID NO: 132). 58. The method of claim 53, wherein at least a portion of the sequence of the amplified region is known. 59. The method of claim 53, wherein the amplified region of the chloroplast genome is from C. reinhardtii, C. vulgaris, S. obliquus, or P. purpurea. 60. The method of claim 53, wherein, the sequence of the amplified region is unknown. 61. The method of claim 53, wherein the amplified region of the chloroplast genome is: from D. tertiolecta and comprises the nucleic acid sequence of SEQ ID NO: 133; from a Dunaliella of unknown species comprising the nucleic acid sequence of SEQ ID NO: 134; from N. abudans and comprising the nucleic acid sequence of SEQ ID NO: 135; from C. vulgaris and comprising the nucleic acid sequence of SEQ ID NO: 136; or from T. suecia and comprising the nucleic acid sequence of SEQ ID NO: 137. 62. The method of claim 53, wherein the amplified region of the chloroplast genome comprises a nucleotide sequence encoding a gene cluster pshB-psbT-psbN-psbH. 63. The method of claim 53, wherein the amplified region of chloroplast genome comprises a nucleotide sequence encoding a gene cluster psbB-psbT. 64. The method of claim 63, wherein a nucleic acid encoding a gene is inserted between the nucleotide sequence encoding psbB and psbT. 65. The method of claim 53, wherein the amplified region of chloroplast genome comprises a nucleotide sequence encoding a gene cluster psbT-psbN. 66. The method of claim 65, wherein a nucleic acid encoding a gene is inserted between the nucleotide sequence encoding psbT and psbN. 67. The method of claim 53, wherein the amplified region of chloroplast genome comprises a nucleotide sequence encoding a gene cluster psbN-psbH. 68. The method of claim 67, wherein a nucleic acid encoding a gene is inserted between the nucleotide sequence encoding psbN and psbH. 69. The method of claim 53, wherein the amplified region of chloroplast genome comprises a nucleotide sequence encoding a gene cluster psbH-psbK. 70. The method of claim 69, wherein a nucleic acid encoding a gene is inserted between the nucleotide sequence encoding psbH and psbK. 71. The method of claim 53, wherein the amplified region of the chloroplast genome comprises a nucleotide sequence encoding a region 3′ of psbK. 72. The method of claim 53, wherein the sequence is a nucleic acid sequence. 73. The method of claim 53, wherein the sequence is an amino acid sequence.

74. A region of a chloroplast genome of a green algae, obtained by the method of: 1) obtaining genomic DNA of the green algae; 2) obtaining a degenerate forward primer, wherein the forward primer is directed towards a psbB gene of the green algae; 3) obtaining a degenerate reverse primer, wherein the reverse primer is directed towards a psbH gene of the green algae; and 4) using the primers of step 2) and step 3) to amplify the region of the chloroplast genome of the green algae, wherein the amplified region is sequenced and comprises a nucleotide sequence, and wherein the nucleotide sequence is modified to comprise a nucleic acid sequence encoding for at least one protein, 75. A vector useful in the transformation of the chloroplast genome of Scenedesmus obliquus, comprising a 5.2 kb region from the Scenedesmus obliquus chloroplast genome (Scenedesmus chloroplast sequence NCBI reference sequence: NC_{—}008101, 057,611-062850 bp), wherein the region comprises the nucleic acid sequence of SEQ ID NO: 125, or comprising a nucleotide sequence that is at least 80%, at least 85%, at least 90%, at least 95%, or at least 99% homologous to at least a 500 bp sequence of the nucleic acid sequence of SEQ ID NO: 125.

76. An isolated nucleotide sequence comprising the nucleic acid of SEQ ID NO: 125, or comprising a nucleic acid sequence that is at least 80%, at least 85%, at least 90%, at least 95%, or at least 99% homologous to at least a 500 bp sequence of the nucleic acid sequence of SEQ ID NO: 125, wherein the isolated nucleotide sequence can be used to transform a chloroplast genome a Scenedesmus sp, 77. The isolated nucleotide sequence of claim 76, wherein the nucleic acid sequence of SEQ ID NO: 125 is modified to comprise a second nucleic acid encoding a protein.

78. A host cell comprising a nucleic acid sequence of SEQ ID NO: 125, or comprising a nucleic acid sequence that is at least 80%, at least 85%, at least 90%, at least 95%, or at least 99% homologous to at least a 500 bp sequence of the nucleic acid sequence of SEQ ID NO: 125. 79. The host cell of claim 78, wherein the host cell is a host cell from a Scenedesmus sp. 80. The host cell of claim 78, wherein the nucleic acid sequence of SEQ ID NO: 125 is modified to comprise a second nucleic acid sequence encoding a protein.

### BRIEF DESCRIPTION OF THE DRAWINGS

These and other features, aspects, and advantages of the present disclosure will become better understood with regard to the following description, appended claims and accompanying figures where:

FIG. 1 shows a graphical representation of the mutated psbA fragment and 3′UTR vector used to engineer DCMU^{r} into S. dimorphus.

FIG. 2 shows amplification and digestion of DNA from psbA 264A DCMU^{r} transformants (3 and 4) and S. dimorphus wildtype (WT). U=uncut DNA and C=cut DNA (digested with Xba1).

FIG. 3 shows a psbA S264 transformant that is DCMU^{r} and atrazine^{r},

FIG. 4 shows a graphical representation of p04-38.

FIG. 5 shows a graphical representation of p04-21.

FIG. 6 shows PCR amplification of DNA from S. dimorphus CAM^{r} transformants.

FIG. 7 shows a graphical representation of vector p04-31 used to transform S. dimorphus.

FIG. 8 shows a multiscreen of BD11 clones.

FIG. 9 shows a Western of SE0070 expressing BD11 (subclones of parent 2 and 4).

FIG. 10 shows endoxylanase activity in clarified lysates from S. dimorphus transformants containing the endoxylanase gene (parent 2 and 4).

FIG. 11 shows a graphical representation of p04-28.

FIG. 12 shows verification of homoplasmicity in several lines of S. dimorphus engineered with FPP-synthase.

FIG. 13 shows an anti-flag Western with farnesyl disphosphate (FPP) synthase protein expression in 7 transformants.

FIG. 14A shows an overlay of the Total Ion Chromatogram (TIC) for wild type negative control (untransformed Scenedesmus dimorphus), the engineered strain (S. dimorphus transformed with FPP synthase (avian), and a positive control of FPP. The y axis is abundance. The x axis is time.

FIG. 14B shows the TIC of the FPP positive control. The retention time of FPP is 11.441 minutes. The y axis is abundance. The x axis is time.

FIG. 14C shows the mass spectrum of the FPP positive control at 11.441 minutes. The y axis is abundance. The x axis is m/z (mass to charge ratio).

FIG. 14D shows the TIC of the engineered strain (S. dimorphus transformed with FPP synthase (avian) incubated with IPP and DMAPP. The retention time of the product (FPP) is 11.441 minutes. The y axis is abundance. The x axis is time.

FIG. 14E shows the mass spectrum of the engineered strain (S. dimorphus transformed with FPP synthase (avian) at 11.441 minutes. The y axis is abundance. The x axis is m/z (mass to charge ratio).

FIG. 14F shows the TIC of the untransformed wild type S. dimorphus strain incubated with IPP and DMAPP. The y axis is abundance. The x axis is time.

FIG. 14G shows the mass spectrum of the untransformed wild type S, dimorphus strain at 11.441 minutes. The y axis is abundance. The x axis is m/z (mass to charge ratio).

FIG. 15A shows an overlay of the Total Ion Chromatogram (TIC) for wild type negative control (untransformed Scenedesmus dimorphus) incubated with IPP and DMAPP, the engineered strain (S. dimorphus transformed with IS09 (FPP synthase)) incubated with IPP and DMAPP, and a positive control of FPP. All three enzymatic reactions were incubated with amorphadiene synthase to form amorpha-4,11-diene in a coupled enzyme assay. The y axis is abundance. The x axis is time.

FIG. 15B shows the TIC of the FPP positive control incubated with amorphadiene synthase. The retention time of amorphadiene is 9.917 minutes. The y axis is abundance. The x axis is time.

FIG. 15C shows the mass spectrum of the amorphadiene positive control at 9.917 minutes. The y axis is abundance. The x axis is m/z (mass to charge ratio).

FIG. 15D shows the TIC of the engineered strain (S. dimorphus transformed with IS09 (FPP synthase), incubated with IPP and DMAPP and amorphadiene synthase. The retention time of the product of the reaction (amorphadiene) is 9.917 minutes. The y axis is abundance. The x axis is time.

FIG. 15E shows the mass spectrum of the product produced by the engineered strain (S. dimorphus transformed with IS09 (FPP synthase)) when incubated with IPP, DMAPP, and amorphadiene synthase. The retention time of the product (amorphadiene) is 9.917 minutes. The y axis is abundance. The x axis is m/z (mass to charge ratio).

FIG. 15F shows the TIC of the untransformed wild type S. dimorphus strain incubated with IPP and DMAPP and amorphadiene synthase. The y axis is abundance. The x axis is time.

FIG. 15G shows the mass spectrum (at 9.917 minutes) of the enzymatic reaction with untransformed wild type S. dimorphus strain incubated with IPP, DMAPP, and amorphadiene synthase. The y axis is abundance. The x axis is m/z (mass to charge ratio).

FIG. 16 shows a graphical representation of p04-196.

FIGS. 17A and 17B show a comparison of SIM monitored GC/MS chromatogram of S. dimorphus transformed with IS-88 (17A) and wild-type S. dimorphus (T7B). Chromatograms were monitored with ions m/z=229, 135, and 122 (diagnostic for fusicoccadiene). The retention time of the peak in (17A) (7.617 min) matches that of purified fusicoccadiene.

FIGS. 18A and 18B show a mass spectra at the retention time of the fusicoccadiene peak (t=7.617 min) for S. dimorphus-IS88 (A) and wild type S. dimorphus (R). The mass spectrum of S. dimorphus-IS88 matches well with the known spectrum of fusicoccadiene. The mass spectrum of wild-type shows only background ions. FIG. 18C shows the mass spectrum of purified fusicoccadiene.

FIG. 19 shows a graphical representation of p04-118.

FIG. 20 shows an anti-flag Western blot of S. dimorphus engineered with a gene encoding phytase (FD6).

FIG. 21 shows a graphical representation of p04-162.

FIG. 22 shows that the EreB gene (SEQ ID NO: 25) is amplified from DNA derived from several potential transformants but not from DNA derived from wild type S. dimorphus. Controls: W=no DNA; +=plasmid DNA; and D and O are S. dimorphus DNA.

FIG. 23 shows a graphical representation of p04-161.

FIG. 24 shows codA plates.

FIG. 25 shows a graphical representation of p04-267.

FIG. 26 shows an anti-flag Western blot of S. dimorphus engineered with FPP synthase (Is09) and bisabolene synthase (Is11) genes showing expression of both proteins.

FIG. 27 shows a graphical representation of p04-116.

FIG. 28 shows that endoxylanase is produced as a single peptide (not a fusion with CAT) in engineered S. dimorphus cells.

FIG. 29 shows endoxylanase activity in engineered S. dimorphus (operon 1_{—}1, 2_{—}1, 2_{—}2, 2_{—}3). + is S. dimorphus engineered with psbD driving xylanase and “wt” is wild type.

FIG. 30A and FIG. 30B show that endoxylanase and CAT are transcribed as a single transcript. FIG. 30A shows the primer design and FIG. 30 B is an agarose gel showing amplification of cDNA from 4 of the 5 transformants corresponding to the endoxylanase-CAT transcript.

FIG. 31 shows a graphical representation of transforming DNA with different RBS sequences. In both cases, the psbD promoter and the psbA3′UTR from S. dimorphus are used to regulate CAT-RBS-BD11 expression. BD11 encodes the endoxylanse gene from T. reesei. These cassettes were subcloned into vector p04-166 between region Homology A and homology region B.

FIG. 32A shows xylanase activity of p04-231 from TAP plates. FIG. 32B shows xylanase activity of p04-232 from TAP plates. Endoxylanase activity was detected in cells engineered with RBS1 linking CAT and endoxylanase (p04-231) but not with RBS2 (p04-232).

FIG. 33 shows a graphical representation of p04-142.

FIG. 34 shows verification of homoplasmicity in clone 52, an engineered S. dimorphus line containing a CAT cassette in the region between psbT and psbN.

FIG. 35 shows a graphical representation of the transforming DNA (A) and loopout product (B) that results from recombination at the identical D2 (psbD) promoter segments.

FIG. 36 shows failure to amplify a CAT fragment in a multiplex PCR of S. dimorphus.

FIG. 37 shows a graphical representation of p04-291 and p04-294. BAD1 and BAD4 are the betaine aldehyde dehydrogenase genes from spinach and sugar beet, respectively.

FIG. 38 shows an anti-HA western blot showing expression of betaine aldehyde dehydrogenase from spinach (291 clones 1, 2, 3, BAD1) or from sugar beet (294 4-1, 5-1, 6-1, 7-1, BAD4) in S. dimorphus.

FIG. 39 shows a graphical representation of p45-5 and p45-6.

FIG. 40 shows an agarose gel of EreB amplification from D. tertiolecta transformants in lanes 4, 5, 6.

FIG. 41 shows a graphical representation of p45-12.

FIG. 42 shows an agarose gel of EreB amplification from D. terliolecta transformant 12-3.

FIG. 43 shows that Xylanase protein (BD11) is detected in D. tertiolecta transformant 12-3 via an anti-flag Western blot.

FIG. 44 shows that Xylanase activity is detected in D. terliolecta transformant 12-3. Positive control is S. dimorphus engineered with endoxylanase.

FIG. 45 shows vector gutless pUC (2,436 bp).

FIG. 46 shows vector p04-35 (4,304 bp).

FIG. 47 shows vector pSS-007 (6,132 bp).

FIG. 48 shows vector pSS-013 (7,970 bp).

FIG. 49 shows vector pSS-023 (10.322 kb).

FIG. 50 shows Gene Vector 1 (5,774 bp).

FIG. 51 shows Gene Vector 2 (10.198 kb).

FIG. 52 shows Gene Vector 3 (7,111 bp).

FIG. 53 shows vector pRS414 (4,784 bp).

FIG. 54 shows vector pBeloBAC 11 (7,507 bp).

FIG. 55 shows vector pLW001 (10.049 kb).

FIG. 56 shows vector pLW092 (13.737 kb).

FIG. 57 shows vector pBeloBAC-TRP (10.524 kb).

FIG. 58 shows vector pLW100 (18.847 kb).

FIG. 59 shows vector p04-198.

FIG. 60 shows vector pSS-035 (6,491 bp).

FIG. 61 shows vector pSS-023 CC93 CC94 (15.083 kb).

FIG. 62 shows vector pSS-023 CC93 CC97 (15.077 kb).

FIG. 63 shows vector pLW100 CC90 CC91 CC92 (26.319 kb).

FIG. 64 shows vector pLW100 four gene assembly (34.509 kb).

FIG. 65 shows pSS-023 restriction digest mapping with NdeI, PacI, PstI, ScaI, SnaBI, and SpeI.

FIG. 66 shows pLW001 restriction digest mapping with EcoRV, NotI, PmlI, PvuI and SnaBI.

FIGS. 67A-E show pLW092 restriction digest mapping with PacI (c), PstI (e), ScaI (b), and XhoI (d), and uncut (a).

FIG. 68 shows pLW100 restriction digest mapping with EcoRV, NdeI, NotI, PacI, PstI, ScaI and XhoI.

FIG. 69 shows pSS-035 restriction digest mapping with EcoRI, EcoRV, KpnI, NotI, PvulI, and ScaI.

FIG. 70 shows plasmid DNA comprising four two-gene contigs digested with NdeI.

FIG. 71 shows plasmid DNA comprising two three-gene contigs digested with NdeI.

FIG. 72 shows plasmid DNA comprising four four-gene contigs digested with NdeI.

FIG. 73 shows PCR amplification of the conserved psbB-psbT-psbH-psbN gene cluster from S. dimorphus.

FIG. 74 shows PCR amplification of the conserved psbB-psbT-psbH-psbN gene cluster from a strain of genus Dunaliella; an unknown species.

FIG. 75 shows PCR amplification of the conserved psbB-psbT-psbH-psbN gene cluster from N. abudans.

FIG. 76 shows vector p04-128.

FIG. 77 shows vector p04-129.

FIG. 78 shows vector p04-130.

FIG. 79 shows vector p04-131.

FIG. 80 shows vector p04-142.

FIG. 81 shows vector p04-143.

FIG. 82 shows vector p04-144.

FIG. 83 shows vector p04-145.

FIG. 84 shows a homoplasmicity PCR screen for clones from S. dimorphus that have a resistance cassette between, either psbT and psbN (p04-142) or between psbN and psbH (p04-143).

FIG. 85 shows a homoplasmicity PCR screen for clones from S. dimorphus that have a resistance cassette between either psbT and psbK (p04-144) or 3′ of psbK (p04-145).

FIG. 86 is a nucleotide alignment of the psbB gene from four different algae species.

FIG. 87 is a nucleotide alignment of the psbH region from four different algae species.

FIG. 88 is an alignment of the genome region from the psbB gene to the psbH gene of four different algae species.

FIG. 89 is vector p04-151.

FIG. 90A-D shows restriction enzyme mapping results.

FIG. 91 shows vector pLW106.

FIGS. 92A and B depict 4 clones that screen PCR positive for both BD11 and IS99.

FIG. 93A-C depict 4 clones that screen PCR positive for CC90, CC91, and CC92.

FIGS. 94A and B depict 2 clones that screen PCR positive for IS61, IS62, IS57 and IS116.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present disclosure.

As used in this specification and the appended claims, the singular forms “a”, “an” and “the” include plural reference unless the context clearly dictates otherwise.

Endogenous

An endogenous nucleic acid, nucleotide, polypeptide, or protein as described herein is defined in relationship to the host organism. An endogenous nucleic acid, nucleotide, polypeptide, or protein is one that naturally occurs in the host organism.

Exogenous

An exogenous nucleic acid, nucleotide, polypeptide, or protein as described herein is defined in relationship to the host organism. An exogenous nucleic acid, nucleotide, polypeptide, or protein is one that does not naturally occur in the host organism or is a different location in the host organism.

Examples of genes, nucleic acids, proteins, and polypeptides that can be used in the embodiments disclosed herein include, but are not limited to:

SEQ ID NO: 1 is a PCR primer.

SEQ ID NO: 2 is a PCR primer.

SEQ ID NO: 3 is a PCR primer.

SEQ ID NO: 4 is a PCR primer.

SEQ ID NO: 5 is a PCR primer.

SEQ ID NO: 6 is a PCR primer.

SEQ ID NO: 7 is a PCR primer.

SEQ ID NO: 8 is a PCR primer.

SEQ ID NO: 9 is a PCR primer.

SEQ ID NO: 10 is a PCR primer.

SEQ ID NO: 11 is a PCR primer.

SEQ ID NO: 12 is a PCR primer.

SEQ ID NO: 13 is a PCR primer.

SEQ ID NO: 14 is a PCR primer.

SEQ ID NO: 15 is a PCR primer (#4682).

SEQ ID NO: 16 is a PCR primer (#4982).

SEQ ID NO: 17 is a PCR primer.

SEQ ID NO: 18 is a PCR primer.

SEQ ID NO: 19 is a PCR primer.

SEQ ID NO: 20 is a nucleotide sequence of an artificial FLAG epitope tag linked to a MAT epitope tag by a TEV protease site.

SEQ ID NO: 21 is a gene encoding an endoxylanase from T. reesei codon optimized for chloroplast expression in C. reinhardtii.

SEQ ID NO: 22 is a nucleotide sequence of an artificial TEV protease site linked to a FLAG epitope tag.

SEQ ID NO: 23 is a gene encoding an FPP synthase from G. gallus codon optimized for chloroplast expression in C. reinhardtii.

SEQ ID NO: 24 is a nucleotide sequence of an artificial streptavidin epitope tag.

SEQ ID NO: 25 is a gene encoding a fusicoccadiene synthase from P. amygdali codon optimized according to the most frequent codons in the C. reinhardtii chloroplast.

SEQ ID NO: 26 is a gene encoding a phytase from E. coli codon optimized for chloroplast expression in C. reinhardtii.

SEQ ID NO: 27 is a nucleotide sequence of an artificial FLAG epitope tag linked to a MAT epitope tag by a TEV protease site.

SEQ ID NO: 28 is a modified chloramphenicol acetyltransferase gene from E. coli with the nucleotide at position 64 changed from an A to a G, the nucleotides at positions 436, 437, and 438 were changed from TCA to AGC, and the nucleotide at position 516 was changed from a C to a T.

SEQ ID NO: 29 is a modified erythromycin esterase gene from E. coli with the nucleotide at position 153 changed from a C to a T, the nucleotide at position 195 changed from a T to a C, the nucleotide at position 198 changed from a A to a C, the nucleotide at position 603 changed from a T to a A, the nucleotide at position 1194 changed from a C to a T, and the nucleotide at position 1203 changed from a T to an A.

SEQ ID NO: 30 is a fragment of genomic DNA from S. dimorphus that encodes a region containing a portion of the 3′ end of the psbA gene and some untranslated region, with nucleotide 1913 of the fragment mutated from a T to a G for the S264A mutation, and nucleotides 1928 to 1930 mutated from CGT to AGA to generate a silent XbaI restriction site.

SEQ ID NO: 31 is a gene encoding a cytosine deaminase from E. coli codon optimized for expression in the chloroplast of C. reinhardtii.

SEQ ID NO: 32 is a gene encoding a betaine aldehyde dehydrogenase from S. oleracea codon optimized according to the tRNA usage of the chloroplast of C. reinhardtii.

SEQ ID NO: 33 is a nucleotide sequence of an artificial 3×HA tag linked to a 6×HIS tag by a TEV protease site.

SEQ ID NO: 34 is a gene encoding a betaine aldehyde dehydrogenase from B. vulgaris codon optimized for expression in the chloroplast of C. reinhardtii.

SEQ ID NO: 35 is a gene encoding an E-alpha-bisabolene synthase from A. grandis codon optimized for expression in the chloroplast of C. reinhardtii.

SEQ ID NO: 36 is a modified nucleotide sequence that is the reverse complement of SEQ ID NO: 37 with extra nucleotides on the 5′ and 3′ ends; nucleotides 1-43 are extra on the 3′ end and nucleotides 532-541 are extra on the 5′ end.

SEQ ID NO: 37 is a nucleotide sequence of the endogenous promoter from the psbA gene of S. dimorphus that was cloned into an integration vector.

SEQ ID NO: 38 is a modified nucleotide sequence that is the reverse complement of SEQ ID NO: 39 with extra nucleotides on the 5′ end and a nucleotide insertion; nucleotides 535-716 are extra 5′ sequence and nucleotides 176-188 are the insertion.

SEQ ID NO: 39 is a nucleotide sequence of the endogenous promoter for the psbB gene of S. dimorphus that was cloned into integration vectors.

SEQ ID NO: 40 is a sequence of the endogenous promoter for the psbD gene of S. dimorphus that was cloned into integration vectors.

SEQ ID NO: 41 is a modified nucleotide sequence that is the reverse complement of SEQ ID NO: 42 with extra sequence on the 5′; nucleotides 537-464 are extra sequences on the 5′ end, nucleotide 308 is changed from a C to a T, nucleotide 310 is changed from a C to a T, and nucleotide 259 is changed from an A to a G.

SEQ ID NO: 42 is a nucleotide sequence of the endogenous promoter for the tufA gene of S. dimorphus that was cloned into integration vectors.

SEQ ID NO: 43 is a modified nucleotide sequence that is the reverse of SEQ ID NO: 44 with extra sequences on the 5′ end; nucleotides 550-557 are extra sequences on the 5′ end.

SEQ ID NO: 44 is a nucleotide sequence for the endogenous promoter of the rpoA of S. dimorphus.

SEQ ID NO: 45 is a nucleotide sequence for the endogenous promote of the cemA gene in S. dimorphus that was cloned into integration vectors.

SEQ ID NO: 46 is a modified nucleotide sequence that is the reverse complement of SEQ ID NO: 47 with an insertion at nucleotides 233-266.

SEQ ID NO: 47 is a nucleotide sequence for the endogenous promoter of the ftsH gene in S. dimorphus that was cloned into integration vectors.

SEQ ID NO: 48 is a modified nucleotide sequence of SEQ ID NO: 49 that has extra sequences on the 5′ end; nucleotides 1-19 are extra sequences, nucleotide 404 has been changed from an A to a T.

SEQ ID NO: 49 is a nucleotide sequence for the endogenous promoter of the rbcL gene in S. dimorphus that was cloned into integration vectors.

SEQ ID NO: 50 is a modified nucleotide sequence of SEQ ID NO: 51 that has 24 nucleotides truncated on the 5′ end; the nucleotide at position 2 is changed from a G to a C, position 5 is changed from an A to a G, at positions 199 and 200 two T's are inserted, and at position 472 it is changed from an A to a G.

SEQ ID NO: 51 is the nucleotide sequence of the endogenous promoter for the chlB gene from S. dimorphus that was cloned into integration vectors.

SEQ ID NO: 52 is a modified nucleotide sequence of SEQ ID NO: 53 where nucleotides 1-3 are extra sequence, the nucleotide at position 442 is a G insertion, and the R at position 482 is a result of poor sequencing.

SEQ ID NO: 53 is a nucleotide sequence for the endogenous promoter of the petA gene in S. dimorphus that was cloned into integration vectors.

SEQ ID NO: 54 is a modified nucleotide sequence that is the reverse complement of SEQ ID NO: 55 where nucleotides 3, 8, 18, 21, 49, 57, and 82 are insertions, nucleotides 484-503 are extra on the 5′ end, nucleotide 26 is changed from a C to a T, and nucleotide 30 is changed from an A to a C.

SEQ ID NO: 55 is the nucleotide sequence of the endogenous promoter for the petB gene from S. dimorphus that was cloned into integration vectors.

SEQ ID NO: 56 is the modified nucleotide sequence of SEQ ID NO: 57 that has 3 nucleotides truncated on the 5′ end.

SEQ ID NO: 57 is the nucleotide sequence of the endogenous terminator region for the rbcL gene from S. dimorphus that was cloned into integration vectors.

SEQ ID NO: 58 is the nucleotide sequence of the endogenous terminator region for the psbA gene from S. dimorphus that was cloned into integration vectors.

SEQ ID NO: 59 is the nucleotide sequence of the endogenous terminator region for the psaB gene from S. dimorphus that was cloned into integration vectors.

SEQ ID NO: 60 is a nucleic acid linker sequence (RBS3).

SEQ ID NO: 61 is a nucleic acid linker sequence (RBS2).

SEQ ID NO: 62 is the nucleotide sequence of the endogenous promoter region for the psbD gene from D. tertiolecta that was cloned into integration vectors.

SEQ ID NO: 63 is the nucleotide sequence of the endogenous promoter region for the tufA gene from D. tertiolecta that was cloned into integration vectors.

SEQ ID NO: 64 is the nucleotide sequence of the endogenous terminator region for the rbcL gene from D. tertiolecta that was cloned into integration vectors.

SEQ ID NO: 65 is the nucleotide sequence of the endogenous terminator region for the psbA gene from a Dunaliella isolate of unknown species that was cloned into integration vectors.

SEQ ID NO: 66 is PCR primer 1.

SEQ ID NO: 67 is PCR primer 2.

SEQ ID NO: 68 is PCR primer 3.

SEQ ID NO: 69 is PCR primer 4.

SEQ ID NO: 70 is PCR primer 5.

SEQ ID NO: 71 is PCR primer 6.

SEQ ID NO: 72 is PCR primer 7.

SEQ ID NO: 73 is PCR primer 8.

SEQ ID NO: 74 is PCR primer 9.

SEQ ID NO: 75 is PCR primer 10.

SEQ ID NO: 76 is PCR primer 11.

SEQ ID NO: 77 is PCR primer 12.

SEQ ID NO: 78 is PCR primer 13.

SEQ ID NO: 79 is PCR primer 14.

SEQ ID NO: 80 is PCR primer 15.

SEQ ID NO: 81 is PCR primer 16.

SEQ ID NO: 82 is PCR primer 17.

SEQ ID NO: 83 is PCR primer 18.

SEQ ID NO: 84 is PCR primer 19.

SEQ ID NO: 85 is PCR primer 20.

SEQ ID NO: 86 is PCR primer 21.

SEQ ID NO: 87 is PCR primer 22.

SEQ ID NO: 88 is PCR primer 23.

SEQ ID NO: 89 is PCR primer 24.

SEQ ID NO: 90 is PCR primer 25.

SEQ ID NO: 91 is PCR primer 26.

SEQ ID NO: 92 is PCR primer 27.

SEQ ID NO: 93 is PCR primer 28.

SEQ ID NO: 94 is PCR primer 29.

SEQ ID NO: 95 is PCR primer 30.

SEQ ID NO: 96 is PCR primer 31.

SEQ ID NO: 97 is PCR primer 32.

SEQ ID NO: 98 is PCR primer 33.

SEQ ID NO: 99 is PCR primer 34.

SEQ ID NO: 100 is PCR primer 35.

SEQ ID NO: 101 is PCR primer 36.

SEQ ID NO: 102 is PCR primer 37.

SEQ ID NO: 103 comprises a nucleic acid sequence encoding for URA3.

SEQ ID NO: 104 comprises a nucleic acid sequence encoding for ADE2.

SEQ ID NO: 105 comprises a nucleic acid sequence encoding for URA3-ADE2.

SEQ ID NO: 106 is a nucleic acid linker sequence with engineered restriction sites.

SEQ ID NO: 107 comprises a nucleic acid sequence encoding for TRP1-ARS1-CEN4 (from pYAC4).

SEQ ID NO: 108 comprises a nucleic acid sequence encoding for LEU2.

SEQ ID NO: 109 comprises a nucleic acid sequence encoding for CC-93.

SEQ ID NO: 110 comprises a nucleic acid sequence encoding for CC-94.

SEQ ID NO: 111 comprises the contig sequence (CC93-CC94) that was inserted into pSS-023.

SEQ ID NO: 112 comprises the contig sequence (CC93-CC97) that was inserted into pSS-023.

SEQ ID NO: 113 comprises a nucleic acid sequence encoding for CC-97.

SEQ ID NO: 114 comprises the contig sequence (CC90-CC91-CC92) that was inserted into pLW100.

SEQ ID NO: 115 comprises a nucleic acid sequence encoding for CC-90.

SEQ ID NO: 116 comprises a nucleic acid sequence encoding for CC-91.

SEQ ID NO: 117 comprises a nucleic acid sequence encoding for CC-92.

SEQ ID NO: 118 comprises a nucleic acid sequence encoding for HIS3.

SEQ ID NO: 119 comprises a nucleic acid sequence encoding for LYS2.

SEQ ID NO: 120 comprises the contig sequence (IS57-IS116-IS62-IS61) that was inserted into pLW100.

SEQ ID NO: 121 comprises a nucleic acid sequence encoding for IS57.

SEQ ID NO: 122 comprises a nucleic acid sequence encoding for IS116.

SEQ ID NO: 123 comprises a nucleic acid sequence encoding for IS62.

SEQ ID NO: 124 comprises a nucleic acid sequence encoding for IS61.

SEQ ID NO: 125 is a 5,240 base pair sequence from Scenedesmus obliquus.

SEQ ID NO: 126 is the A3 homology region.

SEQ ID NO: 127 is the B3 homology region.

SEQ ID NO: 128 comprises a sequence encoding for rblcL-CAT-psbE.

SEQ ID NO: 129 is a degenerate PCR primer.

SEQ ID NO: 130 is a degenerate PCR primer.

SEQ ID NO: 131 is a degenerate PCR primer.

SEQ ID NO: 132 is a degenerate PCR primer.

SEQ ID NO: 133 is genomic sequence of the region encoding the psbB, psbT, psbN, and psbH genes from D. tertiolecta.

SEQ ID NO: 134 is genomic sequence of the region encoding the psbB, psbT, psbN, and psbH genes from a Dunaliella of unknown species.

SEQ ID NO: 135 is a partial genomic sequence of the region encoding the psbB, psbT, psbN, and psbH genes from N. abudans; the stretch of N's represents a gap in the sequence.

SEQ ID NO: 136 is genomic sequence of the region encoding the psbB, psbT, psbN, and psbH genes from an isolate of C. vulgaris.

SEQ ID NO: 137 is genomic sequence of the region encoding the psbB, psbT, psbN, and psbH genes from T. suecica.

SEQ ID NO: 138 is PCR primer (#4682).

SEQ ID NO: 139 is PCR primer (#4982).

SEQ ID NO: 140 is PCR primer 4684.

SEQ ID NO: 141 is PCR primer 4685.

SEQ ID NO: 142 is PCR primer 4686

SEQ ID NO: 143 is PCR primer 4687.

SEQ ID NO: 144 is PCR primer 4688.

SEQ ID NO: 145 is PCR primer 4689.

SEQ ID NO: 146 comprise a nucleotide sequence encoding BD11,

SEQ ID NO: 147 comprise a nucleotide sequence encoding IS99.

SEQ ID NO: 148 comprise a nucleotide sequence encoding CAT.

SEQ ID NO: 149 to SEQ ID NO: 170 are PCR primers.

The present disclosure relates to methods of transforming various species of algae, for example, algae from the genus Scenedesmus and from the genus Dunaliella, vectors and nucleic acid constructs useful in conducting such transformations, and recombinant Scenedesmus and Dunaliella organisms produced using the vectors and methods disclosed herein. In one embodiment, the Scenedesmus sp. utilized is Scenedesmus dimorphus. Scenedesmus sp. are members of the Chlorophyceans a diverse assemblage of green algae. Scenedesmus is a genus consisting of unicells or flat coenobial colonies of 2, 4, 8 or 16 linearly arranged cells. Cells contain a single plastid with pyrenoid and uninucleate. Scenedesmus sp. are common inhabitants of the plankton of freshwaters and brackish waters and occasionally form dense populations. In one embodiment, the organism utilized is from the genus Dunaliella. In another embodiment, the Dunaliella sp, is D. tertiolecta.

One embodiment, the disclosure provides vectors useful in the transformation of Scenedesmus sp., for example, Scenedesmus dimorphus or Scenedesmus obliquus. In another, embodiment, the disclosure provides vectors useful in the transformation of Dunaliella sp., for example, Dunaliella tertiolecta. An expression cassette can be constructed in an appropriate vector. In some instances, the cassette is designed to express one or more protein-coding sequences in a host cell. Such vectors can be constructed using standard techniques known in the art. In a typical expression cassette, the promoter or regulatory element is positioned on the 5′ or upstream side of a coding sequence whose expression is desired. In other cassettes, a coding sequence may be flanked by sequences which allow for expression upon insertion into a target genome (e.g., nuclear or plastid). For example, a nucleic acid encoding an enzyme involved in the synthesis of a compound of interest, for example an isoprenoid, such that expression of the enzyme is controlled by a naturally occurring regulatory element. Any regulatory element which provides expression under appropriate conditions such that the mRNA or protein product is expressed to a level sufficient to produce useful amount of the desired compound can be used.

One or more additional protein coding sequences can be operatively fused downstream or 3′ of a promoter. Coding sequences for single proteins can be used, as well as coding sequences for fusions of two or more proteins. Coding sequences may also contain additional elements that would allow the expressed proteins to be targeted to the cell surface and either be anchored on the cell surface or be secreted to the environment. A selectable marker is also employed in the design of the vector for efficient selection of algae transformed by the vector. Both a selectable marker and another sequence which one desires to introduce may be introduced fused to and downstream of a single promoter. Alternatively, two protein coding sequences can be introduced, each under the control of a promoter.

One approach to construction of a genetically manipulated strain of Scenedesmus or Dunaliella involves transformation with a nucleic acid which encodes a gene of interest, typically an enzyme capable of converting a precursor into a fuel product or precursor of a fuel product (e.g., an isoprenoid or fatty acid), a biomass degrading enzyme, or an enzyme for the improvement of a characteristic of a feedstuff. In some embodiments, a transformation may introduce nucleic acids into any plastid of the host alga cell (e.g., chloroplast). In other embodiments, a transformation may introduce nucleic acids into the nuclear genome of the host cell. In still other embodiments, a transformation introduces nucleic acids into both the nuclear genome and a plastid. In some instances, the nucleic acids encoding proteins of interest (e.g., transporters or enzymes) are codon-biased for the intended site of insertion (e.g., nuclear codon-biased for insertion, in the nucleus, chloroplast codon-biased for insertion, in the chloroplast).

To construct the vector, the upstream DNA sequences of a gene expressed under control of a suitable promoter may be restriction mapped and areas important for the expression of the protein characterized. The exact location of the start codon of the gene is determined and, making use of this information and the restriction map, a vector may be designed for expression of an endogenous or exogenous protein by removing the region responsible for encoding the gene's protein but leaving the upstream region found to contain the genetic material responsible for control of the gene's expression. A synthetic oligonucleotide is typically inserted in the location where the protein sequence once was, such that any additional gene could be cloned in using restriction endonuclease sites in the synthetic oligonucleotide (i.e., a multi cloning site). An unrelated gene (or coding sequence) inserted at this site would then be under the control of an extant start codon and upstream regulatory region that will drive expression of the foreign (i.e., not normally there) protein encoded by this gene. Once the gene for the foreign protein is put into a cloning vector, it can be introduced into the host organism using any of several methods, some of which might be particular to the host organism. Variations on these methods are amply described in the general literature.

The term “exogenous” is used herein in a comparative sense to indicate that a nucleotide sequence (or polypeptide) being referred to is from a source other than a reference source and is different from the sequence of the reference, or is linked to a second nucleotide sequence (or polypeptide) with which it is not normally associated, or is modified such that it is in a form that is not normally associated with a reference material. For example, a polynucleotide encoding an enzyme is exogenous with respect to a nucleotide sequence of a chloroplast, where the polynucleotide is not normally found in the chloroplast (e.g., a mutated polynucleotide encoding a chloroplast sequence or a nuclear sequence). As another example, a polynucleotide encoding an enzyme is exogenous with respect to a host organism where the polynucleotide comprises operatively linked sequences (e.g., promoters, homologous recombination sites, selectable markers, and/or termination sequences), that are not normally found in the reference organism.

Polynucleotides encoding enzymes and other proteins useful in the present disclosure may be isolated and/or synthesized by any means known in the art, including, but not limited to cloning, sub-cloning, and PCR. A vector herein may encode polypeptide(s) having a role in the mevalonate pathway, such as, for example, thiolase, HMG-CoA synthase, HMG-CoA reductase, mevalonate kinase, phosphemevalonate kinase, and mevalonate-5-pyrophosphate decarboxylase. In other embodiments, the polypeptides are enzymes in the non-mevalonate pathway, such as DOXP synthase, DOXP reductase, 4-diphosphocytidyl-2-C-methyl-D-erythritol synthase, 4-diphophocytidyl-2-C-methyl-D-erythritol kinase, 2-C-methyl-D-erythritol 2,4,-cyclodiphosphate synthase, HMB-PP synthase, HMB-PP reductase, or DOXP reductoisomerase.

One embodiment is directed to a vector comprising a nucleic acid encoding an enzyme capable of modulating a fusicoccadiene biosynthetic pathway. Such a vector may further comprise a promoter for expression of the nucleic acid in algae. Nucleic acid(s) included in such vectors may contain a codon biased form of a gene, optimized for expression in a host organism of choice. In one embodiment, the fusicoccadiene produced is fusicocca-2,10(14)-diene. Another aspect of the present disclosure is directed to a vector comprising a nucleic acid encoding an enzyme that produces a fusicoccadiene when the vector is integrated into a genome of an organism, such as photosynthetic algae, wherein She organism does not produce fusicoccadiene without the vector and wherein the fusicoccadiene is metabolically inactive in the organism.

Further provided herein is a method of producing a fuel product, comprising: a) transforming a Scenedesmus sp or Dunaliella sp., wherein the transformation results in the production or increased production of a fusicoccadiene; b) collecting the fusicoccadiene from the organism; and c) using the fusicoccadiene to produce a fuel product.

The present disclosure also contemplates host cells making polypeptides that contribute to the secretion of fatty acids, lipids or oils, by transforming host cells (e.g., algal cells) and/or organisms comprising host cells with nucleic acids encoding one or more different transporters. In some embodiments, the host cells or organisms are also transformed with one or more enzymes that contribute to the production of fatty acids, lipids or oils are anabolic enzymes. Some examples of anabolic enzymes that contribute to the synthesis of fatty acids include, but are not limited to, acetyl-CoA carboxylase, ketoreductase, thioesterase, malonyltransferase, dehydratase, acyl-CoA ligase, ketoacylsynthase, enoylreductase and a desaturase. In some embodiments, the enzymes are catabolic or biodegrading enzymes. In some embodiments, a single enzyme is produced.

Some host cells may be transformed with multiple genes encoding one or more enzymes. For example, a single transformed cell may contain exogenous nucleic acids encoding enzymes that make up an entire synthesis pathway. One example of a pathway might include genes encoding an acetyl CoA carboxylase, a malonyltransferase, a ketoacylsynthase, and a thioesterase. Cells transformed with entire pathways and/or enzymes extracted from those cells, can synthesize complete fatty acids or intermediates of the fatty acid synthesis pathway. Constructs may contain multiple copies of the same gene, multiple genes encoding the same enzyme from different organisms, and/or multiple genes with one or more mutations in the coding sequence(s).

In some instances, the host cell will naturally produce the fatty acid, lipid, triglyceride or oil of interest. Thus, transformation of the host cell wish a polynucleotide encoding a transport protein will allow for secretion or increased secretion of the molecule of interest from the cell. In other instances, the host cell is transformed with a polynucleotide encoding one or more enzymes necessary for the production of the molecule of interest. The enzymes produced by the modified cells result in the production of fatty acids, lipids, triglycerides or oils that may be collected from the cells and/or the surrounding environment (e.g., bioreactor, growth medium). In some embodiments, the collection of the fatty acids, lipids, triglycerides or oils is performed after the product is secreted from the cell via a cell membrane transporter.

Synthesis of fatty acids, lipids or oils can also be accomplished by engineering a cell to express an accessory molecule or modulation molecule. In certain embodiments, the accessory molecule is an enzyme that produces a substrate utilized by a fatty acid synthesizing enzyme. In some embodiments the accessory or modulation molecule contributes to the growth or nourishment of the biomass.

An additional aspect of the present disclosure provides a vector comprising a nucleic acid encoding a biomass degrading enzyme and a promoter configured for expression of the nucleic acids in a non-vascular photosynthetic organism, for example a Scenedesmus sp. and more particularly S. dimorphus or Dunaliella sp. Vectors of the present disclosure may contain nucleic acids encoding more than one biomass degrading enzyme and, in other instances, may contain nucleic acids encoding polypeptides which covalently link biomass degrading enzymes. Biomass degrading enzymes may include cellulolytic enzymes, hemicellulolytic enzymes and ligninolytic enzymes. More specifically, the biomass degrading enzymes may be exo-β-glucanase, endo-β-glucanase, β-glucosidase, endoxylanase, or lignase. Nucleic acids encoding the biomass degrading enzymes may be derived from fungal or bacterial sources, for example, those encoding exo-β-glucanase in Trichoderma viride, exo-β-glucanase in Trichoderma reesei, exo-β-glucanase in Aspergillus aculeatus, endo-β-glucanase in Trichoderma reesei, endo-β-glucanase in Aspergillus niger, β-glucosidase in Trichoderma reesei, β-glucosidase in Aspergillus niger endoxylanase in Trichoderma reesei, and endoxylanase in Aspergillus niger. Other nucleic acids encoding biomass degrading enzymes may be endogenous to the organisms.

Also provided is a composition containing a plurality of vectors each of which encodes a different biomass degrading enzyme and a promoter for expression of said biomass degrading enzymes in a chloroplast. Such compositions may contain multiple copies of a particular vector encoding a particular enzyme. In some instances, the vectors will contain nucleic acids encoding cellulolytic, hemicellulolytic and/or ligninolytic enzymes. More specifically, the plurality of vectors may contain vectors capable of expressing exo-β-glucanase, endo-β-glucanase, β-glucosidase, endoxylanase and/or lignase. Some of the vectors of this embodiment are capable of insertion into a chloroplast genome and such insertion can lead to disruption of the photosynthetic capability of the transformed chloroplast. Insertion of other vectors into a chloroplast genome does not disrupt photosynthetic capability of the transformed chloroplast. Some vectors provide for expression of biomass degrading enzymes which are sequestered in a transformed chloroplast.

Another vector encodes a plurality of distinct biomass degrading enzymes and a promoter for expression of the biomass degrading enzymes in a non-vascular photosynthetic organism. The biomass degrading enzymes may be one or more of cellulollytic, hemicellulolytic or ligninolytic enzymes. In some vectors, the plurality of distinct biomass degrading enzymes is two or more of exo-β-glucanase, endo-β-glucanase, β-glucosidase, lignase and endoxylanase. In some embodiments, the plurality of enzymes is operatively linked. In other embodiments, the plurality of enzymes is expressed as a functional protein complex. Insertion of some vectors into a host cell genome does not disrupt photosynthetic capability of the organism. Vectors encoding a plurality of distinct enzymes, may lead to production of enzymes which are sequestered in a chloroplast of a transformed organism. The present disclosure also provides an algal cell and in particular a Scenedemus sp. or Dunaliella sp. transformed with a vector encoding a plurality of distinct enzymes. For some embodiments, the organism may be grown in the absence of light and/or in the presence of an organic carbon source.

Yet another aspect provides a genetically modified chloroplast of a Scenedemus sp. or Dunaliella sp. producing one or more biomass degrading enzymes. Such enzymes may be cellulolytic, hemicellulolytic or ligninolytic enzymes, and more specifically, may be an exo-β-glucanase, an endo-β-glucanase, a β-glucosidase, an endoxylanase, a lignase and/or combinations thereof. The one or more enzymes are be sequestered in the chloroplast in some embodiments. The present disclosure also provides photosynthetic organisms containing the genetically modified chloroplasts of the present disclosure.

Yet another aspect provides a method for preparing a biomass-degrading enzyme. This method comprises she steps of (1) transforming a photosynthetic, non-vascular organism and in particular a Scenedesmus sp. or Dunaliella sp. to produce or increase production of said biomass-degrading enzyme and (2) collecting the biomass-degrading enzyme from said transformed organism. Transformation may be conducted with a composition containing a plurality of different vectors encoding different biomass degrading enzymes. Transformation may also be conducted with a vector encoding a plurality of distinct biomass degrading enzymes. Any or all of the enzymes may be operatively linked to each other, in some instances, a chloroplast is transformed. This method may have one or more additional steps, including: (a) harvesting transformed organisms; (b) drying transformed organisms; (c) harvesting enzymes from a cell medium; (d) mechanically disrupting transformed organisms; or (e) chemically disrupting transformed organisms. The method may also comprise further purification of an enzyme through performance liquid chromatography.

Still another method of the present disclosure allows for preparing a biofuel. One step of this method includes treating a biomass with one or more biomass degrading enzymes derived from a photosynthetic, nonvascular organism for a sufficient amount of time to degrade at least a portion of said biomass. The biofuel produced may be ethanol. The enzymes of this method may contain at least traces of said photosynthetic nonvascular organism from which they are derived. Additionally, the enzymes useful for some embodiments of this method include cellulolytic, hemicellulolytic and ligninolytic enzymes. Specific enzymes useful for some aspects of this method include exo-β-glucanase, endo-β-glucanase, β-glucosidase, endoxylanase, and/or lignase. Multiple types of biomass including agricultural waste, paper mill waste, corn stover, wheat stover, soy stover, switchgrass, duckweed, poplar trees, woodchips, sawdust, wet distiller grain, dray distiller grain, human waste, newspaper, recycled paper products, or human garbage may be treated with this method of the disclosure. Biomass may also be derived from a high-cellulose content organism, such as switchgrass or duckweed. The enzyme(s) used in this method may be liberated from the organism and this liberation may involve chemical or mechanical disruption of the cells of the organism, in an alternate embodiment, the enzyme(s) are secreted from the organism and then collected from a culture medium. The treatment of the biomass may involve a fermentation process, which may utilize a microorganism other than the organism which produced the enzyme(s). in some instances the non-vascular photosynthetic organism may be added to a saccharification tank. This embodiment may also comprise the step of collecting the biofuel. Collection may be performed by distillation. In some instances, the biofuel is mixed with another fuel.

An additional method provides for making at least one biomass degrading enzyme by transforming a chloroplast to make a biomass degrading enzyme. The biomass degrading enzyme may be a cellulolytic enzyme, a hemicellulolytic enzyme, or a ligninolytic enzyme, and specifically may be exo-β-glucanase, endo-β-glucanase, β-glucosidase, endoxylanase, or lignase. In some instances, she biomass degrading enzyme is sequestered in the transformed chloroplast. The method may further involve disrupting, via chemical or mechanical means, the transformed chloroplast to release the biomass degrading enzyme(s). In some instances, multiple enzymes will be produced by a transformed chloroplast. The biomass degrading enzymes may be of fungal or bacterial origin, for example, exo-β-glucanase, endo-β-glucanase, β-glucosidase, endoxylanase, lignase, or a combination thereof.

Some host cells may be transformed with multiple genes encoding one or more enzymes. For example, a single transformed cell may contain exogenous nucleic acids encoding an entire biodegradation pathway. One example of a pathway might include genes encoding an exo-β-glucanase (acts on the cellulose end chain), an endo-β-glucanase (acts on the interior portion of a cellulose chain), β-glucosidase (avoids reaction inhibitors by/degrades cellobiose), and endoxylanase facts on hemicellulose cross linking). Such cells transformed with entire pathways and/or enzymes extracted from them, can degrade certain components of biomass. Constructs may contain multiple copies of the same gene, and/or multiple genes encoding the same enzyme from different organisms, and/or multiple genes wish mutations in one or more parts of the coding sequences.

Alternately, biomass degradation pathways can be created by transforming host cells with the individual enzymes of the pathway and then combining the cells producing the individual enzymes. This approach, allows for the combination of enzymes to more particularly match the biomass of interest by altering the relative ratios of the multiple transformed strains. For example, two times as many cells expressing the first enzyme of a pathway may be added to a mix where the first step of the reaction pathway is the limiting step.

Following transformation with enzyme-encoding constructs, the host cells and/or organisms are grown. The biomass degrading enzymes may be collected from the organisms/cells. Collection may be by any means known in the art, including, but not limited to concentrating cells, mechanical or chemical disruption, of cells, and purification of enzymes from cell cultures and/or cell lysates. Cells and/or organisms can be grown and then the enzyme(s) collected by any means. One method of extracting the enzyme is by harvesting the host cell or a group of host cells and then drying the host cell(s). The enzyme(s) from the dried host cell(s) are then harvested by crushing the cells to expose the enzyme. The whole product of crushed cells is then used to degrade biomass. Many methods of extracting proteins from intact cells are well known in the art, and are also contemplated herein (e.g., introducing an exogenous nucleic acid construct in which an enzyme-encoding sequence is operably linked to a sequence encoding a secretion signal-excreted enzyme is Isolated from the growth medium).

Extracting and utilizing the biomass-degrading enzyme can also be accomplished by expressing a vector containing nucleic acids that encode a biomass production-modulation molecule in the host cell. In this embodiment, the host cell produces the biomass, and also produces a biomass-degrading enzyme. The biomass-degrading enzyme can then degrade die biomass produced by the host cell, in some instances, vector used for the production of a biomass-degrading enzyme may not be continuously active. Such vectors can comprise one or more inducible promoters and one or more biomass-degrading enzymes. Such promoters activate the production of biomass-degrading enzymes, for example, after the biomass has grown to sufficient density or reached certain maturity.

The present methods can also be performed by introducing a recombinant nucleic acid molecule into a chloroplast, wherein the recombinant nucleic acid molecule includes a first polynucleotide, which encodes at least one polypeptide (i.e., 1, 2, 3, 4, or more). In some embodiments, a polypeptide is operatively linked to a second, third, fourth, fifth, sixth, seventh, eighth, ninth, tenth and/or subsequent polypeptide. For example, several enzymes in a biodegradation pathway may be linked, either directly or indirectly, such that products produced by one enzyme in the pathway, once produced, are in close proximity to the next enzyme in the pathway.

Another aspect provides host organisms or cells disclosed herein (e.g. Scenedesmus sp. or Dunaliella sp.) that have been genetically modified or modified (e.g. by methods disclosed herein) for use as a feedstock. The compositions of genetically modified algae disclosed here can be used directly as a feedstock or can be added to a feedstock to generate a modified or improved feedstock. For example a composition can comprise a feedstock and a genetically modified algae. Genetic modification of an algae can comprise engineering an algae to express one or more enzymes. In some aspects the enzyme can be a biomass degrading enzyme and in some aspects the enzyme can be a biosynthetic enzyme. Genetically modified algae can also express both types of enzymes (e.g. a biomass degrading enzyme and a biosynthetic enzyme). The enzyme expressed can be one that is naturally expressed in the algae or not naturally expressed in the algae. In some aspects the enzyme produced is not naturally expressed in the algae. For example an enzyme (e.g. a biomass degrading enzyme) can be an exogenous enzyme. In another example a composition can comprise a feedstock and a genetically modified algae wherein the algae is modified to increase the expression of a naturally occurring enzyme (e.g. a biomass-degrading enzyme). In some aspects an enzyme can be secreted from a genetically modified algae or added to the feedstock as an independent ingredient.

Biomass degrading enzymes can improve the nutrient value of an existing feedstock by breaking down complex components of the feedstock (e.g. indigestible components) into components that can be absorbed and used by the animal. A biomass-degrading enzyme can be expressed and retained in the algae or secreted or expelled (i.e. produced ex vivo) from the algae. Genetically modified algae that provide the biomass degrading enzymes can also be utilized by the animal for the inherent nutrient value of the algae. For example a composition can comprise a feedstock, a genetically modified algae, and a biomass-degrading enzyme that is ex vivo to the genetically modified algae. In another example a genetically modified algae is modified to increase expression of a naturally occurring biomass-degrading enzyme.

The expression of certain exogenous biosynthetic enzymes in an algae can allow the biosynthesis of nutrient rich lipids, fatty acids and carbohydrates. Genetically modified algae that express such nutrient rich components can be added to an existing feedstock to supplement the nutritional value of the feedstock. In some aspects such genetically modified algae can comprise as much as 100% of the feedstock. Algae can be genetically modified to produce or increase production of one or more fatty acids, lipids or hydrocarbons. In one example a genetically modified algae comprise an exogenous nucleic acid encoding an enzyme in an isoprenoid biosynthesis pathway. In some aspects a genetically modified algae can have a higher content, or an altered content, or a different content of, for example, fatty acids, lipids or hydrocarbons (e.g. isoprenoids) than an unmodified algae of the same species. For example, the modified algae can produce more of a desired isoprenoid, and/or produce an isoprenoid that the algae does not normally produce, and/or produce isoprenoids that are normally produced but at different amounts than are produced in an unmodified algae.

Therefore in one aspect a composition can comprise a feedstock and a genetically modified algae wherein the algae has a higher lipid, fatty acid, or isoprenoid content relative to an unmodified algae of the same species. The biosynthetic enzymes can also be one found in a mevalonate pathway. For example the enzyme can be farnesyl pyrophosphate synthase, geranyl geranyl phosphate synthase, squalene synthase, thioesterase, or fatty acyl-CoA desaturase.

An improved feedstock can be comprised entirely or partially of a genetically modified algae. In some aspects a genetically modified algae can be added to a composition to generate an improved feedstock. The composition may not be considered a feedstock suitable for consumption by animals until after the addition of a genetically modified algae. In some aspects a genetically modified algae can be added to an existing feedstock to generate an improved feedstock. In some aspects a genetically modified algae can be added to an existing feedstock at a ratio of at least 1:20 (weight of algae/wt of feedstock). In some aspects an improved feedstock can comprises up to 10, 20, 30, 40, 50, 60, 70, 80, 90, 95, or 100 percent of a genetically modified algae. In some aspects a viable genetically modified algae can be added to a feedstock (e.g. as a seed culture) at a concentration of less than 5% (w/w) of the feedstock wherein the genetically modified algae multiplies to become up to 10, 20, 30, 40, 50, 60, 70, 80, 90 or 95% percent: of the feedstock (w/w). A feedstock or improved feedstock can also comprise additional nutrients, ingredients or supplements (e.g. vitamins). An improved feedstock comprising a genetically modified algae can also comprise any normal ingredient of an animal feed including but not limited any vegetable, fruit, seed, root, flower, leaf, stem, stalk or plant product of any plant. An improved feedstock comprising a genetically modified algae can also comprise any animal parts or products (e.g. meat, bone, milk, excrement, skin). An improved feedstock comprising a genetically modified NVPO can also comprise any product or bi-product of a manufacturing process (e.g. sawdust or brewers waste). Additional non limiting examples of ingredients of a feedstock or an improved feedstock as disclosed herein include alfalfa, barley, blood meal, grass, legumes, silage, beet, bone meal, brewer grain, brewer's yeast, broom grass, carrot, cattle manure, clover, coffee, corn, corn, glutten meal, distiller grains, poultry fat, grape, hominy feed, hop leaves, spent hops, molasses, oats, algae, peanuts, potato, poultry litter, poultry manure, rape meal, rye, safflower, sorghum, soybean, soy, sunflower meal, timothy hay, or triticale. Therefore in one aspect a composition, can comprise a feedstock and a genetically modified NVPO wherein the feedstock comprises one or more of alfalfa, barley, blood meal, beet, bone meal, brewer grain, brewer's yeast, broom grass, carrot, cattle manure, clover, coffee, coin, corn glutten meal, distiller grains, poultry fat, grape, hominy feed, hop leaves, spent hops, molasses, oats, algae, peanuts, potato, poultry litter, poultry manure, rape meal, rye, safflower, sorghum, soybean, soy, sunflower meal, timothy hay, or triticale.

In some aspect a genetically modified algae can be used for a purpose (e.g. in producing a recombinant product or biofuel) and the remaining portion thereof can be used for an improved feedstock. Therefore an improved feedstock can comprise a portion of a genetically modified algae. For example a composition of an animal feed ingredient can comprise of whole and/or defatted algae (e.g. after removal of fatty acids, lipids or hydrocarbons, e.g. after hexane extraction) or a mixture of whole and defatted algae, which provides both the feed enzyme and the inherent nutritive value of the algae. In another example a genetically modified algae can be washed, dehydrated, centrifuged, filtered, defatted, lysed, dried, processed (e.g. extracted), or milled. The remaining portion thereof can be used as a feedstock, as an improved feedstock or as a supplement to improve a feedstock. For example a composition can comprise a feedstock and a portion of a genetically modified algae wherein the genetically modified algae is at least partially depleted of a lipid, fatty acid, isoprenoid, carotenoid, carbohydrate, or selected protein. The genetically modified algae can also be genetically modified to produce a biomass-degrading enzyme as disclosed herein.

Methods of generating, modifying, supplementing or improving a feedstock composition are also disclosed herein. The methods can comprise combining a genetically modified algae or a portion thereof with a feedstock to generate the improved feedstock, in one example the method comprises removing a lipid, fatty acid, isoprenoid, or carbohydrate from a genetically modified algae. The remaining genetically modified algae, or a portion thereof, can be combined with a feedstock to generate the improved feedstock composition. In one example of the method the modified algae does not express an exogenous phytase. The genetically modified algae or a portion thereof can comprise a nucleic acid (e.g. an exogenous nucleic acid). The nucleic acid can be a vector. In one example of the method, the nucleic acid encodes a biomass degrading enzyme. The biomass-degrading enzyme can be a galactanase, xylanase, protease, carbohydrase, lipase, reductase, oxidase, transglutaminase, or phytase. The biomass-degrading enzyme can be a carbohydrase wherein the carbohydrase is an α-amylase, β-amylase, endo-β-glucanase, endoxylanase, β-mannanase, α-galactosidase, or pullulanase. The biomass-degrading enzyme can be a protease wherein the protease is a subtilisin, bromelain, or fungal acid-stable protease. The biomass-degrading enzyme can be a phytase. In another example of the method the genetically modified NVPO further comprises an exogenous nucleic acid encoding an enzyme in an isoprenoid biosynthesis pathway. The enzyme in the isoprenoid biosynthesis pathway can be farnesyl pyrophosphate synthase, geranyl geranyl phosphate synthase, squalene synthase, thioesterase, or fatty acyl-CoA desaturase. The enzyme in the isoprenoid biosynthesis pathway can be in a mevalonate pathway. In yet another example of the method, the method can further comprise removing a lipid, fatty acid, or isoprenoid, from the genetically modified NVPO prior to combining with a feedstock to generate the improved feedstock.

Candidate genes for directing the expression of proteins (e.g. enzymes) in genetically modified algae for use in animal feeds can be obtained from a variety of organisms including eukaryotes, prokaryotes, or viruses. In some instances, an expressed enzyme is one member of a metabolic pathway (e.g. an isoprenoid biosynthesis pathway). Several enzymes may be introduced into the algae to produce increased levels of desired metabolites, or several enzymes may be introduced to produce a algae containing multiple useful feed enzyme activities (e.g. simultaneous production of xylanase, endo-β-glucanase, and phytase activities).

Feed enzymes can be expressed in host organisms (e.g. Scenedesmus sp.) and purified to a useful level. The purified enzymes can be added to animal feed in a manner similar to current practice. Feed enzymes can also be expressed in host organisms (e.g. algae), and the resulting host organisms can be added as a feed ingredient, adding both nutritive value and desired enzyme activity to the animal feed product. In this application, the genetically modified host organisms can be added to a feedstock alive, whole and non-viable or as a lysate wherein the host organisms are lysed by any suitable means (e.g. physical, chemical or thermal).

Many animal feeds can contain plant seeds, including soybeans, maize, wheat, and barley among others. Plant seeds can contain high levels of myo-inositol polyphosphate (phytic acid). This phytic acid is indigestible to non-ruminant animals, and so feeds with high levels of phytic acid may have low levels of bioavailable phosphorous. The phytic acid can also chelate many important nutritive minerals, such as calcium and magnesium. Incorporation of a phytase into the feed, which can act in the animals upper gut, can release both the chelated mineral nutrients and significant levels of bioavailable phosphorous. The net result is that less free phosphorous needs to be added to the animal feed product. In addition, phosphorous levels in the excreta can be reduced, which can reduce downstream phosphorous pollution.

Genetically modified algae that express phytases or similar enzymes can be added to a feedstock to improve the nutrient or digestible properties of the feed. Phytases contemplated for use herein can be from any organism (e.g. bacterial or fungal derived). Non limiting examples of types of phytases contemplated for use herein include 3-phytase (alternative name 1-phytase; a myo-inositol hexaphosphate 3-phosphohydrolase, EC 3.1.3.8), 4-phytase (alternative name 6-phytase, name based on 1 L-numbering system and not I D-numbering, EC 3.1.3,26), and 5-phytase (EC 3.1.3.72). Additional non limiting examples of phytases include microbial phytases, such as fungal, yeast or bacterial phytases such as disclosed in EP 684313, U.S. Pat. No. 6,139,902, EP 420358, WO 97/35017, WO 98/28408, WO 98/28409, JP 1 1000164, WO98/13480, AU 724094, WO 97/33976, US 6110719, WO 2006/038062, WO 2006/038128, WO 2004/085638, WO 2006/037328, WO 2006/037327, WO 2006/043178, U.S. Pat. No. 5,830,732 and under UniProt designations P34753, P34752, P34755, 000093, 031097, P42094, 066037 and P34754 (UniProt, (2008) http://www.uniprot.org/). Polypeptides having an amino acid sequence of at least 75% identity to an amino acid sequence (comprising the active site) of any one of the phytases disclosed above are also contemplated for use herein. In one example a composition can comprise a feedstock and a genetically modified algae. The genetically modified algae can be genetically modified to produce a biomass-degrading enzyme such as a phytase. In one aspect the phytase is a phytase of bacterial or fungal origin. In one aspect the biomass-degrading enzyme is an enzyme other than a phytase.

Many plant parts (e.g. seeds, fruits, stems, roots, leaves and flowers) from plants such as, for example, soybeans, wheat, and barley contain polysaccharides that are indigestible by some animals (e.g. non-ruminant animals). Non limiting examples of such carbohydrates include xylans, raffinose, stachyose, and glucans. The presence of indigestible carbohydrates in animal feed can reduce nutrient availability. Indigestible carbohydrates in poultry feed can result in sticky feces, which can increase disease levels. The presence of one or more carbohydrate degrading enzymes (e.g. α-amylase) in the animal feed can help break down polysaccharides, increase nutrient availability, increase the bio-available energy content of the animal feed, and reduce health risks. Non limiting examples of carbohydrate degrading enzymes contemplated for use herein include amylases (e.g. α-amylase and β-amylase), β-mannanase, maltase, lactase, β-glucanase, endo-β-glucanase, glucose isomerase, endoxylanase, α-galactosidase, glucose oxidase, pullulanase, invertase and any carbohydrate digesting enzyme of bacterial, fungal, plant or animal origin. In one example a composition can comprise a feedstock and a genetically modified algae. The genetically modified algae can be genetically modified to produce a biomass-degrading enzyme such as a carbohydrase. In one aspect the carbohydrase can be an α-amylase, β-amylase, endo-β-glucanase, endoxylanase, β-mannanase, α-galactosidase, or pullulanase.

Many feedstocks contain plant parts (e.g. seeds) with anti-nutritive proteins (e.g. protease inhibitors, amylase inhibitors and others) that reduce the availability of nutrients in an animal feed. Addition of a broad spectrum protease (e.g. bromelain, subtilisin, or a fungal acid-stable protease) can break down these anti-nutritive proteins and increase the availability of nutrients in the animal's feed. Non limiting examples of proteases contemplated for use herein include endopeptidases and exopeptidases. Non limiting examples of proteases contemplated for use herein include serine proteases (e.g. subtilisin, chymotrypsins, glutamyl peptidases, dipeptidyl-peptidases, carboxypeptidases, dipeptidases, and aminopeptidases), cyteine proteases (e.g. papain, calpain-2, and papain-like peptidases and bromelain), aspartic peptidases (e.g. pepsins and pepsin. A), glutamic proteases, threonine proteases, fungal acid proteases and acid stable proteases such as those disclosed in (U.S. Pat. No. 6,855,548). In one example a composition, can comprise a feedstock and a genetically modified algae. The genetically modified algae can be genetically modified to produce a biomass-degrading enzyme such as a protease. In one aspect the protease can be a subtilisin, bromelain or fungal acid-stable protease.

Non limiting examples of lipases contemplated for use herein, include pancreatic lipase, lysosomal lipase, lysosomal acid lipase, acid cholesteryl ester hydrolase, hepatic lipase, lipoprotein lipase, gastric lipase, endothelial lipase, pancreatic lipase related protein 2, pancreatic lipase related protein 1, lingual lipase and phospholipases (e.g. phospholipase A1(EC 3.1.1.32), phospholipase A2, phospholipase B (lysophospholipase), phospholipase C and phospholipase D).

An improved feedstock can be generated by combining a feedstock with a algae that is genetically altered to produce an enzyme (e.g. a carbohydrase, protease or lipase). In some aspects the enzyme is produced ex vivo to the organism. In some aspects the enzyme is secreted. Enzymes produced ex vivo to the organisms can break down components of a feedstock prior to ingestion by an animal. Therefore an improved feedstock can be generated by combining a feedstock with an algae that is genetically altered to produce an enzyme (e.g. a carbohydrase, protease or lipase) and subjecting the mixture to a holding period. A holding period can allow the genetically altered algae to multiply and to secrete more enzyme into the feedstock. A holding period can be from several hours up to several days. In some aspects a holding period is for up to 1, 2, 3, 4, 5, 6, 7, 8, 9 or 10 days. In some aspects a holding period is for up to several days to several weeks. In some aspects a holding period is indefinite. An indefinite holding period allows intermittent removal and use of the improved feedstock and intermittent addition of the base feedstock

Host Cells or Host Organisms

Biomass useful in the methods and systems described herein can be obtained from host cells or host organisms.

A host cell can contain a polynucleotide encoding a polypeptide of the present disclosure. In some embodiments, a host cell is part of a multicellular organism. In other embodiments, a host cell is cultured as a unicellular organism.

Host organisms can include any suitable host, for example, a microorganism. Microorganisms which are useful for the methods described herein include, for example, photosynthetic bacteria (e.g., cyanobacteria), non-photosynthetic bacteria (e.g., E. coli), yeast (e.g., Saccharomyces cerevisiae), and algae (e.g., microalgae such as Chlamydomonas reinhardtii).

Examples of host organisms that can be transformed with a polynucleotide of interest (for example, a polynucleotide that encodes a protein involved in the isoprenoid biosynthesis pathway) include vascular and non-vascular organisms. The organism can be prokaryotic or eukaryotic. The organism can be unicellular or multicellular. A host organism is an organism comprising a host cell. In other embodiments, the host organism is photosynthetic. A photosynthetic organism is one that naturally photosynthesizes (e.g., an alga) or that is genetically engineered or otherwise modified to be photosynthetic. In some instances, a photosynthetic organism may be transformed with a construct or vector of the disclosure which renders all or part of the photosynthetic apparatus inoperable.

By way of example, a non-vascular photosynthetic microalga species (for example, C. reinhardtii, Nannochloropsis oceania, N. salina, D. salina, H. pluvalis, S. dimorphus, D. viridis, Chlorella sp., and D. tertiolecta) can be genetically engineered to produce a polypeptide of interest, for example a fusicoccadiene synthase or an FPP synthase. Production of a fusicoccadiene synthase or an FPP synthase in these microalgae can be achieved by engineering the microalgae to express the fusicoccadiene synthase or FPP synthase in the algal chloroplast or nucleus.

In other embodiments the host organism is a vascular plant. Non-limiting examples of such plants include various monocots and dicots, including high oil seed plants such as high oil seed Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, barley, oats, amaranth, potato, rice, tomato, and legumes (e.g., peas, beans, lentils, alfalfa, etc.).

The host cell can be prokaryotic. Examples of some prokaryotic organisms of the present disclosure include, but are not limited to, cyanobacteria (e.g., Synechococcus, Synechocystis, Athrospira, Gleocapsa, Oscillatoria, and, Pseudoanabaena). Suitable prokaryotic cells include, but are not limited to, any of a variety of laboratory strains of Escherichia coli, Lactobacillus sp. Salmonella sp., and Shigella sp. (for example, as described in Carrier et al. (1992) J. Immunol. 148:1176-1181; U.S. Pat. No. 6,447,784; and Sizemore et al. (1995) Science 270:299-302). Examples of Salmonella strains which can be employed in the present disclosure include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Pseudomonas pudita, Pseudomonas aeruginosa, Pseudomonas mevalonii, Rhodobacter sphaeroides, Rhodobacter capsulatus, Rhodospirillum rubrum, and Rhodococcus sp.

In some embodiments, the host organism is eukaryotic (e.g. green algae, red algae, brown algae). In some embodiments, the algae is a green algae, for example, a Chlorophycean. The algae can be unicellular or multicellular. Suitable eukaryotic host cells include, but are not limited to, yeast cells, insect cells, plant cells, fungal cells, and algal cells. Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, and Chlamydomonas reinhardtii. In other embodiments, the host cell is a microalga (e.g., Chlamydomonas reinhardtii, Dunaliella salina, Haematococcus pluvialis, Nannochloropsis Oceania, N. salina, Scenedesmus dimorphus, Chlorella spp., D. viridis, or D. tertiolecta).

In some instances the organism is a rhodophyte, chlorophyte, heterokontophyte, tribophyte, glaucophyte, chlorarachniophyte, euglenoid, haptophyte, cryptomonad, dinoflagellum, or phytoplankton.

In some instances a host organism is vascular and photosynthetic. Examples of vascular plants include, but are not limited to, angiosperms, gymnosperms, rhyniophytes, or other tracheophytes.

In some instances a host organism is non-vascular and photosynthetic. As used herein, the term “non-vascular photosynthetic organism,” refers to any macroscopic or microscopic organism, including, but not limited to, algae, cyanobacteria and photosynthetic bacteria, which does not have a vascular system such as that found in vascular plants. Examples of non-vascular photosynthetic organisms include bryophtyes, such as marchantiophytes or anthocerotophytes. In some instances the organism is a cyanobacteria. In some instances, the organism is algae (e.g., macroalgae or microalgae). The algae can be unicellular or multicellular algae. For example, the microalgae Chlamydomonas reinhardtii may be transformed with a vector, or a linearized portion thereof, encoding one or more proteins of interest (e.g., a protein involved in the isoprenoid biosynthesis pathway).

Methods for algal transformation are described in U.S. Provisional Patent Application No. 60/142,091. The methods of the present disclosure can be carried out using algae, for example, the microalga, C. reinhardtii. The use of microalgae to express a polypeptide or protein complex according to a method of the disclosure provides the advantage that large populations of the microalgae can be grown, including commercially (Cyanotech Corp.; Kailua-Kona Hi.), thus allowing for production and, if desired, isolation of large amounts of a desired product.

The vectors of the present disclosure may be capable of stable or transient transformation of multiple photosynthetic organisms, including, but not limited to, photosynthetic bacteria (including cyanobacteria), cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, and phytoplankton. Other vectors of the present disclosure are capable of stable or transient transformation of, for example, C. reinhardtii, N, Oceania, N. salina, D. salina, H. pluvalis, S. dimorphus, D. viridis, or D. tertiolecta.

Examples of appropriate hosts, include but are not limited to: bacterial cells, such as E. coli, Streptomyces, Salmonella tryphimurium; fungal cells, such as yeast; insect cells, such as Drosophila S2 and Spodoptera Sf9; animal cells, such as CHO, COS or Bowes melanoma; adenoviruses; and plant cells. The selection of an appropriate host is deemed to be within the scope of those skilled in the art.

Polynucleotides selected and isolated as described herein are introduced into a suitable host cell. A suitable host cell is any cell which is capable of promoting recombination and/or reductive reassortment. The selected polynucleotides can be, for example, in a vector which includes appropriate control sequences. The host cell can be, for example, a higher eukaryotic cell, such, as a mammalian cell, or a lower eukaryotic cell, such, as a yeast cell, or the host cell can be a prokaryotic cell, such as a bacterial cell. Introduction of a construct (vector) into the host cell can be effected by, for example, calcium phosphate transfection, DEAE-Dextran mediated transfection, or electroporation.

Recombinant polypeptides, including protein complexes, can be expressed in plants, allowing for the production of crops of such, plants and, therefore, the ability to conveniently produce large amounts of a desired product. Accordingly, the methods of the disclosure can be practiced using any plant, including, for example, microalga and macroalgae, (such as marine algae and seaweeds), as well as plants that grow in soil.

In one embodiment, the host cell is a plant. The term “plant” is used broadly herein to refer to a eukaryotic organism containing plastids, such as chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, and roots. A part of a plant useful for propagation includes, for example, seeds, fruits, cuttings, seedlings, tubers, and rootstocks.

A method of the disclosure can generate a plant containing genomic DNA (for example, a nuclear and/or plastid genomic DNA) that is genetically modified to contain a stably integrated polynucleotide (for example, as described in Hager and Bock, Appl. Microbiol. Biotechnol. 54:302-310, 2000). Accordingly, the present disclosure further provides a transgenic plant, e.g. C. reinhardtii, which comprises one or more chloroplasts containing a polynucleotide encoding one or more exogenous or endogenous polypeptides, including polypeptides that can allow for secretion of fuel products and/or fuel product precursors (e.g., isoprenoids, fatty acids, lipids, triglycerides). A photosynthetic organism of the present disclosure comprises at least one host cell that is modified to generate, for example, a fuel product or a fuel product precursor.

Some of the host organisms useful in the disclosed embodiments are, for example, are extremophiles, such as hyperthermophiles, psychrophiles, psychrotrophs, halophiles, barophiles and acidophiles. Some of the host organisms which may be used to practice the present disclosure are halophilic (e.g., Dunaliella salina, D. viridis, or D. tertiolecta). For example, D. salina can grow in ocean water and salt lakes (for example, salinity from 30-300 parts per thousand) and high salinity media (e.g., artificial seawater medium, seawater nutrient agar, brackish water medium, and seawater medium). In some embodiments of the disclosure, a host cell expressing a protein of the present disclosure can be grown in a liquid environment which is, for example, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 31., 3.2, 3.3, 3.4, 3.5, 3.6, 3.7, 3.8, 3.9, 4.0, 4.1, 4.2, 4.3 molar or higher concentrations of sodium chloride. One of skill in the art will recognize that other salts (sodium salts, calcium salts, potassium salts, or other salts) may also be present in the liquid environments.

Where a halophilic organism is utilized for the present disclosure, it may be transformed with any of the vectors described herein. For example, D. salina may be transformed with a vector which is capable of insertion into the chloroplast or nuclear genome and which contains nucleic acids which encode a protein (e.g., an FPP synthase or a fusicoccadiene synthase). Transformed halophilic organisms may then be grown in. high-saline environments (e.g., salt lakes, salt ponds, and high-saline media) to produce the products (e.g., lipids) of interest. Isolation of the products may involve removing a transformed organism from a high-saline environment prior to extracting the product from the organism. In instances where the product is secreted into the surrounding environment, it may be necessary to desalinate the liquid environment prior to any further processing of the product.

The present disclosure further provides compositions comprising a genetically modified host cell. A composition comprises a genetically modified host cell; and will in some embodiments comprise one or more further components, which components are selected based in part on the intended use of the genetically modified host cell. Suitable components include, but are not limited to, salts; buffers; stabilizers; protease-inhibiting agents; cell membrane- and/or cell wall-preserving compounds, e.g., glycerol and dimethylsulfoxide; and nutritional media appropriate to the cell.

For the production of a protein, for example, an isoprenoid or isoprenoid precursor compound, a host cell can be, for example, one that produces, or has been genetically modified to produce, one or more enzymes in a prenyl transferase pathway and/or a mevalonate pathway and/or an isoprenoid biosynthetic pathway. In some embodiments, the host cell is one that produces a substrate of a prenyl transferase, isoprenoid synthase or mevalonate pathway enzyme.

In some embodiments, a genetically modified host cell is a host cell that comprises an endogenous mevalonate pathway and/or isoprenoid biosynthetic pathway and/or prenyl transferase pathway. In other embodiments, a genetically modified host cell is a host cell that does not normally produce mevalonate or IPP via a mevalonate pathway, or FPP, GPP or GGPP via a prenyl transferase pathway, but has been genetically modified with one or more polynucleotides comprising nucleotide sequences encoding one or more mevalonate pathway, isoprenoid synthase pathway or prenyl transferase pathway enzymes (for example, as described in U.S. Patent Publication No. 2004/005678; U.S. Patent Publication No. 2003/0148479; and Martin et al. (2003) Nat. Biotech. 21(71:796-802).

Culturing of Cells or Organisms

An organism may be grown under conditions which permit photosynthesis, however, this is not a requirement (e.g., a host organism may be grown in the absence of light). In some instances, the host organism may be genetically modified in such a way that its photosynthetic capability is diminished or destroyed. In growth conditions where a host organism is not capable of photosynthesis (e.g., because of the absence of light and/or genetic modification), typically, the organism will be provided with the necessary nutrients to support growth in the absence of photosynthesis. For example, a culture medium in (or on) which an organism is grown, may be supplemented with any required nutrient, including an organic carbon source, nitrogen source, phosphorous source, vitamins, metals, lipids, nucleic acids, micronutrients, and/or an organism-specific requirement. Organic carbon sources include any source of carbon which the host organism is able to metabolize including, but not limited to, acetate, simple carbohydrates (e.g., glucose, sucrose, and lactose), complex carbohydrates (e.g., starch and glycogen), proteins, and lipids. One of skill in the art will recognize that not all organisms will be able to sufficiently metabolize a particular nutrient and that nutrient mixtures may need to be modified from one organism to another in order to provide the appropriate nutrient mix.

Optimal growth of organisms occurs usually at a temperature of about 20° C. to about 25° C., although some organisms can still grow at a temperature of up to about 35° C. Active growth, is typically performed in liquid culture. If the organisms are grown, in a liquid medium and are shaken, or mixed, the density of the cells can be anywhere from about 1 to 5×10⁸ cells/ml at the stationary phase. For example, the density of the cells at the stationary phase for Chlamydomonas sp, can be about 1 to 5×10⁷ cells/ml; the density of the cells at the stationary phase for Nannochloropsis sp, can be about 1 to 5×10⁸ cells/ml; the density of the cells at the stationary phase for Scenedesmus sp. can be about 1 to 5×10⁷ cells/ml; and the density of the cells at the stationary phase for Chlorella sp. can be about 1 to 5×10⁸ cells/ml. Exemplary cell densities at the stationary phase are as follows: Chlamydomonas sp. can be about 1×10⁷ cells/ml; Nannochloropsis sp. can be about 1×10⁸ cells/ml; Scenedesmus sp. can be about 1×10⁷ cells/ml; and Chlorella sp. can be about 1×10⁸ cells/ml. An exemplary growth rate may yield, for example, a two to four fold increase in cells per day, depending on the growth conditions. In addition, doubling times for organisms can be, for example, 5 hours to 30 hours. The organism can also be grown on solid media, for example, media containing about 1.5% agar, in plates or in slants.

One source of energy is fluorescent light that can be placed, for example, at a distance of about 3 inch to about two feet from the organism. Examples of types of fluorescent lights includes, for example, cool white and daylight. Bubbling with air or CO₂ improves the growth rate of the organism. Bubbling with CO₂ can be, for example, at 1% to 5% CO₂. If the lights are fumed on and off at regular intervals (for example, 12:12 or 14:10 hours of light:dark) the cells of some organisms will become synchronized.

Long term storage of organisms can be achieved by streaking them onto plates, sealing the plates with, for example, Parafilm™, and placing them in dim light at about 10° C. to about 18° C. Alternatively, organisms may be grown as streaks or stabs into agar tubes, capped, and stored at about 10° C. to about 18° C. Both methods allow for the storage of the organisms for several months.

For longer storage, the organisms can be grown in liquid culture to mid to late log phase and then supplemented with a penetrating cryoprotective agent like DMSO or MeOH, and stored at less than −130° C. An exemplary range of DMSO concentrations that can be used is 5 to 8%. An exemplary range of MeOH concentrations that can be used is 3 to 9%.

Organisms can be grown on a defined minimal medium (for example, high salt medium (HSM), modified artificial sea water medium (MASM), or F/2 medium) with light as the sole energy source. In other instances, the organism can be grown in a medium (for example, tris acetate phosphate (TAP) medium), and supplemented with an organic carbon source.

Organisms, such as algae, can grow naturally in fresh water or marine water. Culture media for freshwater algae can be, for example, synthetic media, enriched media, soil water media, and solidified media, such as agar. Various culture media have been developed and used for the isolation and cultivation of fresh water algae and are described in Watanabe, M. W. (2005). Freshwater Culture Media. In R. A. Andersen (Ed.), Algal Culturing Techniques (pp. 13-20). Elsevier Academic Press. Culture media for marine algae can be, for example, artificial seawater media or natural seawater media. Guidelines for the preparation of media are described in Harrison, P. J. and Berges, J. A. (2005). Marine Culture Media. In R. A. Andersen (Ed.), Algal Culturing Techniques (pp. 21-33). Elsevier Academic Press.

Organisms may be grown in outdoor open water, such as ponds, the ocean, seas, rivers, waterbeds, marshes, shallow pools, lakes, aqueducts, and reservoirs. When grown in water, the organism can be contained in a halo-like object comprised of lego-like particles. The halo-like object encircles the organism and allows it to retain nutrients from the water beneath while keeping it in open sunlight.

In some instances, organisms can be grown in containers wherein, each container comprises one or two organisms, or a plurality of organisms. The containers can be configured to float on water. For example, a container can be filled by a combination of air and water to make the container and the organism(s) in it buoyant. An organism that is adapted to grow in fresh water can thus be grown in salt water (i.e., the ocean) and vice versa. This mechanism allows for automatic death of the organism if there is any damage to the container.

Culturing techniques for algae are well know to one of skill in the art and are described, for example, in Freshwater Culture Media. In R. A. Andersen (Ed.), Algal Culturing Techniques. Elsevier Academic Press.

Because photosynthetic organisms, for example, algae, require sunlight, CO₂ and water for growth, they can be cultivated in, for example, open ponds and lakes. However, these open systems are more vulnerable to contamination than a closed system. One challenge with using an open system is that the organism of interest may not grow as quickly as a potential invader. This becomes a problem when another organism invades the liquid environment in which the organism of interest is growing, and the invading organism has a faster growth rate and takes over the system.

In addition, in open systems there is less control over water temperature, CO₂ concentration, and lighting conditions. The growing season of the organism is largely dependent on location and, aside from tropical areas, is limited to the warmer months of the year. In addition, in an open system, the number of different organisms that can be grown is limited to those that are able to survive in the chosen location. An open system, however, is cheaper to set up and/or maintain than a closed system.

Another approach to growing an organism is to use a semi-closed system, such as covering the pond or pool with a structure, for example, a “greenhouse-type” structure. While this can result in a smaller system, it addresses many of the problems associated with an open system. The advantages of a semi-closed system are that it can allow for a greater number of different organisms to be grown, it can allow for an organism to be dominant over an invading organism by allowing the organism of interest to out compete the invading organism for nutrients required for its growth, and it can extend the growing season for the organism. For example, if the system is heated, the organism can grow year round.

A variation of the pond system is an artificial pond, for example, a raceway pond. In these ponds, the organism, water, and nutrients circulate around a “racetrack.” Paddlewheels provide constant motion to the liquid in the racetrack, allowing for the organism to be circulated back to the surface of the liquid at a chosen frequency. Paddlewheels also provide a source of agitation and oxygenate the system. These raceway ponds can be enclosed, for example, in a building or a greenhouse, or can be located outdoors.

Raceway ponds are usually kept shallow because the organism needs to be exposed to sunlight, and sunlight can only penetrate the pond water to a limited depth. The depth of a raceway pond can be, for example, about 4 to about 12 inches. In addition, the volume of liquid that can be contained in a raceway pond can be, for example, about 200 liters to about 600,000 liters.

The raceway ponds can be operated in a continuous manner, with, for example, CO₂ and nutrients being constantly fed to the ponds, while water containing the organism is removed at the other end.

If the raceway pond is placed outdoors, there are several different ways to address the invasion of an unwanted organism. For example, the pH or salinity of the liquid in which the desired organism is in can be such that the invading organism either slows down its growth, or dies.

Also, chemicals can be added to the liquid, such as bleach, or a pesticide can be added to the liquid, such as glyphosate. In addition, the organism of interest can be genetically modified such that it is better suited to survive in the liquid environment. Any one or more of the above strategies can be used to address the invasion of an unwanted organism.

Alternatively, organisms, such, as algae, can be grown in closed structures such, as photobioreactors, where the environment is under stricter control than, in open systems or semi-closed systems. A photobioreactor is a bioreactor which incorporates some type of light source to provide photonic energy input into the reactor. The term photobioreactor can refer to a system closed to the environment and having no direct exchange of gases and contaminants with the environment. A photobioreactor can be described as an enclosed, illuminated culture vessel designed for controlled biomass production of phototrophic liquid cell suspension cultures. Examples of photobioreactors include, for example, glass containers, plastic tubes, tanks, plastic sleeves, and bags. Examples of light sources that can be used to provide the energy required to sustain photosynthesis include, for example, fluorescent bulbs, LEDs, and natural sunlight. Because these systems are closed everything that the organism needs to grow (for example, carbon dioxide, nutrients, water, and light) must be introduced into the bioreactor.

Photobioreactors, despite the costs to set up and maintain them, have several advantages over open systems, they can, for example, prevent or minimize contamination, permit axenic organism cultivation of monocultures (a culture consisting of only one species of organism), offer better control over the culture conditions (for example, pH, light, carbon dioxide, and temperature), prevent water evaporation, lower carbon dioxide losses due to out gassing, and permit higher cell concentrations.

On the other hand, certain requirements of photobioreactors, such as cooling, mixing, control of oxygen accumulation and biofouling, make these systems more expensive to build and operate than open systems or semi-closed systems.

Photobioreactors can be set up to be continually harvested (as is with the majority of the larger volume cultivation systems), or harvested one batch at a time (for example, as with polyethlyene bag cultivation). A batch photobioreactor is set up with, for example, nutrients, an organism (for example, algae), and water, and the organism is allowed to grow until the batch is harvested. A continuous photobioreactor can be harvested, for example, either continually, daily, or at fixed time intervals.

High density photobioreactors are described in, for example, Lee, et al., Biotech. Bioengineering 44:1161-1167, 1994. Other types of bioreactors, such as those for sewage and waste water treatments, are described in, Sawayama, et al., Appl, Micro. Biotech., 41:729-731, 1994. Additional examples of photobioreactors are described in, U.S. Appl. Publ. No. 2005/0260553, U.S. Pat. No. 5,958,761, and U.S. Pat. No. 6,083,740. Also, organisms, such as algae may be mass-cultured for the removal of heavy metals (for example, as described in Wilkinson, Biotech. Letters, 11:861-864, 1989), hydrogen (for example, as described in U.S. Patent Application Publication No. 2003/0162273), and pharmaceutical compounds from a water, soil, or other source or sample. Organisms can also be cultured in conventional fermentation bioreactors, which include, but are not limited to, batch, fed-batch, cell recycle, and continuous fermentors. Additional methods of culturing organisms and variations of the methods described herein are known to one of skill in the art.

Organisms can also be grown, near ethanol production plants or other facilities or regions (e.g., cities and highways) generating CO₂. As such, the methods herein contemplate business methods for selling carbon credits to ethanol plants or other facilities or regions generating CO₂ while making fuels or fuel products by growing one or more of the organisms described herein near the ethanol production plant, facility, or region.

The organism of interest, grown in any of the systems described herein, can be, for example, continually harvested, or harvested one batch at a time.

CO₂ can be delivered to any of the systems described herein, for example, by bubbling in CO₂ from under the surface of the liquid containing the organism. Also, sparges can be used to inject CO₂ into the liquid. Spargers are, for example, porous disc or tube assemblies that are also referred to as Bubblers, Carbonators, Aerators, Porous Stones and Diffusers.

Nutrients that can be used in the systems described herein include, for example, nitrogen (in the form of NO₃⁻ or NH₄⁺), phosphorus, and trace metals (Fe, Mg, K, Ca, Co, Cu, Mn, Mo, Zn, V, and B). The nutrients can come, for example, in a solid form or in a liquid form. If the nutrients are in a solid form they can be mixed with, for example, fresh or salt water prior to being delivered to the liquid containing the organism, or prior to being delivered to a photobioreactor.

Organisms can be grown in cultures, for example large scale cultures, where large scale cultures refers to growth of cultures in volumes of greater than about 6 liters, or greater than about 10 liters, or greater than about 20 liters. Large scale growth can also be growth of cultures in volumes of 50 liters or more, 100 liters or more, or 200 liters or more. Large scale growth can be growth of cultures in, for example, ponds, containers, vessels, or other areas, where the pond, container, vessel, or area that contains the culture is for example, at lease 5 square meters, at least 10 square meters, at least 200 square meters, at least 500 square meters, at least 1,500 square meters, at least 2,500 square meters, in area, or greater.

Chlamydomonas sp., Nannochloropsis sp., Scenedesmus sp., and Chlorella sp. are exemplary algae that can be cultured as described herein and can grow under a wide array of conditions.

One organism that can be cultured as described herein is a commonly used laboratory species C. reinhardtii. Cells of this species are haploid, and can grow on a simple medium of inorganic salts, using photosynthesis to provide energy. This organism can also grow in total darkness if acetate is provided as a carbon source. C. reinhardtii can be readily grown at room temperature under standard fluorescent lights. In addition, the cells can be synchronized by placing them on a light-dark cycle. Other methods of culturing C. reinhardtii cells are known to one of skill in the art.

Polynucleotides and Polypeptides

Also provided are isolated polynucleotides encoding a protein, for example, an FPP synthase, described herein. As used herein “isolated polynucleotide” means a polynucleotide that is free of one or both of the nucleotide sequences which flank the polynucleotide in the naturally-occurring genome of the organism from which the polynucleotide is derived. The term includes, for example, a polynucleotide or fragment thereof that is incorporated into a vector or expression cassette; into an autonomously replicating plasmid or virus; into the genomic DNA of a prokaryote or eukaryote; or that exists as a separate molecule independent of other polynucleotides. It also includes a recombinant polynucleotide that is part of a hybrid polynucleotide, for example, one encoding a polypeptide sequence.

The novel proteins of the present disclosure can be made by any method known in the art. The protein may be synthesized using either solid-phase peptide synthesis or by classical solution peptide synthesis also known as liquid-phase peptide synthesis. Using Val-Pro-Pro, Enalapril and Lisinopril as starting templates, several series of peptide analogs such as X-Pro-Pro, X-Ala-Pro, and X-Lys-Pro, wherein X represents any amino acid residue, may be synthesized using solid-phase or liquid-phase peptide synthesis. Methods for carrying out liquid phase synthesis of libraries of peptides and oligonucleotides coupled to a soluble oligomeric support have also been described. Bayer, Ernst and Mutter, Manfred, Nature 237:512-513 (1972); Bayer, Ernst, et al., J. Am. Chem. Soc. 96:7333-7336 (1974); Bonora, Gian Maria, et al., Nucleic Acids Res. 18:3155-3159 (1990), liquid phase synthetic methods have the advantage over solid phase synthetic methods in that liquid phase synthesis methods do not require a structure present on a first reactant which is suitable for attaching the reactant to the solid phase. Also, liquid phase synthesis methods do not require avoiding chemical conditions which may cleave the bond between the solid phase and the first reactant (or intermediate product). In addition, reactions in a homogeneous solution may give better yields and more complete reactions than those obtained in heterogeneous solid phase/liquid phase systems such as those present in solid phase synthesis.

In oligomer-supported liquid phase synthesis the growing product is attached to a large soluble polymeric group. The product from, each step of the synthesis can then be separated from unreacted reactants based on the large difference in size between the relatively large polymer-attached product and the unreacted reactants. This permits reactions to take place in homogeneous solutions, and eliminates tedious purification steps associated with traditional liquid phase synthesis. Oligomer-supported liquid phase synthesis has also been adapted to automatic liquid phase synthesis of peptides. Bayer, Ernst, et al., Peptides: Chemistry, Structure, Biology, 426-432.

For solid-phase peptide synthesis, the procedure entails the sequential assembly of the appropriate amino acids into a peptide of a desired sequence while the end of the growing peptide is linked to an insoluble support. Usually, the carboxyl terminus of the peptide is linked to a polymer from which it can be liberated upon treatment with a cleavage reagent. In a common method, an amino acid is bound to a resin particle, and the peptide generated in a stepwise manner by successive additions of protected amino acids to produce a chain of amino acids. Modifications of the technique described by Merrifield are commonly used. See, e.g., Merrifield, J. Am. Chem. Soc. 96: 2989-93 (1964). In an automated solid-phase method, peptides are synthesized by loading the carboxy-terminal amino acid onto an organic linker (e.g., PAM, 4-oxymethylphenylacetamidomethyl), which is covalently attached to an insoluble polystyrene resin cross-linked with divinyl benzene. The terminal amine may be protected by blocking with t-butyloxycarbonyl. Hydroxyl- and carboxyl-groups are commonly protected by blocking with O-benzyl groups. Synthesis is accomplished in an automated peptide synthesizer, such as that available from Applied Biosystems (Foster City, Calif.). Following synthesis, the product may be removed from the resin. The blocking groups are removed by using hydrofluoric acid or trifluoromethyl sulfonic acid according to established methods. A routine synthesis may produce 0.5 mmole of peptide resin. Following cleavage and purification, a yield of approximately 60 to 70% is typically produced. Purification of the product peptides is accomplished by, for example, crystallizing the peptide from an organic solvent such as methyl-butyl ether, then dissolving in distilled water, and using dialysis (if the molecular weight of the subject peptide is greater than about 500 daltons) or reverse high pressure liquid chromatography (e.g., using a C¹⁸ column with 0.1% trifluoroacetic acid and acetonitrile as solvents) if the molecular weight of the peptide is less than 500 daltons. Purified peptide may be lyophilized and stored in a dry state until use. Analysis of the resulting peptides may be accomplished using the common methods of analytical high pressure liquid chromatography (HPLC) and electrospray mass spectrometry (ES-MS).

In other cases, a protein, for example, a protein involved in the isoprenoid biosynthesis pathway or in fatty acid synthesis, is produced by recombinant methods. For production of any of the proteins described herein, host cells transformed with an expression vector containing the polynucleotide encoding such, a protein can be used. The host cell can be a higher eukaryotic cell, such as a mammalian cell, or a lower eukaryotic cell such as a yeast or algal cell, or the host can be a prokaryotic cell such as a bacterial cell. Introduction of the expression vector into the host cell can be accomplished by a variety of methods including calcium phosphate transfection, DEAE-dextran mediated transfection, polybrene, protoplast fusion, liposomes, direct microinjection into the nuclei, scrape loading, biolistic transformation and electroporation. Large scale production of proteins from recombinant organisms is a well established process practiced on a commercial scale and well within the capabilities of one skilled in the art.

It should be recognized that the present disclosure is not limited to transgenic cells, organisms, and plastids containing a protein or proteins as disclosed herein, but also encompasses such cells, organisms, and plastids transformed with additional nucleotide sequences encoding enzymes involved in fatty acid synthesis. Thus, some embodiments involve the introduction of one or snore sequences encoding proteins involved in fatty acid synthesis in addition to a protein disclosed herein. For example, several enzymes in a fatty acid production pathway may be linked, either directly or indirectly, such that products produced by one enzyme in the pathway, once produced, are in close proximity to the next enzyme in the pathway. These additional sequences may be contained in a single vector either operatively linked to a single promoter or linked to multiple promoters, e.g. one promoter for each sequence. Alternatively, the additional coding sequences may be contained in a plurality of additional vectors. When a plurality of vectors are used, they can be introduced into the host cell or organism simultaneously or sequentially.

Additional embodiments provide a plastid, and in particular a chloroplast, transformed with a polynucleotide encoding a protein of the present disclosure. The protein may be introduced into the genome of the plastid using any of the methods described herein or otherwise known in the art. The plastid may be contained in the organism in which it naturally occurs. Alternatively, the plastid may be an isolated plastid, that is, a plastid that has been removed from the cell in which it normally occurs. Methods for the isolation of plastids are known in the art and can be found, for example, in Maliga et al., Methods in Plant Molecular Biology, Cold Spring Harbor Laboratory Press, 1995; Gupta and Singh, J. Biosci., 21:819 (1996); and Camara et al., Plant Physiol., 73:94 (1983). The isolated plastid transformed with a protein of the present disclosure can be introduced into a host cell. The host cell can be one that naturally contains the plastid or one in which the plastid is not naturally found.

Also within the scope of the present disclosure are artificial plastid genomes, for example chloroplast genomes, that contain nucleotide sequences encoding any one or more of the proteins of the present disclosure. Methods for the assembly of artificial plastid genomes can be found in co-pending U.S. patent application Ser. No. 12/287,230 filed Oct. 6, 2008, published as U.S. Publication No. 2009/0123977 on May 14, 2009, and U.S. patent application Ser. No. 12/384,893 filed Apr. 8, 2009, published as U.S. Publication No. 2009/0269816 on Oct. 29, 2009, each of which is incorporated by reference in its entirety.

Introduction of Polynucleotide into a Host Organism or Cell

To generate a genetically modified host cell, a polynucleotide, or a polynucleotide cloned into a vector, is introduced stably or transiently into a host cell, using established techniques, including, but not limited to, electroporation, calcium phosphate precipitation, DEAE-dextran mediated transfection, and liposome-mediated transfection. For transformation, a polynucleotide of the present disclosure will generally further include a selectable marker, e.g., any of several well-known selectable markers such as neomycin resistance, ampicillin resistance, tetracycline resistance, chloramphenicol resistance, and kanamycin resistance.

A polynucleotide or recombinant nucleic acid molecule described herein, can be introduced into a cell (e.g., alga cell) using any method known in the art. A polynucleotide can be introduced into a cell by a variety of methods, which are well known in the art and selected, in part, based on the particular host cell. For example, the polynucleotide can be introduced into a cell using a direct gene transfer method such, as electroporation or microprojectile mediated (biolistic) transformation using a particle gun, or the “glass bead method,” or by pollen-mediated transformation, liposome-mediated transformation, transformation using wounded or enzyme-degraded immature embryos, or wounded or enzyme-degraded embryogenic callus (for example, as described in Potrykus, Ann. Rev. Plant. Physiol. Plant Mol. Biol. 42:205-225, 1991).

As discussed above, microprojectile mediated transformation can be used to introduce a polynucleotide into a cell (for example, as described in Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a cell using a device such as the BIOLISTIC PD-1000 particle gun (BioRad; Hercules Calif.); a Helios Gene Gun (Cat. #165-2431 and 165-2432; BioRad, U.S.A.); or an Accell Gene Gun (Auragen, U.S.A.). Methods for the transformation using biolistic methods are well known in the art (for example, as described in Christou, Trends in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (for example, as described in Duan et al., Nature Biotech. 14:494-498, 1996; and Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, and the glass bead agitation method.

The basic techniques used for transformation and expression in photosynthetic microorganisms are similar to those commonly used for E. coli, Saccharomyces cerevisiae and other species. Transformation methods customized for a photosynthetic microorganisms, e.g., the chloroplast of a strain of algae, are known in the art. These methods have been described in a number of texts for standard molecular biological manipulation (see Packer & Glaser, 1988, “Cyanobacteria”, Meth. Enzymol., Vol. 167; Weissbach & Weissbach, 1988, “Methods for plant molecular biology,” Academic Press, New York, Sambrook, Fritsch & Maniatis, 1989, “Molecular Cloning: A laboratory manual,” 2nd edition Cold Spring Harbor Laboratory Press, Cold Spring Harbor, N.Y.; and Clark M S, 1997, Plant Molecular Biology, Springer, N.Y.). These methods include, for example, biolistic devices (see, for example, Sanford, Trends In Biotech. (1988) δ: 299-302, U.S. Pat. No. 4,945,050; electroporation (Fromm et al., Proc. Nat'l. Acad. Set. (USA) (1985) 82: 5824-5828); use of a laser beam, electroporation, microinjection or any other method capable of introducing DNA into a host cell.

Plastid transformation is a routine and well known method for introducing a polynucleotide into a plant: cell chloroplast (see U.S. Pat. Nos. 5,451,513, 5,545,817, and 5,545,818; WO 95/16783; McBride et al., Proc. Natl. Acad. Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome. In some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation. (Svab et al., Proc. Natl. Acad. Sci., USA 87:8526-8530, 1990), and can result in stable homoplasmic transformants, at a frequency of approximately one per 100 bombardments of target leaves.

A further refinement in chloroplast transformation/expression technology that facilitates control over the timing and tissue pattern, of expression of introduced DNA coding sequences in plant plastid genomes has been described in PCT International Publication WO 95/16783 and U.S. Pat. No. 5,576,198. This method involves the introduction into plant cells of constructs for nuclear transformation that provide for the expression of a viral single subunit RNA polymerase and targeting of this polymerase into the plastids via fusion to a plastid transit peptide. Transformation of plastids with DNA constructs comprising a viral single subunit RNA polymerase-specific promoter specific to the RNA polymerase expressed from the nuclear expression constructs operably linked to DNA coding sequences of interest permits control of the plastid expression constructs in a tissue and/or developmental specific manner in plants comprising both the nuclear polymerase construct and the plastid expression constructs. Expression of the nuclear RNA polymerase coding sequence can be placed under the control of either a constitutive promoter, or a tissue- or developmental stage-specific promoter, thereby extending this control to the plastid expression construct responsive to the plastid-targeted, nuclear-encoded viral RNA polymerase.

When nuclear transformation is utilized, the protein can be modified for plastid targeting by employing plant cell nuclear transformation constructs wherein DNA coding sequences of interest are fused to any of the available transit peptide sequences capable of facilitating transport of the encoded enzymes into plant plastids, and driving expression by employing an appropriate promoter. Targeting of the protein can be achieved by fusing DNA encoding plastid, e.g., chloroplast, leucoplast, amyloplast, etc., transit peptide sequences to the 5′ end of DNAs encoding the enzymes. The sequences that encode a transit peptide region can be obtained, for example, from plant nuclear-encoded plastid proteins, such as the small subunit (SSU) of ribulose bisphosphate carboxylase, EPSP synthase, plant fatty acid biosynthesis related genes including fatty acyl-ACP thioesterases, acyl carrier protein (ACP), stearoyl-ACP desaturase, β-ketoacyl-ACP synthase and acyl-ACP thioesterase, or LHCPII genes, etc. Plastid transit peptide sequences can also be obtained from nucleic acid sequences encoding carotenoid biosynthetic enzymes, such as GGPP synthase, phytoene synthase, and phytoene desaturase. Other transit peptide sequences are disclosed in Von Heijne et al. (1991) Plant Mol. Biol. Rep. 9: 104; Clark et al. (1989) J. Biol. Chem. 264: 17544; della-Cioppa et al. (1987) Plant Physiol. 84: 965; Romer et al. (1993) Biochem. Biophys. Res. Commun. 196: 1414; and Shah et al. (1986) Science 233: 478. Another transit peptide sequence is that of the intact ACCase from Chlamydomonas (genbank EDO96563, amino acids 1-33). The encoding sequence for a transit peptide effective in transport to plastids can include all or a portion of the encoding sequence for a particular transit peptide, and may also contain portions of the mature protein encoding sequence associated with a particular transit peptide. Numerous examples of transit peptides that can be used to deliver target proteins into plastids exist, and the particular transit peptide encoding sequences useful in the present disclosure are not critical as long as delivery into a plastid is obtained. Proteolytic processing within the plastid then produces the mature enzyme. This technique has proven successful with enzymes involved in polyhydroxyalkanoate biosynthesis (Nawrath et al. (1994) Proc. Natl. Acad. Sci. USA 91: 12760), and neomycin phosphotransferase II (NPT-II) and CP4 EPSPS (Padgette et al. (1995) Crop Sci. 35: 1451), for example.

Of interest are transit peptide sequences derived from enzymes known to be imported into the leucoplasts of seeds. Examples of enzymes containing useful transit peptides include those related to lipid biosynthesis (e.g., subunits of the plastid-targeted dicot acetyl-CoA carboxylase, biotin carboxylase, biotin carboxyl carrier protein, α-carboxy-transferase, and plastid-targeted monocot multifunctional acetyl-CoA carboxylase (Mw, 220,000); plastidic subunits of the fatty acid synthase complex (e.g., acyl carrier protein (ACP), malonyl-ACP synthase, KASI, KASII and KASIII); steroyl-ACP desaturase; thioesterases (specific for short, medium, and long chain acyl ACP); plastid-targeted acyl transferases (e.g., glycerol-3-phosphate and acyl transferase); enzymes involved in the biosynthesis of aspartate family amino acids; phytoene synthase; gibberellic acid biosynthesis (e.g., ent-kaurene synthases 1 and 2); and carotenoid biosynthesis (e.g., lycopene synthase).

In some embodiments, an alga is transformed with a nucleic acid which encodes a protein of interest, for example, a prenyl transferase, an isoprenoid synthase, or an enzyme capable of converting a precursor into a fuel product or a precursor of a fuel product (e.g., an isoprenoid or fatty acid).

In one embodiment, a transformation may introduce a nucleic acid into a plastid of the host alga (e.g., chloroplast). In another embodiments a transformation may introduce a nucleic acid into the nuclear genome of the host alga. In still another embodiment, a transformation may introduce nucleic acids into both the nuclear genome and into a plastid.

Transformed cells can be plated on selective media following introduction of exogenous nucleic acids. This method may also comprise several steps for screening. A screen of primary transformants can be conducted to determine which clones have proper insertion of the exogenous nucleic acids. Clones which show the proper integration may be propagated and re-screened to ensure genetic stability. Such methodology ensures that the transformants contain the genes of interest. In many instances, such screening is performed by polymerase chain reaction (PCR); however, any other appropriate technique known in the art may be utilized. Many different methods of PCR are known in the art (e.g., nested PCR, real time PCR). For any given screen, one of skill in the art will recognize that PCR components may be varied to achieve optimal screening results. For example, magnesium concentration may need to be adjusted upwards when PCR is performed on disrupted alga cells to which (which chelates magnesium) is added to chelate toxic metals. Following the screening for clones with the proper integration of exogenous nucleic acids, clones can be screened for the presence of the encoded protein(s) and/or products. Protein expression screening can be performed by Western blot analysis and/or enzyme activity assays. Transporter and/or product screening may be performed by any method known in the art, for example ATP turnover assay, substrate transport assay, HPLC or gas chromatography.

The expression of the protein or enzyme can be accomplished by inserting a polynucleotide sequence (gene) encoding the protein or enzyme into the chloroplast or nuclear genome of a microalgae. The modified strain of microalgae can be made homoplasmic to ensure that the polynucleotide will be stably maintained in the chloroplast genome of all descendents. A microalga is homoplasmic for a gene when the inserted gene is present: in all copies of the chloroplast genome, for example. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit: expression levels that can readily exceed 10% or more of the total soluble plant protein. The process of determining the plasmic state of an organism of the present disclosure involves screening transformants for the presence of exogenous nucleic acids and the absence of wild-type nucleic acids at a given, locus of interest.

Vectors

Construct, vector and plasmid are used interchangeably throughout the disclosure. Nucleic acids encoding the proteins described herein., can be contained in vectors, including cloning and expression vectors. A cloning vector is a self-replicating DNA molecule that serves to transfer a DNA segment into a host cell. Three common types of cloning vectors are bacterial plasmids, phages, and other viruses. An expression vector is a cloning vector designed so that a coding sequence inserted at a particular site will be transcribed and translated into a protein. Both cloning and expression vectors can contain nucleotide sequences that allow the vectors to replicate in one or snore suitable host cells. In cloning vectors, this sequence is generally one that enables the vector to replicate independently of the host cell chromosomes, and also includes either origins of replication or autonomously replicating sequences.

In some embodiments, a polynucleotide of the present disclosure is cloned or inserted into an expression vector using cloning techniques know to one of skill in the art. The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2nd Ed., Cold Spring Harbor Press, (1989) and Ausubel et al., Short Protocols in Molecular Biology, 2nd Ed., John Wiley & Sons (1992).

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, and herpes simplex virus), PI-based artificial chromosomes, yeast plasmids, yeast artificial chromosomes, and any other vectors specific for specific hosts of interest (such as E. coli and yeast). Thus, for example, a polynucleotide encoding an FPP synthase, can be inserted into any one of a variety of expression vectors that are capable of expressing the enzyme. Such vectors can include, for example, chromosomal, nonchromosomal and synthetic DNA sequences.

Suitable expression vectors include chromosomal, non-chromosomal and synthetic DNA sequences, for example, SV 40 derivatives; bacterial plasmids; phage DNA; baculovirus; yeast plasmids; vectors derived from combinations of plasmids and phage DNA; and viral DNA such as vaccinia, adenovirus, fowl pox virus, and pseudorabies. In addition, any other vector that is replicable and viable in the host may be used. For example, vectors such as Ble2A, Arg7/2A, and SEnuc357 can be used for the expression of a protein.

Numerous suitable expression vectors are known to those of skill in the art. The following vectors are provided by way of example; for bacterial host cells: pQE vectors (Qiagen), pBluescript plasmids, pNH vectors, lambda-ZAP vectors (Stratagene), pTrc99a, pKK223-3, pDR540, and pRIT2T (Pharmacia); for eukaryotic host cells: pXT1, pSG5 (Stratagene), pSVK3, pBPV, pMSG, pET21a-d(+) vectors (Novagen), and pSVLSV40 (Pharmacia). However, any other plasmid or other vector may be used so long as it is compatible with the host cell.

The expression vector, or a linearized portion thereof, can encode one or more exogenous or endogenous nucleotide sequences. Examples of exogenous nucleotide sequences that can be transformed into a host include genes from bacteria, fungi, plants, photosynthetic bacteria or other algae. Examples of other types of nucleotide sequences that can be transformed into a host, include, but are not limited to, transporter genes, isoprenoid producing genes, genes which encode for proteins which produce isoprenoids with two phosphates (e.g., GPP synthase and/or FPP synthase), genes which encode for proteins which produce fatty acids, lipids, or triglycerides, for example, ACCases, endogenous promoters, and 5′ UTRs from the psbA, atpA, or rbcL genes. In some instances, an exogenous sequence is flanked by two homologous sequences.

Homologous sequences are, for example, those that have at least 50%, at least 60%, at least 70%, at least 80%, at least 90%, at least 95%, at least 98%, or at least at least 99% sequence identity to a reference amino acid sequence, for example, the amino acid sequence found naturally in the host cell. The first and second sequences enable recombination of the exogenous or endogenous sequence into the genome of the host organism. The first and second homologous sequences can be at least 300, at least 200, at least 300, at least 400, at least 500, or at least 1000, or at least 1500 nucleotides in length.

The polynucleotide sequence may comprise nucleotide sequences that are codon biased for expression in the organism being transformed. The skilled artisan is well aware of the “codon-bias” exhibited by a specific host cell in usage of nucleotide codons to specify a given amino acid. Without being bound by theory, by using a host cell's preferred codons, the rate of translation may be greater. Therefore, when synthesizing a gene for improved expression in a host cell, it may be desirable to design the gene such that its frequency of codon usage approaches the frequency of preferred codon usage of the host cell. In some organisms, codon bias differs between the nuclear genome and organelle genomes, thus, codon optimization or biasing may be performed for the target genome (e.g., nuclear codon biased or chloroplast codon biased). In some embodiments, codon biasing occurs before mutagenesis to generate a polypeptide. In other embodiments, codon biasing occurs after mutagenesis to generate a polynucleotide. In yet other embodiments, codon biasing occurs before mutagenesis as well as after mutagenesis. Codon bias is described in detail herein.

In some embodiments, a vector comprises a polynucleotide operably linked to one or more control elements, such as a promoter and/or a transcription terminator. A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading phase. Linking is achieved by ligation at restriction enzyme sites. If suitable restriction sites are not available, then synthetic oligonucleotide adapters or linkers can be used as is known to those skilled in the art. Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

A vector in some embodiments provides for amplification of the copy number of a polynucleotide. A vector can be, for example, an expression vector that provides for expression of an ACCase, a prenyl transferase, an isoprenoid synthase, or a mevalonate synthesis enzyme in a host cell, e.g., a prokaryotic host cell or a eukaryotic host cell.

A polynucleotide or polynucleotides can be contained in a vector or vectors. For example, where a second (or more) nucleic acid molecule is desired, the second nucleic acid molecule can be contained in a vector, which can, but need not be, the same vector as that containing the first nucleic acid molecule. For example, an algal host cell modified to express two endogenous or exogenous genes may be transformed with a single vector containing both sequences, or two vectors, each, comprising one gene to be expressed. The vector can be any vector useful for introducing a polynucleotide into a genome and can include a nucleotide sequence of genomic DNA (e.g., nuclear or plastid) that is sufficient to undergo homologous recombination with genomic DNA, for example, a nucleotide sequence comprising about 400 to about 1500 or more substantially contiguous nucleotides of genomic DNA.

A regulatory or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, and an IRES. A regulatory element can include a promoter and transcriptional and translational stop signals. Elements may be provided with linkers for the purpose of introducing specific restriction sites facilitating ligation of the control sequences with the coding region of a nucleotide sequence encoding a polypeptide. Additionally, a sequence comprising a cell compartmentalization signal (i.e., a sequence that targets a polypeptide to the cytosol, nucleus, chloroplast membrane or cell membrane) can be attached to the polynucleotide encoding a protein of interest. Such signals are well known in the art and have been widely reported (see, e.g., U.S. Pat. No. 5,776,689).

Promoters are untranslated sequences located generally 100 to 1000 base pairs (bp) upstream from the start codon of a structural gene that regulate the transcription and translation of nucleic acid sequences under their control.

Promoters useful for the present disclosure may come from any source (e.g., viral, bacterial, fungal, protist, and animal). The promoters contemplated herein can be specific to photosynthetic organisms, non-vascular photosynthetic organisms, and vascular photosynthetic organisms (e.g., algae, flowering plants). In some instances, the nucleic acids above are inserted into a vector that comprises a promoter of a photosynthetic organism, e.g., algae. The promoter can be a constitutive promoter or an inducible promoter. A promoter typically includes necessary nucleic acid sequences near the start site of transcription, (e.g., a TATA element). Common promoters used in expression vectors include, but are not limited to, LTR or SV40 promoter, the E. coli lac or trp promoters, and the phage lambda PL promoter. Other promoters known to control the expression of genes in prokaryotic or eukaryotic cells can be used and are known to those skilled in the art. Expression vectors may also contain a ribosome binding site for translation initiation, and a transcription terminator. The vector may also contain sequences useful for the amplification of gene expression.

A “constitutive” promoter is a promoter that is active under most environmental and developmental conditions. An “inducible” promoter is a promoter that is active under controllable environmental or developmental conditions. Examples of inducible promoters/regulatory elements include, for example, a nitrate-inducible promoter (for example, as described in Bock et al, Plant Mol. Biol. 17:9 (1991)), or a light-inducible promoter, (for example, as described in Feinbaum et al, Mol. Gen. Genet. 226:449 (1991); and Lam and Chua, Science 248:471 (1990)), or a heat responsive promoter (for example, as described in Muller et al., Gene 111: 165-73 (1992)).

In many embodiments, a polynucleotide of the present disclosure includes a nucleotide sequence encoding a protein or enzyme of the present disclosure, where the nucleotide sequence encoding the polypeptide is operably linked to an inducible promoter. Inducible promoters are well known in the art. Suitable inducible promoters include, but are not limited to, the pL of bacteriophage λ; Placo; Ptrp; Ptac (Ptrp-lac hybrid promoter); an isopropyl-beta-D-thiogalactopyranoside (IPTG)-inducible promoter, e.g., a lacZ promoter; a tetracycline-inducible promoter; an arabinose inducible promoter, e.g., P_{BAD} (for example, as described in Guzman et al, (1995) I, Bacteriol. 177:4121-4130); a xylose-inducible promoter, e.g., Pxyl (for example, as described in Kim et al. (1996) Gene 181:71-76); a GAL1 promoter; a tryptophan promoter; a lac promoter; an alcohol-inducible promoter, e.g., a methanol-inducible promoter, an ethanol-inducible promoter; a raffinose-inducible promoter; and a heat-inducible promoter, e.g., heat inducible lambda P_{L} promoter and a promoter controlled by a heat-sensitive repressor (e.g., C1857-repressed lambda-based expression vectors; for example, as described in Hoffmann et al. (1999) FEMS Microbiol Lett. 177(2):327-34).

In many embodiments, a polynucleotide of the present disclosure includes a nucleotide sequence encoding a protein or enzyme of the present disclosure, where the nucleotide sequence encoding the polypeptide is operably linked to a constitutive promoter. Suitable constitutive promoters for use in prokaryotic cells are known in the art and include, but are not limited to, a sigma70 promoter, and a consensus sigma70 promoter.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/tac hybrid promoter, a trp/lac promoter, a T7/lac promoter; a trc promoter; a tac promoter; an araBAD promoter; in vivo regulated promoters, such as an ssaG promoter or a related promoter (for example, as described in U.S. Patent Publication No. 20040131637), a pagC promoter (for example, as described in Pulkkinen and Miller, J. Bacteriol., 1991: 173(1): 86-93; and Alpuche-Aranda et al., PNAS, 1992; 89(21): 10079-83), a nirB promoter (for example, as described in Harborne et al. (1992) Mol. Micro. 6:2805-2813; Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (for example, GenBank Accession Nos. AX798980, AX798961, and AX798183); a stationary phase promoter, e.g., a dps promoter, an spv promoter; a promoter derived from the pathogenicity island SPI-2 (for example, as described in WO96/17951); an actA promoter (for example, as described in Shetron-Rama et al. (2002) Infect. Immun. 70:1087-1096); an rpsM promoter (for example, as described in Valdivia and Falkow (1996). Mol. Microbiol. 22:367-378); a tet promoter (for example, as described in Hillen, W. and Wissmann, A. (1989) In Saenger, W. and Heinemann, U. (eds). Topics in Molecular and Structural Biology, Protein-Nucleic Acid Interaction. Macmilian, London, UK, Vol. 10, pp. 143-162); and an SP6 promoter (for example, as described in Melton et al. (1984) Nucl. Acids Res. 12:7035-7056).

In yeast, a number of vectors containing constitutive or inducible promoters may be used. For a review of such vectors see, Current Protocols in Molecular Biology, Vol. 2, 1988, Ed. Ausubel, et al., Greene Publish. Assoc, & Wiley Interscience, Ch. 13; Grant, et al., 1987, Expression and Secretion Vectors for Yeast, in Methods in Enzymology, Eds. Wu & Grossman, 31987, Acad. Press, N.Y., Vol. 153, pp. 516-544; Glover, 1986, DNA Cloning, Vol. II, IRL Press, Wash., D.C., Ch. 3; Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathen et al., Cold Spring Harbor Press, Vols, I and II. A constitutive yeast promoter such as ADH or LEU2 or an inducible promoter such as GAL may be used (for example, as described in Cloning in Yeast, Ch. 3, R. Rothstein In: DNA Cloning Vol. 11, A Practical Approach, Ed. D M Glover, 1986, IRL Press, Wash., D.C.). Alternatively, vectors may be used which promote integration of foreign DNA sequences into the yeast chromosome.

Non-limiting examples of suitable eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

A vector utilized in the practice of the disclosure also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, and sequences that encode a selectable marker. As such, the vector can contain., for example, one or more cloning sites such as a multiple cloning site, which can, but need not, be positioned such that a exogenous or endogenous polynucleotide can be inserted into the vector and operatively linked to a desired element.

The vector also can contain a prokaryote origin of replication (ori), for example, an E. coli ori or a cosmid ori, thus allowing passage of the vector into a prokaryote host cell, as well as into a plant chloroplast. Various bacterial and viral origins of replication are well known to those skilled in the art and include, but are not limited to the pBR322 plasmid origin, the 2u plasmid origin, and the SV40, polyoma, adenovirus, VSV, and BPV viral origins.

A regulatory or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, an IRES. Additionally, an element can be a cell compartmentalization signal (i.e., a sequence that targets a polypeptide to the cytosol, nucleus, chloroplast membrane or cell membrane). In some aspects of the present disclosure, a cell compartmentalization signal (e.g., a cell membrane targeting sequence) may be ligated to a gene and/or transcript, such that translation of the gene occurs in the chloroplast. In other aspects, a cell compartmentalization signal may be ligated to a gene such that, following translation of the gene, the protein is transported to the cell membrane. Cell compartmentalization signals are well known in the art and have been widely reported (see, e.g., U.S. Pat. No. 5,776,689).

A vector, or a linearized portion thereof, may include a nucleotide sequence encoding a reporter polypeptide or other selectable marker. The term “reporter” or “selectable marker” refers to a polynucleotide (or encoded polypeptide) that confers a detectable phenotype. A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation (for example, as described in Giacomin, Plant Sci. 116:59-72, 1996; Scikantha, J. Bacteriol. 378:121, 1996; Gerdes, FEBS Lett. 389:44-47, 1996; and Jefferson, EMBO J. 6:3901-3907, 1997, fl-glucuronidase). A selectable marker generally is a molecule that, when present or expressed in a cell, provides a selective advantage (or disadvantage) to the cell containing the marker, for example, the ability to grow in the presence of an agent that otherwise would kill the cell.

A selectable marker can provide a means to obtain, for example, prokaryotic cells, eukaryotic cells, and/or plant cells that express the marker and, therefore, can be useful as a component of a vector of the disclosure. The selection gene or marker can encode for a protein necessary for the survival or growth of the host cell transformed with the vector. One class of selectable markers are native or modified genes which restore a biological or physiological function to a host cell (e.g., restores photosynthetic capability or restores a metabolic pathway). Other examples of selectable markers include, but are not limited to, those that confer antimetabolite resistance, for example, dihydrofolate reductase, which confers resistance to methotrexate (for example, as described in Reiss, Plant Physiol. (Life Sci. Adv.) 13:143-149, 1994); neomycin phosphotransferase, which confers resistance to the aminoglycosides neomycin, kanamycin and paromycin (for example, as described in Herrera-Estrella, EMBO J, 2:987-995, 1983), hygro, which confers resistance to hygromycin (for example, as described in Marsh, Gene 32:481-485, 1984), trpB, which allows cells to utilize indole in place of tryptophan; hisD, which allows cells to utilize histinol in place of histidine (for example, as described in Hartman, Proc. Natl. Acad. Sci., USA 85:8047, 1988); mannose-6-phosphate isomerase which, allows cells to utilize mannose (for example, as described in PCT Publication Application No. WO 94/20627); ornithine decarboxylase, which confers resistance to the ornithine decarboxylase inhibitor, 2-(difluoromethyl)-DL-ornithine (DFMO; for example, as described in McConlogue, 1987, In: Current Communications in Molecular Biology, Cold Spring Harbor Laboratory ed.); and deaminase from Aspergillus terreus, which confers resistance to Blasticidin S (for example, as described in Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (for example, as described in White et al., Nucl. Acids Res. 18:1062, 1990; and Spencer et al., Theor. Appl. Genet. 79:625-631, 1990), a mutant EPSPV-synthase, which confers glyphosate resistance (for example, as described in Hinchee et al., BioTechnology 91:915-922, 1998), a mutant acetolactate synthase, which confers imidazolione or sulfonylurea resistance (for example, as described in Lee et al., EMBO J. 7:3241-1248, 1988), a mutant psbA, which confers resistance to atrazine (for example, as described in Smeda et al., Plant Physiol. 103:911-917, 1993), or a mutant protoporphyrinogen oxidase (for example, as described in U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells; tetramycin or ampicillin resistance for prokaryotes such as E. coli; and bleomycin, gentamycin, glyphosate, hygromycin, kanamycin, methotrexate, phleomycin, phosphinotricin, spectinomycin, dtreptomycin, streptomycin, sulfonamide and sulfonylurea resistance in plants (for example, as described in Maliga et al., Methods in Plant Molecular Biology, Cold Spring Harbor Laboratory Press, 1995, page 39). Additional selectable markers include a mutation in dichlorophenyl dimethylurea (DCMU) that results in resistance to DCMU. Selectable markers also include chloramphenicol acetyltransferase (CAT) and tetracycline. The selection marker can have its own promoter or its expression can be driven by a promoter driving the expression of a polypeptide of interest.

Reporter genes greatly enhance the ability to monitor gene expression in a number of biological organisms. Reporter genes have been successfully used in chloroplasts of higher plants, and high levels of recombinant protein expression have been reported. In addition, reporter genes have been used in the chloroplast of C. reinhardtii. In chloroplasts of higher plants, β-glucuronidase (uidA, for example, as described in Staub and Maliga, EMBO J. 12:601-606, 1993), neomycin phosphotransferase (nptII, for example, as described in Carrer et al., Mol. Gen. Genet. 241:49-56, 1993), adenosyl-3-adenyltransf-erase (aadA, for example, as described in Svab and Maliga, Proc. Natl. Acad. Sci., USA 90:913-917, 1993), and the Aequorea victoria GFP (for example, as described in Sidorov et al. Plant J. 19:209-216, 1999) have been used as reporter genes (for example, as described in Heifetz, Biochemie 82:655-666, 2000). Each of these genes has attributes that make them useful reporters of chloroplast gene expression, such as ease of analysis, sensitivity, or the ability to examine expression in situ. Based upon these studies, other exogenous proteins have been expressed in the chloroplasts of higher plants such as Bacillus thuringiensis Cry toxins, conferring resistance to insect herbivores (for example, as described in Kota et al., Proc. Natl. Acad. Sci., USA 96:1840-1845, 1999), or human somatotropin (for example, as described in Staub et al., Nat. Biotechnol. 18:333-338, 2000), a potential biopharmaceutical. Several reporter genes have been expressed in the chloroplast of the eukaryotic green alga, C. reinhardtii, including aadA (for example, as described in Goldschmidt-Clermont, Nucl. Acids Res. 19:4083-4089 1991; and Zerges and Rochaix, Mol. Cell. Biol. 14:5268-5277, 1994), uidA (for example, as described in Sakamoto et al., Proc. Natl. Acad. Sci., USA 90:477-501, 1993; and Ishikura et al., J. Biosci. Bioeng. 87:307-314 1999), Renilla luciferase (for example, as described in Minko et al., Mol. Gen. Genet. 262:421-425, 1999) and the amino glycoside phosphotransferase from Acinetobacter baumanii, aphA6 (for example, as described in Bateman and Purton, Mol. Gen. Genet. 263:404-410, 2000). In one embodiment the protein described herein is modified by the addition of an N-terminal strep tag epitope to add in the detection of protein expression.

In some instances, the vectors of the present disclosure will contain elements such as an E. coli or S. cerevisiae origin of replication. Such features, combined with appropriate selectable markers, allows for the vector to be “shuttled” between the target host cell and a bacterial and/or yeast cell. The ability to passage a shuttle vector of the disclosure in a secondary host may allow for more convenient manipulation of the features of the vector. For example, a reaction mixture containing the vector and inserted polynucleotide(s) of interest can be transformed into prokaryote host cells such as E. coli, amplified and collected using routine methods, and examined to identify vectors containing an insert or construct of interest. If desired, the vector can be further manipulated, for example, by performing site directed mutagenesis of the inserted polynucleotide, then again amplifying and selecting vectors having a mutated polynucleotide of interest. A shuttle vector then can be introduced into plant cell chloroplasts, wherein a polypeptide of interest can be expressed and, if desired, isolated according to a method of the disclosure.

Knowledge of the chloroplast or nuclear genome of the host organism, for example, C. reinhardtii, is useful in the construction of vectors for use in the disclosed embodiments. Chloroplast vectors and methods for selecting regions of a chloroplast genome for use as a vector are well known (see, for example, Bock, J. Mol. Biol. 312:425-438, 2001; Staub and Maliga, Plant Cell 4:39-45, 1992; and Kavanagh et al., Genetics 152:1111-1122, 1999, each of which is incorporated herein by reference). The entire chloroplast genome of C. reinhardtii is available to the public on the world wide web, at the URL “biology.duke.edu/chlamy_genome/-chloro.html” (see “view complete genome as text file” link and “maps of the chloroplast genome” link; J. Maid, J. W. Lilly, and D. B. Stern, unpublished results; revised Jan. 28, 2002; to be published as GenBank Acc. No. AF396929; and Maul, J. E., et al. (2002) The Plant Cell, Vol. 14 (2659-2679)). Generally, the nucleotide sequence of the chloroplast genomic DNA that is selected for use is not a portion of a gene, including a regulatory sequence or coding sequence. For example, the selected sequence is not a gene that if disrupted, due to the homologous recombination event, would produce a deleterious effect with respect to the chloroplast. For example, a deleterious effect on the replication of the chloroplast genome or to a plant cell containing the chloroplast. In this respect, the website containing the C. reinhardtii chloroplast genome sequence also provides maps showing coding and non-coding regions of the chloroplast genome, thus facilitating selection of a sequence useful for constructing a vector (also described in Maid, J. E., et al. (2002) The Plant Cell, Vol. 14 (2659-2679)). For example, the chloroplast vector, p322, is a clone extending from the Eco (Eco RT) site at about position 143.1 kb to the Xho (Xho I) site at about position 148.5 kb (see, world wide web, at the URL “biology.duke.edu/chlamy_genome/chloro.html”, and clicking on “maps of the chloroplast genome” link, and “140-150 kb” link; also accessible directly on world wide web at URL “biology.duke.edu/chlam-y/chloro/chloro40.html”).

In addition, the entire nuclear genome of C. reinhardtii is described in Merchant, S. S., et al., Science (2007), 318(5848):245-250, thus facilitating one of skill in the art to select a sequence or sequences useful for constructing a vector.

For expression of the polypeptide in a host, an expression cassette or vector may be employed. The expression vector will provide a transcriptional and translational initiation region, which may be inducible or constitutive, where the coding region is operably linked under the transcriptional control of the transcriptional initiation region, and a transcriptional and translational termination, region. These control regions may be native to the gene, or may be derived from an exogenous source. Expression vectors generally have convenient restriction sites located near the promoter sequence to provide for the insertion of nucleic acid sequences encoding exogenous or endogenous proteins. A selectable marker operative in the expression host may be present.

The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2^{nd} Ed., Cold Spring Harbor Press, (1989) and Ausubel et al., Short Protocols in Molecular Biology, 2nd Ed., John Wiley & Sons (1992).

The description herein provides that host cells may be transformed with vectors. One of skill in the art will recognize that such transformation includes transformation with circular or linearized vectors, or linearized portions of a vector. Thus, a host cell comprising a vector may contain the entire vector in the cell (in either circular or linear form), or may contain a linearized portion of a vector of the present disclosure. In some instances 0.5 to 1.5 kb flanking nucleotide sequences of chloroplast genomic DNA may be used. In some instances 0.5 to 1.5 kb flanking nucleotide sequences of nuclear genomic DNA may be used, or 2.0 to 5.0 kb may be used.

Compounds

The modified or transformed host organism disclosed herein is useful in the production of a desired compound, composition, or product. The present disclosure provides methods of producing, for example, an isoprenoid or isoprenoid precursor compound in a host cell. One such method involves, culturing a modified host cell in a suitable culture medium under conditions that promote synthesis of a product, for example, an isoprenoid compound or isoprenoid precursor compound, where the isoprenoid compound is generated by the expression of an enzyme of the present disclosure, wherein the enzyme uses a substrate present in the host cell. In some embodiments, a method further comprises isolating the isoprenoid compound from the cell and/or from the culture medium.

In some embodiments, the product (e.g. fuel molecule) is collected by harvesting the liquid medium. As some fuel molecules (e.g., monoterpenes) are immiscible in water, they would float to the surface of the liquid medium and could be extracted easily, for example by skimming. In other instances, the fuel molecules can be extracted from the liquid medium. In still other instances, the fuel molecules are volatile. In such instances, impermeable barriers can cover or otherwise surround the growth environment and can be extracted from the air within the barrier. For some fuel molecules, the product may be extracted from both the environment (e.g., liquid environment and/or air) and from the intact host cells. Typically, the organism would be harvested at an appropriate point and the product may then be extracted from the organism. The collection of cells may be by any means known in the art, including, but not limited to concentrating cells, mechanical or chemical disruption of cells, and purification of product(s) from cell cultures and/or cell lysates. Cells and/or organisms can be grown and then the product(s) collected by any means known to one of skill in the art. One method of extracting the product is by harvesting the host cell or a group of host cells and then drying the cell(s). The product(s) from the dried host cell(s) are then, harvested by crushing the cells to expose the product. In some instances, the product may be produced without killing the organisms. Producing and/or expressing the product may not render the organism unviable.

In some embodiments, a genetically modified host cell is cultured in a suitable medium (e.g., Luria-Bertoni broth, optionally supplemented with one or more additional agents, such as an inducer (e.g., where the isoprenoid synthase is under the control of an inducible promoter); and the culture medium is overlaid with an organic solvent, e.g. dodecane, forming an organic layer. The compound produced by the genetically modified host partitions into the organic layer, from which it can then be purified. In some embodiments, where, for example, a prenyl transferase, isoprenoid synthase or mevalonate synthesis-encoding nucleotide sequence is operably linked to an inducible promoter, an inducer is added to the culture medium; and, after a suitable time, the compound is isolated from the organic layer overlaid on the culture medium.

In some embodiments, the compound or product, for example, an isoprenoid compound will be separated from other products which may be present in the organic layer. Separation of the compound from other products that may be present in the organic layer is readily achieved using, e.g., standard chromatographic techniques.

Methods of culturing the host cells, separating products, and isolating the desired product or products are known to one of skill in the art and are discussed further herein.

In some embodiments, the compound, for example, an isoprenoid or isoprenoid compound is produced in a genetically modified host cell at a level that is at least about 2-fold, at least about 5-fold, at least about 10-fold, at least about 25-fold, at least about 50-fold, at least about 100-fold, at least about 500-fold, at least about 1000-fold, at least about 2000-fold, at least about 3000-fold, at least about 4000-fold, at least about 5000-fold, or at least about 10,000-fold, or more, higher than the level of the isoprenoid or isoprenoid precursor compound produced in an unmodified host cell that produces the isoprenoid or isoprenoid precursor compound via the same biosynthetic pathway.

In some embodiments, the compound, for example, an isoprenoid compound is pure, e.g., at least about 40% pure, at least about 50% pure, at least about 60% pure, at least about 70% pure, at least about 80% pure, at least about 90% pure, at least about 95% pure, at least about 98%, or more than 98% pure. “Pure” in the context of an isoprenoid compound refers to an isoprenoid compound that is free from other isoprenoid compounds, portions of compounds, contaminants, and unwanted byproducts, for example.

Examples of products contemplated herein include hydrocarbon products and hydrocarbon derivative products. A hydrocarbon product is one that consists of only hydrogen molecules and carbon molecules. A hydrocarbon derivative product is a hydrocarbon product with one or more heteroatoms, wherein the heteroatom is any atom that is not hydrogen or carbon. Examples of heteroatoms include, but are not limited to, nitrogen, oxygen, sulfur, and phosphorus. Some products can be hydrocarbon-rich, wherein, for example, at least 50%, at least 60%, at least 70%, at least 80%, at least 90%, or at least 95% of the product by weight is made tip of carbon and hydrogen.

In one embodiment, the vector comprises one or more nucleic acid sequences involved in isoprenoid synthesis. The terms “isoprenoid,” “isoprenoid compound,” “terpene,” “terpene compound,” “terpenoid,” and “terpenoid compound” are used interchangeably herein. Isoprenoid compounds include, but are not limited to, monoterpenes, sesquiterpenes, diterpenes, triterpenes, and polyterpenes.

One exemplary group of hydrocarbon products are isoprenoids. Isoprenoids (including terpenoids) are derived from isoprene subunits, but are modified, for example, by the addition of heteroatoms such as oxygen, by carbon skeleton rearrangement, and by alkylation. Isoprenoids generally have a number of carbon atoms which is evenly divisible by five, but this is not a requirement as “irregular” terpenoids are known to one of skill in the art. Carotenoids, such as carotenes and xanthophylls, are examples of isoprenoids that are useful products. A steroid is an example of a terpenoid. Examples of isoprenoids include, but are not limited to, hemiterpenes (C5), monoterpenes (C10), sesquiterpenes (C15), diterpenes (C20), triterpenes (C30), tetraterpenes (C40), polyterpenes (Cₙ, wherein “n” is equal to or greater than 45), and their derivatives. Other examples of isoprenoids include, but are not limited to, limonene, 1,8-cineole, α-pinene, camphene, (+)-sabinene, myrcene, abietadiene, taxadiene, farnesyl pyrophosphate, fusicoccadiene, amorphadiene, (E)-α-bisabolene, zingiberene, or diapophytoene, and their derivatives.

Products, for example fuel products, comprising hydrocarbons, may be precursors or products conventionally derived from crude oil, or petroleum, such as, but not limited to, liquid petroleum gas, naptha (ligroin), gasoline, kerosene, diesel, lubricating oil, heavy gas, coke, asphalt, tar, and waxes.

Useful products include, but are not limited to, terpenes and terpenoids as described above. An exemplary group of terpenes are diterpenes (C20). Diterpenes are hydrocarbons that can be modified (e.g. oxidized, methyl groups removed, or cyclized); the carbon skeleton of a diterpene can be rearranged, to form, for example, terpenoids, such as fusicoccadiene, Fusicoccadiene may also be formed, for example, directly from the isoprene precursors, without being bound by the availability of diterpene or GGDP. Genetic modification of organisms, such as algae, by the methods described herein, can lead to the production of fusicoccadiene, for example, and other types of terpenes, such as limonene, for example. Genetic modification can also lead to the production of modified terpenes, such as methyl squalene or hydroxylated and/or conjugated terpenes such as paclitaxel.

Other useful products can be, for example, a product comprising a hydrocarbon obtained from an organism expressing a diterpene synthase. Such exemplary products include ent-kaurene, casbene, and fusicoccadiene, and may also include fuel additives.

In some embodiments, a product (such as a fuel product) contemplated herein comprises one or more carbons derived from an inorganic carbon source. In some embodiments, at least 10%, at least 20%, at least 30%, at least 40%, at least 50%, at least 60%, at least 70%, at least 80%, at least 90%, at least 95%, or at least 99% of the carbons of a product as described herein are derived from an inorganic carbon source. Examples of inorganic carbon sources include, but are not limited to, carbon dioxide, carbonate, bicarbonate, and carbonic acid. The product can be, for example, an organic molecule with carbons from an inorganic carbon source that were fixed during photosynthesis.

The products produced by the present disclosure may be naturally, or non-naturally (e.g., as a result of transformation) produced by the host cell(s) and/or organism(s) transformed. For example, products not naturally produced by algae may include non-native terpenes/terpenoids such as fusicoccadiene or limonene. A product naturally produced in algae may be a terpene such as a carotenoid (for example, beta-carotene). The host cell may be genetically modified, for example, by transformation of the cell with a sequence encoding a protein, wherein expression of the protein results in the secretion of a naturally or a non-naturally produced product (e.g. limonene) or products. The product may be a molecule not found in nature.

Examples of products include petrochemical products, precursors of petrochemical products, fuel products, petroleum products, precursors of petroleum products, and all other substances that may be useful in the petrochemical industry. The product may be used for generating substances, or materials, useful in the petrochemical industry. The products may be used in a combustor such, as a boiler, kiln, dryer or furnace. Other examples of combustors are internal combustion, engines such, as vehicle engines or generators, including gasoline engines, diesel engines, jet engines, and other types of engines. In one embodiment, a method herein comprises combusting a refined or “upgraded” composition. For example, combusting a refined composition can comprise inserting the refined composition into a combustion, engine, such as an automobile engine or a jet engine. Products described herein may also be used to produce plastics, resins, fibers, elastomers, pharmaceuticals, neutraceuticals, lubricants, and gels, for example.

Useful products can also include isoprenoid precursors. Isoprenoid precursors are generated by one of two pathways; the mevalonate pathway or the methylerythritol phosphate (MEP) pathway. Both pathways generate dimethylallyl pyrophosphate (DMAPP) and isopentyl pyrophosphate (IPP), the common C5 precursor for isoprenoids. The DMAPP and IPP are condensed to form geranyl-diphosphate (GPP), or other precursors, such as farnesyl-diphosphate (FPP) or geranylgeranyl-diphosphate (GGPP), from which higher isoprenoids are formed.

Useful products can also include small alkanes (for example, 1 to approximately 4 carbons) such as methane, ethane, propane, or butane, which may be used for heating (such as in cooking) or making plastics. Products may also include molecules with a carbon backbone of approximately 5 to approximately 9 carbon atoms, such as naptha or ligroin, or their precursors. Other products may include molecules with a carbon background of about 5 to about 12 carbon atoms, or cycloalkanes used as gasoline or motor fuel. Molecules and aromatics of approximately 10 to approximately 18 carbons, such as kerosene, or its precursors, may also be useful as products. Other products include lubricating oil, heavy gas oil, or fuel oil, or their precursors, and can contain alkanes, cycloalkanes, or aromatics of approximately 12 to approximately 70 carbons. Products also include other residuals that can be derived from or found in crude oil, such as coke, asphalt, far, and waxes, generally containing multiple rings with about 70 or more carbons, and their precursors.

Examples of products, which can include the isoprenoids of the present disclosure, are fuel products, fragrance products, and insecticide products. In some instances, a product may be used directly. In other instances, the product may be used as a “feedstock” to produce another product. For example, where the product is an isoprenoid, the isoprenoid may be hydrogenated and “cracked” to produce a shorter chain hydrocarbon (e.g., farnesene is hydrogenated to produce farnesene which is then cracked to produce propane, butane, octane, or other fuel products).

Modified organisms can be grown, in some embodiments in the presence of CO₂, to produce a desired polypeptide. In some embodiments, the products produced by the modified organism are isolated or collected. Collected products, such as terpenes and terpenoids, may then be further modified, for example, by refining and/or cracking to produce fuel molecules or components.

The various products may be further refined to a final product for an end user by a number of processes. Refining can, for example, occur by fractional distillation. For example, a mixture of products, such as a mix of different hydrocarbons with various chain lengths may be separated into various components by fractional distillation.

Refining may also include any one or more of the following steps, cracking, unifying, or altering the product. Large products, such as large hydrocarbons (e.g. C10), may be broken down into smaller fragments by cracking. Cracking may be performed by heat or high pressure, such as by steam, visbreaking, or coking. Products may also be refined by visbreaking, for example by thermally cracking large hydrocarbon molecules in the product by heating the product in a furnace. Refining may also include coking, wherein a heavy, almost pure carbon residue is produced. Cracking may also be performed by catalytic means to enhance the rate of the cracking reaction, by using catalysts such as, but not limited to, zeolite, aluminum hydrosilicate, bauxite, or silica-alumina. Catalysis may be by fluid catalytic cracking, whereby a hot catalyst, such, as zeolite, is used to catalyze cracking reactions. Catalysis may also be performed by hydrocracking, where lower temperatures are generally used in comparison, to fluid catalytic cracking. Hydrocracking can occur in the presence of elevated partial pressure of hydrogen gas. Products may be refined by catalytic cracking to generate diesel, gasoline, and/or kerosene.

The products may also be refined by combining them in a unification step, for example by using catalysts, such as platinum or a platinum-rhenium mix. The unification process can produce hydrogen gas, a by-product, which may be used in cracking.

The products may also be refined by altering, rearranging, or restructuring hydrocarbons into smaller molecules. There are a number of chemical reactions that occur in catalytic reforming processes which are known to one of ordinary skill in the arts. Catalytic reforming can be performed in the presence of a catalyst and a high partial pressure of hydrogen. One common process is alkylation. For example, propylene and butylene are mixed with a catalyst such as hydrofluoric acid or sulfuric acid, and the resulting products are high octane hydrocarbons, which can be used to reduce knocking in gasoline blends.

The products may also be blended or combined into mixtures to obtain an end product. For example, the products may be blended to form gasoline of various grades, gasoline with or without additives, lubricating oils of various weights and grades, kerosene of various grades, jet fuel, diesel fuel, heating oil, and chemicals for making plastics and other polymers. Compositions of the products described herein may be combined or blended with fuel products produced by other means.

Some products produced from the host cells of the disclosure, especially after refining, will be identical to existing petrochemicals, i.e. contain the same chemical structure. For instance, crude oil contains the isoprenoid pristane, which is thought to be a breakdown product of phytol, which is a component of chlorophyll. Some of the products may not be the same as existing petrochemicals. However, although a molecule may not exist in conventional petrochemicals or refining, it may still be useful in these industries. For example, a hydrocarbon could be produced that is in the boiling point range of gasoline, and that could be used as gasoline or an additive, even though the hydrocarbon does not normally occur in gasoline.

A product herein can be described by its Carbon Isotope Distribution (CID). At the molecular level, a CID is the statistical likelihood of a single carbon atom within a molecule to be one of the naturally occurring carbon isotopes (for example, ¹²C, ¹³C, or ¹⁴C). At the bulk level of a product, a CID may be the relative abundance of naturally occurring carbon isotopes (for example, ¹²C, , ¹³C, or ¹⁴C) in a compound containing at least one carbon atom. It is noted that the CID of a fossil fuel may differ based on its source. For example, with CID(fos), the CID of carbon in a fossil fuel, such as petroleum, natural gas, and coal is distinguishable from the CID(atm), the CID of carbon in current atmospheric carbon dioxide. Additionally, the CID(photo-atm) refers to the CID of a carbon-based compound made by photosynthesis in recent history where the source of inorganic carbon was carbon dioxide in the atmosphere. Also, CID(photo-fos) refers to the CID of a carbon based compound made by photosynthesis in recent history where the source of substantially all of the inorganic carbon was carbon dioxide produced by the burning of fossil fuels (for example, coal, natural gas, and/or petroleum). The exact distribution is also a characteristic of 1) the type of photosynthetic organism that produced the molecule, and 2) the source of inorganic carbon. These isotope distributions can be used to define the composition of photosynthetically-derived fuel products. Carbon isotopes are unevenly distributed among and within different, compounds and the isotopic distribution, can reveal information about the physical, chemical, and metabolic processes involved in carbon transformation. The overall abundance of ¹³C relative to ¹²C in a photosynthetic organism is often less than the overall abundance of ¹³C relative to ¹²C in atmospheric carbon dioxide, indicating that carbon isotope discrimation occurs in the incorporation of carbon dioxide into photosynthetic biomass.

A product, either before or after refining, can be identical to an existing petrochemical. Some of the fuel products may not be the same as existing petrochemicals. In one embodiment, a fuel product is similar to an existing petrochemical, except for the carbon isotope distribution. For example, it, is believed that no fossil fuel petrochemicals have a δ¹³C distribution of less than −32%, whereas fuel products as described herein can have a δ¹³C distribution of less than −32%, less than −35%, less than −40%, less than −45%, less than −50%, less than −55%, or less than −60%. In another embodiment, a fuel product or composition is similar but not the same as an existing fossil fuel petrochemical and has a δ¹³C distribution of less than −32%, less than −35%, less than −40%, less than −45%, less than −50%, less than −55%, or less than −60%.

A fuel product, can be a composition comprising, for example, hydrogen and carbon molecules, wherein the hydrogen and carbon molecules are at least about 80% of the atomic weight of the composition, and wherein the 8° C. distribution of the composition is less than about −32%. For some fuel products described herein, the hydrogen and carbon molecules are at least 90% of the atomic weight of the composition. For example, a biodiesel or fatty acid methyl ester (which has less than 90% hydrogen and carbon molecules by weight) may not be part of the composition. In still other compositions, the hydrogen and carbon molecules are at least 95 or at least 99% of the atomic weight of the composition. In yet other compositions, the hydrogen and carbon molecules are 100% of the atomic weight of the composition. In some embodiments, the composition is a liquid. In other embodiments, the composition is a fuel additive or a fuel product.

Also described herein is a fuel product comprising a composition comprising: hydrogen and carbon molecules, wherein the hydrogen and carbon molecules are at least 80% of the atomic weight of the composition, and wherein the δ¹³C distribution of the composition is less than −32%; and a fuel component. In some embodiments, the δ¹³C distribution of the composition is less than about −35%, less than about −40%, less than about −45%, less than about −50%, less than about −55%, or less than about −60%. In some embodiments, the fuel component of the composition is a blending fuel, for example, a fossil fuel, gasoline, diesel, ethanol, jet fuel, or any combination thereof. In still other embodiments, the blending fuel has a δ¹³C distribution of greater than −32%. For some fuel products described herein, the fuel component is a fuel additive which may be MTBE, an anti-oxidant, an antistatic agent, a corrosion inhibitor, or any combination thereof. A fuel product as described herein may be a product generated by blending a fuel product as described and a fuel component. In some embodiments, the fuel product has a δ¹³C distribution of greater than −32%. In other embodiments, the fuel product has a δ¹³C distribution of less than −32%. For example, an oil composition extracted from an organism can be blended with a fuel component prior to refining (for example, cracking) in order to generate a fuel product as described herein. A fuel component, can be a fossil fuel, or a mixing blend for generating a fuel product. For example, a mixture for fuel blending may be a hydrocarbon mixture that is suitable for blending with another hydrocarbon mixture to generate a fuel product. For example, a mixture of light alkanes may not have a certain octane number to be suitable for a type of fuel, however, it can be blended with, a high octane mixture to generate a fuel product. In another example, a composition with, a δ¹³C distribution of less than −32% is blended with a hydrocarbon mixture for fuel blending to create a fuel product. In some embodiments, the composition or fuel component alone are not suitable as a fuel product, however, when combined, they are useful as a fuel product. In other embodiments, either the composition or the fuel component or both individually are suitable as a fuel product. In yet another embodiment, the fuel component is an existing petroleum product, such as gasoline or jet fuel. In other embodiments, the fuel component is derived from a renewable resource, such as bioethanol, biodiesel, and biogasoline.

Oil compositions, derived from biomass obtained from a host cell, can be used for producing high-octane hydrocarbon products. Thus, one embodiment describes a method of forming a fuel product, comprising: obtaining an upgraded oil composition, cracking the oil composition, and blending the resulting one or more light hydrocarbons, having 4 to 12 carbons and an Octane number of 80 or higher, with a hydrocarbon having an Octane number of 80 or less. The hydrocarbons having an Octane number of 80 or less are, for example, fossil fuels derived from refining crude oil.

The biomass feedstock obtained from a host organism can be modified or tagged such that the light hydrocarbon products can be identified or traced back to their original feedstock. For example, carbon isotopes can be introduced into a biomass hydrocarbon in the course of its biosynthesis. The tagged hydrocarbon feedstock can be subjected to the refining processes described herein to produce a light hydrocarbon product tagged with a carbon isotope. The isotopes allow for the identification of the fagged products, either alone or in combination with other untagged products, such that the tagged products can be traced back to their original biomass feedstocks.

TABLE 1
Examples of Enzymes Involved in the Isoprenoid Pathway

| Synthase | Source | NCBI protein ID |
|---|---|---|
| Limonene | M. spicata | 2ONH_A |
| Cineole | S. officinalis | AAC26016 |
| Pinene | A. grandis | AAK83564 |
| Camphene | A. grandis | AAB70707 |
| Sabinene | S. officinalis | AAC26018 |
| Myrcene | A. grandis | AAB71084 |
| Abietadiene | A. grandis | Q38710 |
| Taxadiene | T. brevifolia | AAK83566 |
| FPP | G. gallus | P08836 |
| Amorphadiene | A. annua | AAF61439 |
| Bisabolene | A. grandis | O81086 |
| Diapophytoene | S. aureus | |
| Diapophytoene desaturase | S. aureus | |
| GPPS-LSU | M. spicata | AAF08793 |
| GPPS-SSU | M. spicata | AAF08792 |
| GPPS | A. thaliana | CAC16849 |
| GPPS | C. reinhardtii | EDP05515 |
| FPP | E. coli | NP_414955 |
| FPP | A. thaliana | NP_199588 |
| FPP | A. thaliana | NP_193452 |
| FPP | C. reinhardtii | EDP03194 |
| IPP isomerase | E. coli | NP_417365 |
| IPP isomerase | H. pluvialis | ABB80114 |
| Limonene | L. angustifolia | ABB73044 |
| Monoterpene | S. lycopersicum | AAX69064 |
| Terpinolene | O. basilicum | AAV63792 |
| Myrcene | O. basilicum | AAV63791 |
| Zingiberene | O. basilicum | AAV63788 |
| Myrcene | Q. ilex | CAC41012 |
| Myrcene | P. abies | AAS47696 |
| Myrcene, ocimene | A. thaliana | NP_179998 |
| Myrcene, ocimene | A. thaliana | NP_567511 |
| Sesquiterpene | Z. mays; B73 | AAS88571 |
| Sesquiterpene | A. thaliana | NP_199276 |
| Sesquiterpene | A. thaliana | NP_193064 |
| Sesquiterpene | A. thaliana | NP_193066 |
| Curcumene | P. cablin | AAS86319 |
| Farnesene | M. domestica | AAX19772 |
| Farnesene | C. sativus | AAU05951 |
| Farnesene | C. junos | AAK54279 |
| Farnesene | P. abies | AAS47697 |
| Bisabolene | P. abies | AAS47689 |
| Sesquiterpene | A. thaliana | NP_197784 |
| Sesquiterpene | A. thaliana | NP_175313 |
| GPP Chimera | | |
| GPPS-LSU + SSU fusion | | |
| Geranylgeranyl reductase | A. thaliana | NP_177587 |
| Geranylgeranyl reductase | C. reinhardtii | EDP09986 |
| Chlorophyllidohydrolase | C. reinhardtii | EDP01364 |
| Chlorophyllidohydrolase | A. thaliana | NP_564094 |
| Chlorophyllidohydrolase | A. thaliana | NP_199199 |
| Phosphatase | S. cerevisiae | AAB64930 |
| FPP A118W | G. gallus | |

The enzymes utilized may be encoded by nucleotide sequences derived from any organism, including bacteria, plants, fungi and animals. In some instances, the enzymes are isoprenoid producing enzymes. As used herein, an “isoprenoid producing enzyme” is a naturally or non-naturally occurring enzyme which produces or increases production of an isoprenoid. In some instances, an isoprenoid producing enzyme produces isoprenoids with two phosphate groups (e.g., GPP synthase, FPP synthase, DMAPP synthase). In other instances, isoprenoid producing enzymes produce isoprenoids with zero, one, three or more phosphates or may produce isoprenoids with other functional groups. Non-limiting examples of such enzymes and their sources are shown in Table 1.

Codon Optimization

As discussed above, one or more codons of an encoding polynucleotide can be “biased” or “optimized” to reflect the codon usage of the host organism. For example, one or more codons of an encoding polynucleotide can be “biased” or “optimized” to reflect chloroplast codon usage (Table 2) or nuclear codon usage (Table 3). Most amino acids are encoded by two or more different (degenerate) codons, and it is well recognized that various organisms utilize certain codons in preference to others, “Biased” or codon “optimized” can be used interchangeably throughout the specification. Codon bias can be variously skewed in different plants, including, for example, in alga as compared to tobacco. Generally, the codon bias selected reflects codon usage of the plant, (or organelle therein) which is being transformed with the nucleic acids of the present disclosure.

A polynucleotide that is biased for a particular codon usage can be synthesized de novo, or can be genetically modified using routine recombinant DNA techniques, for example, by a site directed mutagenesis method, to change one or more codons such that, they are biased for chloroplast codon usage.

Such preferential codon usage, which is utilized in chloroplasts, is referred to herein as “chloroplast codon usage.” Table 2 (below) shows the chloroplast codon usage for C. reinhardtii (see U.S. Patent Application Publication No.: 2004/0014174, published Jan. 22, 2004).

TABLE 2
Chloroplast Codon Usage in Chlamydomonas reinhardtii

| | | | |
|---|---|---|---|
| UUU 34.1*(348**) | UCU 19.4(198) | UAU 23.7(242) | UGU 8.5(87) |
| UUC 14.2(145) | UCC 4.9(50) | UAC 10.4(106) | UGC 2.6(27) |
| UUA 72.8(742) | UCA 20.4(208) | UAA 2.7(28) | UGA 0.1(1) |
| UUG 5.6(57) | UCG 5.2(53) | UAG 0.7(7) | UGG 13.7(140) |
| CUU 14.8(151) | CCU 14.9(152) | CAU 11.1(113) | CGU 25.5(260) |
| CUC 1.0(10) | CCC 5.4(55) | CAC 8.4(86) | CGC 5.1(52) |
| CUA 6.8(69) | CCA 19.3(197) | CAA 34.8(355) | CGA 3.8(39) |
| CUG 7.2(73) | CCG 3.0(31) | CAG 5.4(55) | CGG 0.5(5) |
| AUU 44.6(455) | ACU 23.3(237) | AAU 44.0(449) | AGU 16.9(172) |
| AUC 9.7(99) | ACC 7.8(80) | AAC 19.7(201) | AGC 6.7(68) |
| AUA 8.2(84) | ACA 29.3(299) | AAA 61.5(627) | AGA 5.0(51) |
| AUG 23.3(238) | ACG 4.2(43) | AAG 11.0(112) | AGG 1.5(15) |
| GUU 27.5(280) | GCU 30.6(312) | GAU 23.8(243) | GGU 40.0(408) |
| GUC 4.6(47) | GCC 11.1(113) | GAC 11.6(118) | GGC 8.7(89) |
| GUA 26.4(269) | GCA 19.9(203) | GAA 40.3(411) | GGA 9.6(98) |
| GUG 7.1(72) | GCG 4.3(44) | GAG 6.9(70) | GGG 4.3(44) |

*Frequency of codon usage per 1,000 codons.
**Number of times observed in 36 chloroplast coding sequences (10,193 codons).

The chloroplast codon bias can, but need not, be selected based on a particular organism in which a synthetic polynucleotide is to be expressed. The manipulation can be a change to a codon, for example, by a method such as site directed mutagenesis, by a method such as PCR using a primer that is mismatched for the nucleotide(s) to be changed such that the amplification product is biased to reflect chloroplast codon usage, or can be the de novo synthesis of polynucleotide sequence such that the change (bias) is introduced as a consequence of the synthesis procedure.

In addition to utilizing chloroplast. codon bias as a means to provide efficient translation of a polypeptide, it will be recognized that an alternative means for obtaining efficient translation of a polypeptide in a chloroplast is to re-engineer the chloroplast genome (e.g., a C. reinhardtii chloroplast genome) for the expression of tRNAs not otherwise expressed in the chloroplast genome. Such an engineered algae expressing one or more exogenous tRNA molecules provides the advantage that it would obviate a requirement, to modify every polynucleotide of interest, that is to be introduced into and expressed from a chloroplast genome; instead, algae such as C. reinhardtii that, comprise a genetically modified chloroplast genome can be provided and utilized for efficient translation, of a polypeptide according to any method of the disclosure. Correlations between tRNA abundance and codon usage in highly expressed genes is well known (for example, as described in Franklin et al., Plant J. 30:733-744, 2002; Dong et al., J. Mol. Biol. 260:649-663, 1996; Duret, Trends Genet. 16:287-289, 2000; Goldman et al, J. Mol. Biol. 245:467-473, 1995; and Komar et, ah, Biol. Chem. 379:1295-1300, 1998). In E. coli, for example, re-engineering of strains to express underutilized tRNAs resulted in enhanced expression of genes which utilize these codons (see Novy et al., in Novations 12:3-3, 2001). Utilizing endogenous tRNA genes, site directed mutagenesis can be used to make a synthetic tRNA gene, which can be introduced into chloroplasts to complement rare or unused tRNA genes in a chloroplast genome, such as a C. reinhardtii chloroplast genome.

Generally, the chloroplast codon bias selected for purposes of the present disclosure, including, for example, in preparing a synthetic polynucleotide as disclosed herein reflects chloroplast codon usage of a plant chloroplast, and includes a codon bias that, with respect to the third position of a codon, is skewed towards A/T, for example, where the third position has greater than about 66% AT bias, or greater than about 70% AT bias, in one embodiment, the chloroplast codon usage is biased to reflect alga chloroplast codon usage, for example, C. reinhardtii, which has about 74.6% AT bias in the third codon position. Preferred codon usage in the chloroplasts of algae has been described in US 2004/0014174.

Table 3 exemplifies codons that are preferentially used in algal nuclear genes. The nuclear codon bias can, but need not, be selected based on a particular organism in which a synthetic polynucleotide is to be expressed. The manipulation can be a change to a codon, for example, by a method such as site directed mutagenesis, by a method such as PCR using a primer that is mismatched for the nucleotide(s) to be changed such that the amplification product is biased to reflect nuclear codon usage, or can be the de novo synthesis of polynucleotide sequence such that the change (bias) is introduced as a consequence of the synthesis procedure.

In addition to utilizing nuclear codon bias as a means to provide efficient translation of a polypeptide, it will be recognized that an alternative means for obtaining efficient translation, of a polypeptide in a nucleus is to re-engineer the nuclear genome (e.g., a C. reinhardtii nuclear genome) for the expression of tRNAs not otherwise expressed in the nuclear genome. Such an engineered algae expressing one or more exogenous tRNA molecules provides the advantage that it would obviate a requirement to modify every polynucleotide of interest that is to be introduced into and expressed from a nuclear genome; instead, algae such as C. reinhardtii that comprise a genetically modified nuclear genome can be provided and utilized for efficient translation of a polypeptide according to any method of the disclosure. Correlations between tRNA abundance and codon usage in highly expressed genes is well known (for example, as described in Franklin et al., Plant J. 30:733-744, 2002; Dong et al., J. Mol. Biol. 260:649-663, 3996; Duret, Trends Genet. 16:287-289, 2000; Goldman et. Al., I. Mol. Biol. 245:467-473, 1995; and Komar et. Al., Biol. Chem. 379:1295-1300, 1998). In E. coli, for example, re-engineering of strains to express underutilized tRNAs resulted in enhanced expression of genes which utilize these codons (see Novy et al., in Novations 12:1-3, 2001). Utilizing endogenous tRNA genes, site directed mutagenesis can be used to make a synthetic tRNA gene, which can be introduced into the nucleus to complement rare or unused tRNA genes in a nuclear genome, such as a C. reinhardtii nuclear genome.

Generally, the nuclear codon bias selected for purposes of the present disclosure, including, for example, in preparing a synthetic polynucleotide as disclosed herein, can reflect nuclear codon usage of an algal nucleus and includes a codon bias that results in the coding sequence containing greater than 60% G/C content.

TABLE 3
Nuclear Codon Usage in Chlamydomonas reinhardtii

| | | | |
|---|---|---|---|
| UUU 5.0 (2110) | UCU 4.7 (1992) | UAU 2.6 (1085) | UGU 1.4 (601) |
| UUC 27.1 (11411) | UCC 16.1 (6782) | UAC 22.8 (9579) | UGC 13.1 (5498) |
| UUA 0.6 (247) | UCA 3.2 (1348) | UAA 1.0 (441) | UGA 0.5 (227) |
| UUG 4.0 (1673) | UCG 16.1 (6763) | UAG 0.4 (183) | UGG 13.2 (5559) |
| CUU 4.4 (1869) | CCU 8.1 (3416) | CAU 2.2 (919) | CGU 4.9 (2071) |
| CUC 13.0 (5480) | CCC 29.5 (12409) | CAC 17.2 (7252) | CGC 34.9 (14676) |
| CUA 2.6 (1086) | CCA 5.1 (2124) | CAA 4.2 (1780) | CGA 2.0 (841) |
| CUG 65.2 (27420) | CCG 20.7 (8684) | CAG 36.3 (15283) | CGG 11.2 (4711) |
| AUU 8.0 (3360) | ACU 5.2 (2171) | AAU 2.8 (1157) | AGU 2.6 (1089) |
| AUC 26.6 (11200) | ACC 27.7 (11663) | AAC 28.5 (11977) | AGC 22.8 (9590) |
| AUA 1.1 (443) | ACA 4.1 (1713) | AAA 2.4 (1028) | AGA 0.7 (287) |
| 0AUG 25.7 (10796) | ACG 15.9 (6684) | AAG 43.3 (18212) | AGG 2.7 (1150) |
| GUU 5.1 (2158) | GCU 16.7 (7030) | GAU 6.7 (2805) | GGU 9.5 (3984) |
| GUC 15.4 (6496) | GCC 54.6 (22960) | GAC 41.7 (17519) | GGC 62.0 (26064) |
| GUA 2.0 (857) | GCA 10.6 (4467) | GAA 2.8 (1172) | GGA 5.0 (2084) |
| GUG 46.5 (19558) | GCG 44.4 (18688) | GAG 53.5 (22486) | GGG 9.7 (4087) |

fields: [triplet] [frequency: per thousand] ([number])
Coding GC 66.30% 1^{st} letter GC 64.80% 2^{nd} letter GC 47.90% 3^{rd} letter GC 86.21%

Table 4

Table 4 lists the codon selected at each position for backtranslating the protein to a DNA sequence for synthesis. The selected codon is the sequence recognized by the tRNA encoded in the chloroplast genome when present; the stop codon (TAA) is the codon most frequently present in the chloroplast encoded genes. If an undesired restriction site is created, the next best choice according to the regular Chlamydomonas chloroplast usage table that eliminates the restriction site is selected.

TABLE 4

| Amino acid | Codon utilized |
|---|---|
| F | TTC |
| L | TTA |
| I | ATC |
| V | GTA |
| S | TCA |
| P | CCA |
| T | ACA |
| A | GCA |
| Y | TAC |
| H | CAC |
| Q | CAA |
| N | AAC |
| K | AAA |
| D | GAC |
| E | GAA |
| C | TGC |
| R | CGT |
| G | GGC |
| W | TGG |
| M | ATG |
| STOP | TAA |

Percent Sequence Identity

One example of an algorithm that is suitable for determining percent sequence identity or sequence similarity between nucleic acid or polypeptide sequences is the BLAST algorithm, which is described, e.g., in Altschul et al., J. Mol. Biol. 215:403-410 (1990). Software for performing BLAST analysis is publicly available through the National Center for Biotechnology Information. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, She BLASTP program uses as defaults a word length (W) of 3, an expectation (E) of 10, and the BLOSUM62 scoring matrix (as described, for example, in Henikoff & Henikoff (1989) Proc. Natl. Acad. Sci. USA, 89:10915). In addition to calculating percent, sequence identity, the BLAST algorithm also can perform a statistical analysis of the similarity between two sequences (for example, as described in Karlin & Altschul, Proc. Nat'l. Acad. Sci. USA, 90:5873-5787 (1993)). One measure of similarity provided by the BLAST algorithm is the smallest sum probability (P(N)), which, provides an indication, of the probability by which, a match between two nucleotide or amino acid sequences would occur by chance. For example, a nucleic acid is considered similar to a reference sequence if the smallest sum probability in a comparison of the test nucleic acid to the reference nucleic acid is less than about 0.1, less than about 0.01, or less than about 0.001.

Fatty Acids and Glycerol Lipids

The present, disclosure describes host cells capable of making polypeptides that contribute to the accumulation and/or secretion of fatty acids, glycerol lipids, or oils, by transforming host, cells (e.g., alga cells such as C. reinhardtii, D. salina, H. pluvalis, and cyanobacterial cells) with nucleic acids encoding one or more different enzymes. Examples of such enzymes include acetyl-CoA carboxylase, ketoreductase, thioesterase, malonyltransferase, dehydratase, acyl-CoA ligase, ketoacylsynthase, enoylreductase, and desaturase. The enzymes can be, for example, catabolic or biodegrading enzymes.

In some instances, the host cell will naturally produce the fatty acid, glycerol lipid, triglyceride, or oil of interest. Therefore, transformation of the host cell with a polynucleotide encoding an enzyme, for example an ACCase, will allow for the increased activity of the enzyme and/or increased accumulation and/or secretion of a molecule of interest (e.g., a lipid) in the cell.

A change in the accumulation and/or secretion of a desired product, for example, fatty acids, glycerol lipids, or oils, by a transformed host cell can include, for example, a change in the total oil content over that normally present in the cell, or a change in the type of oil that: is normally present in the cell.

A change in the accumulation and/or secretion of a desired product, for example, fatty acids, glycerol lipids, or oils, by a transformed host cell can include, for example, a change in the total lipid content over that normally present in the cell, or a change in the type of lipids that are normally present in the cell.

Increased malonyl CoA production is required for increased. Increased fatty acid biosynthesis is required for increased accumulation of fatty acid based lipids. An increase in fatty acid based lipids can be measured by methyl tert-butyl ether (MTBE) extraction.

Some host cells may be transformed with multiple genes encoding one or more enzymes. For example, a single transformed cell may contain exogenous nucleic acids encoding enzymes that make up an entire glycerolipid synthesis pathway. One example of a pathway might include genes encoding an acetyl CoA carboxylase, a malonyltransferase, a ketoacylsynthase, and a thioesterase. Cells transformed with an entire pathway and/or enzymes extracted from those cells, can synthesize, for example, complete fatty acids or intermediates of the fatty acid synthesis pathway. Constructs may contain, for example, multiple copies of the same gene, multiple genes encoding the same enzyme from different organisms, and/or multiple genes with one or more mutations in the coding sequence(s).

The enzyme(s) produced by the modified cells may result in the production of fatty acids, glycerol lipids, triglycerides, or oils that may be collected from the cells and/or the surrounding environment (e.g., bioreactor or growth medium). In some embodiments, the collection of the fatty acids, glycerol lipids, triglycerides, or oils is performed after the product is secreted from the cell via a cell membrane transporter.

Examples of candidate Chlamydomonas genes encoding enzymes of glycerolipid metabolism that can be used in the described embodiments are described in The Chlamydomonas Sourcebook Second Edition, Organellar and Metabolic Processes, Vol. 2, pp. 41-68, David B. Stern (Ed.), (2009), Elsevier Academic Press.

For example, enzymes involved in plastid, mitochondrial, and cytosolic pathways, along with plastidic and cytosolic isoforms of fatty acid desaturases, and triglyceride synthesis enzymes are described (and their accession numbers provided). An exemplary chart of some of the genes described is provided below:

| | | |
|---|---|---|
| Acyl-ACP thioesterase | FAT1 | EDP08596 |
| Long-chain acyl-CoA synthetase | LCS1 | EDO96800 |
| CDP-DAG: Inositol phosphotransferase | PIS1 | EDP06395 |
| Acyl-CoA: Diacylglycerol acyltransferase | DGA1 | EDO96893 |
| Phospholipid: Diacylglycerol | LRO1(LCA1) | EDP07444 |
| acyltransferase | | |

Examples of the types of fatty acids and/or glycerol lipids that a host cell or organism can produce, are described below.

Lipids are a broad group of naturally occurring molecules which includes fats, waxes, sterols, fat-soluble vitamins (such as vitamins A, D, E and K), monoglycerides, diglycerides, phospholipids, and others. The main biological functions of lipids include energy storage, as structural components of cell membranes, and as important signaling molecules.

Lipids may be broadly defined as hydrophobic or amphiphilic small molecules; the amphiphilic nature of some lipids allows them to form structures such as vesicles, liposomes, or membranes in an aqueous environment. Biological lipids originate entirely or in part from two distinct types of biochemical subunits or “building blocks”: ketoacyl and isoprene groups. Lipids may be divided into eight categories: fatty acyls, glycerolipids, glycerophospholipids, sphingolipids, saccharolipids and polyketides (derived from condensation of ketoacyl subunits); and sterol lipids and prenol lipids (derived from condensation of isoprene subunits). For this disclosure, saccharolipids will not be discussed.

Fats are a subgroup of lipids called triglycerides. Lipids also encompass molecules such as fatty acids and their derivatives (including tri-, di-, and monoglycerides and phospholipids), as well as other sterol-containing metabolites such as cholesterol. Humans and other mammals use various biosynthetic pathways to both break down and synthesize lipids.

Fatty Acyls

Fatty acyls, a generic term for describing fatty acids, their conjugates and derivatives, are a diverse group of molecules synthesized by chain-elongation of an acetyl-CoA primer with malonyl-CoA or methylmalonyl-CoA groups in a process called fatty acid synthesis. A fatty acid is any of the aliphatic monocarboxylic acids that can be liberated by hydrolysis from naturally occurring fats and oils. They are made of a hydrocarbon chain that terminates with a carboxylic acid group; this arrangement confers the molecule with a polar, hydrophilic end, and a nonpolar, hydrophobic end that is insoluble in water. The fatty acid structure is one of the most fundamental categories of biological lipids, and is commonly used as a building block of more structurally complex lipids. The carbon chain, typically between four to 24 carbons long, may be saturated or unsaturated, and may be attached to functional groups containing oxygen, halogens, nitrogen and sulfur; branched fatty acids and hydroxyl fatty acids also occur, and very long chain acids of over 30 carbons are found in waxes. Where a double bond exists, there is the possibility of either a cis or trans geometric isomerism, which, significantly affects the molecule's molecular configuration. Cis-double bonds cause the fatty acid chain to bend, an effect that is more pronounced the more double bonds there are in a chain. This in turn, plays an important role in the structure and function of cell membranes. Most naturally occurring fatty acids are of the cis configuration, although the trans form does exist in some natural and partially hydrogenated fats and oils.

Examples of biologically important fatty acids are the eicosanoids, derived primarily from arachidonic acid and eicosapentaenoic acid, which include prostaglandins, leukotrienes, and thromboxanes. Other major lipid classes in the fatty acid category are the fatty esters and fatty amides. Fatty esters include important biochemical intermediates such as wax esters, fatty acid thioester coenzyme A derivatives, fatty acid thioester ACP derivatives and fatty acid carnitines. The fatty amides include N-acyl ethanolamines.

Glycerolipids

Glycerolipids are composed mainly of mono-, di- and tri-substituted glycerols, the most well-known being the fatty acid esters of glycerol (triacylglycerols), also known as triglycerides. In these compounds, the three hydroxyl groups of glycerol are each esterified, usually by different fatty acids. Because they function as a food store, these lipids comprise the bulk of storage fat in animal tissues. The hydrolysis of the ester bonds of triacylglycerols and the release of glycerol and fatty acids from adipose tissue is called fat mobilization.

Additional subclasses of glycerolipids are represented by glycosylglycerols, which are characterized by the presence of one or more sugar residues attached to glycerol via a glycosidic linkage. An example of a structure in this category is the digalactosyldiacylglycerols found in plant membranes.

Exemplary Chlamydomonas glycerolipids include: DGDG, digalactosyldiacylglycerol; DGTS, diacylglyceryl-N,N,N-trimethylhomoserine; MGDG, monogalactosyldiacylglycerol; PtdEtn, phosphatidylethanolamine; PtdGro, phosphatidylglycerol; PtdIns, phosphatidylinositol; SQDG, sulfoquinovosyldiacylglycerol; and TAG, triacylglycerol.

Glycerophospholipids

Glycerophospholipids are any derivative of glycerophosphoric acid that contains at least one O-acyl, O-alkyl, or O-alkenyl group attached to the glycerol residue. The common glycerophospholipids are named as derivatives of phosphatidic acid (phosphatidyl choline, phosphatidyl serine, and phosphatidyl ethanolamine).

Glycerophospholipids, also referred to as phospholipids, are ubiquitous in nature and are key components of the lipid bilayer of cells, as well as being involved in metabolism and cell signaling. Glycerophospholipids may be subdivided into distinct classes, based on the nature of the polar headgroup at the sn-3 position of the glycerol backbone in eukaryotes and eubacteria, or the sn-1 position in the case of archaebacteria.

Examples of glycerophospholipids found in biological membranes are phosphatidylcholine (also known as PC, GPCho or lecithin), phosphatidylethanolamine (PE or GPEtn) and phosphatidylserine (PS or GPSer). In addition to serving as a primary component of cellular membranes and binding sites for intra- and intercellular proteins, some glycerophospholipids in eukaryotic cells, such as phosphatidylinositols and phosphatidic acids are either precursors of, or are themselves, membrane-derived second messengers. Typically, one or both of these hydroxyl groups are acylated with long-chain fatty acids, but there are also alkyl-linked and 1Z-alkenyl-linked (plasmalogen) glycerophospholipids, as well as dialkylether variants in archaebacteria.

Sphingolipids

Sphingolipids are any of class of lipids containing the long-chain, amino diol, sphingosine, or a closely related base (i.e. a sphingoid). A fatty acid is bound in an amide linkage to the amino group and the terminal hydroxyl may be linked to a number of residues such as a phosphate ester or a carbohydrate. The predominant base in animals is sphingosine while in plants it is phytosphingosine.

The main classes are: (1) phosphosphigolipids (also known as sphingophospholipids), of which the main representative is sphingomyelin; and (2) glycosphingolipids, which contain at least one monosaccharide and a sphingoid, and include the cerebrosides and gangliosides. Sphingolipids play an important structural role in cell membranes and may be involved in the regulation of protein kinase C.

As mentioned above, sphingolipids are a complex family of compounds that share a common structural feature, a sphingoid base backbone, and are synthesized de novo from the amino acid serine and a long-chain fatty acyl CoA, that are then converted into ceramides, phosphosphingolipids, glycosphingolipids and other compounds. The major sphingoid base of mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms.

The major phosphosphingolipids of mammals are sphingomyelins (ceramide phosphocholines), whereas insects contain mainly ceramide phosphoethanolamines, and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol Lipids

Sterol lipids, such as cholesterol and its derivatives, are an important component of membrane lipids, along with the glycerophospholipids and sphingomyelins. The steroids, all derived from the same fused four-ring core structure, have different biological roles as hormones and signaling molecules. The eighteen-carbon (C18) steroids include the estrogen family whereas the C19 steroids comprise the androgens such as testosterone and androsterone. The C21 subclass includes the progestogens as well as the glucocorticoids and mineralocorticoids. The secosteroids, comprising various forms of vitamin D, are characterized by cleavage of the B ring of the core structure. Other examples of sterols are the bile acids and their conjugates, which in mammals are oxidized derivatives of cholesterol and are synthesized in the liver. The plant equivalents are the phytosterols, such as β-sitosterol, stigmasterol, and brassicasterol; the latter compound is also used as a biomarker for algal growth. The predominant sterol in fungal cell membranes is ergosterol.

Prenol Lipids

Prenol lipids are synthesized from the 5-carbon precursors isopentenyl diphosphate and dimethylallyl diphosphate that are produced mainly via the mevalonic acid (MVA) pathway. The simple isoprenoids (for example, linear alcohols and diphosphates) are formed by the successive addition of C5 units, and are classified according to the number of these terpene units. Structures containing greater than 40 carbons are known as polyterpenes. Carotenoids are important simple isoprenoids that function as antioxidants and as precursors of vitamin A. Another biologically important class of molecules is exemplified by the quinones and hydroquinones, which contain an isoprenoid tail attached to a quinonoid core of non-isoprenoid origin. Prokaryotes synthesize polyprenols (called bactoprenols) in which the terminal isoprenoid unit attached to oxygen remains unsaturated, whereas in animal polyprenols (dolichols) the terminal isoprenoid is reduced.

Polyketides

Polyketides or sometimes acetogenin are any of a diverse group of natural products synthesized via linear poly-β-ketones, which are themselves formed by repetitive head-to-tail addition of acetyl (or substituted acetyl) units indirectly derived from acetate (or a substituted acetate) by a mechanism similar to that for fatty-acid biosynthesis but without the intermediate reductive steps. In many case, acetyl-CoA functions as the starter unit and malonyl-CoA as the extending unit. Various molecules other than acetyl-CoA may be used as starter, often with methoylmalonyl-CoA as the extending unit. The poly-β-ketones so formed may undergo a variety of further types of reactions, which include alkylation, cyclization, glycosylation, oxidation, and reduction. The classes of product formed—and their corresponding starter substances—comprise inter alia: coniine (of hemlock) and orsellinate (of lichens)—acetyl-CoA; flavanoids and stilbenes—cinnamoyl-CoA; tetracyclines—amide of malonyl-CoA; urushiols (of poison ivy)—palmitoleoyl-CoA; and erythonolides—propionyl-CoA and methyl-malonyl-CoA as extender.

Polyketides comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, and/or other processes. Many commonly used anti-microbial, anti-parasitic, and anti-cancer agents are polyketides or polyketide derivatives, such as erythromycins, tetracyclines, avermectins, and antitumor epothilones.

The following examples are intended to provide illustrations of the application of the present disclosure. The following examples are not intended to completely define or otherwise limit the scope of the disclosure. One of skill in the art will appreciate that many other methods known in the art may be substituted in lieu of the ones specifically described or referenced herein.

### EXAMPLES

### Example 1

### Transformation and Screening Methods

In this example, a method for transformation of Scenedesmus sp. is described. Algae cells are grown to log phase (approximately 0.5-1.0×10⁷ cells/mL) in TAP medium (Gorman and Levine, Proc. Natl. Acad. Sci., USA 54:1665-1669, 1965, which is incorporated herein by reference) at 23° C. under constant illumination of 50-100 uE on a rotary shaker set at 100 rpm. Cells are harvested at 1000×g for 5 min. The supernatant is decanted and cells are resuspended in TAP media at 10⁸ cells/mL. 5×10⁷ cells are spread on selective agar medium and transformed by particle bombardment with 550 nm or 1000 nm diameter gold particles carrying the transforming DNA@375-500 psi with the Helios Gene Gun (Bio-Rad) from a shot distance of 2-4 cm. Desired algae clones are those that grow on selective media.

PCR is used to identify transformed algae strains. For PCR analysis, colony lysates are prepared by suspending algae cells (from agar plate or liquid culture) in lysis buffer (0.5% SDS, 100 mM NaCl, 10 mM EDTA, 75 mM Tris-HCl, pH 7.5) and heating to 98° C. for 10 minutes, followed by cooling to near 23° C. Lysates are diluted 50-fold in 100 mM Tris-HCl pH 7.5 and 2 μL is used as template in a 25 μL reaction. Alternatively, total genomic DNA preparations may be substituted for colony lysates. A PCR cocktail consisting of reaction buffer, dNTPs, PCR primer pair(s) (indicated in each example below), DNA polymerase, and water is prepared. Algal DNA is added to provide template for the reaction. Annealing temperature gradients are employed to determine optimal annealing temperature for specific primer pairs. In many cases, algae transformants are analyzed by PCR in two ways. First, primers are used that are specific for the transgene being introduced into the chloroplast genome. Desired algae transformants are those that give rise to PCR product(s) of expected size(s). Second, two sets of primer pairs are used to determine the degree to which the transforming DNA was integrated into the chloroplast genome (heteroplasmic vs. homoplasmic). The first pair of primers amplifies a region spanning the site of integration. The second pair of primers amplifies a constant, or control region, that is not targeted by the transforming DNA, so should produce a product of expected size in all cases. This reaction, confirms that the absence of a PCR product, from the region spanning the site of integration did not result from cellular and/or other contaminants that inhibited the PCR reaction. Concentrations of the primer pairs are varied so that both amplicons are amplified in the same reaction. The number of cycles used is <30 to increase sensitivity. The most desired clones are those that yield a product for the constant region but not for the region spanning the site of integration. Once identified, clones are analyzed for changes in phenotype.

One of skill in the art will appreciate that many other transformation methods known in the art may be substituted in lieu of the ones specifically described or referenced herein.

### Example 2

### Chloroplast Transformation of S. dimorphus Using 3-(3,4-Dichlorophenyl)-1,1-dimethylurea (DCMU) Selection

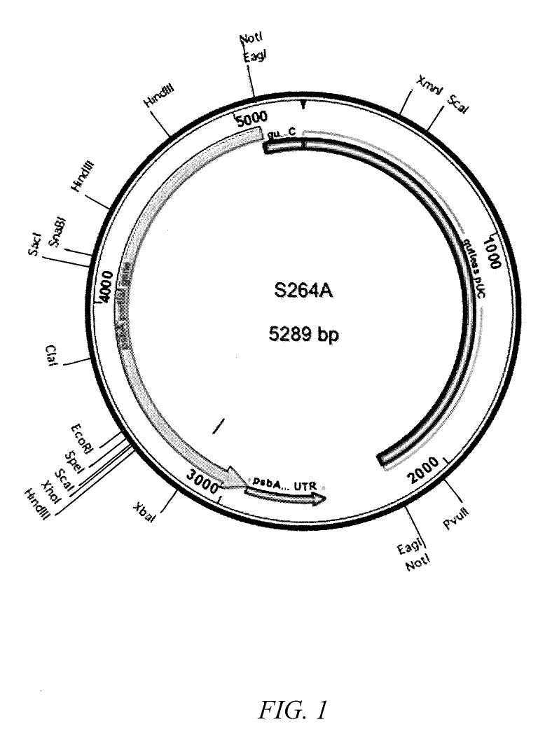

In this example, DCMU resistance was established as a selection method for transformation of S. dimorphus. Transforming DNA (SEQ ID NO: 30, S264A fragment) is shown graphically in FIG. 1. In this instance, a DNA fragment encompassing the 3′ end of the gene encoding psbA and it's 3′ UTR from S. dimorphus was amplified by PCR, subcloned into pUC18, and mutated via Quikchange PCR (Stratagene) to generate a S264A mutation along with a silent XbaI restriction site. Nucleotide 1913 of the fragment was mutated from a T to a G for the S264A mutation, and nucleotides 1928 to 1930 were mutated from CGT to AGA to generate the silent XbaI restriction site.

Transforming DNA was introduced into S. dimorphus via particle bombardment (as described in EXAMPLE 1) with DNA carried on 1000 nm gold particles, @375 psi and a shooting distance of 2 cm. Transformants were selected by growth on HSM media+0.5 uM DCMU under constant light 100-200 uE @23° C. for approximately 3 weeks.

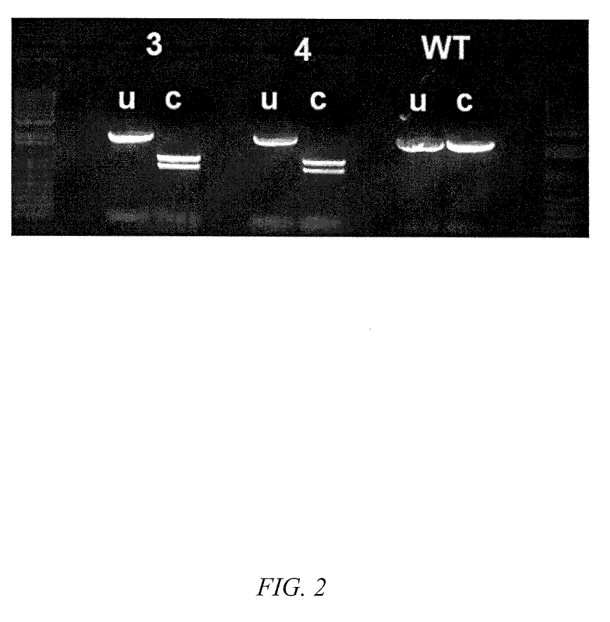

Transformants were verified by PCR screening (as described in EXAMPLE 1) using primers (SEQ ID NO: 17 and SEQ ID NO: 14) specific for a 2.1 kb region surrounding the bases changed for the S264A mutation. The PCR products were then digested with XbaI to distinguish transformants from spontaneous mutants that may arise as a result of plating cells onto media containing DCMU. FIG. 2 shows that DNA amplified from clones 3 and 4 is completely digested by XbaI (indicating that clones 3 and 4 are bonafide transformants while DNA amplified from wildtype cells (WT) is not. These data were confirmed by DNA sequencing of the PCR product.

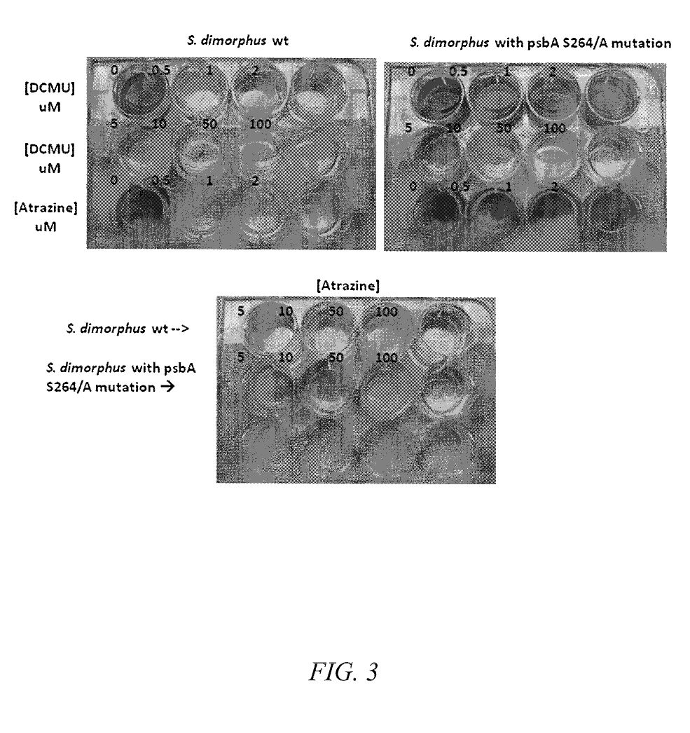

Transformants were grown to saturation in TAP media, diluted 1:100 in HSM+ various concentrations of DCMU and grown under constant light 50-100 uE with CO2 enrichment for 4 days. FIG. 3 shows that transformants with the psbA S264A imitation grow in up to 10 uM DCMU or 10 uM Atrazine whereas wild type S. dimporphus (wt) fails to grow in 0.5 uM DCMU or 0.5 uM Atrazine.

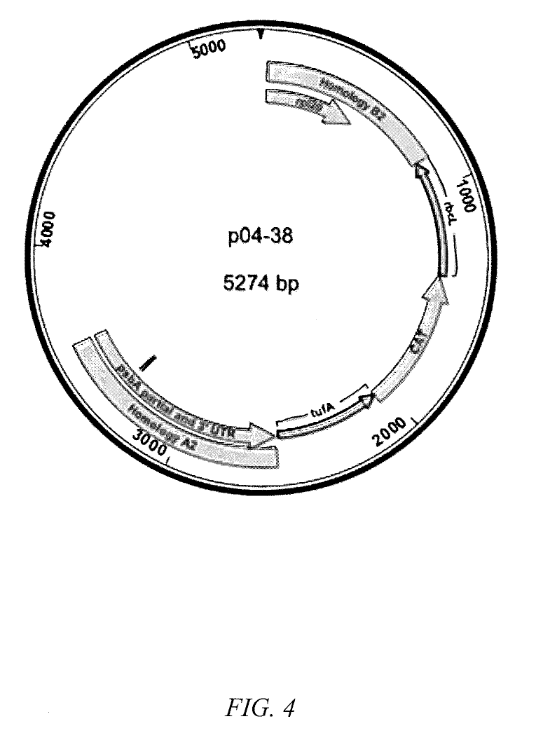

In order to determine if DCMU selection could result in incorporation of an expression cassette downstream of the psbA gene, A vector was constructed containing an expression cassette consisting of an endogenous promoter, a chloramphenicol acetyltransferase (CAT) gene, and an endogenous terminator cloned ˜500 bp downstream of the S264A/XbaI mutated psbA gene fragment from S. dimorphus and including the rpl20 gene. Transforming DNA is shown graphically in FIG. 4. In this instance the DNA segment labeled “CAT” is the chloramphenicol acetyl transferase gene from E. coli, the segment labeled “tufA” is the promoter and 5′ UTR sequence for the tufA gene from S. dimorphus, and the segment labeled “rbcL” is the 3′ UTR for the rbcL gene from S. dimorphus. The selection marker cassette is targeted to the S. dimorphus chloroplast genome via the segments labeled “Homology A2” and “Homology B2” which are 1000 bp fragments homologous to sequences of DNA adjacent to nucleotide 065,353 and include an S264A/XbaI mutated partial psbA coding sequence, its 3′UTR, and the rpl20 coding sequence. This vector targets integration of the selection marker cassette approximately 400 bp 3′ of the stop codon of the psbA gene.

Transforming DNA was introduced into S. dimorphus via particle bombardment (as described in EXAMPLE 1) with DNA carried on 550 nm gold particles, @500 psi and a shooting distance of 4 cm. Transformants were selected by growth on HSM media+1 uM DCMU under constant light 100-200 uE @RT for approximately 3 weeks.

To determine if the transformants were resistant to chloramphenicol (CAM), they were patched onto TAP agar medium containing 25 μg/mL CAM. In all cases, the DCMU transformants were also resistant to CAM indicating that the CAT cassette was incorporated into the genome.

One of skill in the art will appreciate that many other methods known in the art may be substituted in lieu of the ones specifically described or referenced.

### Example 3

### Use of Chloramphenical Acetyl Transferase as a Selection Marker in S. dimorphus

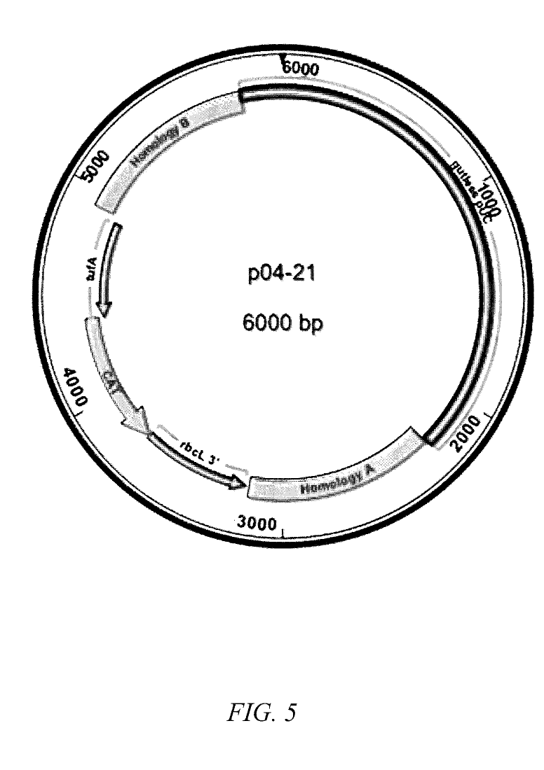

In this example, a nucleic acid encoding chloramphenicol acetyl transferase gene (CAT) from E. coli was introduced into S. dimorphus. Transforming DNA is shown graphically in FIG. 5. In this instance the DNA segment labeled “CAT” is the chloramphenicol acetyl transferase gene (SEQ ID NO: 28), the segment labeled “tufA” is the promoter and 5′ UTR sequence for the psbD (SEQ ID NO: 40) or tufA gene (SEQ ID NO: 42) from S. dimorphus, and the segment labeled “rbcL 3” is the 3′ UTR for the rbcL gene from S. dimorphus (SEQ ID NO: 57). The selection marker cassette is targeted to the S. dimorphus chloroplast genome via the segments labeled “Homology A” and “Homology B” which are approximately 1000 bp fragments homologous to sequences of DNA adjacent to nucleotide 035,138 (Site 2; nucleotide locations according to the sequence available from NCBI for S. obliquus, NC_{—}008101) on the 5′ and 3′ sides, respectively. All DNA segments were subcloned into pUC 18. All DNA manipulations carried out in the construction of this transforming DNA were essentially as described by Sambrook et ah. Molecular Cloning: A Laboratory Manual (Cold Spring Harbor Laboratory Press 1989) and Cohen et al., Meth. Enzymol. 297, 192-208, 1998, Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP agar medium+25 μg/mL chloramphenicol (TAP-CAM) under constant light 50-100 uE @RT for approximately 2 weeks. Transformants were patched onto TAP-CAM agar medium, grown for 4 days under constant light.

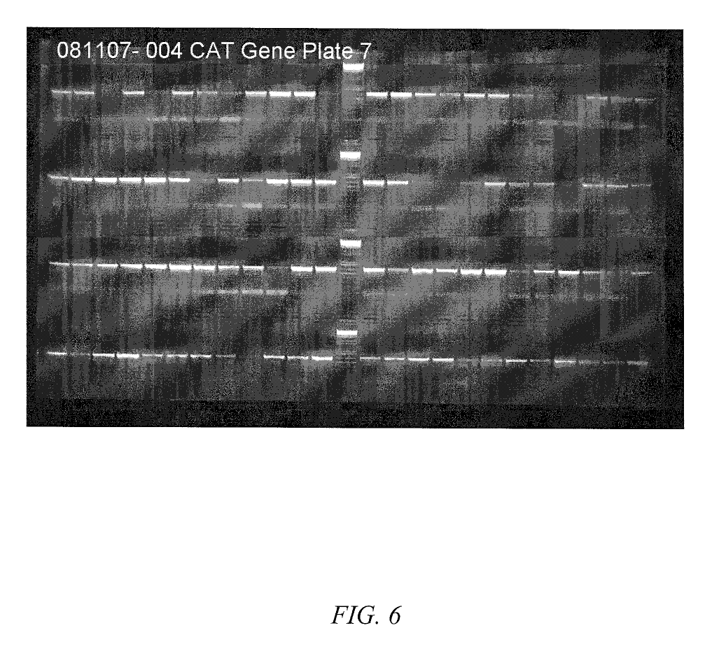

Cells from the patched transformants were analyzed by PCR screening (as described in EXAMPLE 1). The presence of the CAT selection marker was determined using primers that amplify the entire 660 bp gene (SEQ ID NO: 18 and SEQ ID NO: 19). FIG. 6 shows that a 660 bp fragment (representing the CAT gene) is amplified from DNA of several transformants (all lanes except +, − and ladders) while it is not amplified from DNA of wild type cells (−). DNA ladder is a 1 kb ladder.

One of skill in the art will appreciate that many other methods known in the art may be substituted in lieu of the ones specifically described or referenced.

### Example 4

### Production of Endoxylanase in S. dimorphus

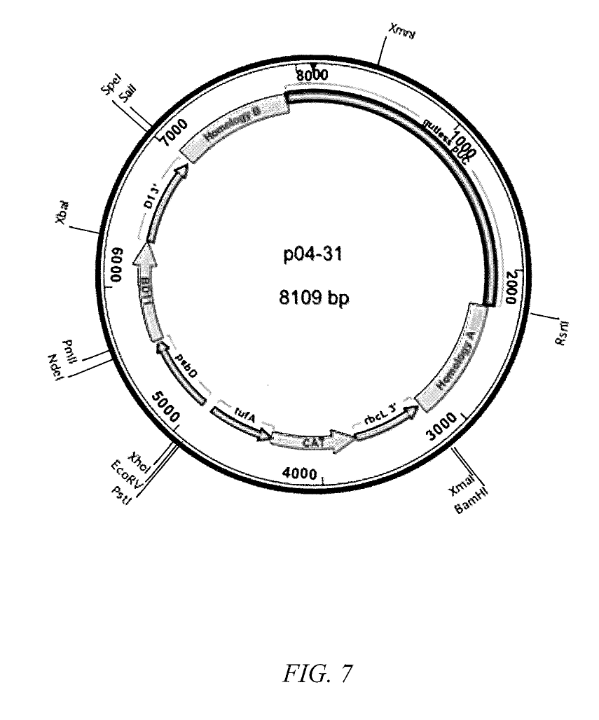

In this example a nucleic acid encoding endoxylanase from T. reesei was introduced into S. dimorphus. Transforming DNA (p04-31) is shown graphically in FIG. 7. In this instance the DNA segment labeled “BD11” is the endoxylanase encoding gene (SEQ ID NO: 21, BD11), the segment labeled “psbD” is the promoter and 5′ UTR for the psbD gene from S. dimorphus, the segment labeled “D1 3′” is the 3′ UTR for the psbA gene from S. dimorphus, and the segment labeled “CAT” is the chloramphenicol acetyl transferase gene (CAT) from E. coli, which is regulated by the promoter and 5′ UTR sequence for the tufA gene from S. dimorphus and the 3′ UTR sequence for the rbcL gene from S. dimorphus. The transgene expression cassette and selection marker are targeted to the S. dimorphus chloroplast genome via the segments labeled “Homology A” and “Homology B” which are approximately 1000 bp fragments homologous to sequences of DNA adjacent to nucleotide 071,366 (Site 1; nucleotide locations according to She sequence available from NCBI for S. obliquus, NC_{—}008101) on the 5′ and 3′ sides, respectively. All DNA segments were subcloned into pUC 18 (gutless pUC). All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into S. dimorphus via particle bombardment according to the method described in EXAMPLE 1 with DNA carried on 550 nm gold particles @500 psi and a shooting distance of 4 cm, Transformants were selected by growth on TAP-CAM agar medium under constant light 50-100 uE @RT for approximately 2 weeks. Transformants were streaked onto TAP-CAM agar medium to ensure single colony isolation and grown for 4 days under constant light.

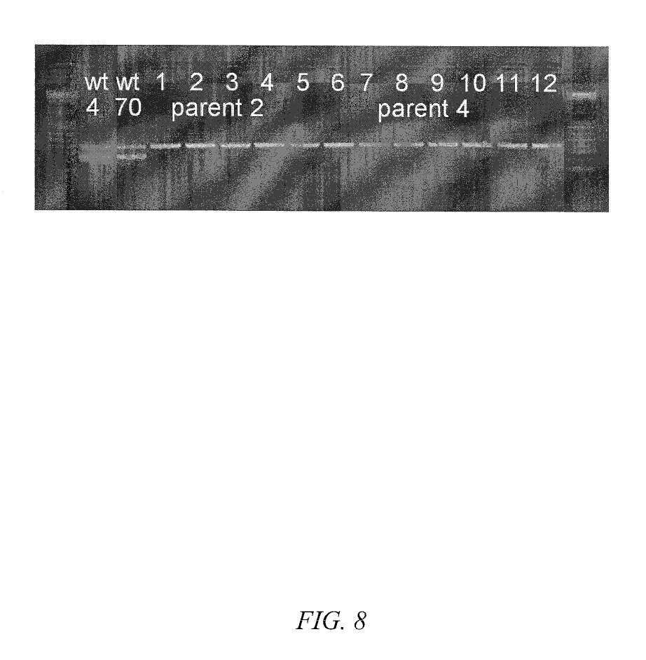

Transformants were analyzed by PCR screening (as described in EXAMPLE 1). The degree to which the transforming DNA was integrated into the chloroplast genome was determined using primers that amplify a 400 bp constant region (SEQ ID NO: 1 and SEQ ID NO: 2) and a 250 bp region spanning the integration site (SEQ ID NO: 3 and SEQ ID NO: 4). Integration occurs approximately 1000 bp 5′ of the start codon of the psbA gene. FIG. 8 shows that subclones from two independent transformants (parent 2 and 4) are homoplasmic, i.e., only the constant region (400 bp product) was amplified, while in the control reactions (wt) both the constant region and the region spanning the integration site (250 bp) were amplified.

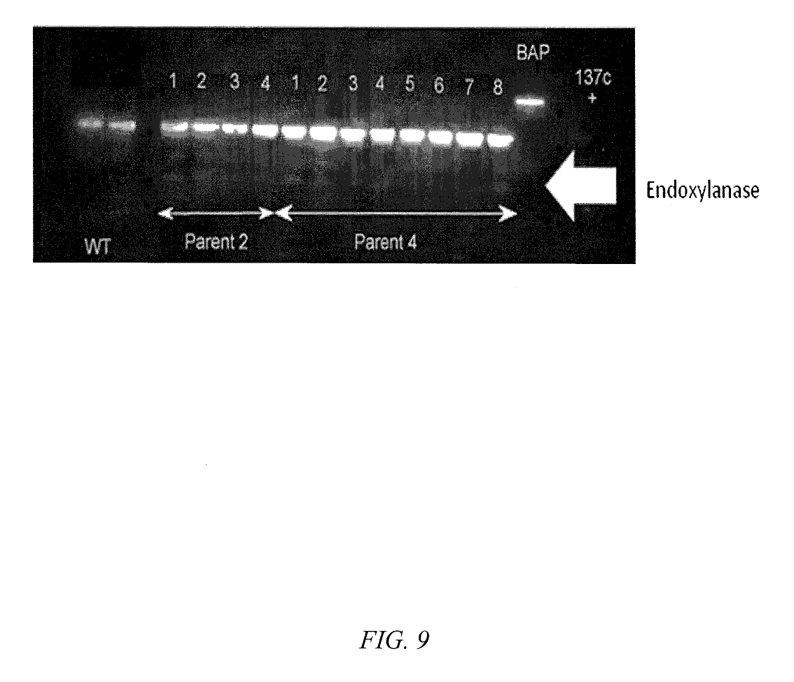

To ensure that the presence of the endoxylanase-encoding gene led to expression of the endoxylanase protein, a Western blot was performed. Briefly, approximately 1×10⁸ to 2×10⁸ algae cells were collected from TAP agar medium and resuspended in approximately 1 mL BugBuster solution (Novagen) in a 1.5 mL eppendorf tube. 1.0 mm Zirconia beads (BioSpec Products, inc) were then added to fill the tube with minimal headspace, ˜500 μL of beads. Cells were lysed in a bead beating apparatus (Mini Beadbeater™, BioSpec Products, Inc.) by shaking for 3-5 minutes three times. Cell lysates were clarified by centrifugation for 15 minutes at 20,000 g and the supernatants were normalized for total soluble protein (Coomassie Plus Protein Assay Kit, Thermo Scientific). Samples were mixed 1:4 with loading buffer (XT Sample Buffer with β-mercaptoethanol, Bio-Rad), heated to 98° C. for 5 min, cooled to 23° C., and proteins were separated by SDS-PAGE, followed by transfer to PVDF membrane. The membrane was blocked with Starting Block T20 Blocking Buffer (Thermo Scientific) for 15 min, incubated with horseradish peroxidase-linked anti-FLAG antibody (diluted 1:2,500 in Starting Block T20 Blocking Buffer) at 23° C. for 2 hours, washed three times with TBST. Proteins were visualized with chemiluminescent detection. Results from multiple clones (FIG. 9, parent 2 and 4) show that expression of the endoxylanase gene in S. dimorphus cells resulted in production of the protein.

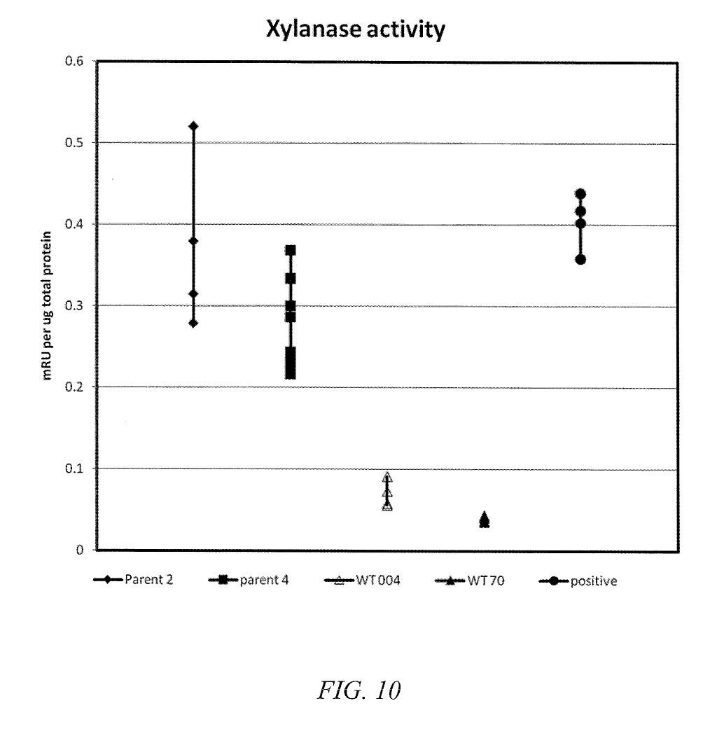

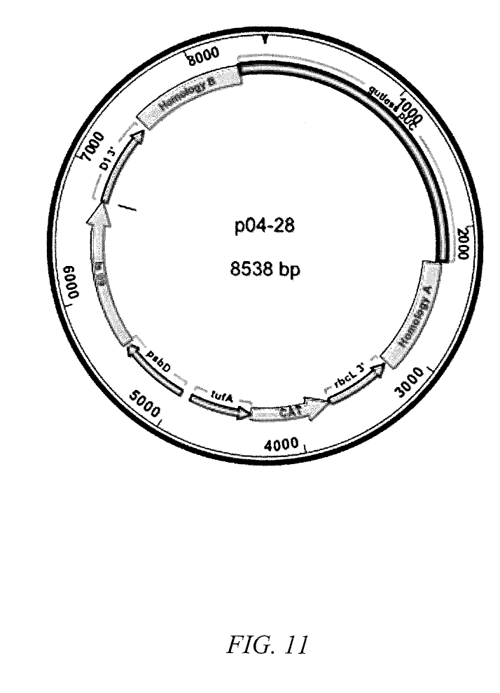

To determine if the endoxylanase produced by transformed algae cells was functional, endoxylanase activity was tested using an enzyme function assay. Briefly, algae cells were collected from TAP agar medium and suspended in BugBuster solution (Novagen). Cells were lysed by bead beating using zirconium beads. Cell lysates were clarified by centrifugation and the supernatants were normalized for total soluble protein (Coomassie Plus Protein Assay Kit, Thermo Scientific). 100 μL of each sample was mixed with 10 μL of 10× xylanase assay buffer (1M sodium acetate, pH=4.8) and 50 μL of the sample mixture was added to one well in a black 96-well plate. EnzCheck Ultra Xylanase substrate (Invitrogen) was dissolved at a concentration of 50 ug/ml in 100 mM sodium acetate pH 4.8, and 50 μL of substrate was added to each well of the microplate. The fluorescent signal was measured in a SpectraMax M2 microplate reader (Molecular Devices), with an excitation wavelength of 360 nm and an emission wavelength of 460 nm, without a cutoff filter and with the plate chamber set to 42 degrees Celsius. The fluorescence signal was measured for 15 minutes, and the enzyme velocity was calculated with Softmax Pro v5.2 (Molecular Devices). Enzyme velocities were recorded as RFU/minute. Enzyme specific activities were calculated as milliRFU per minute per μg of total soluble protein. FIG. 10 shows that endoxylanse activity is at least 4 fold higher in transformants than in wild type cells and similar in velocity to a positive control (Chlamydomonas expressing endoxylanse algae cells).

These data demonstrate that the chloroplast of S. dimorphus can be transformed with foreign DMA containing an expression cassette with a selectable marker and a separate expression cassette with a gene encoding an endoxylanase, and the expressed proteins are functional. One of skill in the art will appreciate that many other methods known in the art may be substituted in lieu of the ones specifically described or referenced.

### Example 5

### Production of FPP Synthase in S. dimorphus

In this example a nucleic acid encoding FPP synthase from G. gallus was introduced into S. dimorphus. Transforming DNA is shown graphically in FIG. 11. In this instance the DNA segment labeled “Is09” is the FPP synthase encoding gene (SEQ ID NO: 23 Is09), the segment labeled “psbD” is the promoter and 5′ UTR for the psbD gene from S. dimorphus, the segment: labeled “D1 3′UTR” is the 3′ UTR for the psbA gene from S. dimorphus, and the segment labeled “CAT” is the chloramphenicol acetyl transferase gene (CAT) from is E. coli, which is regulated by the promoter and 5′ UTR sequence for the tufA gene from S. dimorphus and the 3′ UTR sequence for the rbcL gene from S. dimorphus. The transgene expression cassette and selection marker are targeted to the S. dimorphus chloroplast genome via the segments labeled “Homology A” and “Homology B” which are approximately 1000 bp fragments homologous to sequences of DNA adjacent to nucleotide 071,366 (Site 1; nucleotide locations according to the sequence available from NCBI for S. obliquus, NC_{—}008101) on the 5′ and 3′ sides, respectively. All DNA segments were subcloned into pUC 18 (gutless pUC). All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP-CAM agar medium under constant light 50-100 uE @RT for approximately 2 weeks. Transformants were streaked onto TAP-CAM agar medium to ensure single colony isolation and grown for 4 days under constant light.

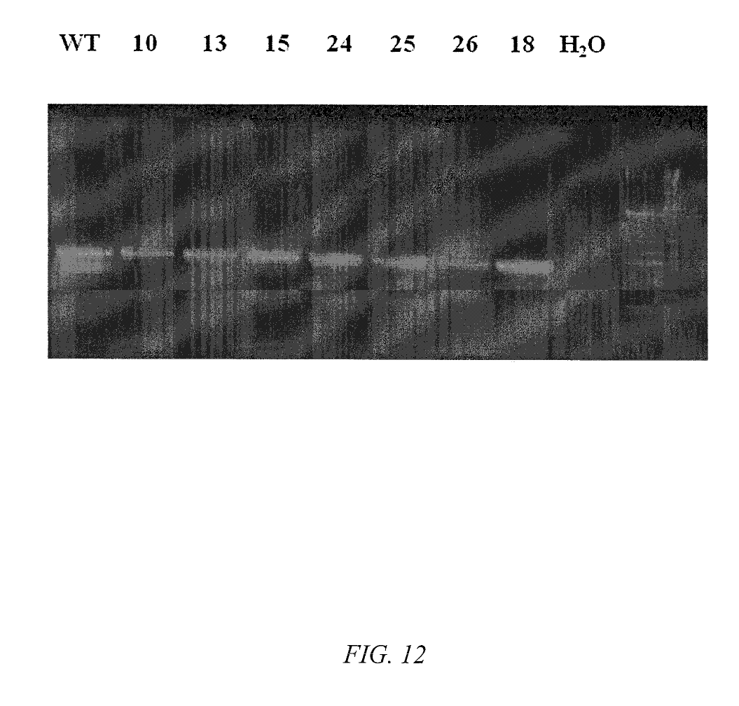

Transformants were analyzed by PCR screening (as described in EXAMPLE 1). The degree to which the transforming DNA was integrated into the chloroplast genome was determined using primers that amplify a 400 bp constant region (SEQ ID NO: 1 and SEQ ID NO: 2) and a 250 bp region spanning the integration site (SEQ ID NO: 3 and SEQ ID NO: 4). FIG. 12 shows that seven independent transformants are homoplasmic, i.e., only the constant region (400 bp product) was amplified, while in the control reactions (WT) both the constant region and the region spanning the integration, site (250 bp) were amplified.

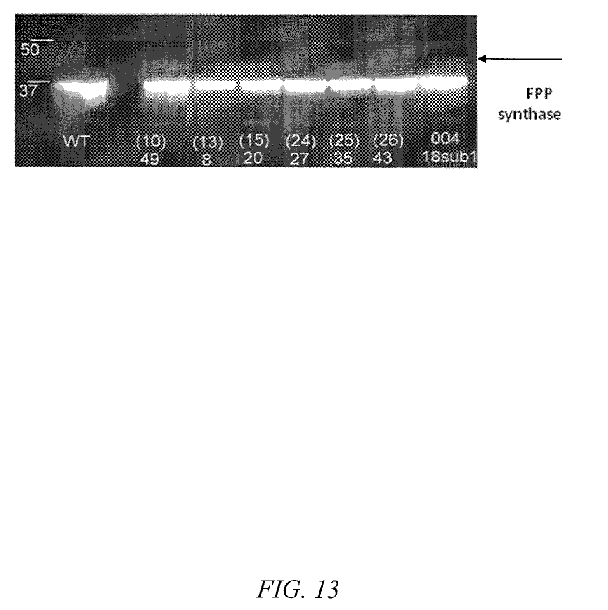

To ensure that the presence of the FPP synthase-encoding gene led to expression of the FPP synthase protein, a Western, blot was performed (as described in EXAMPLE 4). Results from multiple clones (FIG. 13) show that, expression of the FPP synthase gene in S. dimorphus cells resulted in production of the protein.

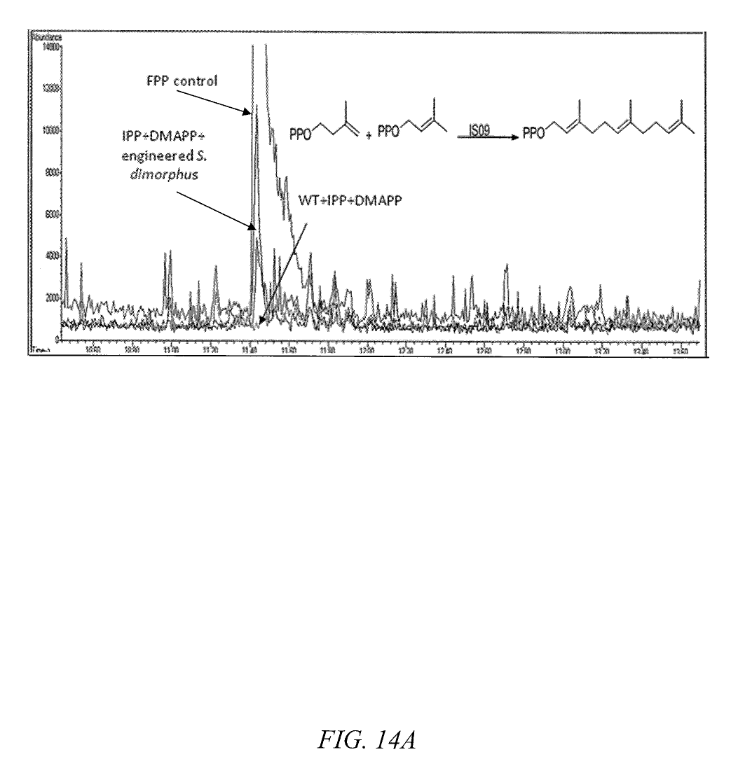

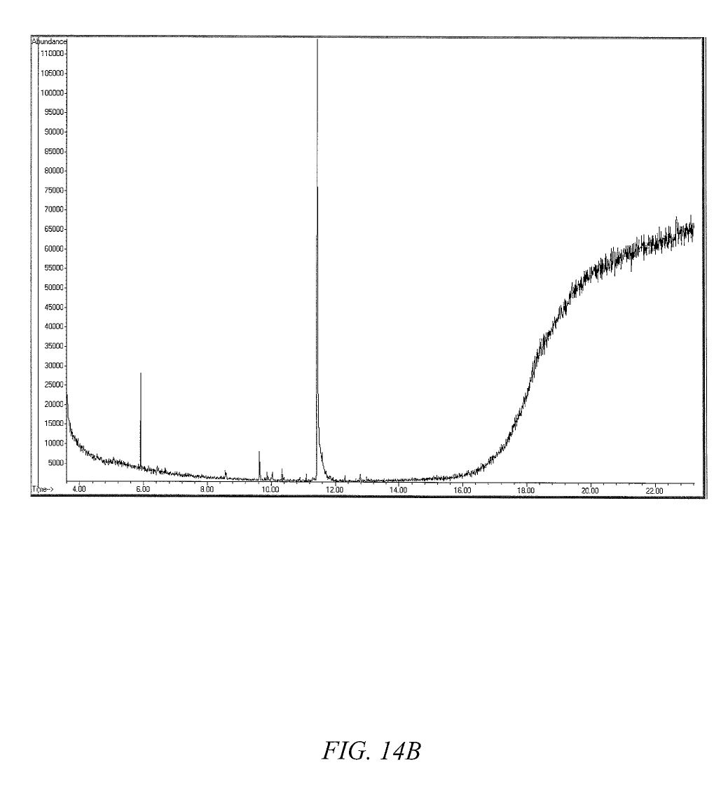

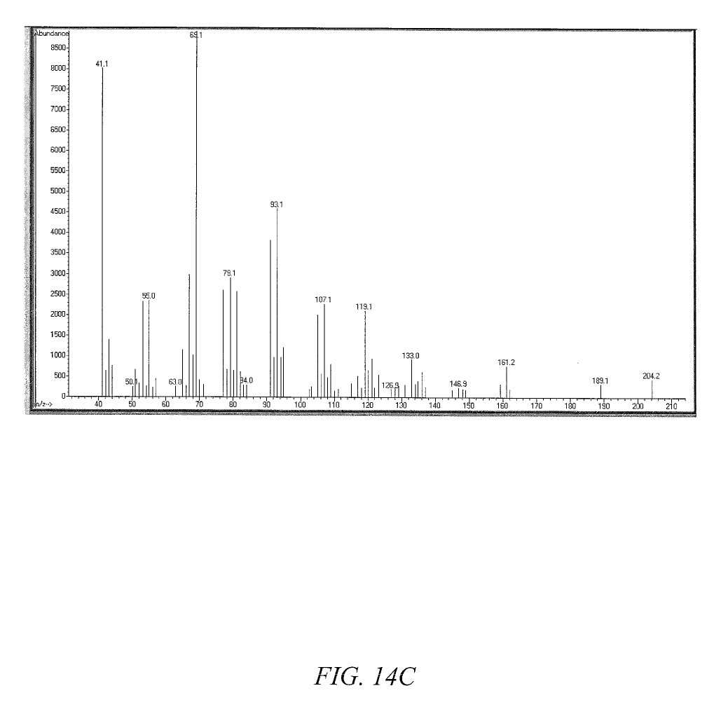

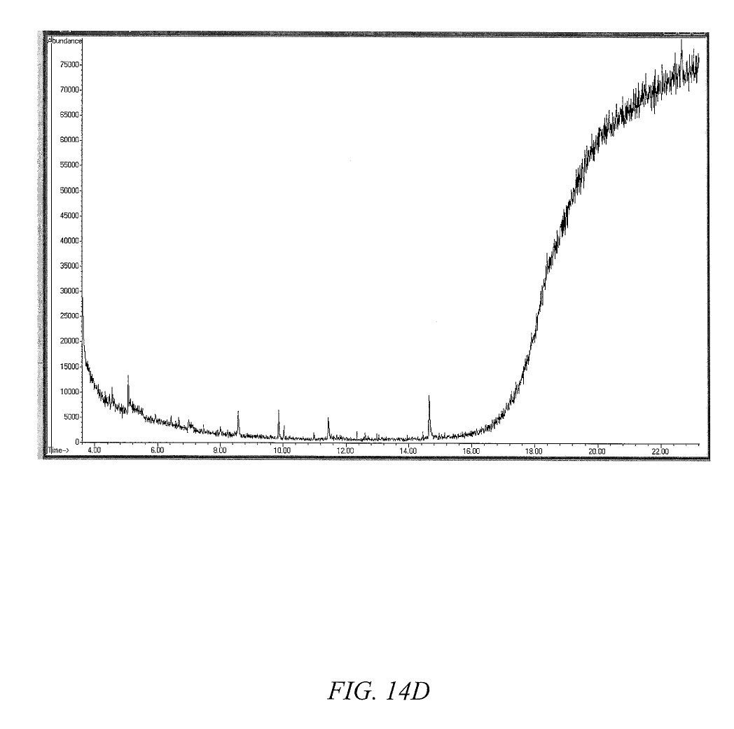

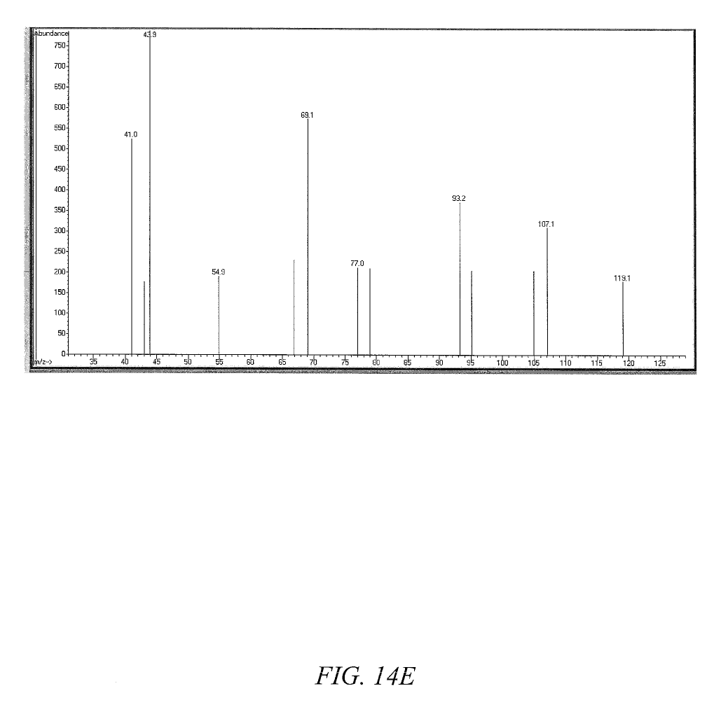

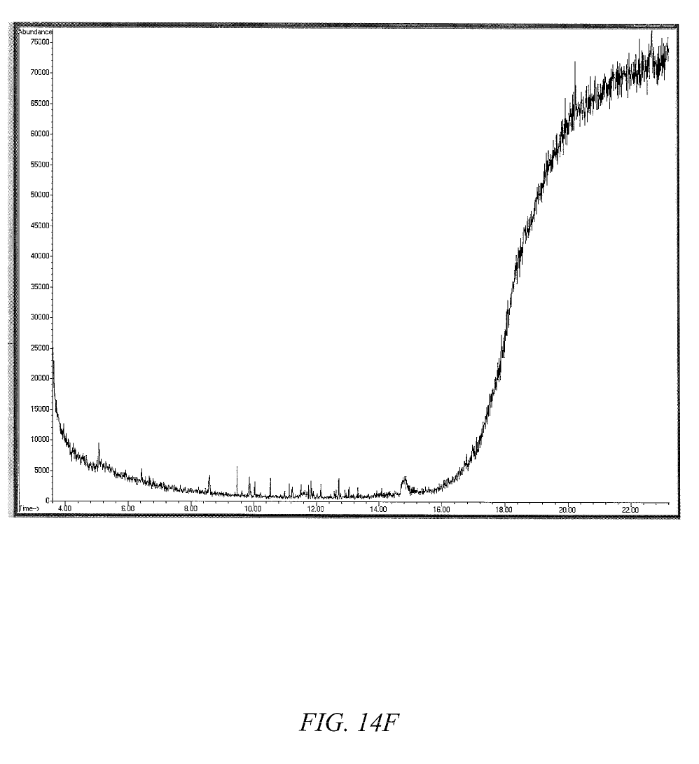

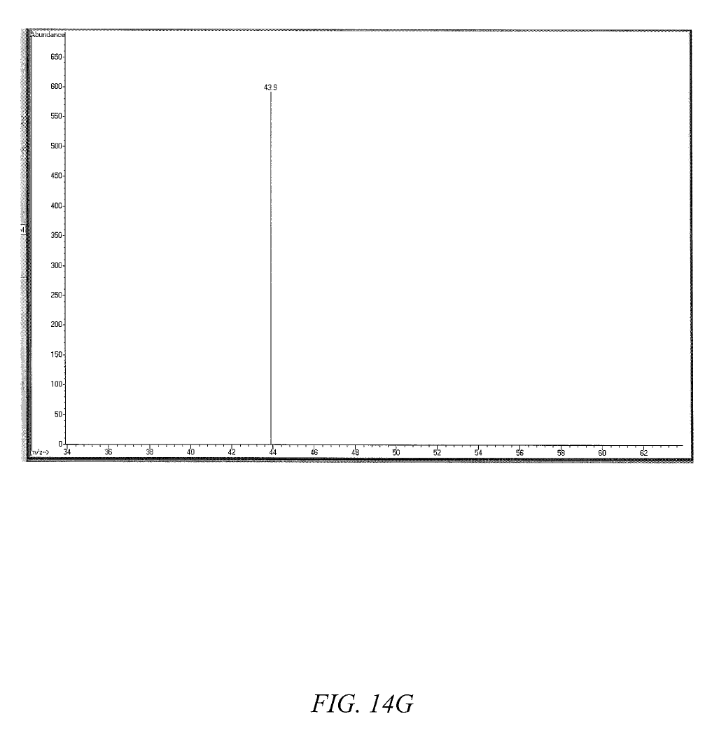

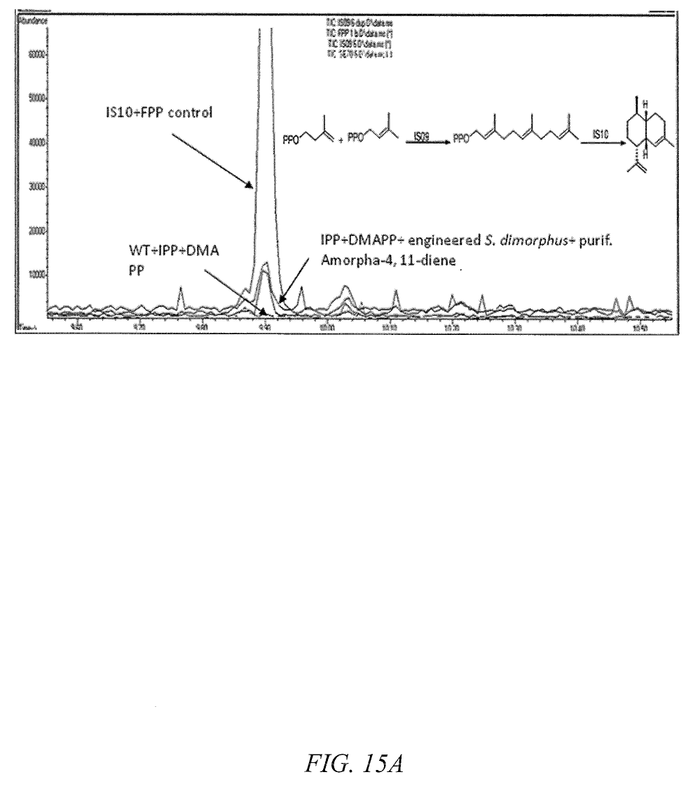

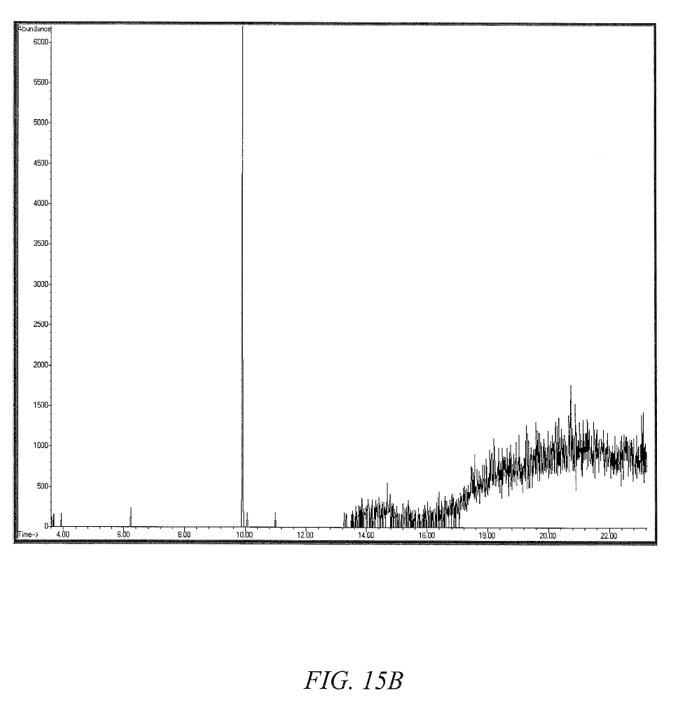

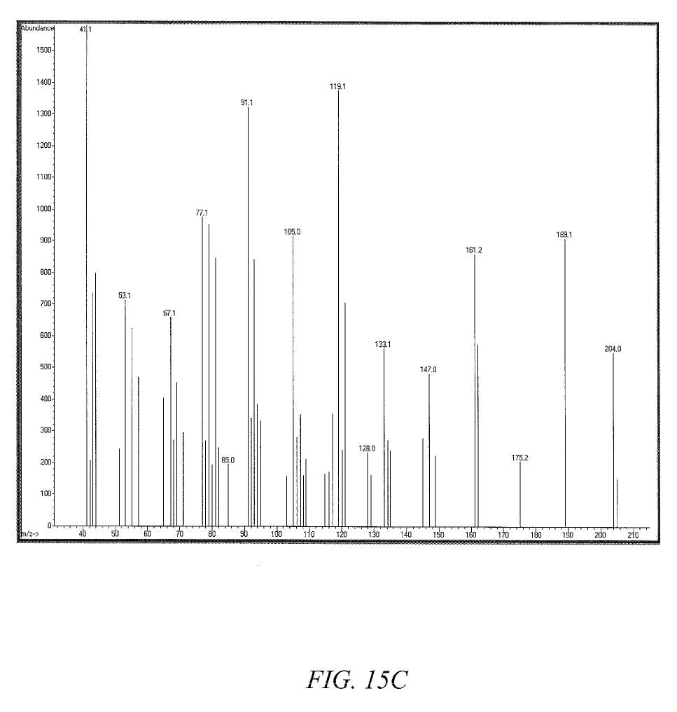

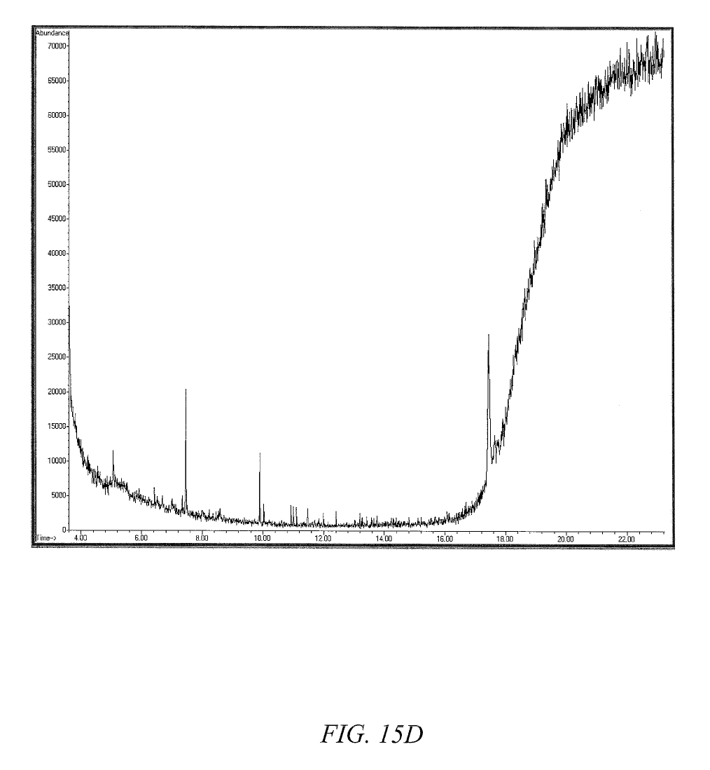

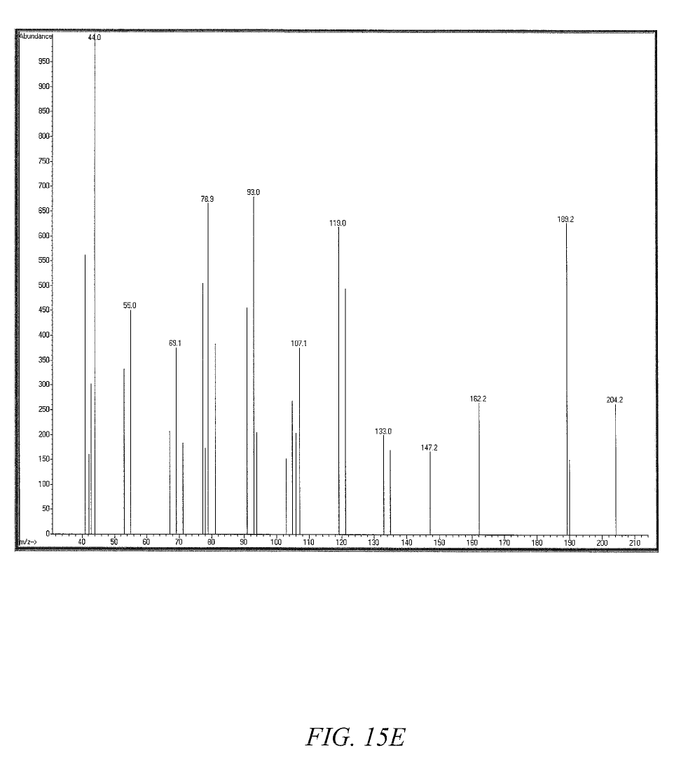

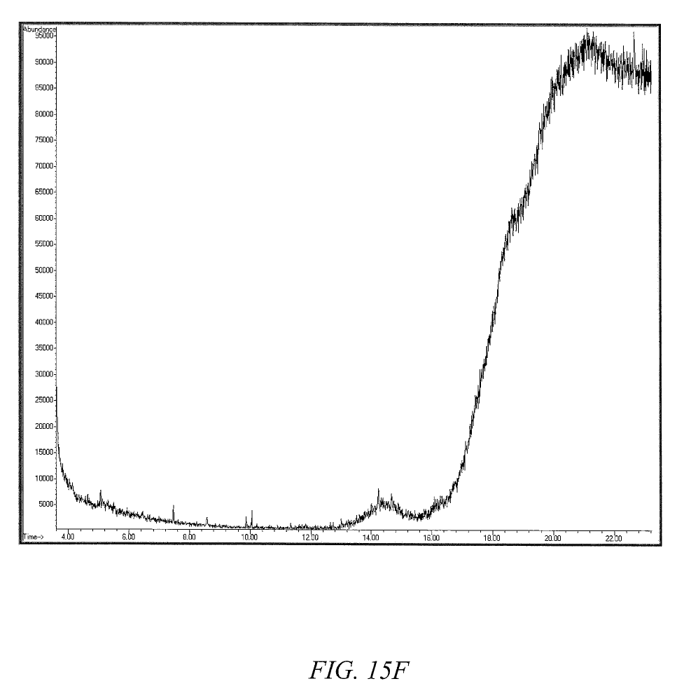

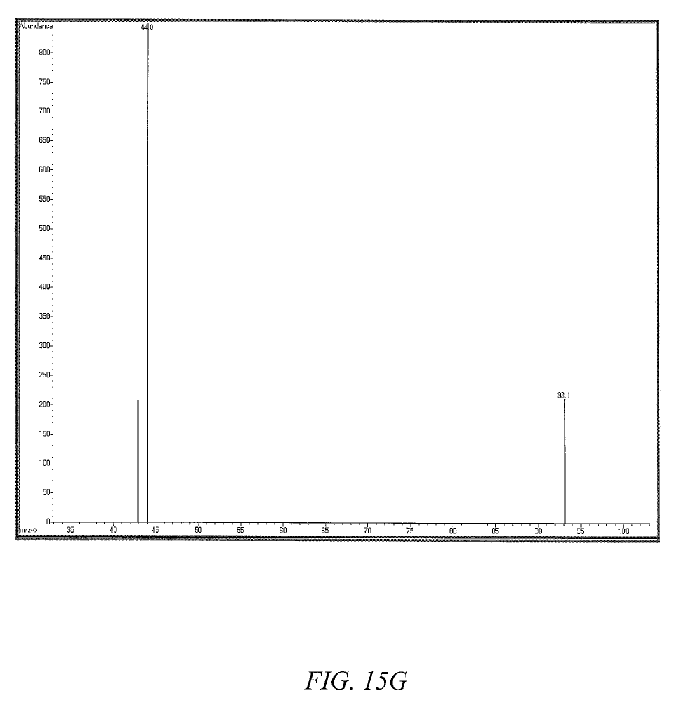

To determine if the FPP synthase produced by transformed algae cells was functional, FPP synthase activity was tested using an enzyme function assay. Algae cells were harvested from TAP media, resuspended in assay buffer (35 mM HEPES, pH 7.4, 10 mM MgCl₂, 5 mM DTT) and lysed using zirconium beads in a bead beater. Crude lysate was clarified by centrifugation at 15,000 rpm for 20 min. Isopentenyl diphosphate (IPP) and dimemthylallyl diphosphate (DMAPP) were added to clarified lysates and the reaction allowed to proceed at 30C overnight. Reactions were then CIP treated for 4-6 hours @37C in glycine buffer, pH 10.6, 5 mM ZnCl₂. The samples were then overlayed with heptane and analyzed via GC/MS (FIGS. 14A to G). Additionally, IPP, DMAPP and E. coli purified amorpha-4,11-diene were added to clarified lysates, the reactions allowed to proceed at 30° C. overnight, overlayed with heptane and analyzed via GC/MS (Figures ISA to G). For both methods, the diagnostic ions at m/Z 204 and 189 were detected in the engineered S. dimorphus, but not in the wt samples.

These data demonstrate that the chloroplast of S. dimorphus can be transformed with foreign DNA containing an expression cassette with a selectable marker and a separate expression cassette with a gene encoding an FPP synthase, and the expressed proteins are functional. One of skill in the art will appreciate that many other methods known in the art may be substituted in lieu of the ones specifically described or referenced.

### Example 6

### Production of Fusicoccadiene Synthase in S. dimorphus

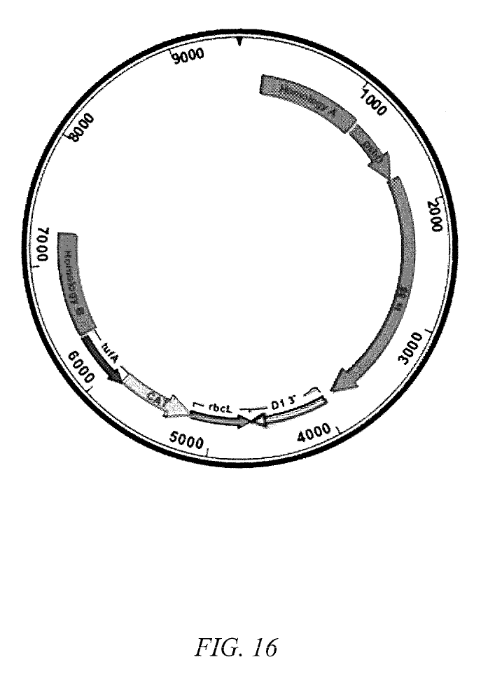

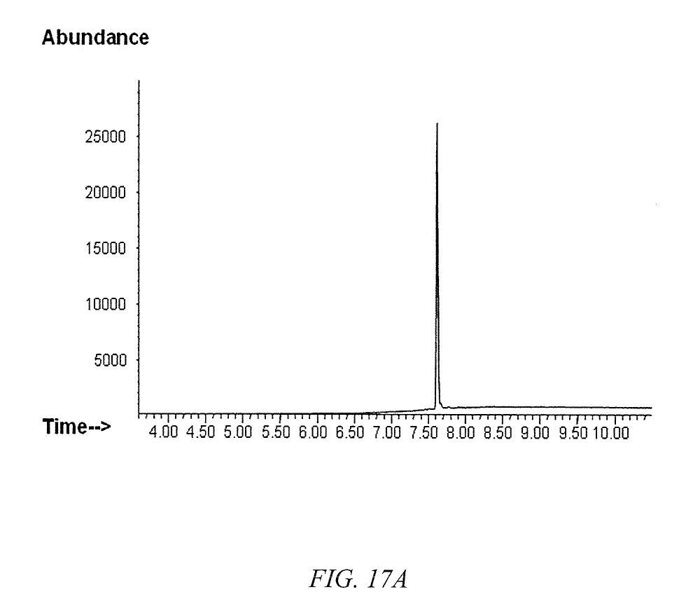

In this example a nucleic acid encoding fusicoccadiene synthase from P. amygdali was introduced into S. dimorphus. Transforming DNA is shown graphically in FIG. 16. In this instance the DNA segment labeled “Is88” is the fusicoccadiene synthase encoding gene (SEQ ID NO: 25, Is88), the segment labeled “psbD” is the promoter and 5′ UTR for the psbD gene from S. dimorphus, the segment labeled “D1 3′” is the 3′ UTR for the psbA gene from S. dimorphus, and the segment labeled “CAT” is the chloramphenicol acetyl transferase gene (CAT) from E. coli, which is regulated by the promoter and 5′ UTR sequence for the tufA gene from S. dimorphus and the 3′ UTR sequence for the rbcL gene from S. dimorphus. The transgene expression cassette and selection marker are targeted to the S. dimorphus chloroplast genome via the segments labeled “Homology A” and “Homology B” which are approximately 1000 bp fragments homologous to sequences of DNA adjacent to nucleotide 071,366 (Site 1; nucleotide locations according to the sequence available from NCBI for S. obliquus, NC_{—}008101) on the 5′ and 3′ sides, respectively. All segments were subcloned into pUC19. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP-CAM agar medium under constant light 50-100 uE @RT for approximately 2 weeks. Transformants were streaked onto TAP-CAM agar medium to ensure single colony isolation and grown for 4 days under constant light.

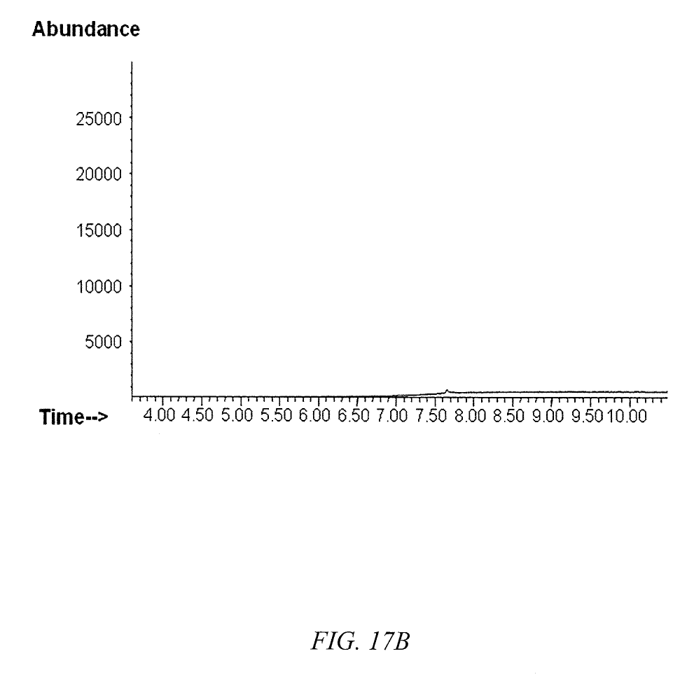

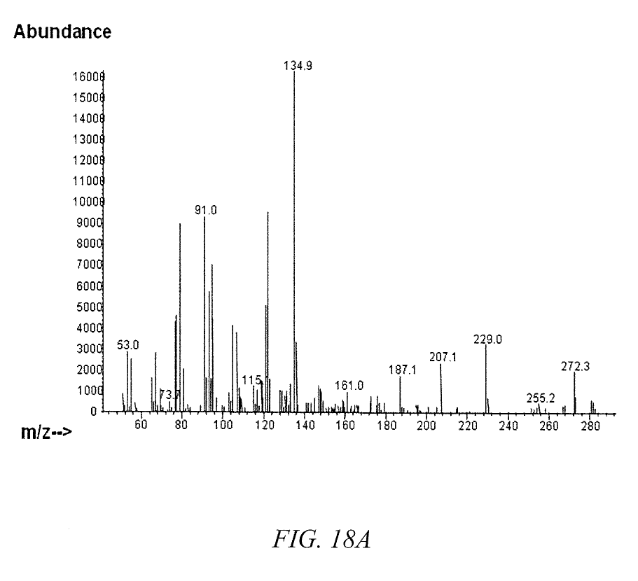

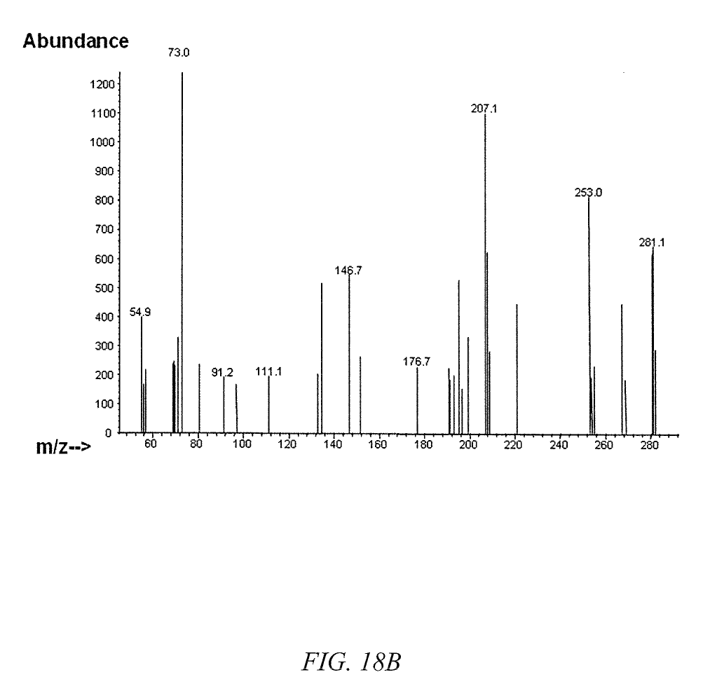

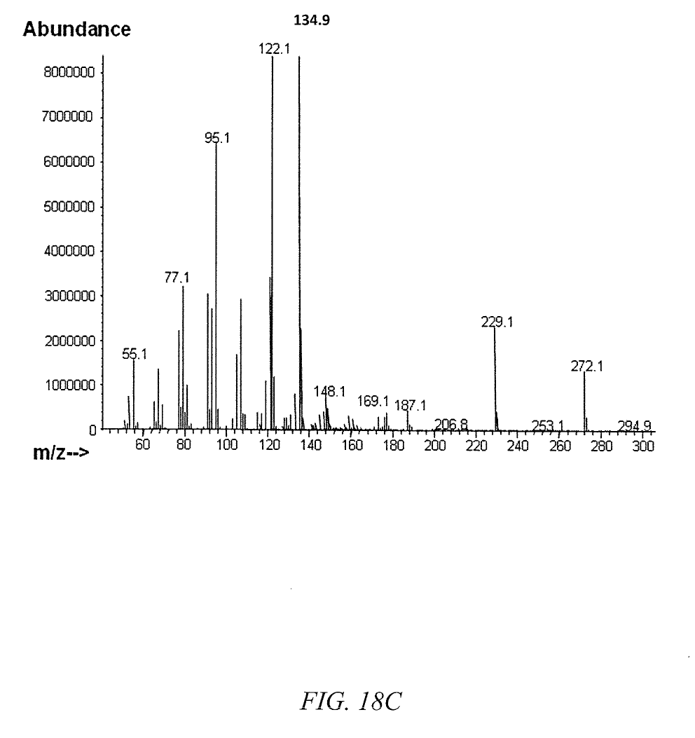

To determine if functional fusicoccadiene synthase is produced by transformed algal cells, cultures (2 ml) of gene positive, homoplasmic algae were collected by centrifugation, resuspended in 250 μl of methanol, and 500 μl of saturated NaCl in water and 500 μl of petroleum ether were added. The solution was vortexed for three minutes, then centrifuged at 14,000 g for five minutes at room temperature to separate the organic and aqueous layers. The organic layer (100 μl) was transferred to a vial insert in a standard 2 ml sample vial and analyzed using GC/MS. The mass spectrum at. 7.6±7 minutes for the sample from the engineered S. dimorphus is obtained. The diagnostic ions at m/Z=, 229, 135, and 122 are present in this spectrum, demonstrating the presence of fusicocca-2,10 (14)-diene and indole (FIG. 17 and FIG. 18).

These data demonstrate that the chloroplast of S. dimorphus can be transformed with foreign DNA containing an expression cassette with a selectable marker and a separate expression cassette with a gene encoding a fusicoccadiene synthase that produces a novel hydrocarbon in vivo. One of skill in the art will appreciate that many other methods known in the art may be substituted in lieu of the ones specifically described or referenced.

### Example 7

### Production of Phytase in S. dimorphus

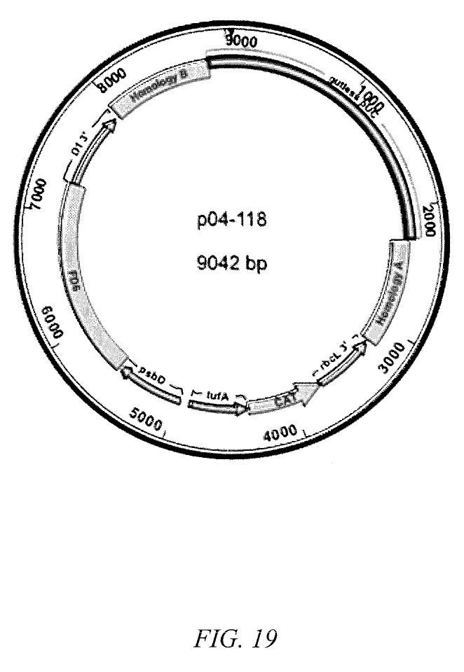

In this example a nucleic acid encoding phytase from E. coli was introduced into S. dimorphus. Transforming DNA is shown graphically in FIG. 19. In this instance the DNA segment labeled “FD6” is the phytase encoding gene (SEQ ID NO: 26, FD6), the segment labeled “psbD” is the promoter and 5′ UTR for the psbD gene from S. dimorphus, the segment labeled “D1 3′” is the 3′ UTR for the psbA gene from S. dimorphus, and the segment labeled “CAT” is the chloramphenicol acetyl transferase gene (CAT) from E. coli, which is regulated by the promoter and 5′ UTR sequence for the tufA gene from S. dimorphus and the 3′ UTR sequence for the rbcL gene from S. dimorphus. The transgene expression cassette and selection marker are targeted to the S. dimorphus chloroplast genome via the segments labeled “Homology A” and “Homology B” which are approximately 1000 bp fragments homologous to sequences of DNA adjacent to nucleotide 071,366 (Site 1; nucleotide locations according to the sequence available from NCBI for S. obliquus, NC_{—}008101) on the 5′ and 3′ sides, respectively. All DNA segments were cloned into pUC19. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP-CAM agar medium under constant light 50-100 uE @RT for approximately 2 weeks. Transformants were streaked onto TAP-CAM agar medium to ensure single colony isolation and grown for 4 days under constant light.

Transformants were analyzed by PCR screening (as described in EXAMPLE 1) and homoplasmic clones were identified and subcultured for further studies.

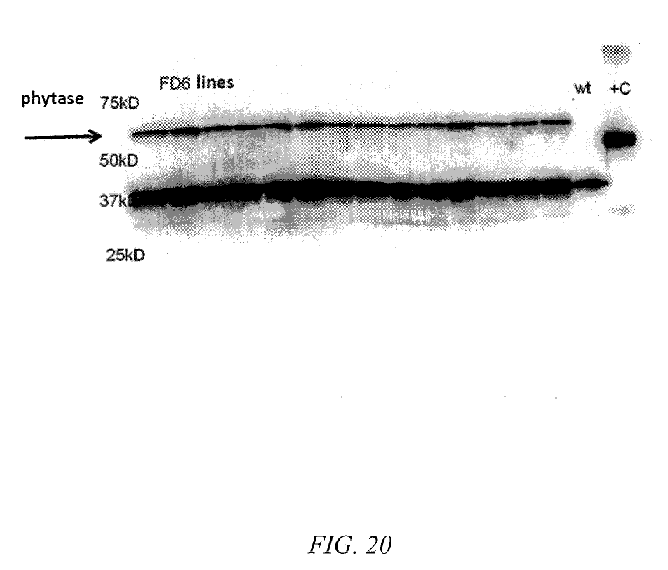

To ensure that the presence of the phytase-encoding gene led to expression of the phytase protein, a Western blot was performed (as described in EXAMPLE 4). Results from multiple clones (FIG. 20) show that expression of the phytase gene in S. dimorphus cells resulted in production of the protein.

One of skill in the art will appreciate that many other methods known in the art may be substituted in lieu of the ones specifically described or referenced.

### Example 8

### Use of Erythromycin Esterase as a Selection Marker in S. dimorphus and S. obliquus

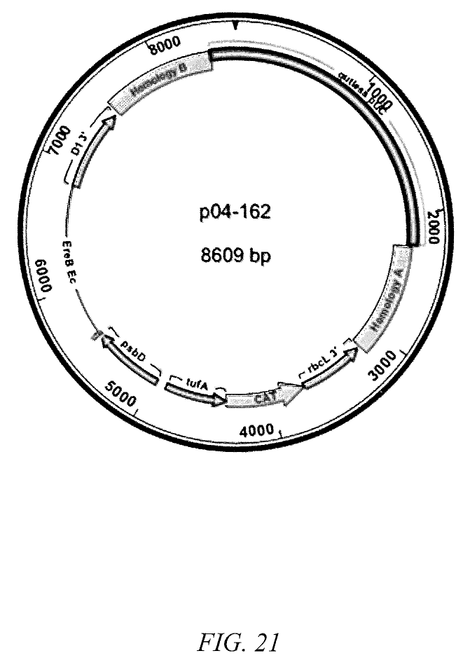

In this example, a nucleic acid encoding erythromycin esterase gene (EreB) (SEQ ID NO: 29) from E. coli was introduced into S. dimorphus. Transforming DNA is shown graphically in FIG. 21. In this instance the DNA segment labeled “EreB ec” is the erythromycin esterase gene (EreB) from E. coli, the segment labeled “psbD” is the promoter and 5′ UTR. sequence for the psbD gene from S. dimorphus, and the segment labeled “D1 3′” is the 3′ UTR for the psbA gene from S. dimorphus. The selection marker cassette is targeted to the S. dimorphus chloroplast genome via the segments labeled “Homology A” and “Homology B” which are approximately 1000 bp fragments homologous to sequences of DNA adjacent to nucleotide 071,366 (Site 1; nucleotide locations according to the sequence available from NCBI for S. obliquus, NC_{—}008103) on the 5′ and 3′ sides, respectively. All segments were cloned into pUC19. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 392-208, 1998.

Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP agar medium+50 μg/mL erythromycin (TAP-ERM50) under constant light 50-100 uE @RT for approximately 2 weeks. Transformants were streaked onto TAP-ERM50 agar medium to ensure single colony isolation and grown for 4 days under constant light.

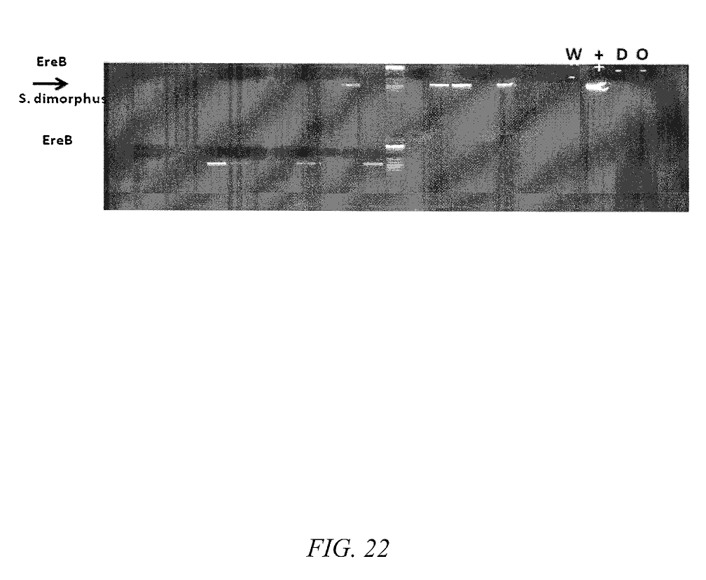

Transformants were analyzed by PCR screening (as described in EXAMPLE 1). The presence of the EreB selection marker was determined using primers that amplify a 555 bp region within the gene (SEQ ID NO: 7 and SEQ ID NO: 8). FIG. 22 shows that the EreB gene (SEQ ID NO: 29) was amplified from DNA from several transformants but not from wildtype DNA from S. dimorphus.

One of skill in the art will appreciate that many other methods known in the art may be substituted in lieu of the ones specifically described or referenced.

### Example 9

### Use of codA as a Selection Marker in S. dimorphus

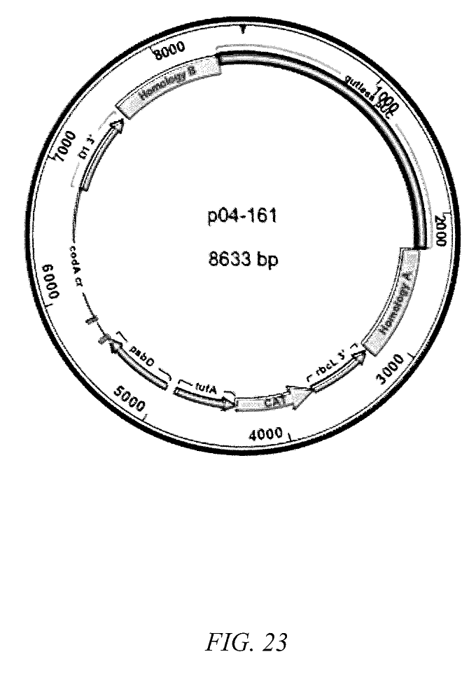

In this example, a nucleic acid encoding cytosine deaminase gene (codA) from E. coli was introduced into S. dimorphus. Transforming DNA is shown graphically in FIG. 23. In this instance the DNA segment labeled “codA cr” is the codA encoding gene (SEQ ID NO: 31, codA), the segment labeled “psbD” is the promoter and 5′ UTR for the psbD gene from S. dimorphus, the segment labeled “D1 3′” is the 3′ UTR for the psbA gene from S. dimorphus, and the segment labeled “CAT” is the chloramphenicol acetyl transferase gene (CAT) from E. coli, which is regulated by the promoter and 5′ UTR sequence for the tufA gene from S. dimorphus and the 3′ UTR sequence for the rbcL gene from S. dimorphus. The transgene expression cassette and selection marker are targeted to the S. dimorphus chloroplast genome via the segments labeled “Homology A” and “Homology B” which are approximately 1000 bp fragments homologous to sequences of DNA adjacent to nucleotide 071,366 (Site 1; nucleotide locations according to the sequence available from NCBI for S. obliquus, NC_{—}008101) on the 5′ and 3′ sides, respectively. All DNA segments were cloned into pUC19. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP-CAM agar medium under constant light 50-100 uE @RT for approximately 2 weeks. Transformants were streaked onto TAP-CAM agar medium to ensure single colony isolation and grown for 4 days under constant light.

Transformants were analyzed by PCR screening (as described in EXAMPLE 1) and homoplasmic clones were identified and subcultured for further studies.

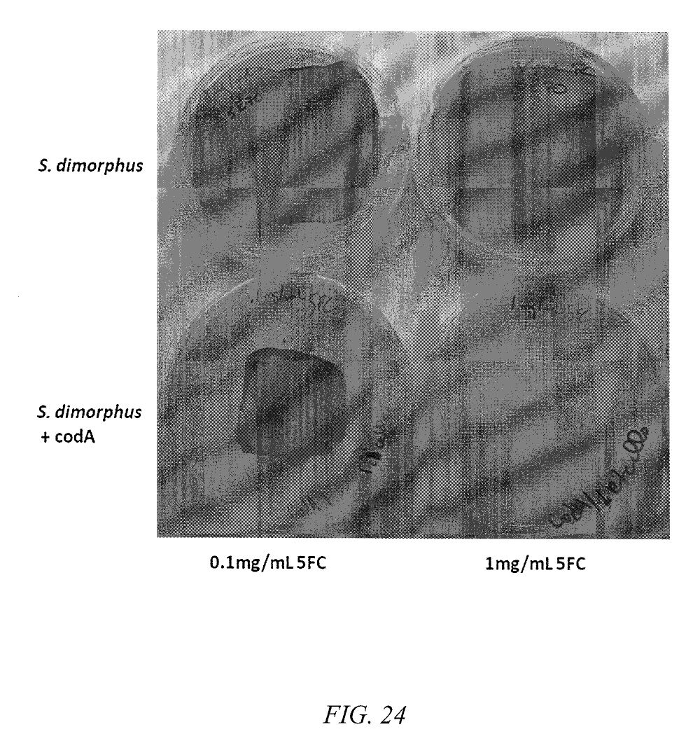

To determine if functional codA is produced by transformed algae cells, cells were grown in TAP media to log phase, pelleted and resuspended to 10⁸ cells/mL and plated onto TAP agar medium containing 1 mg/mL 5-fluorocytosine (5FC). Wildtype S. dimorphus, survives on TAP agar containing 1 mg/mL 5FC, while transformants containing the transgene do not (FIG. 24). These data demonstrate that the chloroplast of S. dimorphus can be transformed with foreign DNA containing an expression cassette with a selectable marker and a separate expression cassette with a gene encoding a cytosine deaminase producing a cell with a negatively selectable phenotype.

This S. dimorphus homoplasmic codA line can now be transformed with either 1) a vector containing a gene of interest cassette without a selection marker in site 1 (the same site that the codA cassette is located within the genome) and after a recovery period on nonselective medium, selected for on medium containing 5FC, or 2) a vector containing a gene of interest cassette linked with an EreB cassette at site 1 and selected on medium containing erythromycin. In this instance, transformants can be streaked onto TAP medium+50 μg/mL erythromycin for single colony isolation and subclones can be patched onto TAP+1 mg/mL 5FC to select for clones homoplasmic for the EreB cassette.

One of skill in the art will appreciate that many other methods known in the art may be substituted in lieu of the ones specifically described or referenced.

### Example 10

### Else of codA as a Selection Marker of S. obliquus

In this example, a nucleic acid encoding cytosine deaminase gene (codA) from E. coli was introduced into S. obliquus. Transforming DNA is shown graphically in FIG. 23. In this instance the DNA segment labeled “codA cr” is the codA encoding gene (SEQ ID NO: 27, codA), the segment labeled “psbD” is the promoter and 5′ UTR for the psbD gene from S. dimorphus, the segment labeled “D1 3′” is the 3′ UTR for the psbA gene from 6′. dimorphus, and the segment labeled “CAT” is the chloramphenicol acetyl transferase gene (CAT) from E. coli, which is regulated by the promoter and 5′ UTR sequence for the tufA gene from S. dimorphus and the 3′ UTR sequence for the rbcL gene from S. dimorphus. The transgene expression cassette and selection marker are targeted to the S. obliquus chloroplast genome via the segments labeled “Homology A” and “Homology B” which are approximately 1000 bp fragments homologous to sequences of DNA adjacent to nucleotide 071,366 (Site 1; nucleotide locations according to the sequence available from NCBI for S. obliquus, NC_{—}008101) on the 5′ and 3′ sides, respectively. All DNA segments were cloned into pUC19. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into S. obliquus via particle bombardment according to the method described in EXAMPLE 1 with DNA carried on 550 nm gold particles @500 psi and a shooting distance of 4 cm. Transformants were selected by growth on TAP-CAM agar medium under constant light 50-100 uE RT for approximately 2 weeks. Transformants were streaked onto TAP-CAM agar medium to ensure single colony isolation and grown for 4 days under constant light.

Transformants were analyzed by PCR screening (as described in EXAMPLE 1) and homoplasmic clones were identified and subcultured for further studies.

To determine if functional codA is produced by transformed algae cells, cells were plated onto TAP agar medium containing 1 mg/mL 5-fluorocytosine (5FC). Wild type S. dimorphus survived on TAP agar containing 5FC, while transformants containing the transgene did not (FIG. 24).

These data demonstrate that the chloroplast of S. obliquus can be transformed with foreign. DNA containing an expression cassette with a selectable marker and a separate expression cassette with a gene encoding a cytosine deaminase producing a cell with a negatively selectable phenotype

This S. obliquus homoplasmic codA line can now be transformed with either 1) a vector containing a gene of interest cassette without a selection marker in site 1 (the same site that the codA cassette is located within the genome) and after a recover period on nonselective medium, selected for on medium containing 5FC or 2) a vector containing a gene of interest cassette linked with an EreB cassette at site 1 and selected on medium containing erythromycin. In this instance, transformants can be streaked onto TAP medium+50 μg/mL erythromycin for single colony isolation and subclones can be patched onto TAP+1 mg/mL 5FC to select for clones homoplasmic for the EreB cassette.

One of skill in the art will appreciate that many other methods known in the art may be substituted in lieu of the ones specifically described or referenced.

### Example 11

### Identification of Functional Promoters for Gene Expression in S. dimorphus

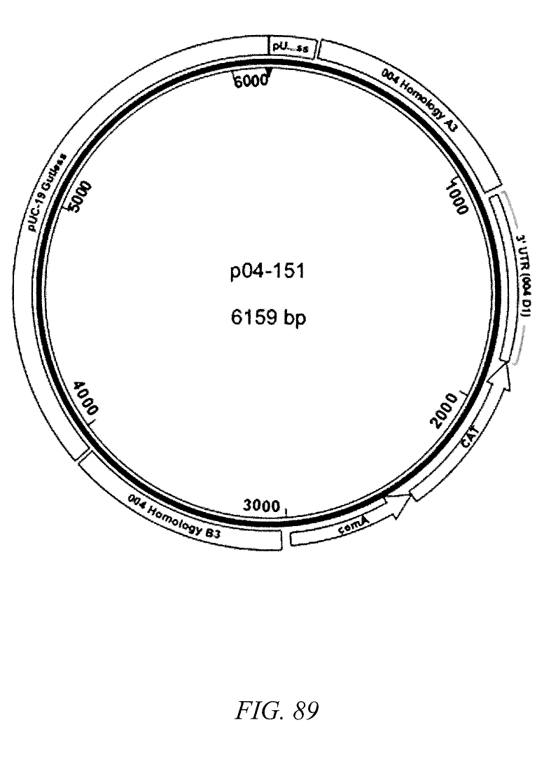

In this example, 8 promoters were amplified from S. dimorphus DNA and cloned upstream of the E. coli CAT gene. Transforming DNA (p04-151) is shown graphically in FIG. 89. In this instance the DNA segment labeled “CAT” is the chloramphenicol acetyl transferase gene (CAT) from E. coli (SEQ ID NO: 28, CAT), the segment labeled “tufA” is the promoter consisting of 500 bp of the 5′ UTR sequence for the chlB (SEQ ID NO: 51), psbB (SEQ ID NO: 39), psbA (SEQ ID NO: 37), rpoA (SEQ ID NO: 44), rbcL (SEQ ID NO: 49), cemA (SEQ ID NO: 45), ftsH (SEQ ID NO: 47), petA (SEQ ID NO: 53), petB (SEQ ID NO: 55) genes from S. dimorphus, and the segment labeled “D1 3” is the 3′ UTR for the psbA gene from S. dimorphus. The selection marker cassette is targeted to the S. dimorphus chloroplast genome via the segments labeled “Homology A” and “Homology B” which are approximately 1000 bp fragments homologous to sequences of DNA adjacent to nucleotide 071,366 (Site 1; nucleotide locations according to the sequence available from NCBI for S. obliquus, NC_{—}008101) on the 5′ and 3″ sides, respectively. All DNA segments were subcloned into pUC19. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP agar medium+25 μg/mL chloramphenicol (TAP-CAM) under constant light 50-100 uE @RT for approximately 2 weeks. Each promoter chlB (SEQ ID NO: 51), psbB (SEQ ID NO: 39), psbA (SEQ ID NO: 37), rpoA (SEQ ID NO: 44), rbcL (SEQ ID NO: 49), cemA (SEQ ID NO: 45), ftsH (SEQ ID NO: 47), petA (SEQ ID NO: 53), petB (SEQ ID NO: 55) gave rise to chloramphenicol resistant transformants indicating that these promoter/5′ UTR fragments were able to drive expression of the CAT gene.

### Example 12

### Multiple Gene Expression in S. dimorphus

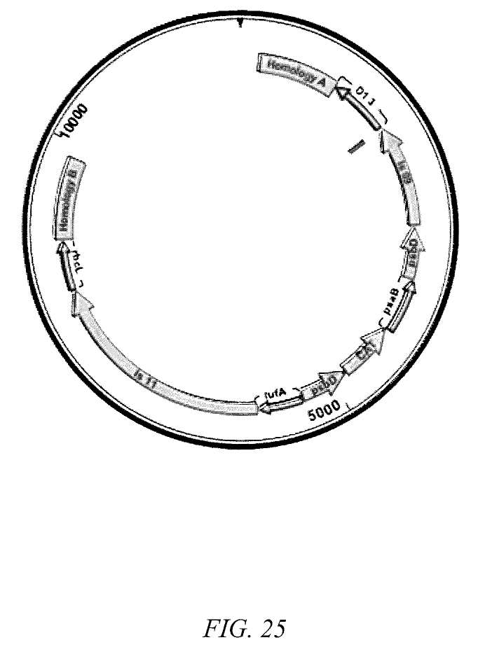

In this example a nucleic acid encoding FPP synthase from G. gallus and a nucleic acid encoding bisabolene synthase from A. grandis was introduced into S. dimorphus. Transforming DNA is shown graphically in FIG. 25. In this instance the DNA segment labeled “Is09” is the FPP synthase encoding gene (SEQ ID NO: 23, Is09), the segment labeled “psbD” is the promoter and 5′ UTR for the psbD gene from S. dimorphus, the segment labeled “D1 3′” is the 3′ UTR for the psbA gene from S. dimorphus, the segment labeled “Is11” is the bisabolene synthase encoding gene (SEQ ID NO: 35, Is011), the segment labeled “tufA” is the promoter and 5′ UTR for the tufA gene from S. dimorphus, the segment labeled “rbcL” is the 3′ UTR for the rbcL gene from S. dimorphus, and the segment labeled “CAT” is the chloramphenicol acetyl transferase gene (CAT) from E. coli, which is regulated by the promoter and 5′ UTR. sequence for the psbD gene from S. dimorphus and the 3′ UTR sequence for the psaB gene (SEQ ID NO: 59) from S. dimorphus. The transgene expression, cassette and selection marker are targeted to the S. dimorphus chloroplast genome via the segments labeled “Homology A” and “Homology B” which, are approximately 1000 bp fragments homologous to sequences of DNA adjacent. to nucleotide 071,366 (Site 1; nucleotide locations according to the sequence available from NCBI for S. obliquus, NC_{—}008101) on the 5′ and 3′ sides, respectively. All DNA segments were subcloned into pUC19. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP-CAM agar medium under constant light 50-100 uE @RT for approximately 2 weeks. Transformants were streaked onto TAP-CAM agar medium to ensure single colony isolation and grown for 4 days under constant light.

Transformants were analyzed by PCR screening (as described in EXAMPLE 1) and homoplasmic clones were identified and subcultured for further studies.

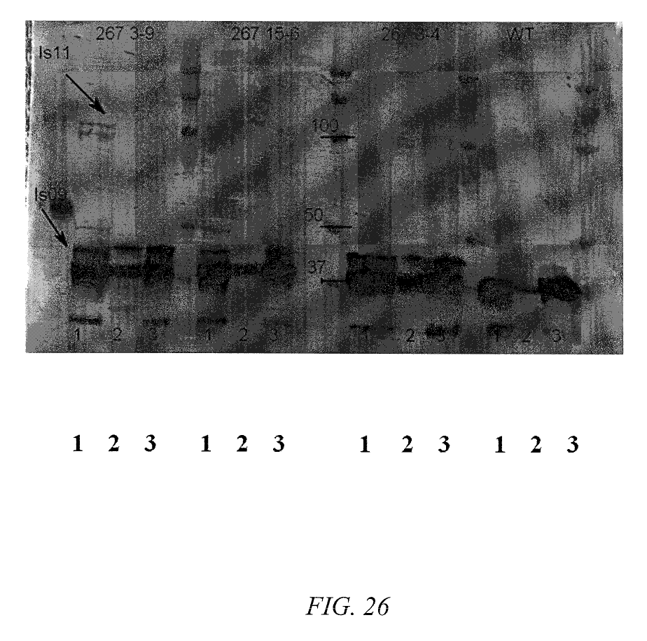

To ensure that the presence of the FPP synthase-encoding gene and the bisabolene-encoding gene led to expression of the FPP synthase and bisabolene synthase proteins, a Western blot was performed (as described in EXAMPLE 4). Proteins were visualized by a colormetric assay as per manufacturers instructions (1-step TMB blotting. Pierce). Results from multiple clones (267 3-9; 267 15-6; and 367 3-4) (FIG. 26) show that expression of the FPP synthase gene (Is09) and bisabolene synthase (Is11) in S. dimorphus cells resulted in production of both proteins. WT is untransformed S. dimorphus. These data demonstrate that the chloroplast of S. dimorphus can be transformed with a vector of foreign DNA containing an expression cassette with a selectable marker and two separate expression cassette with a gene encoding an FPP synthase and an E-alpha-bisabolene synthase, and that both proteins are expressed.

### Example 13

### Multiple Gene Expression in S. dimorphus

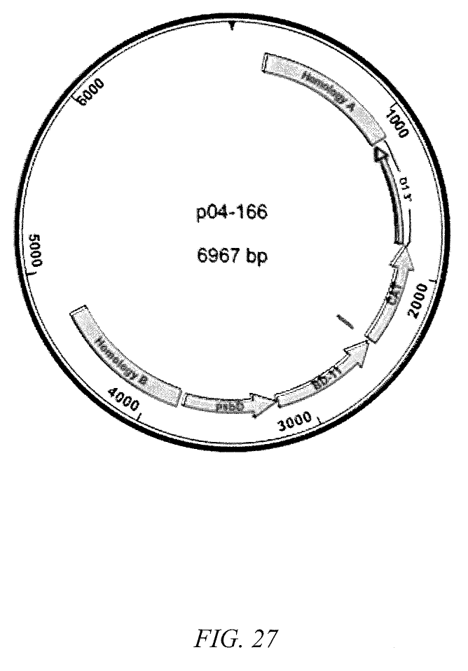

In this example, a nucleic acid encoding endoxylanase from T. reesei and chloramphenicol acetyl transferase gene (CAT) from E. coli linked together by a ribosome binding sequence from E. coli was introduced into S. dimorphus. Transforming DNA (BD11-RBS-CAT) is shown graphically in FIG. 27. In this instance the DNA segment labeled “BD11” is the endoxylanase encoding gene (SEQ ID NO: 21, BD11), the segment labeled “CAT” is the chloramphenicol acetyl transferase encoding gene (SEQ ID NO: 28, CAT), the segment labeled “RBS1” is the ribosome binding sequence (SEQ ID NO: 60, RBS1), the segment labeled “psbD” is the promoter and 5′ UTR for the psbD gene from S. dimorphus, the segment labeled “psbA” is the 3′ UTR for the psbA gene from S. dimorphus, and the segment labeled “CAT” is the chloramphenicol acetyl transferase gene (CAT) from E. coli, which is regulated by the promoter and 5′ UTR sequence for the tufA gene from S. dimorphus and the 3′ UTR sequence for the rbcL gene from S. dimorphus. The transgene expression cassette and selection marker are targeted to the S. dimorphus chloroplast genome via the segments labeled “Homology A” and “Homology B” which are approximately 1000 bp fragments homologous to sequences of DNA adjacent to nucleotide 071,366 (Site 1; nucleoside locations according to the sequence available from NCBI for S. obliquus, NC_{—}008101) on the 5′ and 3′ sides, respectively. All DNA segments were subcloned into pUC19. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP agar medium+25 μg/mL chloramphenicol, Transformants were streaked onto TAP-CAM agar medium to ensure single colony isolation and grown for 4 days under constant light.

Transformants were analyzed by PCR screening (as described in EXAMPLE 1) and homoplasmic clones were identified and subcultured for further studies.

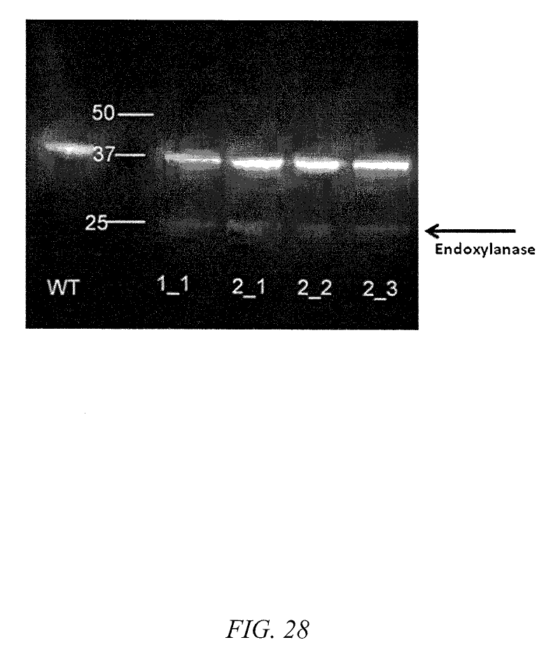

To ensure that the presence of the endoxylanase-encoding gene led to expression of the endoxylanase protein, a Western blot was performed (as described in EXAMPLE 4). Results from multiple clones (FIG. 28) show that expression of the endoxylanase gene in S. dimorphus cells resulted in production of the protein of expected molecular weight and not of an endoxylanse-CAT fusion protein.

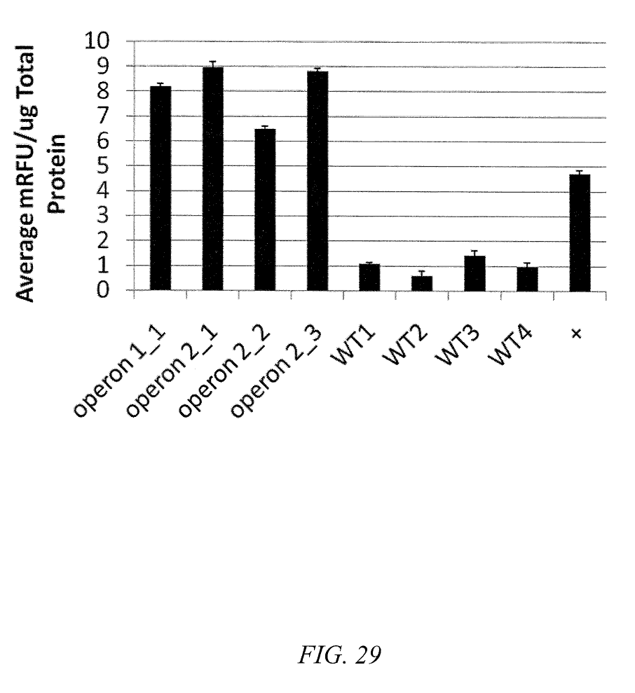

To determine if the endoxylanase produced by transformed algae cells was functional, endoxylanase activity was tested using an enzyme function assay (as described in EXAMPLE 4). FIG. 29 shows that endoxylanase activity is detected in clarified lysates of S. dimorphus engineered with the endoxylanase-RBS-CAT construct (operon 1_{—}1, 2_{—}1, 2_{—}2, 2_{—}3) and not in lysates of wt.

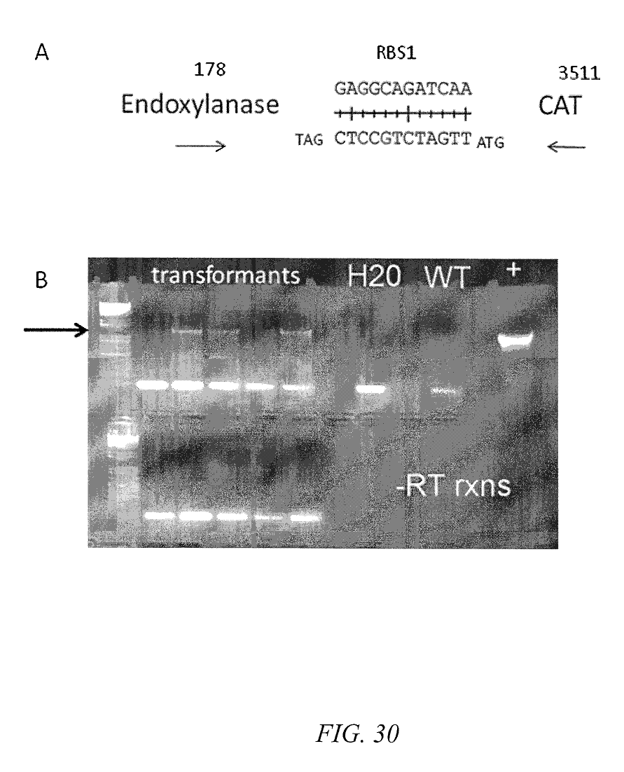

To determine whether both enzymes are produced from the same transcript, RNA was isolated from wildtype and engineered algae cells using the Concert Plant RNA Reagent kit (Invitrogen). RNA was DNase treated and cleaned using the RNeasy clean up kit (Qiagen). cDNA was synthesized from each of RNA using the iScrip kit (Biorad) and -reverse transcriptase (-RT) controls were included. cDNA (and -RT controls) was used as template in PCR with primers that hybridize to the endoxylanase gene and the CAT gene (FIG. 30A) (SEQ ID NO: 11 and SEQ ID NO: 12, respectively) and amplify a product of 1.3 kb. FIG. 30B shows that a product of appropriate size was amplified from cDNA templates from 4 of the 5 transformants indicating that in these lines, the endoxylanase and the CAT gene are transcribed on a single transcript.

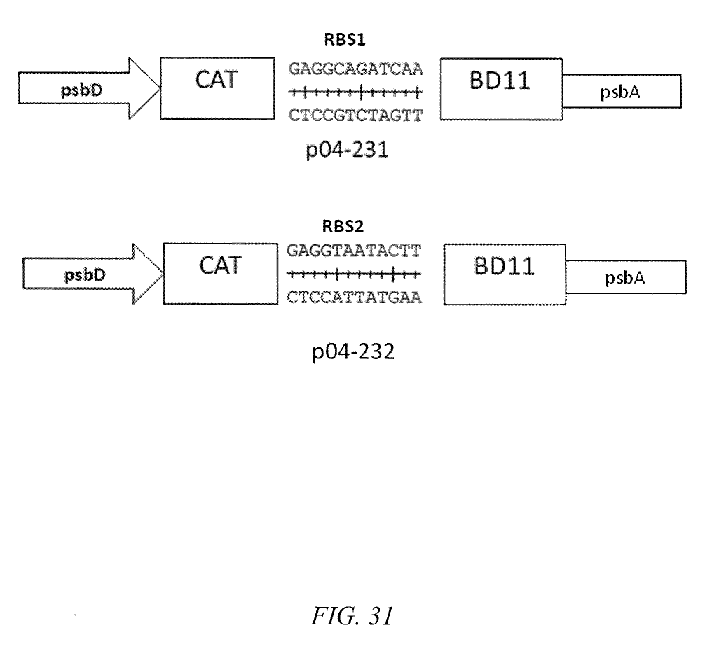

To further investigate variants of RBS1 (e.g., RBS2) and to understand the strength of these RBS sequences to recruit ribosomes, a nucleic acid encoding chloramphenicol acetyl transferase gene (CAT) from E. coli and endoxylanase from T. reesei linked together by two distinct ribosome binding sequences from E. coli were introduced into S. dimorphus. Transforming DNA (p04-231 or p04-232) is shown graphically in FIG. 31. In this instance the DNA segment labeled “CAT” is the chloramphenicol acetyl transferase encoding gene (SEQ ID NO: 28, CAT), the segment labeled “BD11” is the endoxylanase encoding gene (SEQ ID NO: 21, BD11), the segment labeled “RBS1” is the ribosome binding sequence (SEQ ID NO: 60, RBS1), the segment labeled “RBS2” is the ribosome binding sequence (SEQ ID NO: 61, RBS2) the segment labeled “psbD” is the promoter and 5′ UTR for the psbD gene from S. dimorphus, the segment labeled “D1 3′” is the 3′ UTR for the psbA gene from S. dimorphus. The transgene expression cassette and selection marker are targeted to the S. dimorphus chloroplast genome via the segments labeled “Homology A” and “Homology B” which are approximately 1000 bp fragments homologous to sequences of DNA adjacent to nucleotide 071,366 (Site 1; nucleoside locations according to the sequence available from NCBI for S. obliquus, NC_{—}008101) on the 5′ and 3′ sides, respectively. All DNA segments were subcloned into pUC19. All DNA manipulations carried out in the construction, of this transforming DNA were essentially as described by Sambrook et. al., Molecular Cloning: A Laboratory Manual (Cold Spring Harbor Laboratory Press 1989) and Cohen et al., Meth. Enzymol. 297, 192-208, 1998.

Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP agar medium+25 μg/mL chloramphenicol. Transformants were streaked onto TAP-CAM agar medium to ensure single colony isolation and grown for 4 days under constant light.

Transformants were analyzed by PCR screening (as described in EXAMPLE 1) and homoplasmic clones were identified and subcultured for further studies.

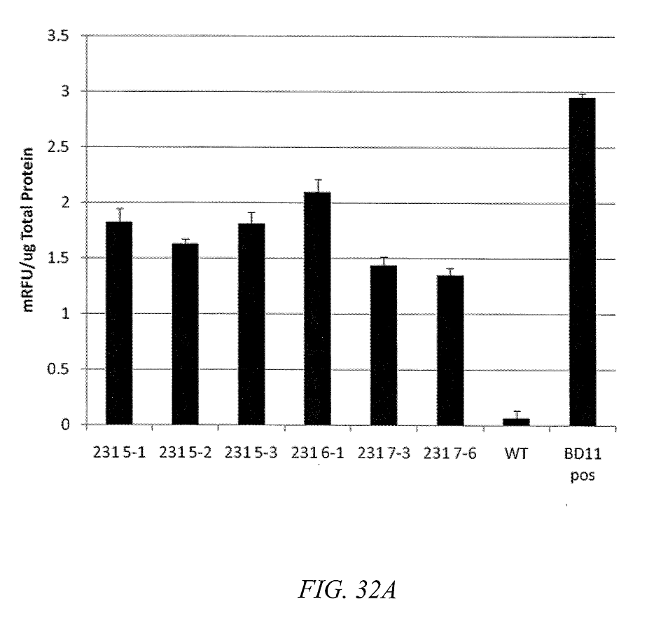

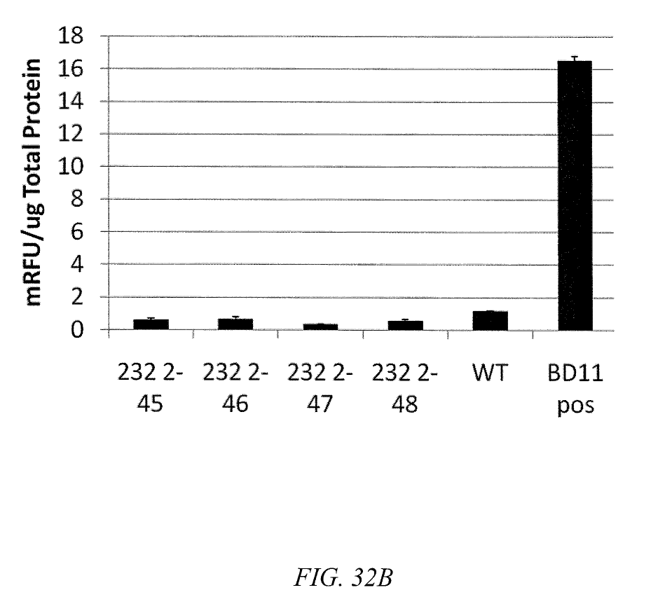

To determine if the endoxylanase produced by transformed algae cells was functional, endoxylanase activity was tested using an enzyme function assay (as described in EXAMPLE 4). FIG. 32A shows that RBS1 between the two genes produces xylanase activity, however RBS2 does not produce active xylanase (FIG. 32B).

These data demonstrate that the chloroplast of S. dimorphus can be transformed with a vector of foreign DNA containing an expression cassette that consists of a gene of interest linked to a selectable marker by a nucleotide sequence, allowing for the expression of multiple genes from, one transcript, in this case a gene encoding an endoxylanase and a gene encoding chloramphenicol acetyl transferase. One of skill in the art will appreciate that many other methods known in the art may be substituted in lieu of the ones specifically described or referenced.

### Example 34

### Use of Conserved Gene Cluster for an integration Site in S. dimorphus

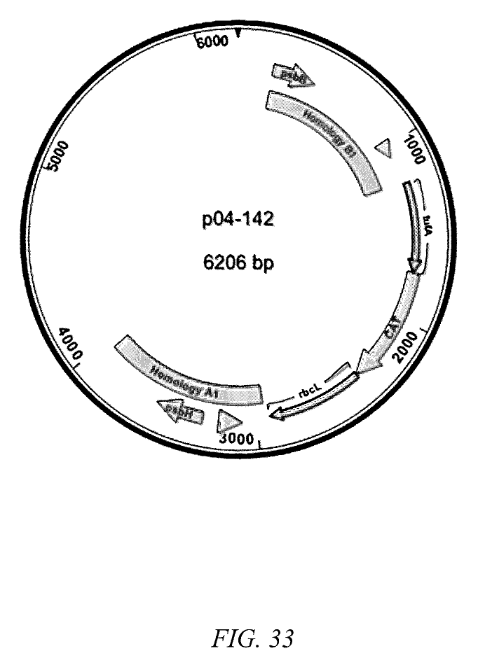

In this example, a nucleic acid encoding chloramphenicol acetyl transferase gene from E. coli was introduced into S. dimorphus. Transforming DNA is shown graphically in FIG. 33. In this instance the DNA segment labeled “CAT” is the chloramphenicol acetyl transferase gene from E. coli, the segment labeled “tufA” is the promoter and 5′ UTR sequence for the tufA gene from S. dimorphus, and the segment labeled “rbcL” is the 3′ UTR for the rbcL gene from S. dimorphus. The selection marker cassette is targeted to the S. dimorphus chloroplast genome via the segments labeled “Homology A1” and “Homology B1” which are approximately 1000 bp fragments homologous to sequences of DNA in the psbB-psbT-pshN-psbH cluster wherein the CAT cassette is inserted between psbT and psbN. All DNA segments were subcloned into pUC19. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP agar medium+25 μg/mL chloramphenicol (TAP-CAM) under constant light 50-100 uE @RT for approximately 2 weeks. Transformants were streaked onto TAP-CAM agar medium to ensure single colony isolation and grown for 4 days under constant light.

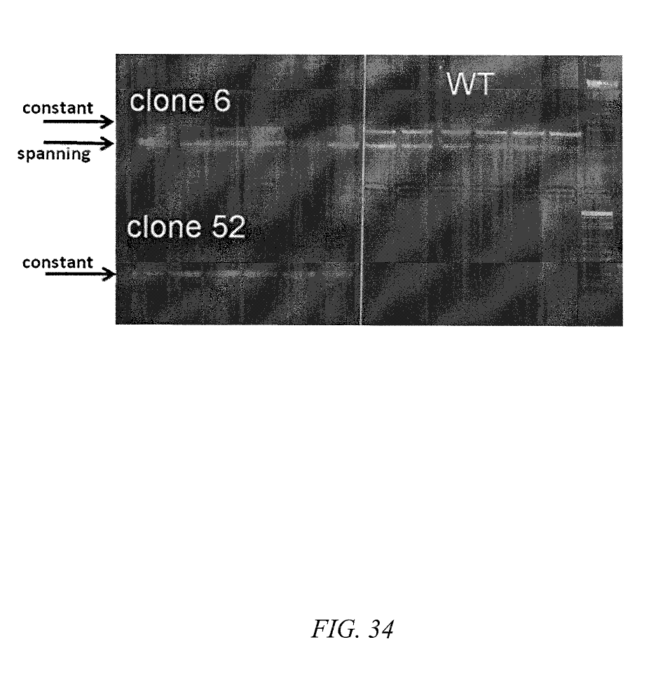

Cells from the transformants were analyzed by PCR screening (as described in EXAMPLE 1). The degree to which the transforming DNA was integrated into the chloroplast genome was determined using primers that amplify a 250 bp constant region (SEQ ID NO: 3 and SEQ ID NO: 4) and a 400 bp region spanning the integration site (SEQ ID NO: 15 and SEQ ID NO: 16). The homology regions target the integration site, the region of the chloroplast genome between psbT and psbN, approximately nucleotide 059,687 (nucleotide locations according to the sequence available from NCBI for S. obliquus, NC_{—}008101). FIG. 34 shows that subclones from clone 52 are homoplasmic, i.e., only the constant region (250 bp product) was amplified, while in the control reactions (WT) both the constant region and the region spanning the integration site (400 bp) were amplified. Clone 6 is another parental clone, however subclones from clone 6 are not completely homoplasmic as the spanning region is still amplified. These data indicate that the psbB-psbH cluster can be utilized as an integration site in engineering S. dimorphus.

One of skill in the art will appreciate that many other methods known in the art may be substituted in lieu of the ones specifically described or referenced.

### Example 15

### Strategy to Generate Markerless Transgenic S. dimorphus

In this example, the transgenic line generated in EXAMPLE 12, was used to inoculate nonselective media. A saturated culture was diluted 1:300 in nonselective media, allowed to grow to saturation and diluted 1:4 in nonselective media. Once saturated, the culture was plated onto nonselective TAP medium to ensure single colony formation. Single clones were then patched to 1) nonselective TAP medium and 2) TAP-CAM medium. Clones that failed to grow on TAP-CAM were further analyzed by PCR.

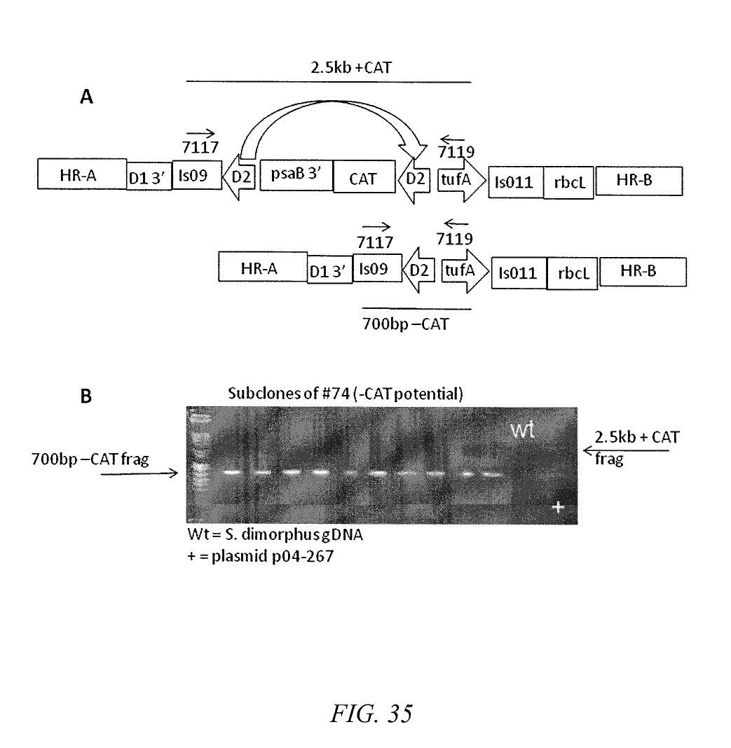

FIG. 35 A is a graphical representation, of the transforming DNA (top) and loopout product (bottom) that results from recombination at the identical D2 (psbD) promoter segments. HR-A & HR-B represent the homology regions. D1 3′, psaB 3′ and rbcL represent, the psbA 3′UTR, psaB 3′UTR, and rbcL 3′UTR, respectively. D2 and tufA is the psbD and tufA promoter, respectively. Is09 is FPP synthase and Is011 is bisabolene synthase.

To confirm the absence of the CAT gene, two methods were employed. First, PCR was performed rising primers that amplify a 2.5 kb +CAT fragment and/or a 700 bp-CAT fragment (SEQ ID NO: 9 and SEQ ID NO: 10). FIG. 35 B is an agarose gel showing that in subclones of the #74 transformant only the 700 bp-CAT product was amplified while in the plasmid DNA control, the 2.5 kb+CAT fragment was amplified. The presence of the 700 bp product in the plasmid DNA control is likely the result of recombination in the E. coli host as it is RecB+. Primers 7117 & 7119 (SEQ ID NO: 9 and SEQ ID NO: 10) were used to amplify the products. The “markerless” transgenic S. dimorphus shows amplification of 700 bp-CAT loopout fragment and failure to amplify the 2.5 kb +CAT fragment in subclones of clone #74.

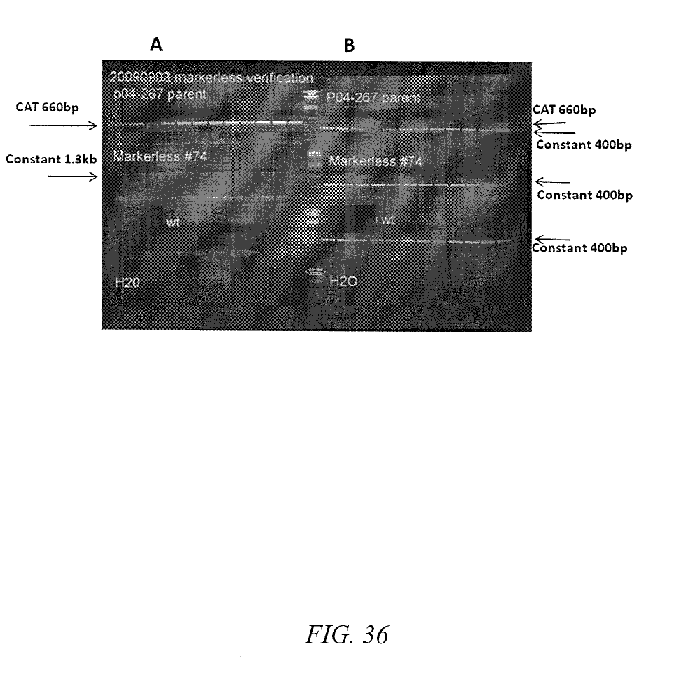

Second, PCR was performed using primers that amplify the 660 bp CAT gene (SEQ ID NO: 18 and SEQ ID NO: 19), and either primers that amplify a 1.3 kb constant region of the psbA gene (SEQ ID NO: 13 and SEQ ID NO: 14) or those that amplify a 400 bp constant region of the psbA gene (SEQ ID NO: 1 and SEQ ID NO: 2). FIG. 36 shows that only the constant fragment was amplified in the #74 markerless line, while the CAT gene was amplified in the parental line that was always kept on CAT selection. Panel A shows multiplex PCR using primers that amplify a 660 bp CAT fragment and primers that amplify a 1.3 kb constant region of the endogenous psbA gene. Only the 1.3 kb constant region is amplified in the #74 markerless potential. Panel B shows multiplex PCR using primers that amplify a 660 bp CAT fragment and primers that amplify a 400 bp constant region of the endogenous psbA gene. Only the 400 bp constant region is amplified in the #74 markerless potential. The PCR reactions in both panel A and panel B had a 50-60 degree Celcius annealing gradient to ride out the possibility that the annealing of the primers was temperature sensitive.

These data demonstrate that S. dimorphus clones can be obtained consisting of a genetically engineered chloroplast and without an antibiotic resistance marker.

One of skill in the art will appreciate that many other methods known in the art may be substituted in lieu of the ones specifically described or referenced.

### Example 16

### Use of Betaine Aldehyde Dehydrogenase to Confer Salt Tolerance and/or as a Negative Selection Mechanism

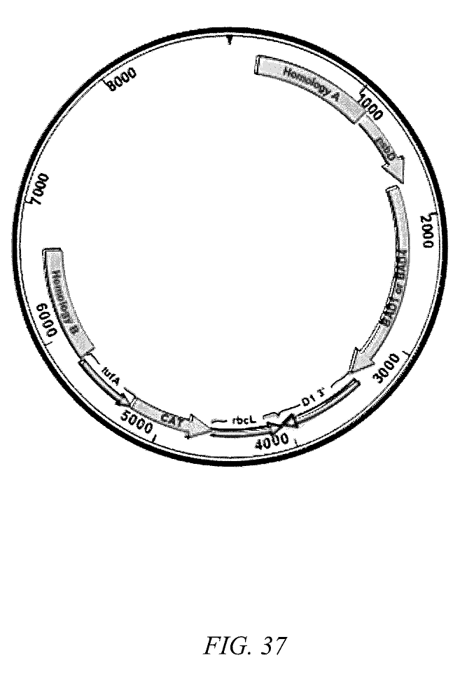

In this example, a nucleic acid sequence encoding betaine aldehyde dehydrogenase from spinach or sugar beet was engineered into S. dimorphus (as described in EXAMPLE 4). Transforming DNA is shown graphically in FIG. 37. In this instance the DNA segment labeled “BAD1 or BAD4” is the betaine aldehyde dehydrogenase encoding gene from spinach (BAD1) or sugar beet (BAD4) (SEQ ID NO: 32, BAD1 or SEQ ID NO: 34, BAD4), the segment labeled “psbD” is the promoter and 5′ UTR for the psbD gene from S. dimorphus, the segment labeled “rbcL” is the 3′ UTR for the psbA gene from S. dimorphus, and the segment labeled “CAT” is the chloramphenicol acetyl transferase gene from E. coli, which is regulated by the promoter and 5′ UTR sequence for the tufA gene from S. dimorphus and the 3′ UTR sequence for the rbcL gene from S. dimorphus. The transgene expression cassette and selection marker are targeted to the S. dimorphus chloroplast genome via the segments labeled “Homology A” and “Homology B” which are approximately 1000 bp fragments homologous to sequences of DNA adjacent to nucleotide 071,366 (Site 1; nucleotide locations according to the sequence available from NCBI for S. obliquus, NC_{—}008101) on the 5′ and 3′ sides, respectively. All DNA segments are subcloned into pUC19. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into S. dimorphus via particle bombardment according to the method described in EXAMPLE 1 with DNA carried on 550 nm gold particles @500 psi and a shooting distance of 4 cm, Transformants were selected by growth on TAP-CAM agar medium under constant light 50-100 uE @RT for approximately 2 weeks. Transformants were streaked onto TAP-CAM agar medium to ensure single colony isolation and grown for 4 days under constant light.

Transformants were analyzed by PCR screening (as described in EXAMPLE 1) and homoplasmic clones were identified and subcultured for further studies.

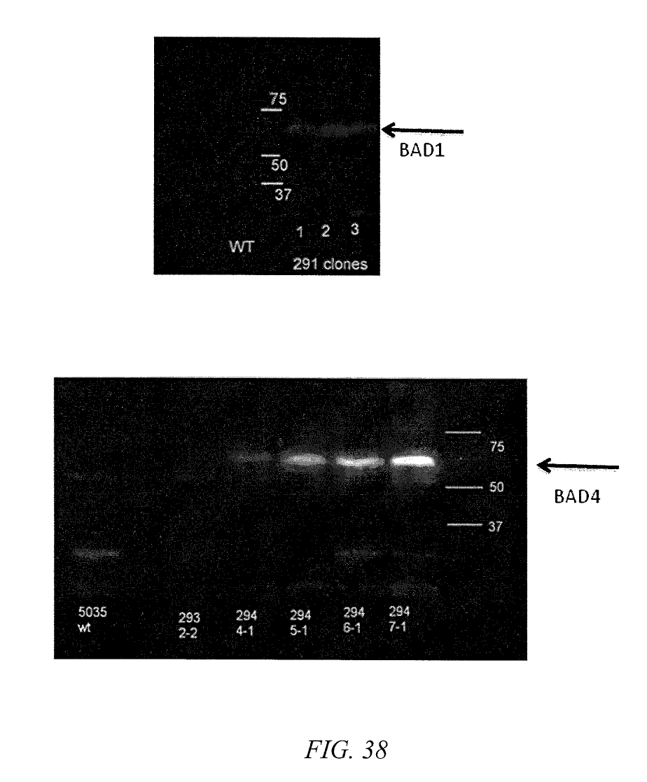

To ensure that the presence of the betaine aldehyde dehydrogenase encoding gene led to expression of the protein, a Western blot was performed (as described in EXAMPLE 4). In this instance, the BAD genes were tagged with an HA tag and the primary antibody was an anti-HA HRP conjugated antibody (clone 3F10, Roche) in which a 1:10,000 dilution of a 50 U/mL stock was used as the antibody solution. Results from multiple clones (FIG. 38) show that expression of the BAD gene from spinach and from sugar beet gene in S. dimorphus cells resulted in production of the protein.

To determine if this protein confers salt tolerance or causes the cells to become sensitive to betaine aldehyde (and therefore allows this strain to be used in negative selection experiments as proposed in examples 9 and 10), cells expressing the BAD genes can be grown side-by-side with wildtype cells and the media supplemented with increasing concentrations of salt and/or betaine aldehyde.

One of skill in the art will appreciate that many other methods known in the art may be substituted in lieu of the ones specifically described or referenced.

### Example 17

### Development of a Transformation System for D. tertiolecta

In this example, a method for transformation of D. tertiolecta is described. Algae cells are grown to log phase (approximately 5.0×10⁶ cells/mL) in G32 medium (32 g/L NaCl, 0.0476 mM CaCl₂, 0.162 mM H₃BO₃, 0.406 mM Mg₂SO₄, 0.00021 mM NaVO₃, 5 g/L bicarbonate, 12.9 mL/L each of F/2 A and B algae food (Aquatic Eco-systems, Inc.)) at. 23° C. under constant illumination of 50-100 uE on a rotary shaker set at 100 rpm. Cells are harvested at 1000×g for 5 min. The supernatant is decanted and cells are resuspended in G32 media at 10⁸ cells/mL, 5×10⁷ cells are spread on selective agar medium and transformed by particle bombardment, with 550 nm diameter gold particles carrying the transforming DNA @300-400 psi with the Helios Gene Gun. (Bio-Rad) from a shot distance of 4 cm, Desired algae clones are those that grow on selective media.

PCR is used to identify transformed algae strains. For PCR analysis, colony lysates are prepared by suspending algae cells (from agar plate or liquid culture) in lysis buffer (0.5% SDS, 100 mM NaCl, 10 mM EDTA, 75 mM Tris-HCl, pH 7.5) and heating to 98° C. for 10 minutes, followed by cooling to near 23° C. Lysates are diluted 50-fold in 100 mM Tris-HCl pH 7.5 and 2 μL is used as template in a 25 μL reaction. Alternatively, total genomic DNA preparations may be substituted for colony lysates. A PCR cocktail consisting of reaction buffer, dNTPs, PCR primer pair(s) (indicated in each example below), DNA polymerase, and water is prepared. Algae DNA is added to provide template for the reaction. Annealing temperature gradients are employed to determine optimal annealing temperature for specific primer pairs. In many cases, algae transformants are analyzed by PCR with primers that are specific for the transgene being introduced info the chloroplast genome. Desired algae transformants are those that give rise to PCR product(s) of expected size(s).

One of skill in the art will appreciate that many other transformation methods known in the art may be

substituted in lieu of the ones specifically described or referenced herein.

### Example 18

### Else of Conserved Gene Cluster for an Integration site in P. tertiolecta

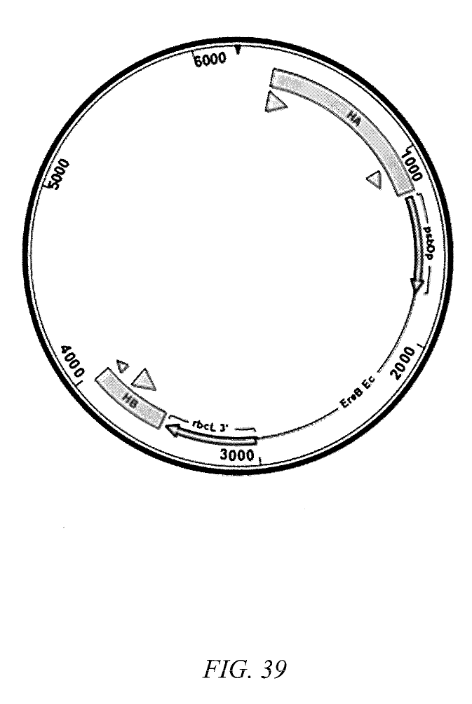

In this example, a nucleic acid encoding erythromycin esterase gene (EreB) (SEQ ID NO: 29) from E. coli was introduced into D. tertiolecta. Transforming DNA is shown graphically in FIG. 39. In this instance the DNA segment labeled “EreB ec” is the erythromycin esterase gene (EreB) from E. coli, the segment labeled “psbDp” is the promoter and 5′ UTR sequence for the psbD or tufA gene from a D. tertiolecta (SEQ ID NO: 62, psbD2, SEQ ID NO: 63, tufA2), and the segment labeled “rbcL 3′” is the 3′ UTR for the rbcL gene from D. tertiolecta (SEQ ID NO: 64, 2rbcL 3′). The selection marker cassette is targeted to the D. tertiolecta chloroplast genome via the segments labeled “HA” and “HB” which are approximately 1000 bp fragments homologous to sequences of DNA in the psbB-psbT-psbN-psbH cluster (SEQ ID NO: 133) wherein the EreB cassette is inserted between psbT and psbN at approximately nucleotide 2383 of SEQ ID NO: 133. All DNA segments were subcloned info pUC19. All DNA manipulations carried out in the construction of this transforming DNA were essentially as described by Sambrook et al. Molecular Cloning: A Laboratory Manual (Cold Spring Harbor Laboratory Press 1989) and Cohen et al., Meth. Enzymol. 297, 192-208, 1998.

Transforming DNA was introduced into D. tertiolecta via particle bombardment according to the method described in EXAMPLE 17 with DNA carried on 550 nm gold particles @300 psi and a shooting distance of 4 cm. Transformants were selected by growth on G32 agar medium+75 μg/mL erythromycin (G32-Erm) under constant light 50-100 uE @RT for approximately 4 weeks. Transformants were inoculated into nonselective G32 media and grown for ˜1 week under constant light (50-100 uE).

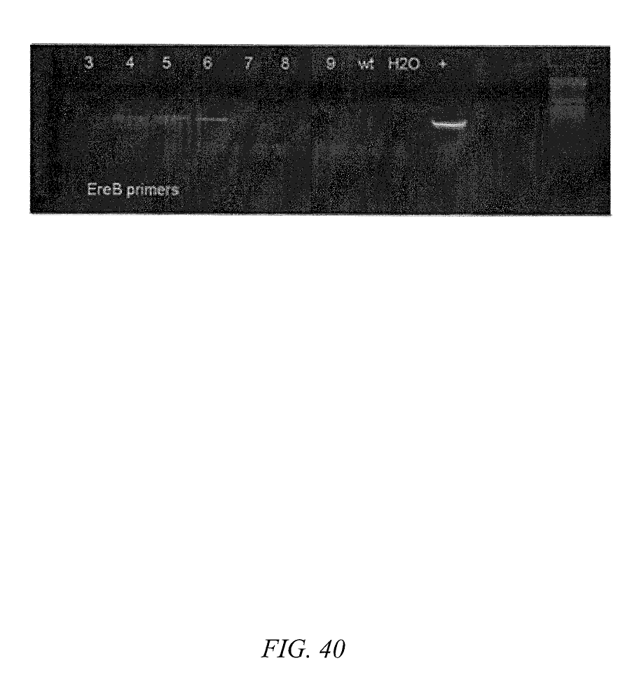

Cells from the transformants were analyzed by PCR screening (as described in EXAMPLE 17). The presence of the EreB selection marker was determined using primers that amplify a 555 bp region within the gene (SEQ ID NO: 7 and SEQ ID NO: 8). FIG. 40 shows that the EreB gene was amplified from DNA from transformants 4, 5, and 6 but not from wildtype DNA from D. tertiolecta.

These data demonstrate that the chloroplast of D. tertiolecta can be transformed with foreign DNA containing an expression cassette with a selectable marker. One of skill in the art will appreciate that many other methods known in the art may be substituted in lieu of the ones specifically described or referenced.

### Example 19

### Production of Endoxylanase in D. tertiolecta

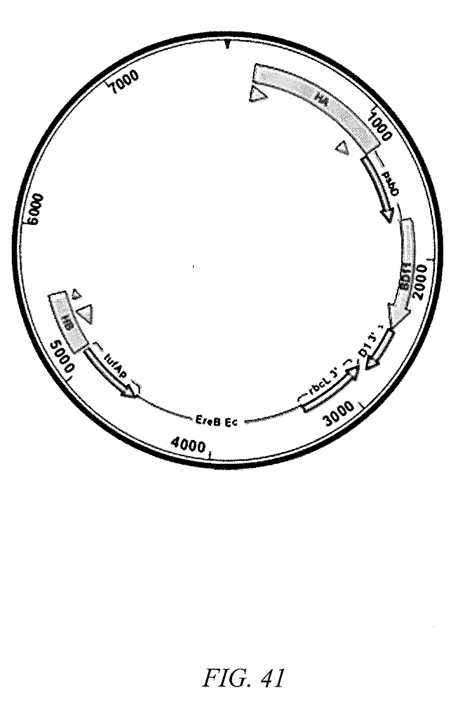

In this example a nucleic acid encoding endoxylanase from T. reesei was introduced into D. tertiolecta. Transforming DNA is shown, graphically in FIG. 41. In this instance the DNA segment, labeled “BD11” is the endoxylanase encoding gene (SEQ ID NO: 21, BD11), the segment labeled “psbD” is the promoter and 5′ UTR for the psbD gene from D. tertiolecta, the segment labeled “D1 3′” is the 3′ UTR for the psbA gene from D. viridis (SEQ ID NO: 65, 3 psbA 3′), and the segment labeled “EreB ec” is the erythromycin esterase gene from E. coli, which is regulated by the promoter and 5′ UTR sequence for the tufA gene from D. tertiolecta and the 3′ UTR sequence for She rbcL gene from D. tertiolecta. The transgene expression cassette and selection masker are targeted to the D. tertiolecta chloroplast genome via the segments labeled “HA” and “HB” which are approximately 1000 bp fragments homologous to sequences of DNA in the psbB-psbT-psbN-psbH cluster wherein the transgene cassette is inserted between psbT and psbN. All DNA segments were subcloned into pUC19. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Transforming DNA was introduced into D. tertiolecta via particle bombardment according to the method described in EXAMPLE 17 with DNA carried on 550 nm gold particles @400 and a shooting distance of 4 cm, Transformants were selected by growth on G32 agar medium+75 μg/mL erythromycin (G32-Erm) under constant light 50-100 uE @RT for approximately 4 weeks. Transformants were inoculated into G32 media+100 μg/mL erythromycin and grown for ˜1 week under constant light (50-100 uE).

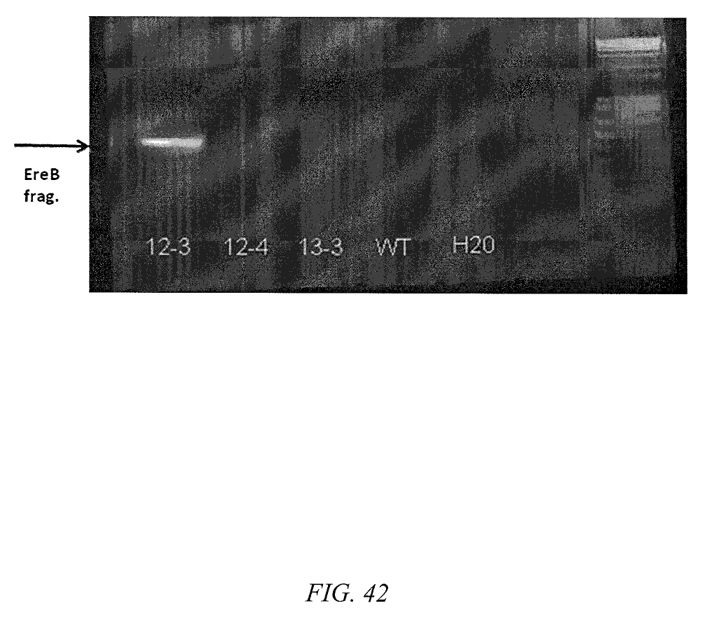

Cells from the transformants were analyzed by PCR screening (as described in EXAMPLE 17). The presence of the EreB selection marker was determined using primers that amplify a 555 bp region within the gene (SEQ ID NO: 7 and SEQ ID NO: 8). FIG. 42 shows that the EreB gene was amplified from DMA from transformant 12-3 but not from wildtype DNA from D. tertiolecta.

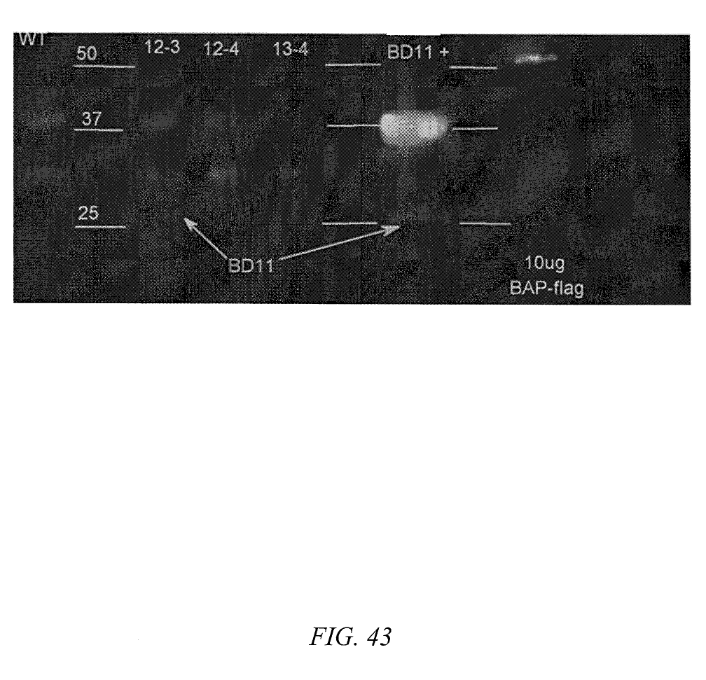

To ensure that the presence of the endoxylanase-encoding gene led to expression of the endoxylanase protein, a Western blot was performed (as described in EXAMPLE 4). Results from transformant 12-3 (FIG. 43) show that expression of the endoxylanase gene in D. tertiolecta cells resulted in production of the protein.

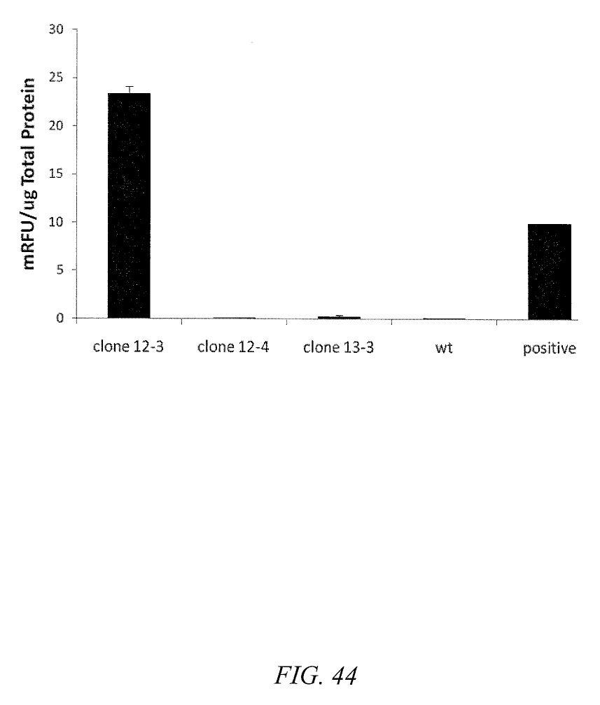

To determine if the endoxylanase produced by transformed algae cells was functional, endoxylanase activity was tested using an enzyme function assay (as described in EXAMPLE 4). FIG. 44 shows that endoxylanse activity is detected in the 12-3 transformant but not in wildtype cells.

These data demonstrate that the chloroplast of D. tertiolecta can be transformed with foreign DNA containing an expression cassette with a selectable marker and a separate expression cassette with a gene encoding an endoxylanase, and that the proteins expressed were functional. One of skill in the art will appreciate that many other methods known in the art may be substituted in lieu of the ones specifically described or referenced.

### Example 20

### Overview of Genetic Engineering

To engineer the chloroplast of an algae three things are required: a cassette expressing a selectable marker; a delivery method to deliver the plasmid DNA into the chloroplast; and a vector containing regions of DNA homologous to the chloroplast genome to be used in targeted homologous recombination (a homologous integration vector or homologous recombination vector).

In strains of algae that have little or no known chloroplast sequence information available, the identification of homologous regions of DMA and the construction of a vector containing those regions, are significant and time consuming tasks. Current methods for obtaining unknown sequence information., such as Inverse PCR(PCR Cloning Protocols Series: Methods in Molecular Biology, Volume: 192, Pub. Date: Apr. 1, 2002, Page Range: 301-307, DOI: 10.1385/1-59259-177-9:301) and Adaptor Ligated PCR (Nature Protocols 2, pp. 2910-2917 (2007) Published online: 8 Nov. 2007) are time consuming in that they take multiple iterations in order to generate a DNA sequence that is long enough to be used in a homologous integration vector.

A method that allows for the quick identification of a large piece of chloroplast DNA sequence, sufficient in size to build a homologous integration vector, would be very useful in the engineering of algal genomes. The methods described herein, can be applied to all strains of an algae, for example, a green algae, for which there is little or no known DNA sequence information available. The methods described herein, can also be applied to an algae, for which there is incomplete sequence information available.

### Example 23

### Use of a Conserved Gene Cluster to Generate Sequence Information

Across the chloroplast genomes sequenced to date, there are only a few clusters of genes that are consistently found adjacent to each other. Two examples of such gene clusters are ycf3-ycf4 and psbF-psbL. However, these two clusters are too small is size to yield enough DNA sequence information to be useful for homologous recombination.

Another gene cluster, psbB-psbT-psbN-psbH, is found together in the same orientation in most algae and plants. Knowledge of the presence of this gene cluster allows one to amplify a large region of chloroplast DNA that provides enough DNA sequence information to construct a vector for homologous recombination. This vector can then be used to modify the chloroplast genome of algal strains and plants that have not yet been genetically engineered.

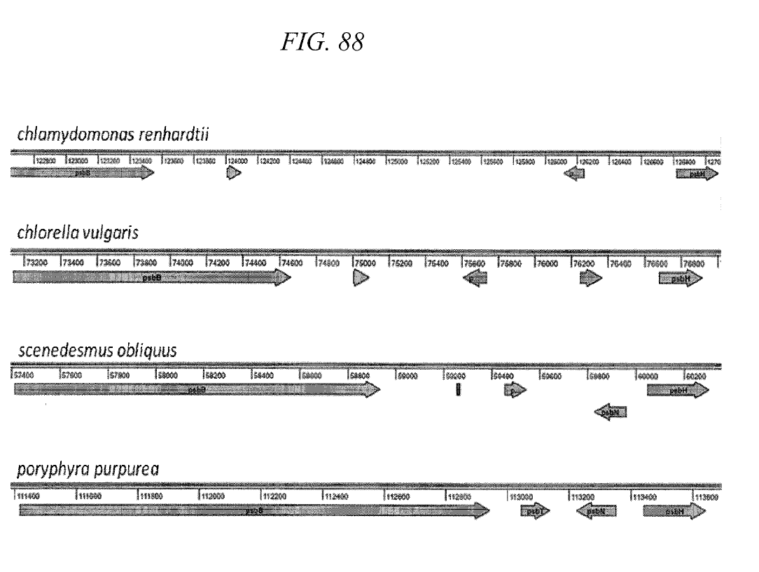

The gene cluster psbB-psbT-psbN-psbH is a region of chloroplast DNA that is highly conserved amongst algae and plants. However, this cluster may not be conserved at the nucleic acid level or in the spacing between the genes (the intergenic regions). In addition, the nucleic acid contents of the intergenic regions may vary. While at the nucleotide level there may be significant diversity, at the protein level this region is quite conserved. FIG. 88 is an alignment of 4 algae that have had their chlorolast genomes sequenced: C. reinhardtii (NCBI NC_{—}005353). C. vulgaris (NCBI NC_{—}001865), S. obliquus (NCBI NC_{—}008101), and P. purpurea NCBI NC_{—}000925). This figure shows the high degree of conservation in terms of gene placement and orientation.

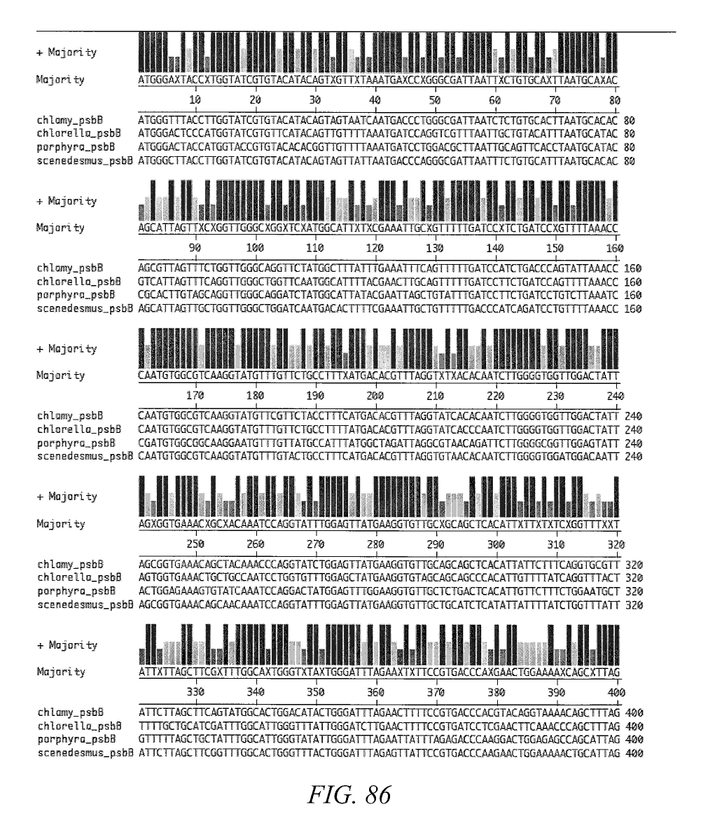

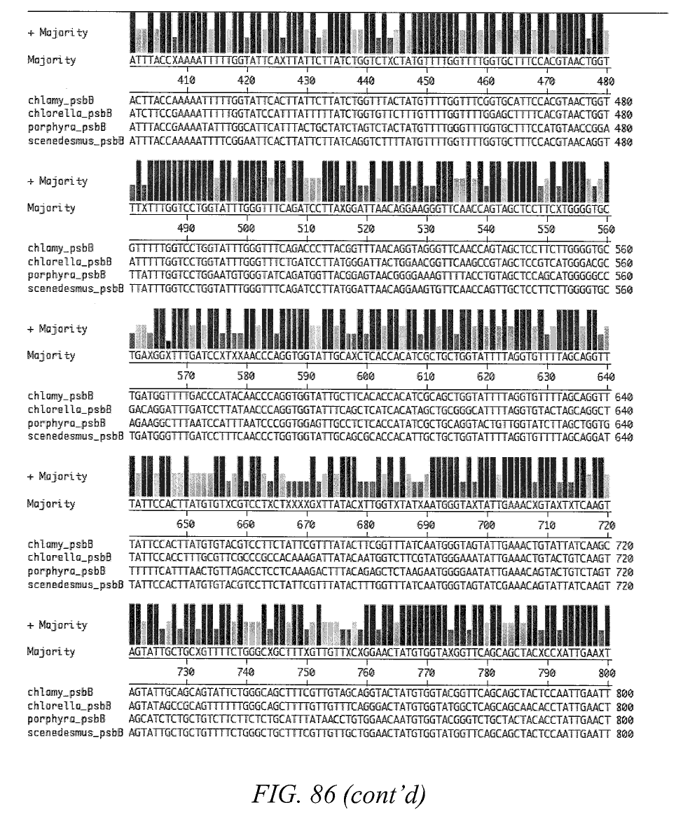

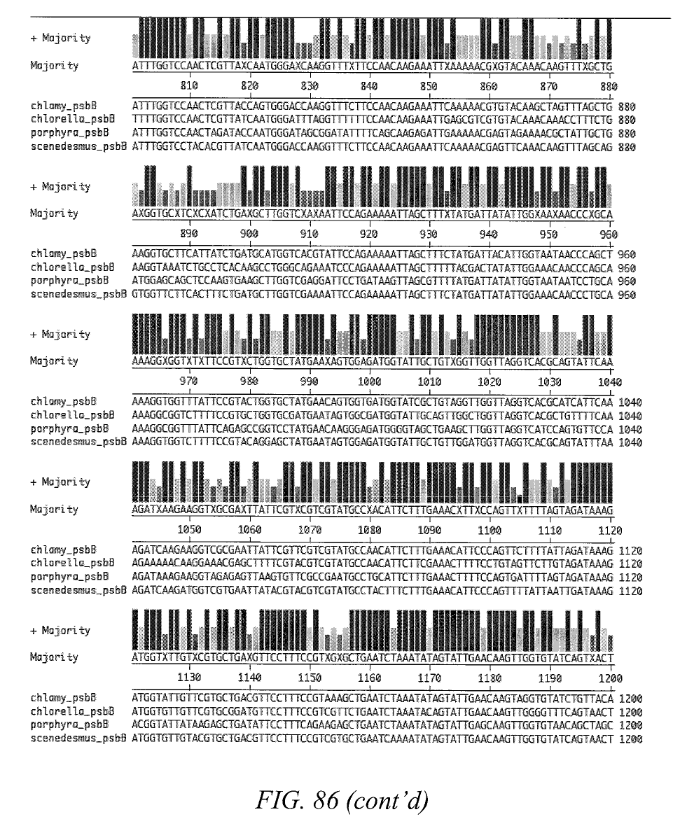

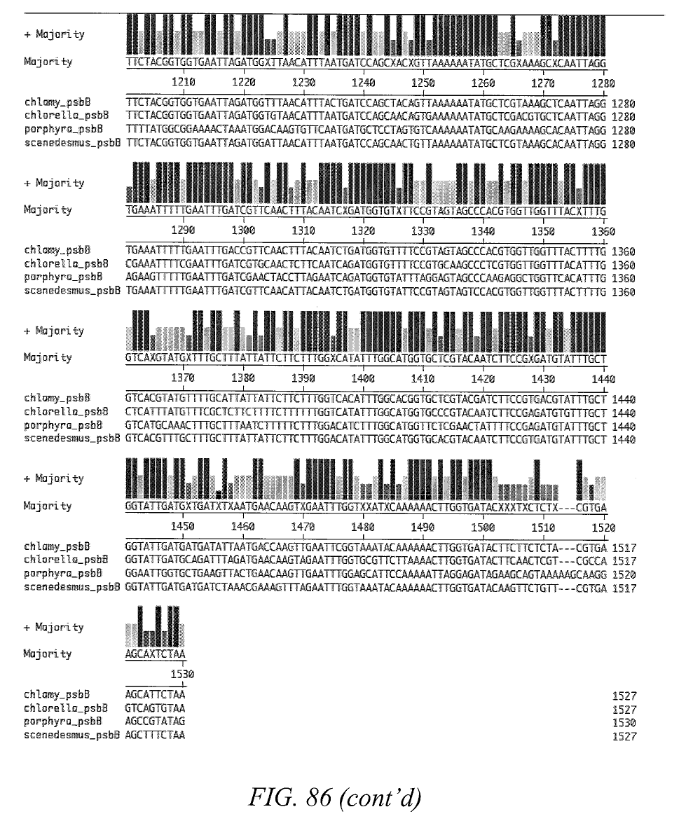

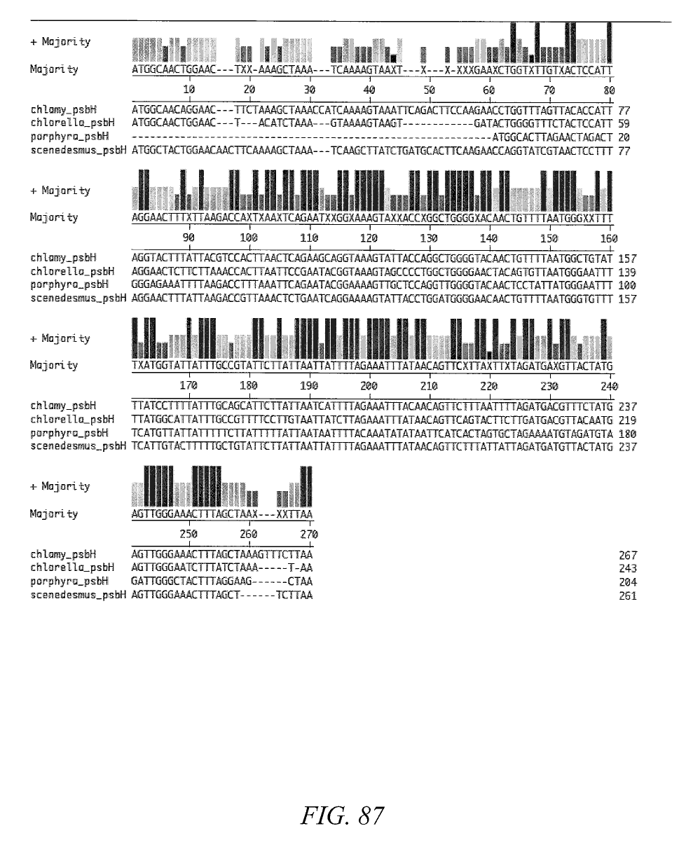

Although the gene cluster, psbB-psbT-psbN-psbH, may not be conserved at the nucleic acid level, the proteins on the terminal ends of this region, psbB and psbH, are highly conserved at the amino acid level and contain regions of high conservation at the nucleotide level. FIG. 86 is an alignment of the psbB gene from four algae that have had their chlorolast genomes sequenced: C. reinhardtii, C. vulgaris, S. obliquus, and P. purpurea, and FIG. 87 is an alignment of the psbH gene from the same algae. Both figures show regions of high nucleic acid homology. This allows for the design of degenerate primers that will anneal to regions within the nucleic acid sequences encoding for the proteins psbB and psbH, resulting in the amplification of the whole gene cluster in one step. This double stranded product can then be quickly sequenced directly from both ends, and enough sequence information can then be generated to construct a homologous recombination vector. The time it takes to generate the sequence data is much less than with other methods.

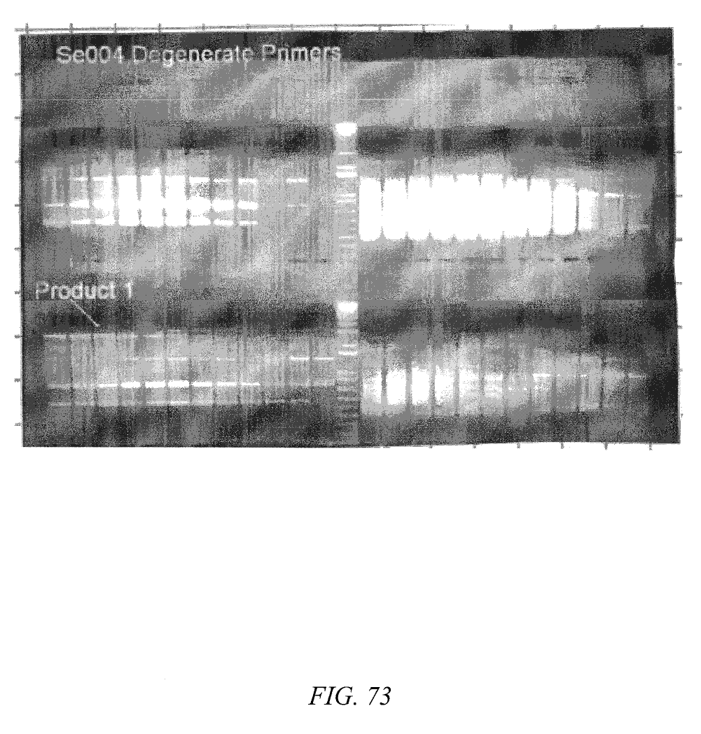

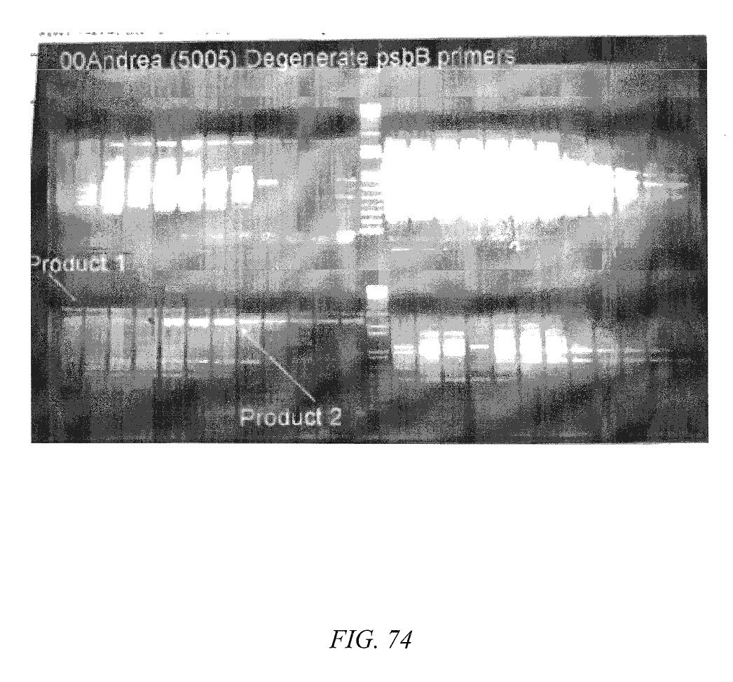

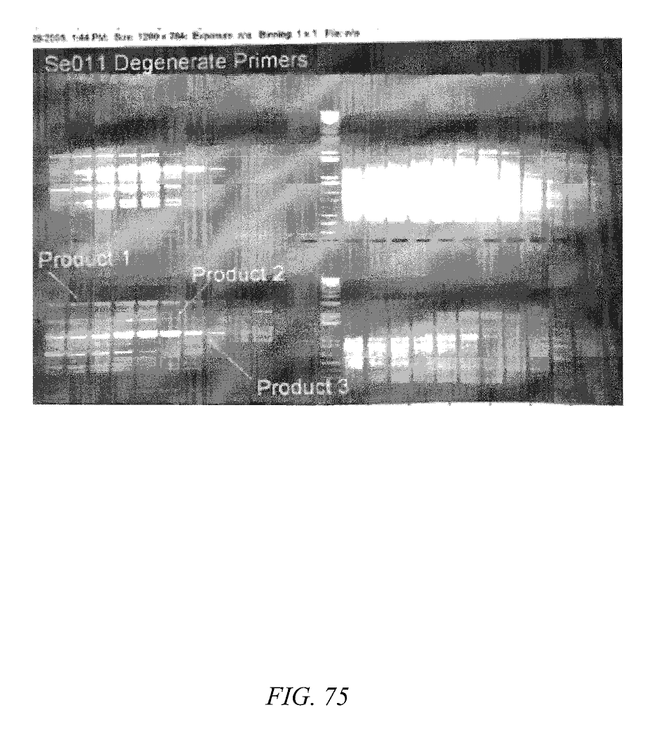

Two degenerate primers (forward primers 4099 and 4100) specific to the psbB gene (reverse primers SEQ ID NO: 129 and SEQ ID NO: 130) and 2 two degenerate primers (4101 and 4102) specific to the psbH gene (SEQ ID NO: 131 and SEQ ID NO: 132) were designed from the conserved nucleotide regions of the psbB and psbH. These primers have been used to generate the sequence of the psbB-psbT-psbN-psbH gene cluster from different species of algae that have little or no sequence information available in public databases including D. tertiolecta (SEQ ID NO: 133), an alga from the genus Dunaliella of unknown species (SEQ ID NO: 134), N. abudans (SEQ ID NO: 135), an isolate of C. vulgaris differing from the published genome (SEQ ID NO: 136), and T. suecica (SEQ ID NO: 137). FIGS. 74 and 75 show the degenerate primers amplifying a large fragment from the Dunaliella isolate and N. abudans, respectively. FIG. 73 shows the amplification from S. dimorphus. In each of these figures the center lane is occupied by a 1 kb Plus ladder (Invitrogen). In each figure four different combinations of primers were used. The top left panel shows amplification with primers 4099 and 4101. The bottom left panel shows amplification with primers 4099 and 4102. The top right panel shows amplification with primers 4100 and 4101. The bottom, left panel shows amplification with primers 4100 and 4102. After amplification the desired fragments are gel purified using the Qiaquick Gel Extraction Kit (Qiagen) and sequenced. In each of FIGS. 73 to 75, Product 1 represents the full length psbB-pbsBH gene cluster.

An integration vector built from this region has been shown to transform Dunaliella tertiolecta (see EXAMPLE 18).

### Example 22

### Additional Vectors constructed for Scenedesmus dimorphus

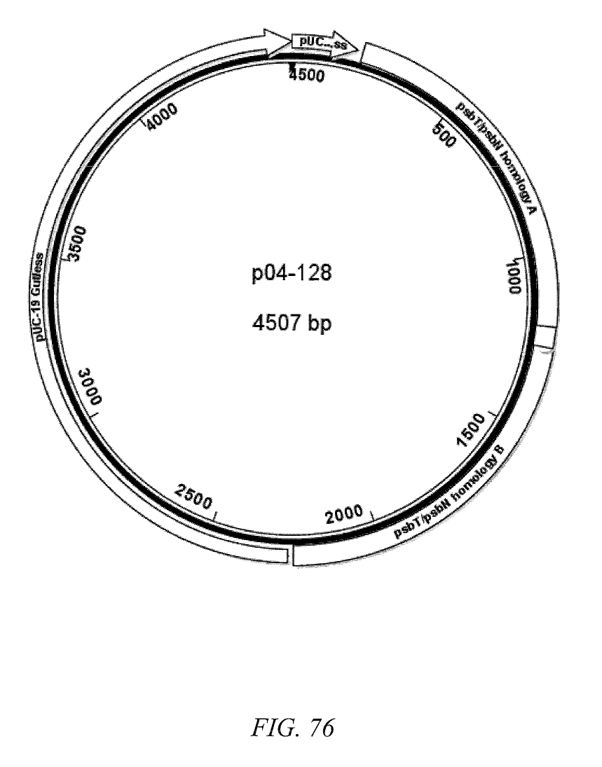

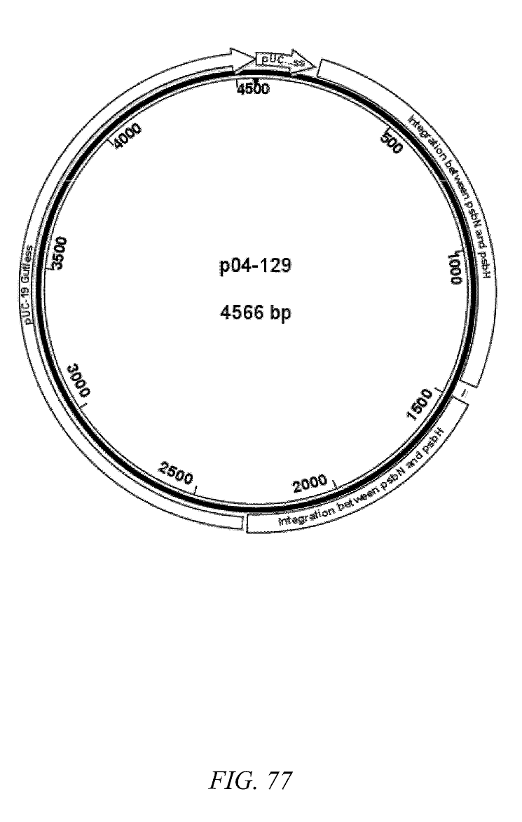

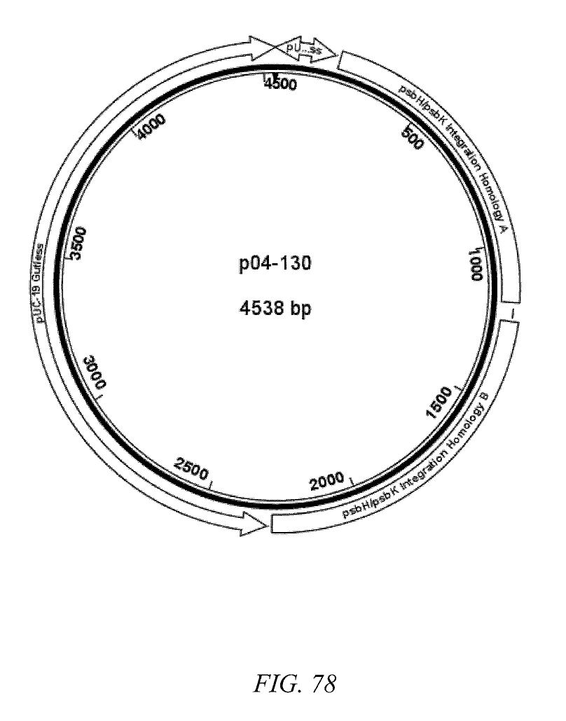

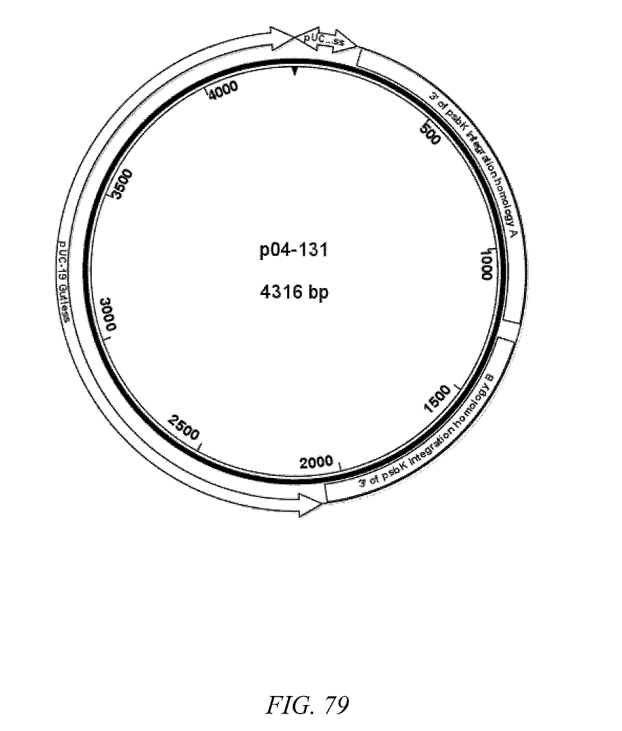

Additional vectors were constructed for Scenedesmus dimorphus since the sequence of a closely related species Scenedesmus obliquus is publicly available (NCBI for S. obliquus, NC_{—}008101). These vectors were made to test integration sites and homoplasmicity along the entire region of psbB-psbT-psbN-psbH, as well as the next adjacent protein in S. dimorphus, psbK. This set of vectors targeted integration into the intergenic region between psbT and psbN, psbN and psbH, psbH and psbK, and the region 3′ of psbK (p04-128, p04-129, p04-130, and p04-131 respectively) (FIGS. 76 to 79 respectively). p04-128 targets integration at approximately nucleotide 059,587. p04-129 targets integration at approximately nucleotide 059,999. p04-130 targets integration at approximately nucleotide 060,429. p04-131 targets integration at approximately nucleotide 060,961 (nucleotide locations according to the sequence available from NCBI for S. obliquus, NC_{—}008101).

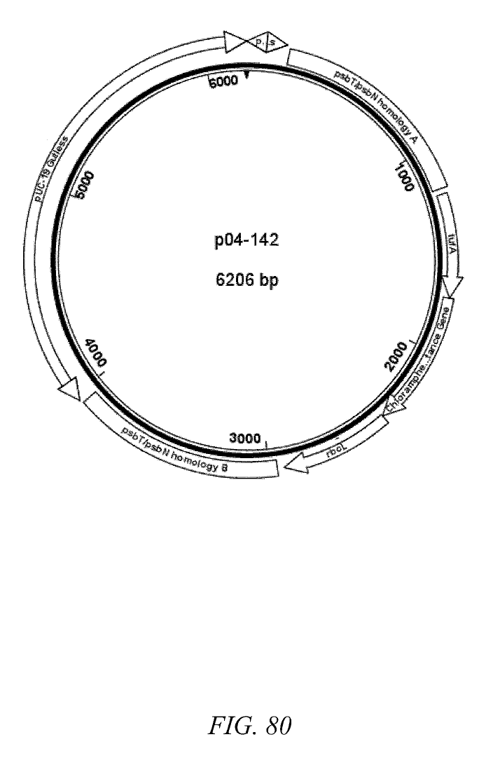

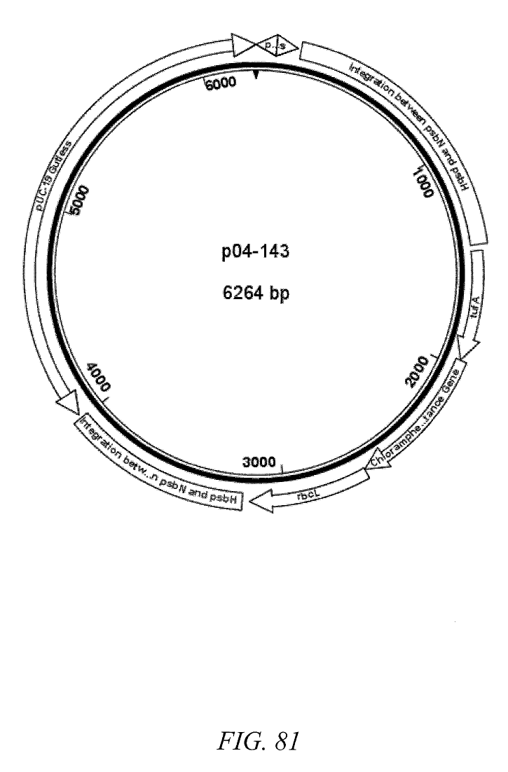

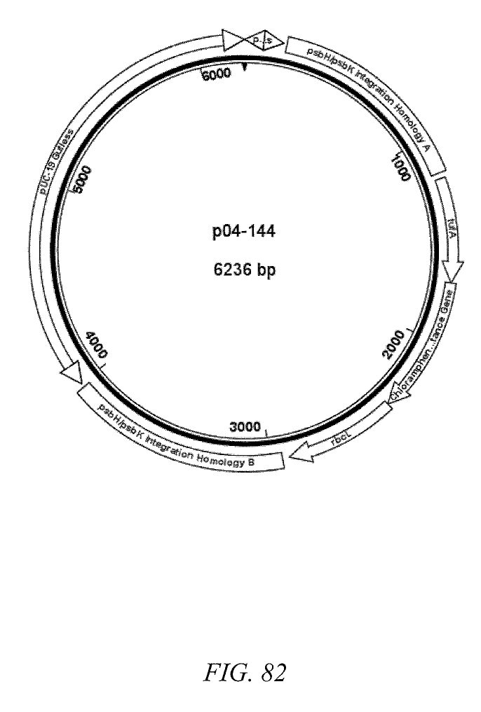

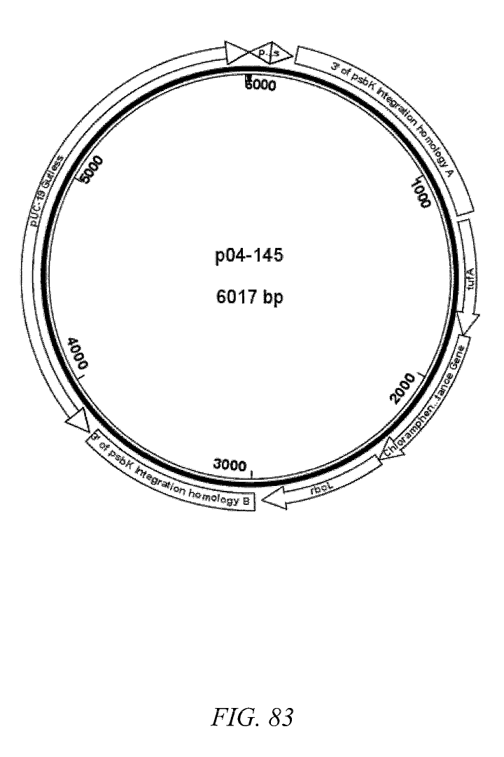

All vectors have an expression cassette consisting of a chloramphenicol (CAT) selectable marker, an endogenous promoter, and an endogenous terminator cloned between the Homology A and Homology B fragments. p04-128 had tufA-CAT-rbcL cloned between the Homology A and Homology B fragments (p04-142)(FIG. 80). p04-129 had tufA-CAT-rbcL cloned between the Homology A and Homology B fragments (p04-143) (FIG. 81). p04-130 had tufA-CAT-rbcL cloned between the Homology A and Homology B fragments (p04-144) (FIG. 82). p04-131 had tufA-CAT-rbcL cloned between the Homology A and Homology B fragments (p04-145) (FIG. 83). Vectors are shown graphically in their corresponding figures. In this instance the DNA segment labeled “CAT” is the chloramphenicol acetyl transferase gene from is. coli, the segment labeled “tufA” is the promoter and 5′ UTR sequence for the tufA gene from S. dimorphus, and the segment labeled “rbcL” is the 3′ UTR for the rbcL gene from S. dimorphus.

Transforming DNA was introduced into S. dimorphus via particle bombardment according to the method described in EXAMPLE 1 with DNA carried on 550 nm gold particles @500 psi and a shooting distance of 4 cm. Transformants were selected by growth on TAP-CAM agar medium under constant light 50-100 uE @RT for approximately 2 weeks. Transformants were streaked onto TAP-CAM agar medium to ensure single colony isolation and grown for 4 days under constant light.

To test for integration, of the CAT gene in between the psbT and psbN genes (p04-142), clones were screened for homoplasmicity using primers 3160 and 3162 (that amplify a 200 bp constant band from the genome), and primers 4682 and 4982 (that amplify a 400 bp band that spanning the integration site).

To test for integration of the CAT gene in between the psbN and psbH genes (p04-143), clones were screened for homoplasmicity using primers 2922 and 2923 that, amplify a 400 bp constant band from the genome, and primers 4684 and 4685, that amplify a 200 bp band that spans the integration site.

To test for integration of the CAT gene in between the psbH and psbK genes (p04-144), clones were screened for homoplasmicity using primers 2922 and 2923 that, amplify a 400 bp constant band from the genome, and primers 4686 and 4687, that amplify a 300 bp band that spans the integration site.

To test for integration of the CAT gene 3′ of the psbK gem (p04-345), clones were screened for

homoplasmicity using primers 3160 and 3162 that amplify a 200 bp constant band from the genome, and primers 4688 and 4689 amplify a 300 bp band that spans the integration site.

Primers used for each of the PCR screens are listed in Table 5.

TABLE 5

| | |
|---|---|
| p04-142 | 3160 (SEQ ID NO: 3) GAACTACAACTAATTATTTTC |
| | 3162 (SEQ ID NO: 4) TGAAACCAGTCTTTGTAAAGCT |
| | CA |
| | 4682 (SEQ ID NO: 15) CCACCTCGTATGGTAAAATAA |
| | TTG |
| | 4982 (SEQ ID NO: 16) GAAAGAATTATGGACAGTCCT |
| | GCT |
| p04-143 | 2922 (SEQ ID NO: 1) AGAAGGAGCTTCTACAGATGC |
| | 2923 (SEQ ID NO: 2) TCATTAGTTACTTCATCTTTAA |
| | TCCG |
| | 4684 (SEQ ID NO: 140) GAAGGAGGTCCAAAACTCAC |
| | A |
| | 4685 (SEQ ID) NO: 141) CCTGGTTCTTGAAGTGCAT |
| | C |
| p04-144 | 2922 (see above) |
| | 2923 (see above) |
| | 4686 (SEQ DD NO: 142) TGAGTTGGGAAACTTTAGCT |
| | TCTT |
| | 4787 (SEQ ID NO: 143) AAAAGATTGCCAAGACCAAA |
| p04-145 | 3160 (see above) |
| | 3162 (see above) |
| | 4688 (SEQ ID NO: 144) AAAAAGAATGAAATTTTTAT |
| | GTTCG |
| | 4689 (SEQ ID NO: 145) ATGGATGTCGTCCTCCAAAA |

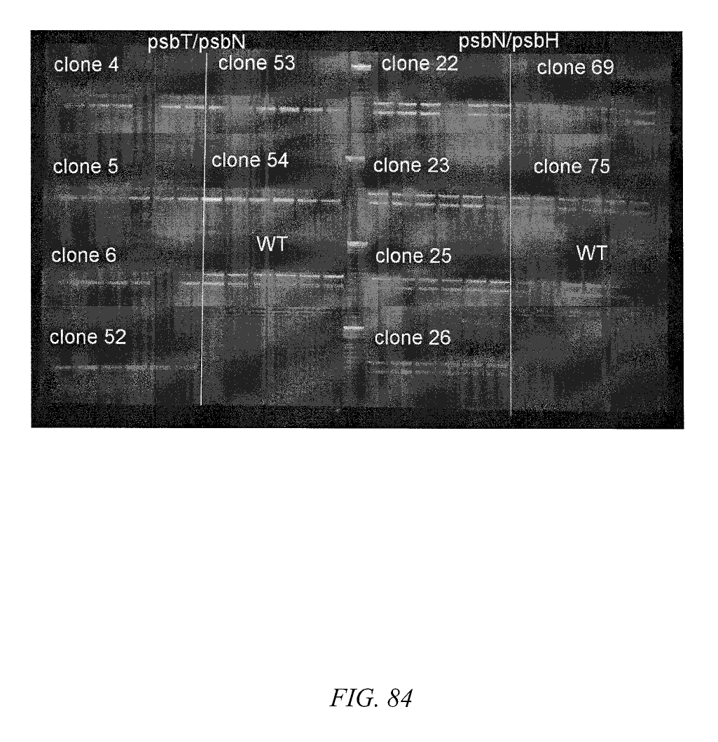

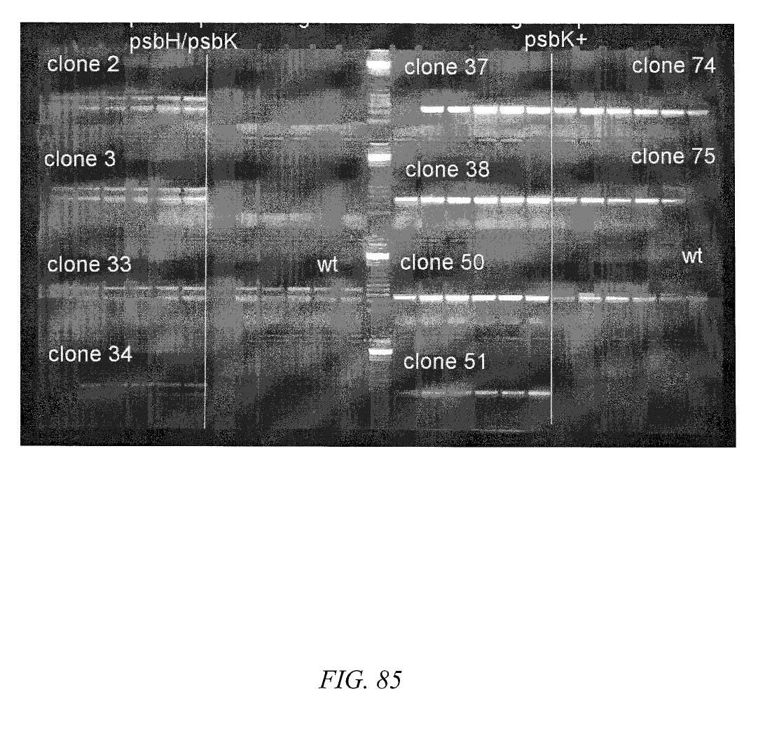

FIG. 84 and FIG. 85 shows that homoplasmic clones are recovered from integration between psbT and psbN (p04-142) and integration 3′ of psbK (p04-145).

### Example 23

### Creation of a Yeast-bacteria Shuttle Vector

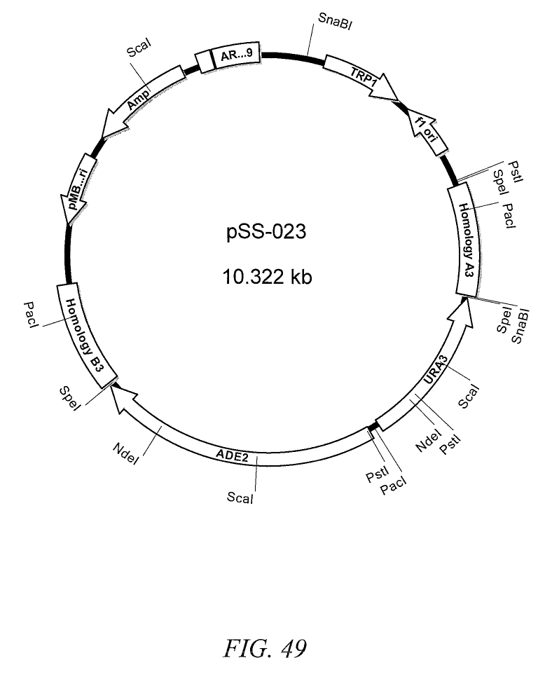

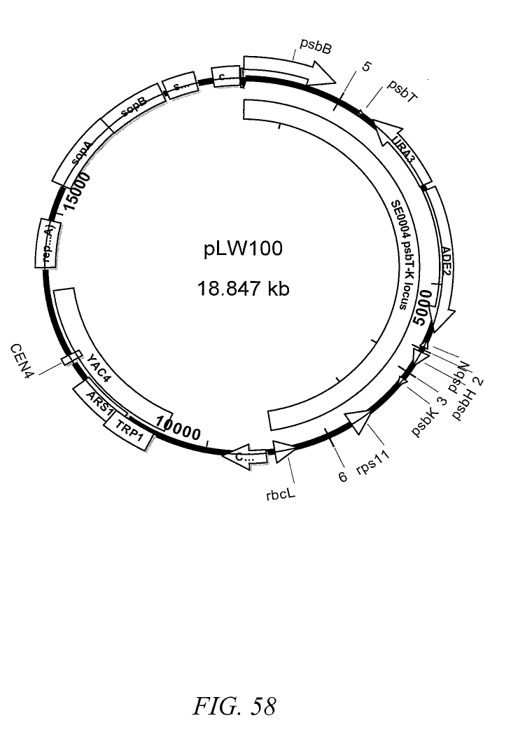

Heterologous (exogenous) gene introduction into the chloroplast by homologous recombination is efficient when a selectable marker and the gene of interest is flanked by 5′ and 3′ homology to a locus that can tolerate integration. To integrate more than one gene, one can target a separate locus and use a second selectable marker. Integration of two or more genes is problematic from a time and labor standpoint. In addition, availability of selectable markers becomes an issue. To contend with these issues, a yeast-based system was created wherein, in a single step, several exogenous genes can be assembled along with an algal selectable marker, and placed into a yeast-bacteria shuttle vector. Two versions of this vector were created. One version contains a 5.2 kb region from the Scenedesmus obliquus chloroplast (Scenedesmus chloroplast sequence NCBI reference sequence: NC_{—}008101, 057,611-062850 bp) (SEQ ID NO: 125). This 5.2 kb region is highly conserved (at the amino acid level) amongst algae species, and spans a region comprising psbB to rbcL genes. The second version of this vector contains two 1,000 bp “homology A3” (070,433-071,342 bp) (SEQ ID NO: 126) and “homology B3” (071,379-072,254 bp) (SEQ ID NO: 127) regions which target a locus immediately downstream of the psbA gene. The two shuttle vectors (FIGS. 49 and 58) comprise the above-mentioned sequences from the chloroplast genome of Scenedesmus obliquus, bacterial replication/selection elements, and yeast replication, segregation, and selection/counter-selection elements.

There are at least four advantages of the yeast-based system over the existing technology: 1) each of the 1, 2, 3, 4, or more gene expression cassettes can be amplified with primers containing 5′ and 3′ homology to adjacent cassettes, thereby alleviating the requirement to clone flanking homology into the gene cassette design; 2) several gene cassettes (for example, 2, 3, 4, 5, 6, or 20) can be assembled together as a contig in a single step and require a single selectable marker for chloroplast introduction; 3) this technology can be applied to other algal species due to the conserved nature of the psbB-rbcL locus across algae species; and 4) the 5.2 kb of homology contained within the shuttle vector (FIG. 58) and the 2 kb of homology as shown in FIG. 49, ensures that homologous recombination is accurate and efficient within the chloroplast.

It should be noted that, for example, more than 2, more than 5, more than 10, more than 15, more than 20, or more man 25 gene cassettes can be assembled in the shuttle vector.

### Example 24

### Plasmid Construction

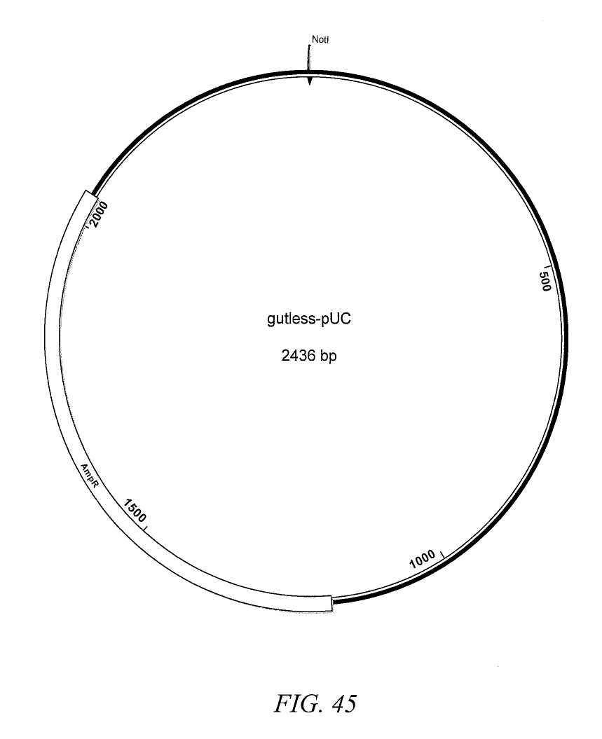

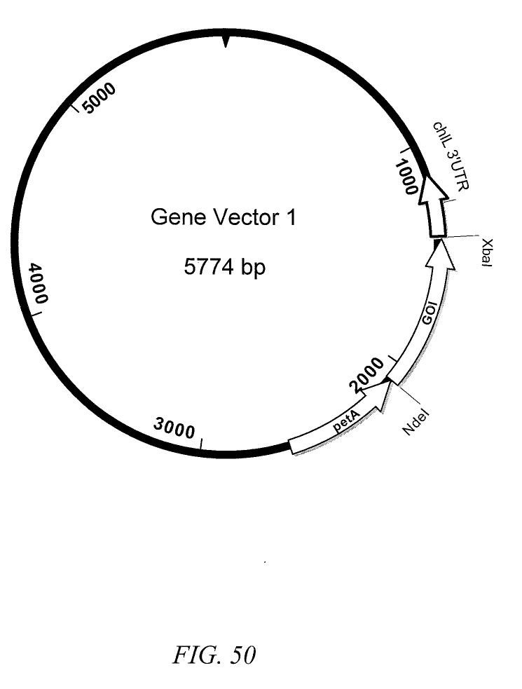

A derivative of plasmid vector pUC19 (New England Biolabs, U.S.A.; Yanisch-Perron, C, et al. (1985) Gene, 33, 103-119) lacking a multiple cloning site (herein referred to as gutless pUC) (FIG. 45) was used to create the backbones for three gene expression cassettes. Three different gene expression cassettes comprising the promoter-terminator pairs: petA-ch1L, D2-D3, and tufA-psaB, respectively were cloned into gutless pUC (FIGS. 50, 51 and 52).

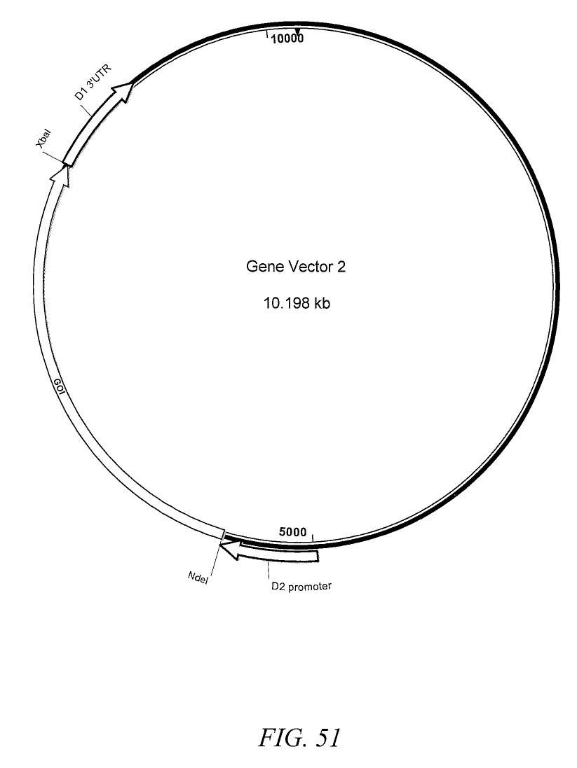

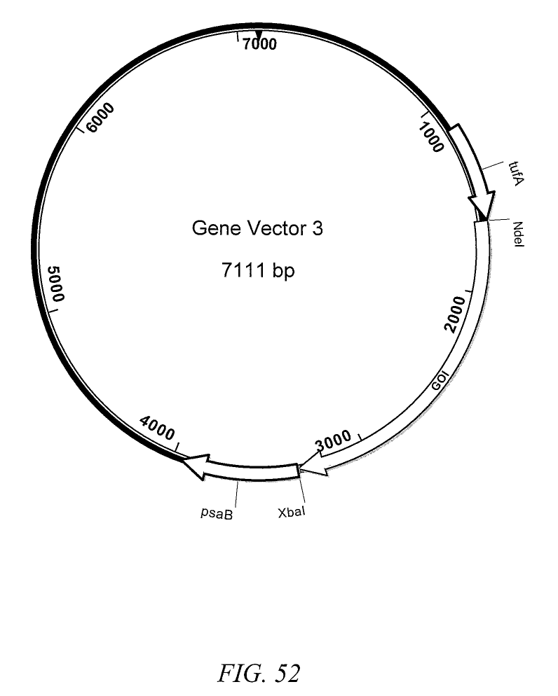

To insert the genes of interest (“GOI”)(CC90, SEQ ID NO: 115; CC91, SEQ ID NO: 116; CC92, SEQ ID NO: 117; CC93, SEQ ID NO: 109; CC94, SEQ ID NO: 110; CC97, SEQ ID NO: 113; IS57, SEQ ID NO: 121; IS61, SEQ ID NO: 124; IS62, SEQ ID NO: 123; IS116, SEQ ID NO: 122; BD11, SEQ ID NO: 146; and IS99, SEQ ID NO: 147), each of the three vectors (Gene Vector 1 (FIG. 50), Gene Vector 2 (FIG. 51), and Gene Vector 3 (FIG. 52)), along with the genes of interest, were double-digested with the restriction enzymes NdeI and XbaI, and ligated together resulting in 36 different vectors. Several of the 36 vectors served as PCR templates for the gene amplifications used in the 2-, 3-, or 4-gene contig assemblies described below.

The genes of interest are as follows:

CC90 glcD—glycolate oxidase subunit, FAD-linked NP_{—}417453;

CC91 glcE—glycolate oxidase FAD binding subunit YP_{—}026191;

CC92 glcF—glycolate oxidase iron-sulfur subunit YP_{—}026190;

CC93 glyoxylate carboligase NP_{—}415040;

CC94 tartronate semialdehyde reductase NP_{—}417594; and

CC97 tartronate semialdehyde reductase—NADH dependent NP_{—}415042.

These genes are described in Kebeish, R., et al., Nature Biotechnology (2007) 25(5) 593-599, All six genes are codon-optimized for the chloroplast genome of Chlamydomonas reinhardtii.

Additional genes of interest are as follows:

BD11 is an endoxylanase from T. reesei; and

IS99 is a mevalonate pyrophosphate decarboxylase from S. cerevisiae, codon optimized according to the tRNA usage of the C. reinhardtii chloroplast.

Other genes of interest are as follows:

IS57 is 1-Deoxy-D-xylulose 5-phosphate reductoisomerase (DXR);

IS-61 is Chlamydomonas chlorophyll synthase;

IS-62 is the same protein as IS-9, the chicken FPP synthase; the difference is that the C-terminal tag has been removed, and replaced with an N-terminal FLAG tag; and

IS-116 is 4-diphosphocytidyl-2-C-methylerythritol synthetase (CDP-ME synthase, it is the E. coli version of the gene).

These above four genes were all codon biased for expression in the Chlamydomonas chloroplast genome.

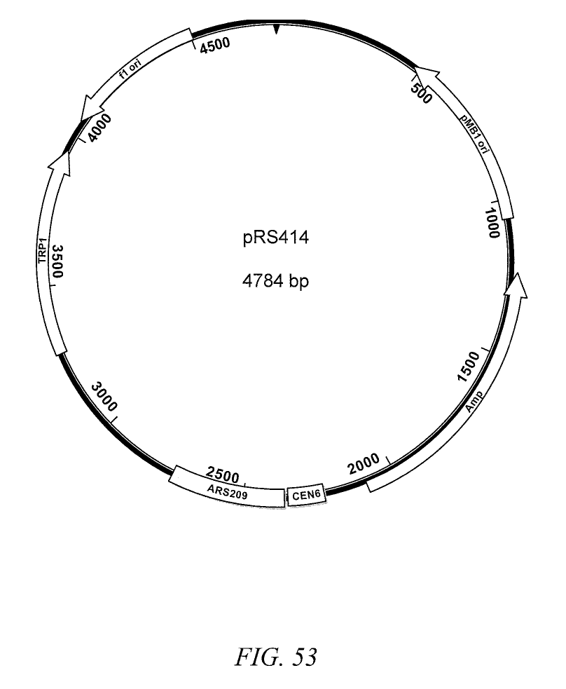

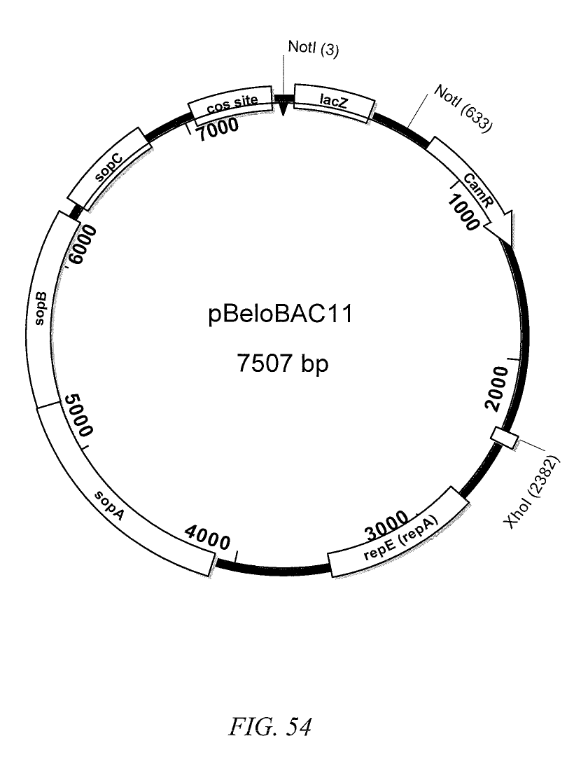

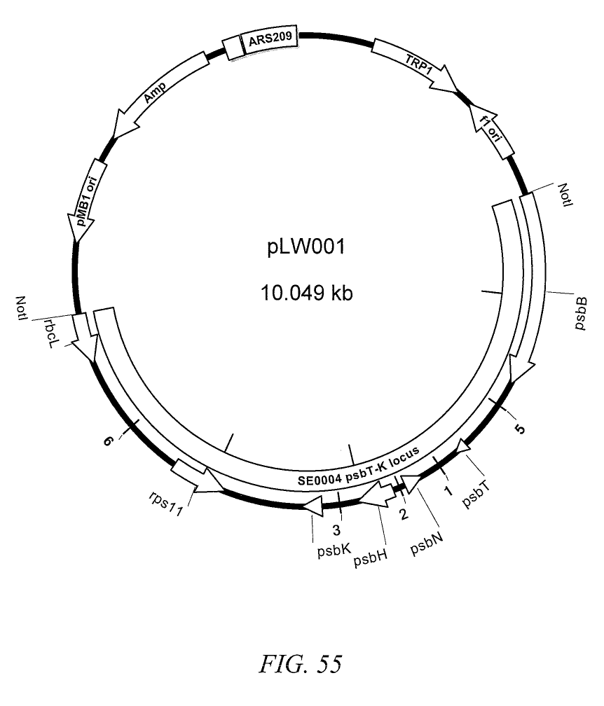

Plasmid vectors pRS414 (Sikorski and Hieter, Genetics. 1989 May; 122(i):19-27) (FIG. 53) and pBeloBAC11 (NEB)(FIG. 54) were used to construct transformation platform vectors. In all instances, pRS414, and Gene Vectors 1, 2, and 3 were selectively maintained in DH10B cells (Invitrogen, U.S.A.) by growth in Luria Bertani (LB) medium supplemented with 100 μg/ml ampicillin. Similarly, the plasmid pBeloBAC11 was selectively maintained in its host bacterium, DH10B, by growth in LB medium supplemented with 12.5 μg/ml chloramphenicol.

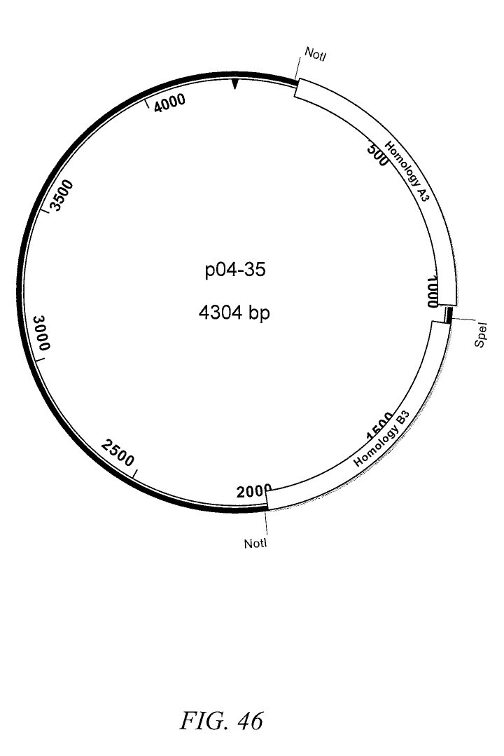

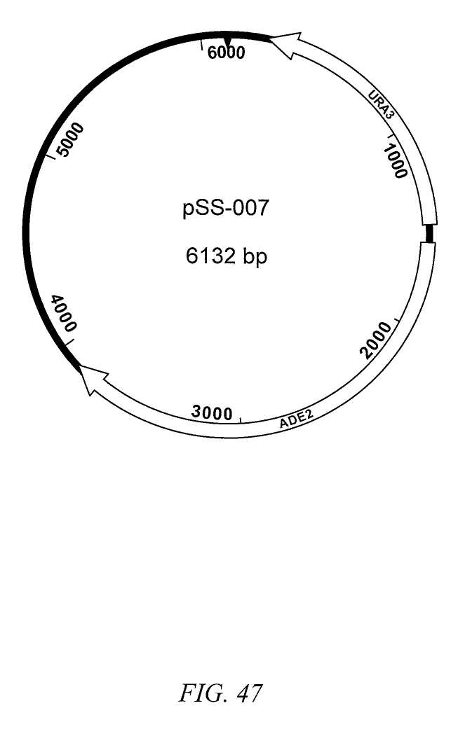

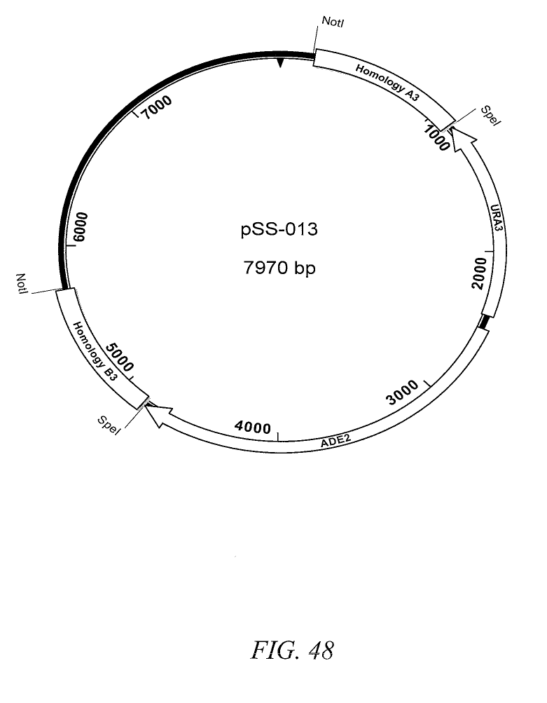

To construct the first of the two base platform vectors (FIG. 49) that can be used for the introduction of two genes into the chloroplast of Scenedesmus obliquus, the homology region A3 (SEQ ID NO: 126) and the homology region B3 (SEQ ID NO: 127) were amplified from Scenedesmus chloroplast DNA using primers 34 (SEQ ID NO: 99) and 35 (SEQ ID NO: 100), and 36 (SEQ ID NO: 101) and 37 (SEQ ID NO: 102), respectively, digested with NotI and SpeI, and ligated into NotI digested gutless pUC (FIG. 45), Plasmid p04-35 (FIG. 46) was then linearized with SpeI and ligated to a PCR product comprising the nucleotide sequence encoding the yeast genes URA3-ADE2 (SEQ ID NO: 105). The nucleotide sequence encoding the yeast genes URA3-ADE2 was obtained by PCR rising as a DNA template, plasmid pSS-007 (FIG. 47), and primers 30 (SEQ ID NO: 95) and primer 31 (SEQ ID NO: 96), which both contain SpeI restriction sites at their 5′ termini. The resulting vector comprising the homology regions flanking the yeast genes (pSS-013) is shown in FIG. 48.

The URA3-ADE2 cassette allows for positive selection in yeast that are deficient for URA3 or ADE2 gene function, respectively. Similarly, expression of the URA3 gene can be negatively selected against in the presence of 5-floroorotic acid (5-FOA) as URA3 converts 5-FOA to 5-fluorouracil, which is toxic to the cell. In addition, the presence or absence of a functional ADE2 gene results in white or red yeast colonies, respectively—thereby allowing for another level of selection when picking colonies.

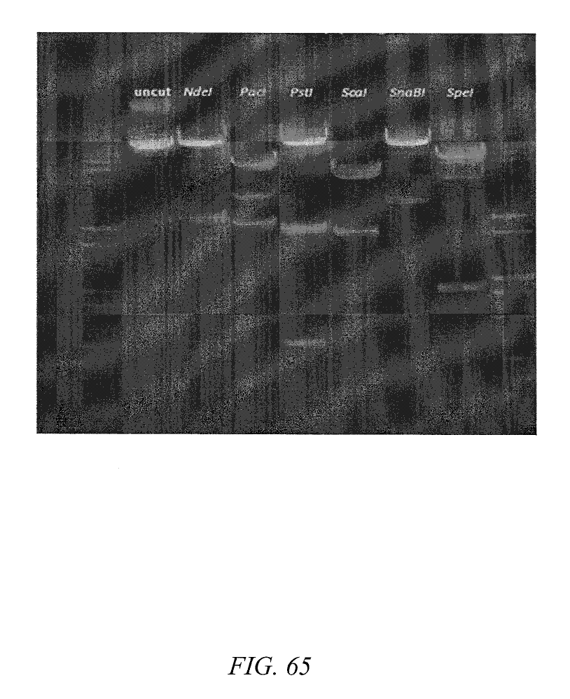

To create the yeast-bacterial shuttle vector for two-gene contig assembly, which targets the A3-B3 region, pSS-013 (FIG. 48) was digested with NotI, liberating the fragment, containing A3-URA3-ADE2-B3, which was then ligated into Nod digested pRS414 (FIG. 53), resulting in the vector pSS-023 (FIG. 49). pSS-023 was confirmed by sequencing and restriction digest, mapping with NdeI PacI, PstI, ScaI, SnaBI, and SpeI (FIG. 65). Order of lanes from left to right: 1 kb DNA plus ladder (Invitrogen), uncut pSS-023, NdeI, PacI, PstI, ScaI, SnaBI, SpeI, 1 kb DNA plus ladder (Invitrogen, U.S.A.). Expected bands are as follows: NdeI, 2187 bp and 8135 bp; PacI, 2051 bp, 2981 bp, and 5290 bp; PstI, 493 bp, 1872 bp, and 7957 bp; ScaI, 1761 bp, 4050 bp, and 4511 bp; SnaBI, 2587 bp and 7735 bp; and SpeI, 950 bp, 3694 bp, and 5678 bp. pSS-023 was used in all two-gene contig assemblies that target homology A3 and homology B3 regions.

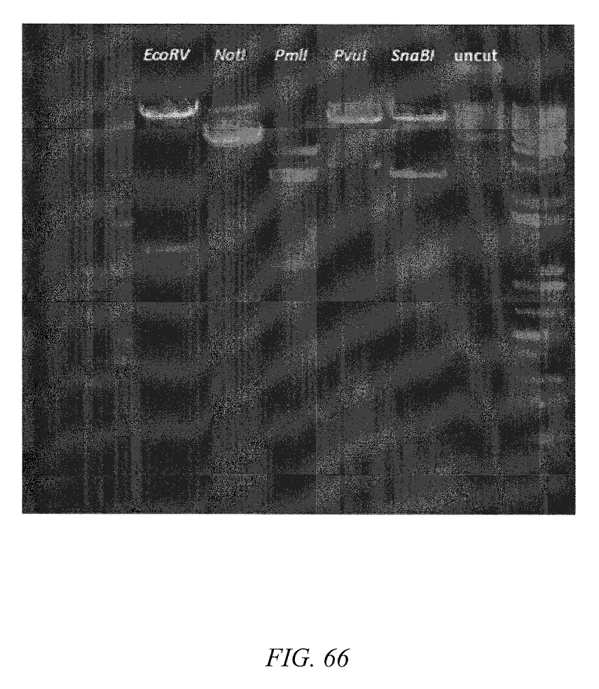

To construct the base platform vector used for the three-gene, four-gene, and the second two-gene contig assembly (which all target the psbB-rbcL locus in Scenedesmus), primer 1 (SEQ ID NO: 66) and primer 2 (SEQ ID NO: 67), both of which contain NotI restriction sites at their 5′ termini, were used to amplify the 5.2 kb sequence (SEQ ID NO: 125) spanning from the psbB gene to the rbcL gene. The resultant 5.2 kb PCR product and plasmid vector pRS414 (FIG. 53) were both digested with NotI and ligated together, resulting in pLW001 (FIG. 55). pLW001 was confirmed by sequencing and restriction digest mapping with EcoRV, NotI, PmlI, PvuI, and SnaBI (FIG. 66). Order of lanes from left to right: 1 kb DNA plus ladder (Invitrogen, U.S.A.), EcoRV, NotI, PmlI, PvuI, SnaBI, uncut, and 1 kb DNA plus ladder (Invitrogen, U.S.A.). Expected bands are as follows: EcoRV, 1182 bp and 8867 bp; NotI, 4784 bp and 5265 bp; PmlI, 995 bp, 2644 bp, and 2695 bp; PvuI, 2868 bp and 7181 bp; and SnaBI, 2526 bp and 7523 bp.

To assemble contigs of two, three, and four genes in pLW001, using negative selection, a PCR product containing the Saccharomyces cerevisiae genes URA3-ADE2 (SEQ ID NO: 305) was amplified with primer 27 (SEQ ID NO: 92) and primer 28 (SEQ ID NO: 93), which contain 5′ tails homologous to the locus in the chloroplast sequence between psbT and psbN. This PCR product, along with pLW001 (FIG. 55), were simultaneously transformed info S. cerevisiae. Transformants were selected for on complete synthetic media (CSM) lacking tryptophan, uracil, and adenine (CSM-TRP-URA-ADE) using a standard lithium acetate transformation protocol (for example, as described in Gietz, R. D. and Woods, R. A., Methods Enzymol. (2002) 350:87-96).

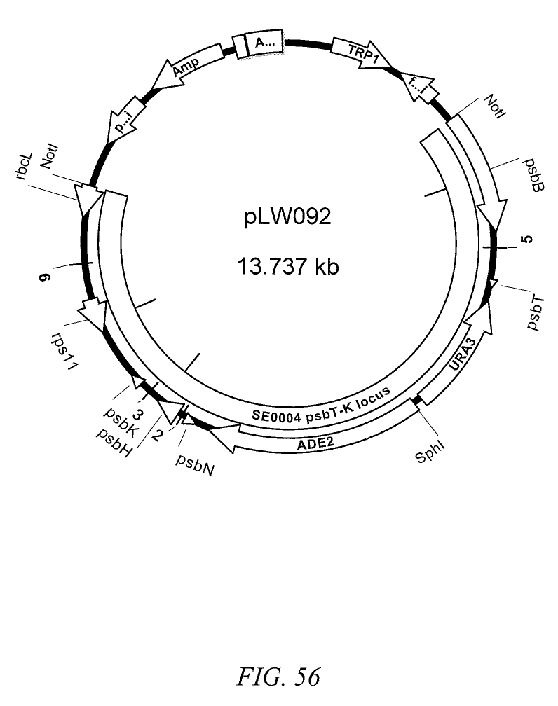

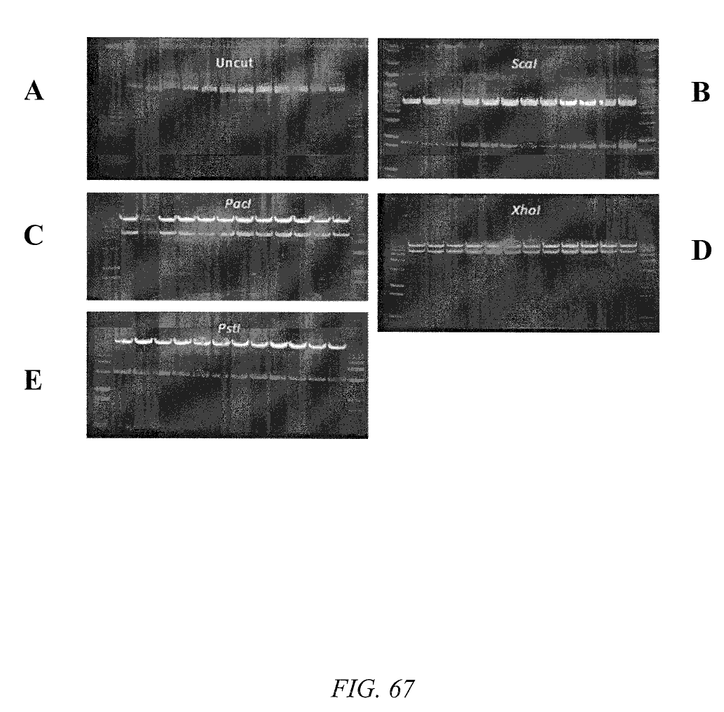

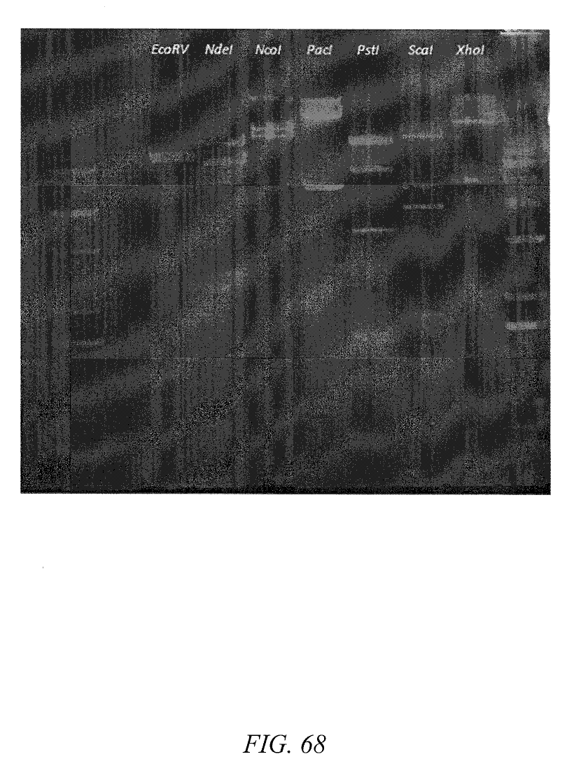

Resultant yeast colonies were patched to CSM-TRP-URA-ADE and PCR screened for the correct homologous insertion of the URA3-ADE2 construct. Plasmid DNA was then harvested from PCR positive yeast clones and electroporated into E. coli DH10B cells (invitrogen). Bacterial colonies were PCR screened. PCR positive clones were then harvested for plasmid DNA (Qiagen miniprep protocol). Twelve independent plasmid isolates from the above-mentioned yeast colonies were sequence confirmed and restriction enzyme mapped with PacI, PstI, ScaI, and XhoI (FIGS. 67A-E). FIG. 67A is uncut plasmid DNA. FIG. 67B is the plasmid DNA digested with ScaI; the expected fragments are 1761 bp, 5646 bp, and 6330 bp. FIG. 67C is the plasmid DNA digested with PacI; the expected fragments are 4847 bp and 8890 bp. FIG. 67D is the plasmid DNA digested with XhoI; expected fragments are 5830 bp and 7907 bp. FIG. 67E is the plasmid DNA digested with PstI; the resulting fragments are 493 bp, 3011 bp, and 10233 bp. The resulting platform construct was designated as pLW092 (FIG. 56).

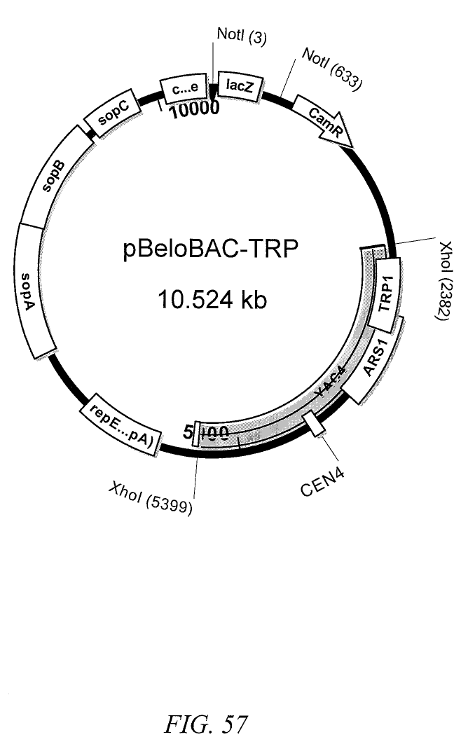

The size of the contig becomes an issue in assembling contigs of three or more genes as the colE1 origin present in the pLW092 backbone (FIG. 56) is unable to support faithful duplication of plasmids greater than 20 kb. To contend with this issue, a platform vector was created that is capable of larger assemblies based on the BAC cloning vector, pBeloBAC11 (FIG. 54), which, contains the OriS origin capable of maintaining very large DNA fragments, for example, upwards of 300 kb. Briefly, pBeloBAC11 was linearized using the restriction, enzyme XhoI. The TRP1-ARS1-CEN4 gene sequences (SEQ ID NO: 107) was PCR-amplified from pYAC4 (ATCC; GenBank number U01086; Burke, D. T. et al., Science (1987) 236: 806-812) wish primer 3 (SEQ ID NO: 68) and primer 4 (SEQ ID NO: 69), which both contain XhoI ends. The XhoI-digested BeloBAC11 and pYAC4 sequences were ligated together. Resultant bacterial colonies were PCR screened for the correct ligation event, restriction enzyme mapped, and sequence confirmed. The resultant plasmid was designated pBeloBAC-TRP (FIG. 57).

pBeloBAC-TRP was further modified to incorporate the Scenedesmus psbB-rbcL locus (containing URA3-ADE2 between psbT and psbN, from pLW092). Briefly, the Scenedesmus psbB-rbcL locus was digested away from pLW092 (FIG. 56) using NotI and ligated into pBeloBAC11-TRP (FIG. 57) (also digested with NotI), Resultant bacterial clones were sequence confirmed and restriction enzyme mapped with EcoRV, NdeI, NotI, PacI, PstI, ScaI and XhoI (FIG. 68). Order of lanes from left, to right: 1 kb DNA plus ladder (Invitrogen, U.S.A.), empty, EcoRV, NdeI, NcoI, PacI, PstI, ScaI, XhoI, and 1 kb DNA plus ladder (Invitrogen, U.S.A.). Expected bands are as follows: EcoRV, 229 bp, 1290 bp, 1461 bp, 2261 bp, 6558 bp, and 7048 bp; NdeI, 2187 bp, 2470 bp, 6183 bp, and 8007 bp; NotI, 8953 bp and 9894 bp; PacI, 4847 bp and 14000 bp; PstI, 493 bp, 1541 bp, 3179 bp, 5559 bp, and 8075 bp; ScaI, 1761 bp, 3835 bp, 4704 bp, and 8547 bp; and XhoI, 3017 bp, 4942 bp, and 10888 bp. The resultant platform construct was designated as pLW100 (FIG. 58) and is used in all of the 3- and 4-gene contig assemblies.

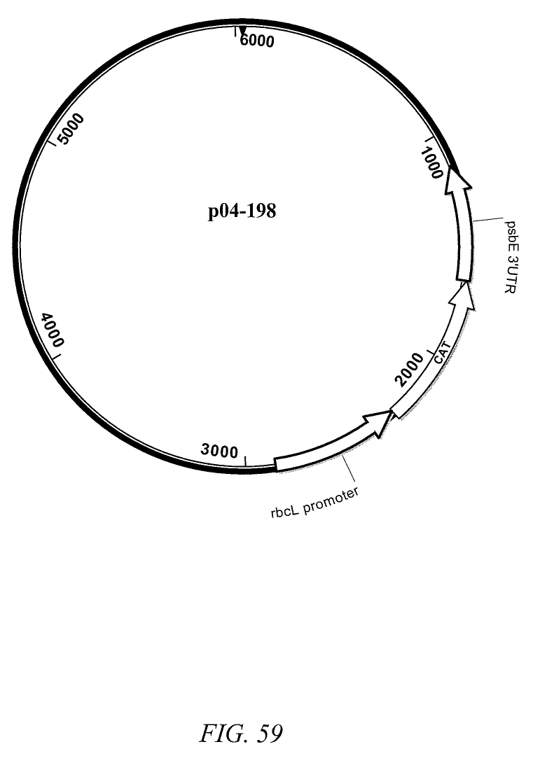

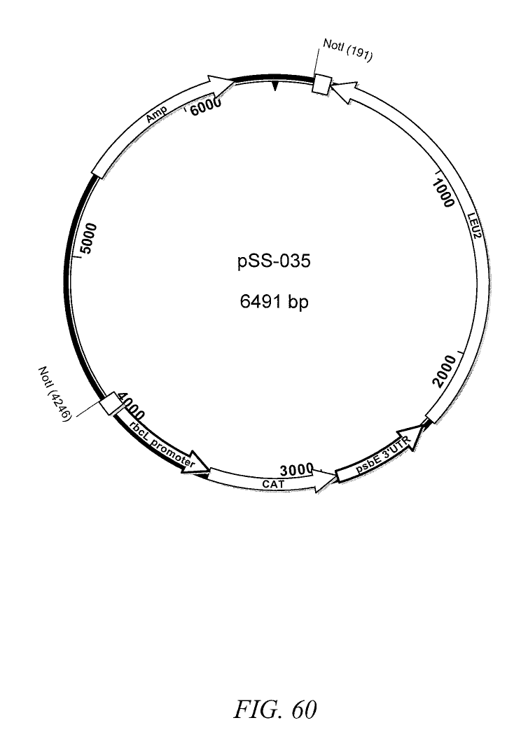

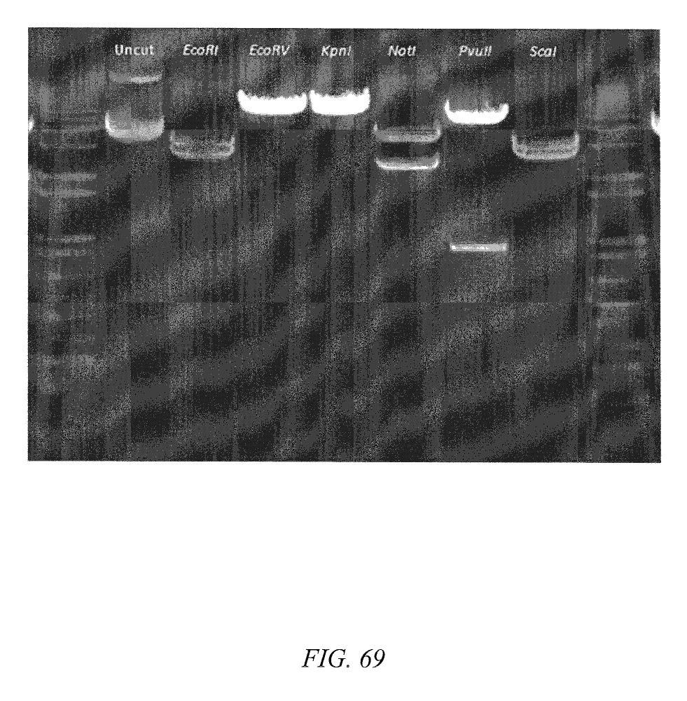

In addition to the genes of interest assembled into the 2-3- and 4-gene contigs, a yeast positive selection marker and a Scenedesmus positive selection marker were also included. The yeast auxotrophic marker, LEU 2 (SEQ ID NO: 108), along with the chlorampenicol acetyltransferase (CAT) gene (SEQ ID NO: 148) driven by the rbcL promoter (which confers resistance to chloramphenicol in Scenedesmus) (FIG. 59) were ligated into gutless-pUC. Homology regions flanking these two genes were also cloned, which correspond to the adjacent genes of interest in contig assembly. Briefly, the Saccharomyces cerevisiae gene LEU2 (SEQ ID NO: 108), was amplified from total genomic DNA with primer 5 (SEQ ID NO; 70), which contains a PstI restriction site, and primer 6 (SEQ ID NO: 71), which contains a NotI restriction site (at the 5′ terminus) and 80 bp of DNA, which are homologous to adjacent genes in 2, 3-, and 4-gene contig assembly. In addition, the rbcL-CAT-psbE gene (SEQ ID NO: 128) was amplified from vector p04-198 (FIG. 59) using primer 7 (SEQ ID NO: 72), which contains a NotI restriction site (at the 5′ terminus) and 80 bp of DNA which are homologous to adjacent genes in 2-, 3-, and 4-gene contig and primer 8 (SEQ ID NO: 73), which contains a PstI restriction site. The LEU2 and rbcL-CAT-psbE fragments were digested with PstI and NotI and ligated to NotI digested gutless-pUC. Resultant bacterial clones were sequence confirmed and restriction enzyme mapped with EcoRI, EcoRV, KpnI, NotI, PvulI, and ScaI (FIG. 69). The order of lanes is as follows: 1 kb DNA plus ladder (Invitrogen, U.S.A.), uncut DNA, EcoRI, EcoRV, KpnI, NotI, PvulI, ScaI, and 1 kb DNA plus ladder (Invitrogen, U.S.A.). Expected bands are as follows: EcoRI, 3033 bp and 3458 bp; EcoRV, 6491 bp; KpnI, 6491 bp; NotI, 2436 bp and 4055 bp; PvulI, 958 bp and 5533 bp; ScaI 3023 bp and 3468 bp. This construct was designated as pSS-035 (FIG. 60) and is used in all of the gene contigs to promote proper assembly and also to provide for a positive selection element during Scenedesmus transformation.

### Example 25

### Contig. Assemblies

The Saccharomyces cerevisiae strain, YPH858 (MATa, ura3-52, lys2-801, ade2-101, trplΔ63, his3Δ200, leu2Δ1, cyh2R), was used in all contig assembly reactions.

For two-gene contig assemblies targeting the A3-B3 region, the following were combined:
1) 1 μg of pSS-023 (FIG. 49) linearized between URA3 and ADE2 with SphI;
2) 500 ng of a gel purified fragment, obtained by digesting pSS-035 (FIG. 60) with NotI, and comprising the rbcL-CAT-psbE/LEU2 construct;
3) 500 ng of PCR amplified petA-CC94-chlL (gene vector 1) (FIG. 50), amplified with a forward primer, primer 9 (SEQ ID NO: 74), which is comprised of 60 bp of homology to the NotI digestion product, from pSS-035, and a reverse primer, primer 32 (SEQ ID NO: 97), which is comprised of 60 bp of homology to pSS-023 just downstream of the nucleotide sequence encoding for ADE2; and
4) 500 ng of PCR amplified tufA-CC93-psaB (gene vector 3) (FIG. 52), amplified with a forward primer, primer 33 (SEQ ID NO: 98), which comprises 60 bp of homology to pSS-023 just upstream of the nucleotide sequence encoding for URA3, and a reverse primer, primer 12 (SEQ ID NO: 77), which comprises 60 bp of homology to the NotI digestion product described in step 2 above.

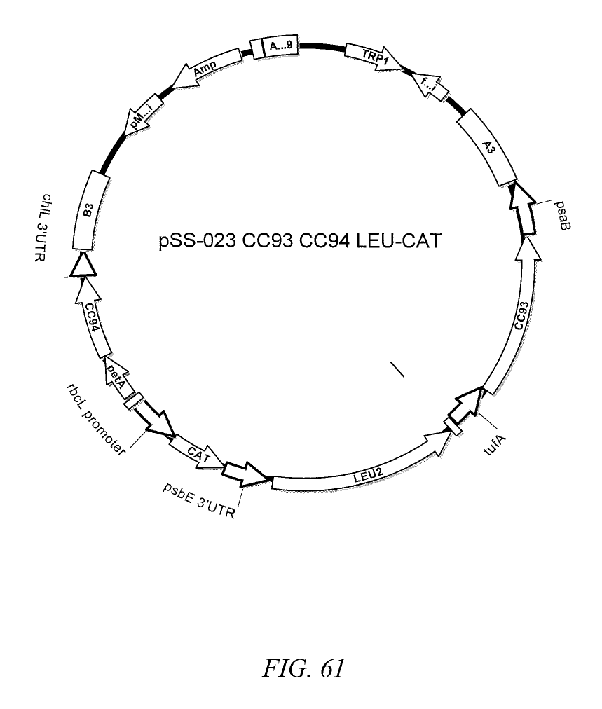

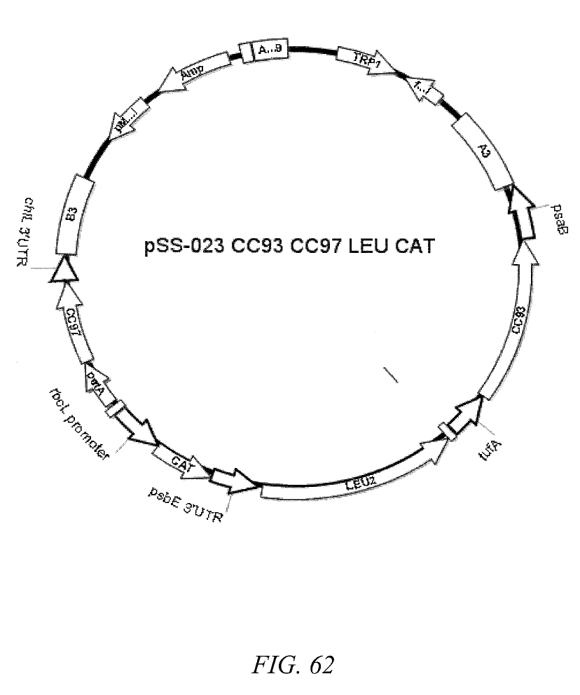

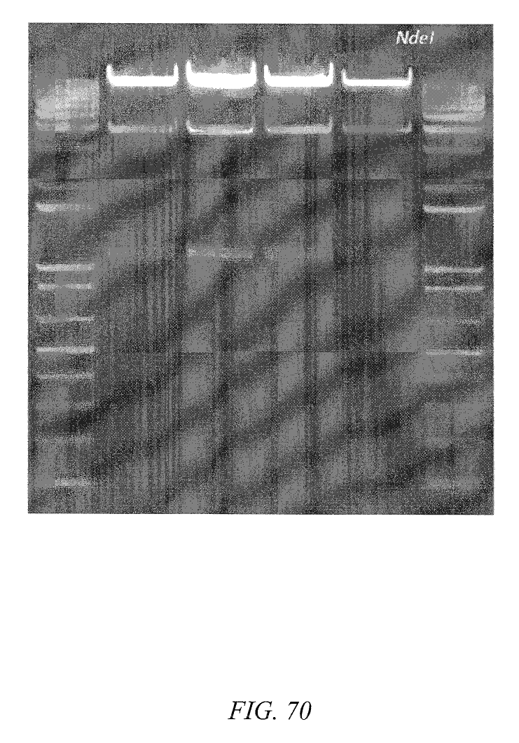

Cells were transformed with the mixture of DNA described above, using a standard lithium acetate transformation protocol. Transformants were selected for on CSM-TRP-LEU +5-FOA plates. After two days at 30° C., yeast colonies were picked and patched to a CSM-TRP-LEU plate. The next day, yeast patches were PCR screened for the correct gene assembly. Plasmid DNA was then harvested from PCR positive yeast clones and electroporated into E. coli DH10B cells (Invitrogen). Bacterial colonies were also PCR screened. Four PCR positive clones were then harvested for the preparation of plasmid DNA (Qiagen miniprep protocol), which were subsequently restriction enzyme mapped with NdeI (FIG. 70; expected band sizes, 1097 bp, 3703 bp, and 10283 bp; and 1 kb DNA plus ladder (Invitrogen, U.S.A.). One of the four clones was picked and the sequence of that clone was confirmed. The resulting two-gene contig assembly is shown in FIG. 61. Another embodiment of this assembly is shown in FIG. 62.

For two-gene contig assemblies targeting the 5.2 kb psbB-rbcL region, the following were combined:
1) 1 μg of pLW092 (FIG. 56) linearized between URA3 and ADE2 with SphI;
2) 500 ng of a gel purified fragment, obtained by digesting pSS-035 (FIG. 60) with NotI, and comprising the rbcL-CAT-psbE/LEU2 construct;
3) 500 ng of PCR amplified petA-BD11-chL (gene vector 1) (FIG. 50), amplified with a reverse primer, primer 1001 (SEQ ID NO: 150), which is comprised of 60 bp of homology to the NotI digestion product from pSS-035, and a forward primer, primer 1000 (SEQ ID NO: 149), which is comprised of 60 bp of homology to pLW092 just upstream of the nucleotide sequence encoding for URA3; and
4) 500 ng of PCR amplified tufA-IS99-psaB (gene vector 3) (FIG. 52), amplified with a reverse primer, primer 1002 (SEQ ID NO: 151), which comprises 60 bp of homology to pLW092 just downstream of the nucleotide sequence encoding for ADE2, and a forward primer, primer 1003 (SEQ ID NO: 152), which comprises 60 bp of homology to the NotI digestion product described in step 2 above.

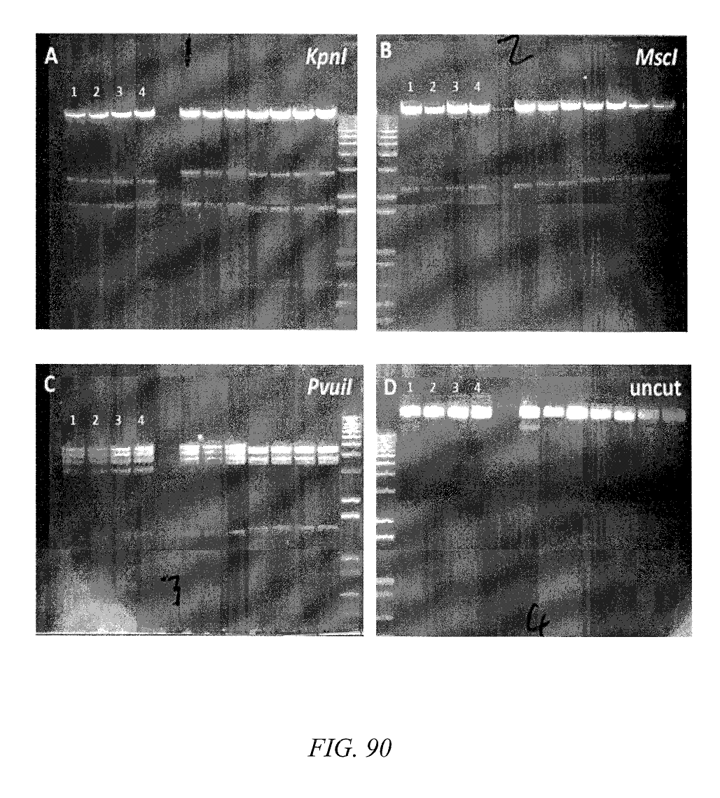

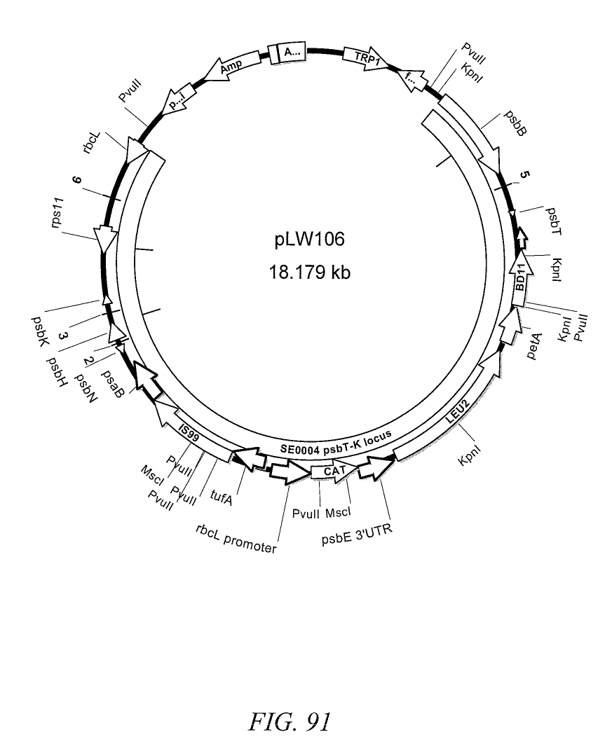

Cells were transformed with the mixture of DNA described above, using a standard lithium acetate transformation protocol. Transformants were selected for on CSM-TRP-LEU +5-FOA plates. After two days at 30° C., yeast colonies were picked and patched to a CSM-TRP-LEU plate. The next day, yeast patches were PCR screened for the correct gene assembly. Plasmid DNA was then harvested from PCR positive yeast clones and electroporated into E. coli DH10B cells (Invitrogen), Bacterial colonies were also PCR screened. Four PCR positive clones were then harvested for the preparation of plasmid DNA (Qiagen miniprep protocol), which were subsequently restriction enzyme mapped. FIG. 90A-D depicts mapping of the two gene contig assembly with the restriction enzymes: KpnI (A), MscI (B), PvuII (C), and also uncut DNA (D). Expected band sizes are as follows: KpnI: 670 bp, 1791 bp, 2555 bp, and 13163 bp; MscI: 2206 bp and 15973 bp; and PvulI: 21 bp, 195 bp, 1421 bp, 3289 bp, 3908 bp, 4336 bp, and 5009 bp (note: the 21 bp and 195 bp bands have run off the gel in FIG. 90C). One of the four clones was picked and the sequence of that clone was confirmed. The resulting two-gene contig assembly targeting the psbB-rbcL locus is shown in FIG. 91.

For a three-gene contig assembly, the following were combined:
1) 1 μg of pLW100 (FIG. 58) linearized between URA3 and ADE2 with SphI;
2) 500 ng of a gel purified fragment, obtained by digesting pSS-035 (FIG. 60) with NotI, and comprising the rbcL-CAT-psbE/LEU2 construct;
3) 500 ng of PCR amplified petA-CC90-chlL (gene vector 1) (FIG. 50), amplified with a forward primer, primer 13 (SEQ ID NO: 78), which comprises 60 bp of homology to the NotI digestion product from pSS-035, and a reverse primer, primer 14 (SEQ ID NO: 79), which comprises 60 bp of homology to pLW100 just upstream of the nucleotide sequence encoding for URA3;
4) 500 ng of PCR amplified tufA-CC91-psaB (gene vector 3) (FIG. 52), amplified with a forward primer, primer 15 (SEQ ID NO: 80), which comprises 60 bp of homology to the NotI digestion product from step 2, and a reverse primer, primer 16 (SEQ ID NO: 81), which comprises 60 bp of homology to the PCR amplified gene vector 2 (FIG. 51); and
5) 500 ng of PCR amplified D2-CC92-D1 (gene vector 2) (FIG. 51), amplified with a forward primer, primer 29 (SEQ ID NO: 94), which comprises 60 bp of homology to PCR amplified gene vector 2, and a reverse primer, primer 17 (SEQ ID NO: 82), which comprises 60 bp of homology to pLW100, just downstream of the nucleotide sequence encoding for ADE2.

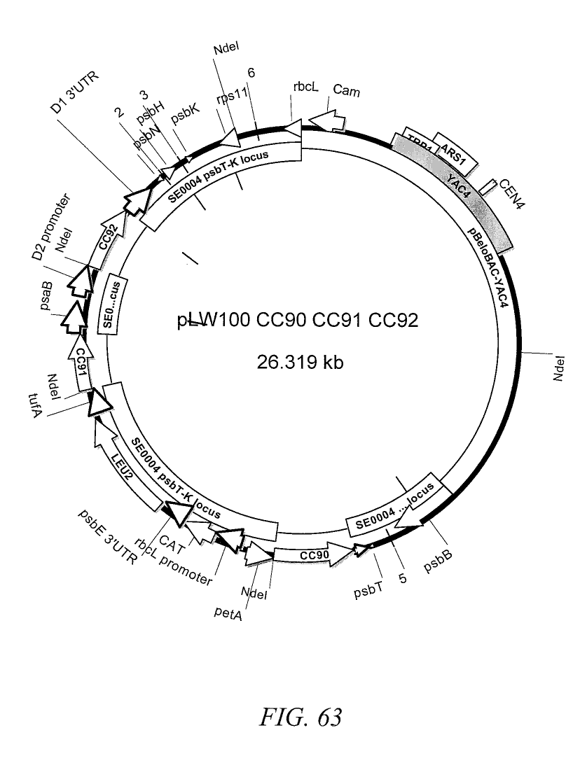

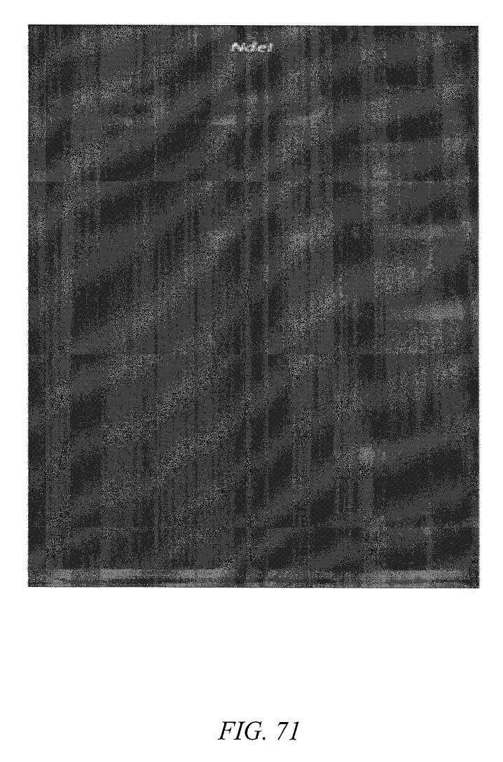

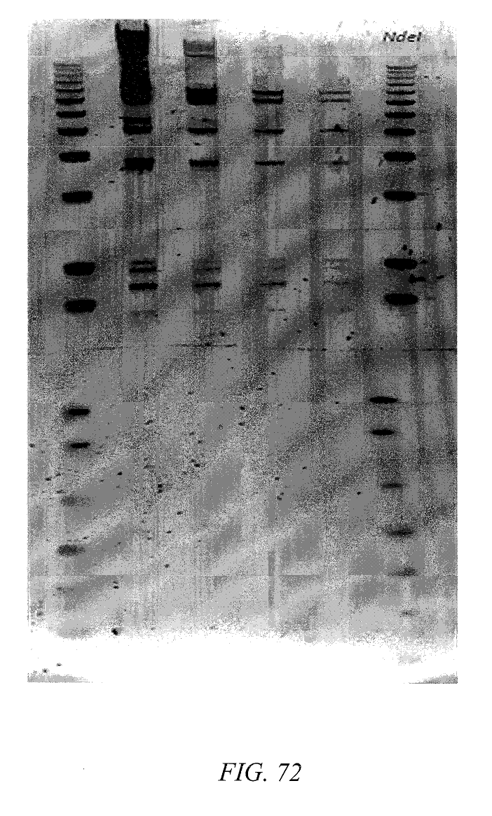

Cells were transformed with the mixture of DNA described above, using a standard lithium acetate transformation protocol. Transformants were selected for on CSM-TRP-LEU +5-FOA plates. After two days at 30° C., yeast colonies were picked and patched to a CSM-TRP-LEU plate. The next day, yeast patches were PCR screened for the correct gene assembly. Plasmid DNA was then harvested from PCR positive yeast clones and electroporated into E. coli DH10B cells (Invitrogen). Bacterial colonies were also PCR screened. Two PCR positive clones were then harvested for plasmid DNA (Qiagen maxiprep protocol), which were subsequently restriction enzyme mapped with NdeI (FIG. 71; expected bands, 2396 bp, 3873 bp, 5114 bp, 6929 bp, and 8007 bp; and 1 kb DNA plus ladder (Invitrogen, U.S.A.)). One of the two clones was picked and the sequence of that clone was confirmed. The resulting three-gene contig assembly is shown in FIG. 63.

To facilitate proper assembly of the 4-gene contig assembly, two positive selection yeast auxotrophic markers, HIS3 (SEQ ID NO: 118) and LYS2 (SEQ ID NO: 119), were added to the contig assembly.

For four-gene contig assemblies, the following were combined:
1) 1 μg of pLW100 (FIG. 58) linearized between URA3 and ADE2 with SphI;
2) 500 ng of a gel purified fragment, obtained by digesting pSS-035 (FIG. 60) with NotI, and comprising the rbcL-CAT-psbE/LEU2 construct;
3) 500 ng of PCR amplified tufA-1857-psaB (gene vector 3) (FIG. 52), amplified with a forward primer, primer 19 (SEQ ID NO: 84, which contains 60 bp of homology to PCR amplified HIS3, and a reverse primer, primer 20 (SEQ ID NO; 85), which, contains 60 bp of homology to pLW100 just upstream of the nucleotide sequence encoding for URA3;
4) 500 ng of PCR amplified HIS3, amplified with a forward primer, primer 21 (SEQ ID NO: 86), which contains 60 bp of homology to PCR amplified gene vector 3 and a reverse primer, primer 22 (SEQ ID NO: 87), which contains 60 bp of homology to PCR amplified gene vector 1 (FIG. 50);
5) 500 ng of PCR amplified petA-IS116-chlL (gene vector 1), amplified with a forward primer, primer 13 (SEQ ID NO: 78), which contains 60 bp of homology to the NotI digestion product from step 2, and a reverse primer, primer 23 (SEQ ID NO: 88), which contains 60 bp of homology to PCR amplified HIS3;
6) 500 ng of PCR amplified tufA-IS62-psaB (gene vector 3), amplified with a forward primer, primer 16 (SEQ ID NO: 81), which contains 60 bp of homology to the NotI digestion product from step 2 and a reverse primer, primer 15 (SEQ ID NO: 80), which contains 60 bp of homology to PCR amplified LYS2;
7) 500 ng of PCR amplified LYS2, amplified with a forward primer, primer 24 (SEQ ID NO: 89), which contains 60 bp of homology to PCR amplified gene vector 3 and a reverse primer, primer 25 (SEQ ID NO: 90), which contains 60 bp of homology to PCR amplified gene vector 2 (FIG. 51); and
8) 500 ng of PCR amplified D2-IS61-D1 (gene vector 2), amplified with a forward primer, primer 26 (SEQ ID NO: 91), which contains 60 bp of homology to PCR amplified LYS2 and a reverse primer, primer 18 (SEQ ID NO: 83), which contains 60 bp of homology to pLW100 just downstream of ADE2.

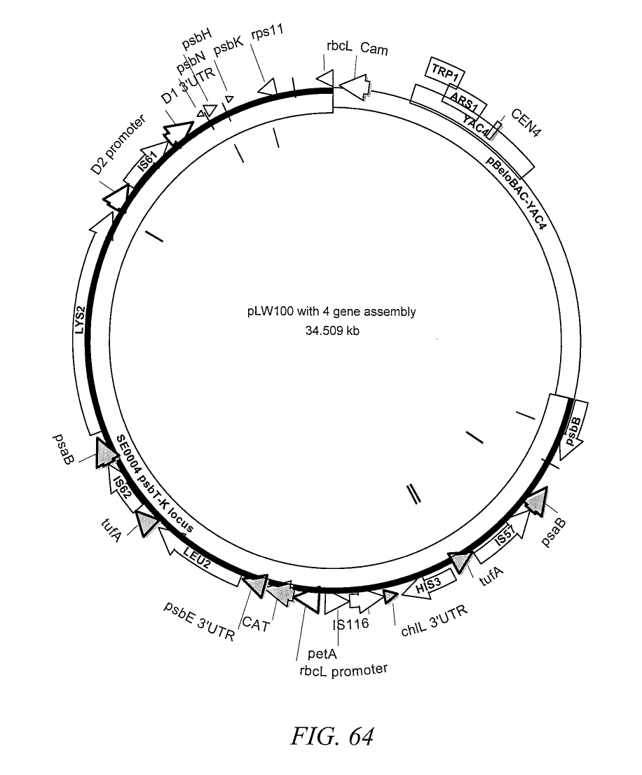

Cells were transformed with this mixture of DNA using a standard lithium acetate transformation protocol, Transformants were selected for on CSM-TRP-LEU-HIS-LYS +5-FOA plates. After two days at 30° C., yeast colonies were picked and patched to a CSM-TRP-LEU-HIS-LYS plate. The next day, yeast patches were PCR screened for the correct gene assembly. Plasmid DNA was then harvested from PCR positive yeast clones and electroporated into E. coli DH10B cells (Invitrogen). Bacterial colonies were also PCR screened. Four PCR positive clones were then harvested for plasmid DNA (Qiagen maxiprep protocol), which were subsequently restriction enzyme mapped with NdeI (FIG. 72; expected bands, 553 bp, 564 bp, 1570 bp, 1791 bp, 1824 bp, 1969, 2040 bp, 3858 bp, 5114 bp, 7219 bp, and 8007 bp; and 1 kb DNA plus ladder (Invitrogen, U.S.A.)). One of the four clones was picked and the sequence of that clone was confirmed. The resulting four-gene contig assembly is shown in FIG. 64.

### Example 26

### Scenedesmus Chloroplast Transformation

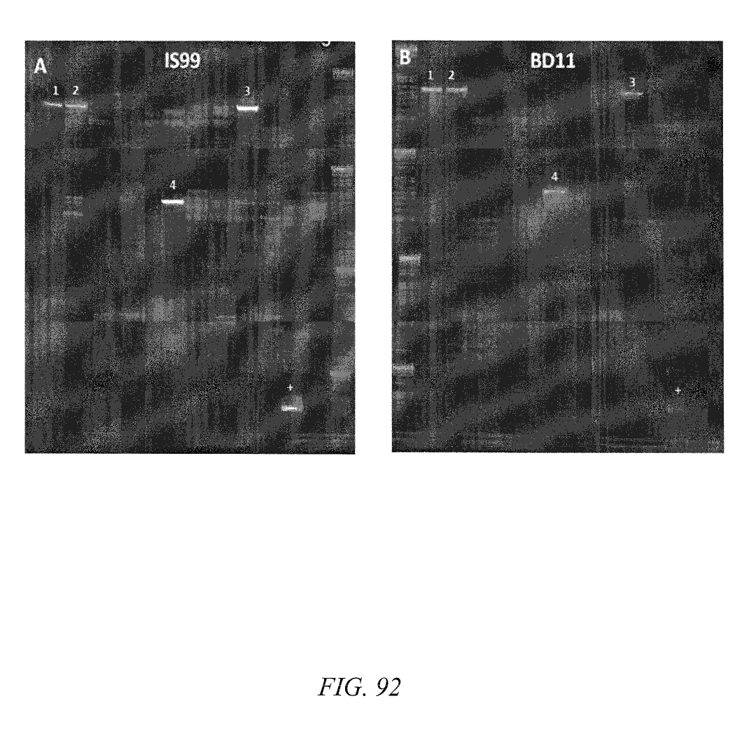

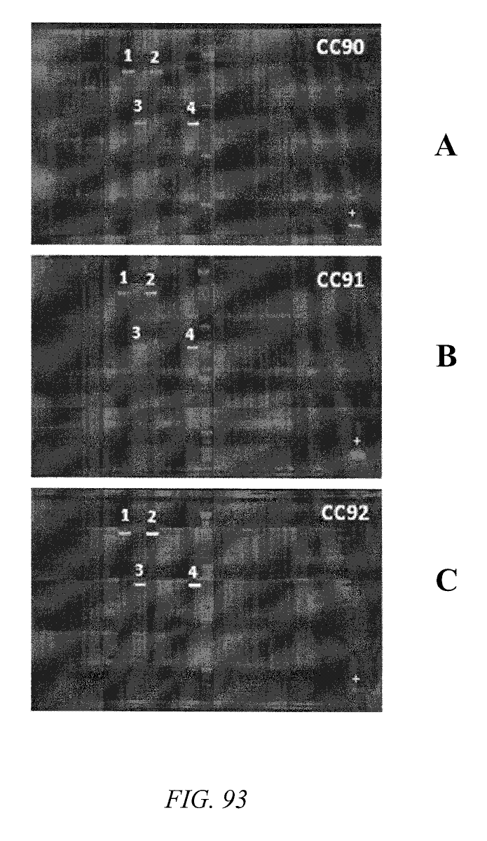

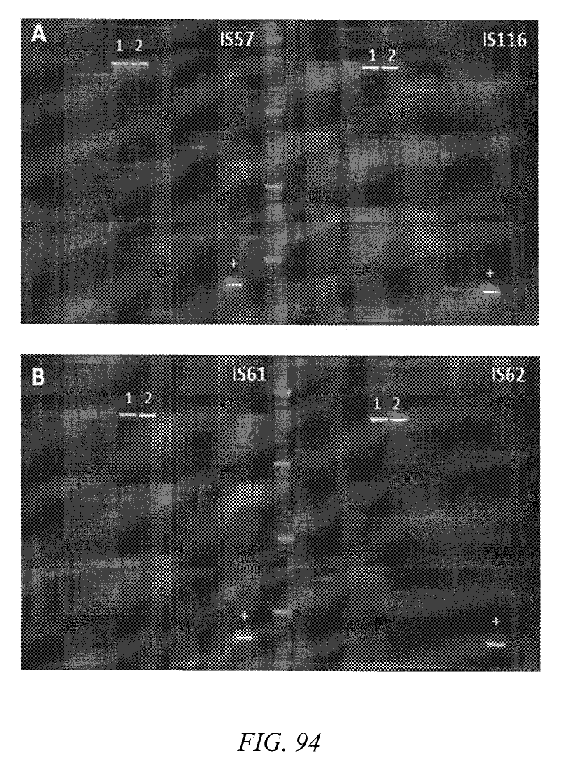

Once construct integrity was confirmed for each of the gene assemblies (2-, 3-, and 4-gene contigs), each of the gene assemblies were individually transformed into Scenedesmus obliquus. Briefly, cells were grown to mid-log phase and harvested. Approximately 5×10⁷ cells were spread onto TAP plates containing 25 μg/ml chloramphenicol and allowed to dry in a sterile culture hood. While plates were drying, 10 μg of plasmid DNA (from each of the contig assemblies) was bound to gold beads and transformation was conducted using a biolistic gene gun (Bio-rad) at 500 psi. 2 μg of DNA was loaded into each, shot and each plate was shot five times. Plates were placed under constant light for about 10 days. After which, chloramphenicol resistant colonies were picked and patched to a TAP plate containing 25 μg/ml chloramphenicol. Three to four days later, algae patches were picked into 10 mM EDTA, boiled for 10 minutes and then used in a standard PCR reaction to screen for the introduction of the genes into the chloroplast. Chloramphenicol resistant transformants potentially containing the 2-gene contig, targeting the psbB-rbcL locus, were screened for the presence of BD11 and IS99. Primers 1004 (SEQ ID NO: 353) and 1005 (SEQ ID NO: 154) screen for the presence of BD31, while primers 1006 (SEQ ID NO: 155) and 1007 (SEQ ID NO: 156) screen for the presence of IS99. FIGS. 92A and 92B depict 4 clones that screen PCR positive for both IS99 and BD31, respectively. Chloramphenicol resistant transformants potentially containing the 3-gene contig, targeting the psbB-rbcL locus, were screened for the presence of CC90, CC93, and CC92. Primers 1008 (SEQ ID NO: 157) and 3009 (SEQ ID NO: 158) screen for the presence of CC90, primers 1010 (SEQ ID NO: 159) and 1011 (SEQ ID NO: 160) screen for the presence of CC91, and primers 1012 (SEQ ID NO: 361) and 1013 (SEQ ID NO: 162) screen for the presence of CC92. FIGS. 93A-C depict 4 clones that screen PCR positive for CC90, CC91, and CC92. Chloramphenicol resistant transformants potentially containing the 4-gene contig, targeting the psbB-rbcL locus, were screened for the presence of IS61, IS62, IS57, and IS116. Primers 1014 (SEQ ID NO: 163) and 1015 (SEQ ID NO: 164) screen for the presence of IS61, primers 1016 (SEQ ID NO: 165) and 1017 (SEQ ID NO: 166) screen for the presence of IS62, primers 1018 (SEQ ID NO: 167) and 1019 (SEQ ID NO: 168) screen for the presence of IS57, and primers 1020 (SEQ ID NO: 169) and 1021 (SEQ ID NO: 170) screen for the presence of IS116. FIGS. 94A and 94B depict 2 clones that screen PCR positive for IS57, IS116 (A), and IS61, IS62 (B). Taken together these data demonstrate that one skilled in the art can integrate multiple gene contigs of varying sizes (2 gene: 8.1 kb, 3 gene: 31.2 kb, and 4 gene: 19.4 kb) into the chloroplast genome of Scenedesmus in a single step.

One of skill in the art will appreciate that many other methods known in the art may be substituted in lieu of the ones specifically described or referenced.

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1-80. (canceled)

81. An isolated Scenedesmus sp. or Dunaliella sp. comprising a chloroplast genome that has been transformed with an exogenous polynucleotide sequence, wherein the exogenous polynucleotide sequence comprises a nucleic acid sequence encoding a selection marker protein that is a chloramphenicol acetyltransferase (CAT), an erythromycin esterase (EreB), a cytosine deaminase (codA), a 3-(3,4-Dichlorophenyl)-1,1-dimethylurea (DCMU) resistant protein, or a betaine aldehyde dehydrogenase (BAD).

82. The isolated Scenedesmus sp. or Dunaliella sp. of claim 81, wherein the nucleic acid sequence encoding the selection marker protein comprises at least one mutation or modification to create a mutated nucleic acid sequence encoding a mutated selection marker protein with a change in at least one amino acid, wherein the selection marker protein and the mutated selection marker protein have amino acid sequences with at least 95% sequence identity to one another and the selection marker protein and mutated selection marker protein can be used in the same manner.

83. The isolated Scenedesmus sp. or Dunaliella sp. of claim 81, wherein the nucleic acid sequence encoding the selection marker protein is codon optimized for the chloroplast of Chlamydomonas reinhardtii.

84. The isolated Scenedesmus sp. or Dunaliella sp. of claim 81, wherein the nucleic acid sequence is a nucleotide sequence of SEQ ID NO: 28, SEQ ID NO: 29, SEQ ID NO: 30, SEQ ID NO: 31, SEQ ID NO: 32, SEQ ID NO: 34, or SEQ ID NO: 148.

85. The isolated Scenedesmus sp. or Dunaliella sp. of claim 81, wherein the Scenedesmus sp. is S. dimorphus or S. obliquus.

86. The isolated Scenedesmus sp. or Dunaliella sp. of claim 81, wherein the Dunaliella sp. is D. tertiolecta.

87. The isolated Scenedesmus sp. or Dunaliella sp. of claim 81, wherein the selection marker protein is expressed in the Scenedesmus sp. or Dunaliella sp.

88. A method of selecting for the expression of a selection marker protein in an isolated Scenedesmus sp. or Dunaliella sp. comprising, (a) obtaining the isolated Scenedesmus sp. or Dunaliella sp. of claim 87, and (b) determining if expression of the selection marker protein results in either a positive or negative selection of the transformed Scenedesmus sp. or Dunaliella sp.

89. The method of claim 88, wherein expression of the selection marker protein results in positive selection of the transformed Scenedesmus sp. or Dunaliella sp., and positive selection is determined if:
(a) the transformed Scenedesmus sp. or Dunaliella sp. grows in the presence of chloramphenicol when the expressed protein is CAT;
(b) the transformed Scenedesmus sp. or Dunaliella sp. grows in the presence of erythromycin when the expressed protein is EreB; or
(c) the transformed Scenedesmus sp. or Dunaliella sp. grows in the presence of DCMU or Atrazine when the expressed protein is DCMU resistant.

90. The method of claim 88, wherein expression of the selection marker protein results in negative selection of the transformed Scenedesmus sp. or Dunaliella sp., and negative selection is determined if:
(a) the transformed Scenedesmus sp. or Dunaliella sp. does not grow as well as a wild-type Scenedesmus sp. or Dunaliella sp. in the presence of 5-fluorocytosine (5FC) when the expressed protein is codA; or
(b) the transformed Scenedesmus sp. or Dunaliella sp. does not grow as well as a wild-type Scenedesmus sp. or Dunaliella sp. in the presence of betaine aldehyde when the expressed protein is BAD.

91. A method of transforming a chloroplast genome of a Scenedesmus sp. or a Dunaliella sp. with at least one exogenous nucleotide sequence, comprising:
i) obtaining the exogenous nucleotide sequence, wherein the exogenous nucleotide sequence comprises a nucleic acid sequence encoding a protein;
ii) binding the exogenous nucleotide sequence onto a particle; and
iii) shooting the exogenous nucleotide sequence into the Scenedesmus sp. or Dunaliella sp. by particle bombardment, wherein the chloroplast genome is transformed with the exogenous nucleotide sequence.

92. The method of claim 91, wherein the exogenous nucleotide sequence is at least 0.5 kb, at least 1.0 kb, at least 2 kb, at least 3 kb, at least 5 kb, at least 8 kb, at least 11 kb, or at least 19 kb in size.

93. The method of claim 91, wherein the particle is a gold particle or a tungsten particle.

94. The method of claim 93, wherein the gold particle is about 550 nm to about 1000 nm in diameter.

95. The method of claim 91, wherein the particle bombardment is carried out by a biolistic device.

96. The method of claim 95, wherein the biolistic device has a helium pressure of about 300 psi to about 500 psi.

97. The method of claim 95, wherein the biolistic device has a helium pressure of at least 300 psi, at least 350 psi, at least 400 psi, at least 425 psi, at least 450 psi, or at least 500 psi.

98. The method of claim 91, wherein the exogenous nucleotide sequence bound to the particle is shot at a distance of about 2 to about 4 cm from the Scenedesmus sp. or Dunaliella sp.

99. The method of claim 91, wherein the Scenedesmus sp. is S. dimorphus or S. obliquus, or the Dunaliella sp. is D. tertiolecta.

100. The method of claim 91, wherein the protein is a chloramphenicol acetyltransferase (CAT), an erythromycin esterase (EreB), a cytosine deaminase (codA), a 3-(3,4-Dichlorophenyl)-1,1-dimethylurea (DCMU) resistant protein, or a betaine aldehyde dehydrogenase (BAD).

101. A transformed chloroplast genome of a Scenedesmus sp. or Dunaliella sp. transformed by the method of claim 91.


SEQUENCE LISTING (170 sequences)

SEQ ID NO: 1 - DNA, 21 bp, Artificial Sequence
  PCR primer
agaaggagcttctacagatgc

SEQ ID NO: 2 - DNA, 26 bp, Artificial Sequence
  PCR primer
tcattagttacttcatctttaatccg

SEQ ID NO: 3 - DNA, 21 bp, Artificial Sequence
  PCR primer
gaactacaactaattattttc

SEQ ID NO: 4 - DNA, 24 bp, Artificial Sequence
  PCr primer
tgaaaccagtctttgtaaagctca

SEQ ID NO: 5 - DNA, 22 bp, Artificial Sequence
  PCR primer
ctaaattccagcaaccagcatt

SEQ ID NO: 6 - DNA, 22 bp, Artificial Sequence
  PCR primer
cgttcttctgagaaatggctta

SEQ ID NO: 7 - DNA, 27 bp, Artificial Sequence
  PCR primer
tgtaaatttaaggctgcctgtgatgtg

SEQ ID NO: 8 - DNA, 28 bp, Artificial Sequence
  PCR primer
gaggttcgaagaatgggtcaaagataag

SEQ ID NO: 9 - DNA, 24 bp, Artificial Sequence
  PCR primer
caactaccactggagataaatttc

SEQ ID NO: 10 - DNA, 25 bp, Artificial Sequence
  PCR primer
aatcactctaccaactgagttatgg

SEQ ID NO: 11 - DNA, 23 bp, Artificial Sequence
  PCR primer
gcactacctgatgaaaaataacc

SEQ ID NO: 12 - DNA, 33 bp, Artificial Sequence
  PCR primer
ttggtttctagattacgccccgccctgccactc

SEQ ID NO: 13 - DNA, 20 bp, Artificial Sequence
  PCR primer
gtgaatcaacaactgattgg

SEQ ID NO: 14 - DNA, 24 bp, Artificial Sequence
  PCR primer
tgaattgcataaatttacacatac

SEQ ID NO: 15 - DNA, 24 bp, Artificial Sequence
  PCR primer
ccacctcgtatggtaaaataattg

SEQ ID NO: 16 - DNA, 24 bp, Artificial Sequence
  PCR primer
gaaagaattatggacagtcctgct

SEQ ID NO: 17 - DNA, 42 bp, Artificial Sequence
  PCR primer
ttgttgcggccgcttttgaagccgaaatactttatttttatg

SEQ ID NO: 18 - DNA, 35 bp, Artificial Sequence
  PCR primer
catcattaatggagaaaaaaatcactggatatacc

SEQ ID NO: 19 - DNA, 35 bp, Artificial Sequence
  PCR primer
aattcctaggttacgccccgccctgccactcatcg

SEQ ID NO: 20 - DNA, 87 bp, Artificial Sequence
  FLAG epitope tag linked to a MAT epitope tag by a TEV protease site
ggtaccggtgattacaaagatgatgacgataaaagtggtgaaaacctttattttcaaggccataatcaccgtcacaaacacaccggt

SEQ ID NO: 21 - DNA, 762 bp, Artificial Sequence
  codon optimized endoxylanase from T. reesei
atggtaccagtatctttcacaagtcttttagcagcatctccaccttcacgtgcaagttgccgtccagctgctgaagtggaatcagttgcagtagaaaaacgtcaaacaattcaaccaggtacaggttacaataacggttacttttattcttactggaatgatggacacggtggtgttacatatactaatggacctggtggtcaatttagtgtaaattggagtaactcaggcaattttgttggaggaaaaggttggcaacctggtacaaagaataaggtaatcaatttctctggtagttacaaccctaatggtaattcttatttaagtgtatacggttggagccgtaacccattaattgaatattatattgtagagaactttggtacatacaacccttcaacaggtgctactaaattaggtgaagttacttcagatggatcagtttatgatatttatcgtactcaacgcgtaaatcaaccatctataattggaactgccactttctaccaatactggagtgtaagacgtaatcatcgttcaagtggtagtgttaatacagcaaaccactttaatgcatgggctcaacaaggtttaacattaggtacaatggactatcaaattgtagctgttgaaggttatttttcatcaggtagtgcttctatcactgttagcggtaccggtgattacaaagatgatgacgataaaagtggtgaaaacctttattttcaaggccataatcaccgtcacaaacacaccggttaa

SEQ ID NO: 22 - DNA, 81 bp, Artificial Sequence
  TEV protease site linked to a FLAG epitope tag
ggtaccggtgaaaacttatactttcaaggctcaggtggcggtggaagtgattacaaagatgatgatgataaaggaaccggt

SEQ ID NO: 23 - DNA, 1191 bp, Artificial Sequence
  codon optimized FPP synthase from G. gallus
atggtaccacacaagttcacaggtgttaacgctaaattccagcaaccagcattaagaaatttatctccagtggtagttgagcgcgaacgtgaggaatttgtaggattctttccacaaattgttcgtgacttaactgaagatggtattggtcatccagaagtaggtgacgctgtagctcgtcttaaagaagtattacaatacaacgcacctggtggtaaatgcaatagaggtttaacagttgttgcagcttaccgtgaactttctggaccaggtcaaaaagacgctgaaagtcttcgttgtgctttagcagtaggatggtgtattgaattattccaagcctttttcttagttgctgacgatataatggaccagtcattaactagacgtggtcaattatgttggtacaagaaagaaggtgttggtttagatgcaataaatgattcttttcttttagaaagctctgtgtatcgcgttcttaaaaagtattgccgtcaacgtccatattatgtacatttattagagctttttcttcaaacagcttaccaaacagaattaggacaaatgttagatttaatcactgctcctgtatctaaggtagatttaagccatttctcagaagaacgttacaaagctattgttaagtataaaactgctttctattcattctatttaccagttgcagcagctatgtatatggttggtatagattctaaagaagaacatgaaaacgcaaaagctattttacttgagatgggtgaatacttccaaattcaagatgattatttagattgttttggcgatcctgctttaacaggtaaagtaggtactgatattcaagataacaaatgttcatggttagttgtgcaatgcttacaaagagtaacaccagaacaacgtcaacttttagaagataattacggtcgtaaagaaccagaaaaagttgctaaagttaaagaattatatgaggctgtaggtatgagagccgcctttcaacaatacgaagaaagtagttaccgtcgtcttcaagagttaattgagaaacattctaatcgtttaccaaaagaaattttcttaggtttagctcagaaaatatacaaacgtcaaaaaggtaccggtgaaaacttatactttcaaggctcaggtggcggtggaagtgattacaaagatgatgatgataaaggaaccggttaa

SEQ ID NO: 24 - DNA, 36 bp, Artificial Sequence
  streptavidin epitope tag
accggtagtgcttggtcacaccctcaatttgagaaa

SEQ ID NO: 25 - DNA, 2196 bp, Artificial Sequence
  codon optimized fusicoccadiene synthase from P. amygdali
atggaatttaaatattcagaagttgttgaaccatcaacatattatacagaaggtttatgtgaaggtattgatgttcgtaaatcaaaatttacaacattagaagatcgtggtgctattcgtgctcatgaagattggaataaacatattggtccatgtggtgaatatcgtggtacattaggtccacgtttttcatttatttcagttgctgttccagaatgtattccagaacgtttagaagttatttcatacgctaatgaatttgcttttttacatgatgatgttacagatcatgttggtcatgatacaggtgaagttgaaaatgatgaaatgatgacagtttttttagaagctgctcatacaggtgctattgatacatcaaataaagttgatattcgtcgtgctggtaaaaaacgtattcaatcacaattatttttagaaatgttagctattgatccagaatgtgctaaaacaacaatgaaatcatgggctcgttttgttgaagttggttcatcacgtcaacatgaaacacgttttgttgaattagctaaatatattccatatcgtattatggatgttggtgaaatgttttggtttggtttagttacatttggtttaggtttacatattccagatcatgaattagaattatgtcgtgaacttatggctaatgcttggattgctgttggtttacaaaatgatatttggtcatggccaaaagaacgtgatgctgctacattacatggtaaagatcatgttgttaatgctatttgggttttaatgcaagaacatcaaacagatgttgatggtgctatgcaaatttgtcgtaaacttattgttgaatatgttgctaaatatttagaagttattgaagctacaaaaaatgatgaatcaatttcattagatttacgtaaatatttagatgctatgttatattcaatttcaggtaatgttgtttggtcattagaatgtccacgttataatccagatgtttcatttaataaaacacaattagaatggatgcgtcaaggtttaccatcattagaatcatgtccagttttagctcgttcaccagaaattgattcagatgaatcagcagtttcaccaactgctgatgaatcagattcaacagaagattcattaggttcaggttcacgtcaagattcatcattatcaacaggtttatcattatcaccagttcattcaaatgaaggtaaagatttacaacgtgttgatacagatcatattttttttgaaaaagctgttttagaagctccatacgattatattgcttcaatgccatcaaaaggtgttcgtgaccaatttattgatgctttaaatgattggttacgtgttccagatgttaaagttggtaaaattaaagatgctgttcgtgttttacataattcatcattattattagatgattttcaagataattcaccattacgtcgtggtaaaccatcaacacataatatttttggttcagctcaaacagttaatacagctacatattcaattattaaagctattggtcaaattatggaattttctgctggtgagtcagttcaagaagttatgaactcaattatgattttatttcaaggtcaagctatggatttattttggacatataatggtcatgttccatcagaagaagaatattatcgtatgattgaccaaaaaacaggtcaattattttcaattgctacatcattattattaaatgctgctgataatgaaattccacgtacaaaaattcaatcatgtttacatcgtttaacacgtttattaggtcgttgttttcaaattcgtgatgattatcaaaatttagtttctgctgattacactaaacaaaaaggattctgtgaagatttagatgaaggtaaatggtcattagctttaattcacatgattcataaacaacgttcacacatggctttattaaatgttttatcaacaggtcgtaaacatggtggtatgacattagaacaaaaacaatttgttttagatattattgaagaagaaaaatcattagattatacacgttcagttatgatggatcttcatgttcaattacgtgctgaaattggtcgtattgaaattttattagattcaccaaatccagctatgcgtttattattagaattattacgtgttaccggtagtgcttggtcacaccctcaatttgagaaataa

SEQ ID NO: 26 - DNA, 1704 bp, Artificial Sequence
  codon optimized phytase from E. coli
atggtaccaatgcgtatctctcttaaaaaatcaggaatgttaaaacttggtttatctcttgtagctatgacagtagctgcttctgtacaagctaaaacattagtatattgttcagaaggctctccagaaggttttaatcctcaactttttacttcaggtacaacttatgacgcttcttcagtacctttatacaaccgtttagtagagttcaaaatcggtacaacagaagttattccaggtttagctgaaaaatgggaagtatcagaagacggtaaaacttacacattccatttaagaaaaggtgttaaatggcacgataataaagagtttaaaccaacaagagaattaaatgctgatgatgtagttttctcatttgaccgtcagaaaaatgctcaaaatccatatcataaagtttcaggaggatcttacgaatatttcgaaggcatgggattaccagaacttatttctgaagttaaaaaagttgatgataacacagttcaatttgttttaactagaccagaagctccatttttagctgatttagctatggatttcgctagtattttatcaaaagaatatgcagatgctatgatgaaagctggtacacctgaaaaacttgatcttaatccaattggtactggtccattccaattacaacaatatcagaaagattcacgtattcgttacaaagcatttgacggctattggggtacaaaacctcaaattgatactttagtattttcaattacaccagacgcatcagttcgttacgcaaaattacagaaaaatgaatgccaagtaatgccatatccaaatccagctgatattgcacgtatgaaacaagataaatctatcaatttaatggaaatgcctggtttaaatgttggttatttatcatataacgttcaaaaaaaaccattagatgatgtaaaagttcgtcaagcattaacttatgcagttaataaagacgcaatcattaaagcagtatatcaaggtgctggagttagtgctaaaaatcttattccaccaacaatgtggggttacaacgatgacgttcaagattatacttacgaccctgagaaagctaaagcattacttaaagaagcaggtttagaaaaaggtttctcaattgatttatgggcaatgccagtacaacgtccttacaatccaaatgctagacgtatggcagaaatgattcaagcagactgggctaaagtaggtgttcaagcaaaaattgttacatacgaatggggtgaatacttaaaacgtgctaaagatggtgaacaccaaactgttatgatgggttggacaggtgataatggtgatcctgacaatttctttgcaacattattttcatgtgctgcttcagaacaaggttcaaattattcaaaatggtgttataaaccttttgaagacttaattcaacctgctcgtgctacagacgatcacaataaacgtgttgaattatacaaacaagcacaagttgtaatgcacgaccaagctccagctcttattattgctcattcaacagtattcgaaccagttagaaaagaagttaaaggttatgtagtagatccattaggtaaacatcactttgaaaacgtatcaattgaaggtaccggtgactataaagatgatgatgacaaaagtggagagaacttatactttcaaggtcataatcaccgtcacaaacacaccggttaa

SEQ ID NO: 27 - DNA, 87 bp, Artificial Sequence
  FLAG epitope tag linked to a MAT epitope tag by a TEV protease site
ggtaccggtgactataaagatgatgatgacaaaagtggagagaacttatactttcaaggtcataatcaccgtcacaaacacaccggt

SEQ ID NO: 28 - DNA, 660 bp, Artificial Sequence
  modified chloramphenicol acetyl transferase gene from E. coli
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtcagcgccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 29 - DNA, 1260 bp, Artificial Sequence
  modified erythromycin esterase gene from E. coli
atgaggttcgaagaatgggtcaaagataagcatattcctttcaaactgaatcaccctgatgataattacgatgattttaagccattaagaaaaataattggagatacccgagttgtagcattaggtgaaaattctcatttcataaaagaattttttttgttacgacatacgcttttgcgtttttttatcgaagacctcggttttactacgtttgcttttgaatttggttttgctgagggtcaaatcatcaataactggatacatggacaaggaactgacgatgaaataggcagattcttaaaacacttctattatccagaagagctcaaaaccacatttctatggctaagggagtacaataaagcagcaaaagaaaaaatcacatttcttggcattgatatacccagaaatggaggttcatacttaccaaatatggagatagtgcatgacttttttagaacagcggataaagaagcactacacattatcgatgatgcatttaatattgcaaaaaagattgattacttctccacatcacaggcagccttaaatttacatgagctaacagattctgagaaatgccgtttaactagccaattagcacgagtaaaagttcgccttgaagctatggctccaattcacattgaaaaatatgggattgataaatatgagacaattctgcattatgccaacggtatgatatacttggactataacattcaagctatgtcgggctttatttcaggaggcggaatgcagggcgatatgggtgcaaaagacaaatacatggcagattctgtgctgtggcatttaaaaaacccacaaagtgagcagaaagtgatagtagtagcacataatgcacatattcaaaaaacacccattctgtatgatggatttctaagttgcctaccaatgggccaaagacttaaaaatgccattggtgatgattatatgtctttaggtattacttcttatagtgggcatactgcagccctctatccggaagttgatacaaaatatggttttcgagttgataacttccaactgcaggaaccaaatgaaggttctgtcgagaaagctatttctggttgtggagttactaattcttttgtcttttttagaaatattcctgaagatttacaatccatcccgaacatgattcgatttgattctatttacatgaaagcagaacttgagaaagcattcgatggaatatttcaaattgaaaagtcatctgtatctgaggtcgtttatgaataa

SEQ ID NO: 30 - DNA, 2844 bp, Artificial Sequence
  modified genomic DNA from S. dimorphus
gcctttaggtatttcaggaactttcaacttcatgaaaataaacgtgacaaataaaaaccttttttatttgaaaattggggaaattgatgaaaaattgaaaaaaataaataaaatataaaatactttatgacaaaaaaatgaacaaaatttgagatagaacaaattgcaattctttttttcaaaaattgttttttcaataatcatcaaccgagcttatttttgtaagcaggttcagagactaaaaaaatagagaaattttttttgttcccaaaaacagaaaaagaaacaaaagatttcttcacctgtttgtgagatagtccgatcttttttgaaaaaaaaagagaaacttaagctttcataacaaaatggatcgttttccaagctgaacacaacattttaatgcaccctttccacatgttaggtgtagctggtgtatttggtggttcattattctctgcaatgcataggtactgagaaactgtgccctttgttttttttgtgttttttctcttctttttttctttgtgcctgctctctttttttctacaaacaagtaaaaaaaaagcaaaaacgcataaaaatgcacgcaaaaacgtatcgttgtgcatctaaaaagtataaaaaagaaacaaaaacgctttgcttcgctactttttttcttcttcattttttgcaaagcaaaaaatgataaaaagtagcgaagcgtagcaaaaaaagcttctaaaaacaattgaaaatcgagtgaattgcataaatttacacatacaaaaaaataaaaaacaagaataaaaaatatgaaaacaacaaaagcacttgacgattttaacaaaaacgcgtcaaacaagtatttagattttattgctctctgcaaacaaaaagcagaagagtattgaccagaacaacgcggaacctgtacgtatcaccatattgtgcctcgccatcactacaaggcaaatgagctctcttgggaaaactttgagagtccagaaaatttagttttggtgacctttgaagaccacattaaagcacacgaacttcgttttgaagtttatggtgaaaacggggacaatattgctgctcttcgaatggctggtcacaaggaagagagcatgcgagctatgcaacaagcaggtggtcaagcagtaaacaaaattttcaaagaaaaaaaacaactaatgcataatcaagaatttcaaaaagaaatggctcgccgctcaatggcccgtccagatgctcgtcaaattcgcagcgaaggaggaaagaagggtgcgcatgttcgtcaccaaaaccgaactgttcgagcaacagatcgatttctttggcgttttaaaagagaagactttttatgtacctttggttttgacaatgctggtgatctttgttgagaattgaatttggcaaagccagaaatttttgttggtcgtttgagcggttttcttactggaaaacgaaagacgaacaggggttggtcgtgtgaaaaaattgaagaatcttccgaaaacgaagaacaaacatgacccaatccacactaaatttttacaaaaaagtgtacgctctttcctttttgtttttgtgcaaagttctttgtttttatttttttttgtgaaaaatatgcaacgaattccagttgtaaaagactggactcgttcagagactagttttatttttatgaaacaaagtactcgagagaaaaaataagctttattttttcttatgatatagtcctcaagaaacttttttcttttaaaaaaaaagaaagttcttgaagggttcattagttacttcatctttaatccgtgaaacaactgaaaacgaatctgctaacgaaggatacaaattcggtcaagaagaagaaacttacaacatcgtagctgcacacggttactttggtcgtttaatcttccaatacgctgctttcaacaactctagatcattacacttcttcttagctgcttggccagttgtaggtatttggttcactgctttaggtatttcaactatggctttcaaccttaacggtttcaacttcaaccaatcagttgttgattcacaaggtcgtgttttaaacacatgggctgacatcatcaaccgtgctaacttaggtatggaagttatgcacgaacgtaacgctcacaacttcccattagacttagcatctgtagaagctccttctgtaaacgcttagttttattttttatgaaaaactcaggcttaatttaggcttgagtttttcattctttttgaagctctgaaattttaaaatttctagtcttctttaatgtttttaaattttaaaaaataaatttcttctctgctgtgtttttctttttttttgaaaaaacaaagaaaaaaaatttttttgttttcttctttgtttttttatttctttttgttttgtttattttttagtttcagaatctttgattcaaaaaaaaatttagtccgattactccataggagcaagcagtaaaaaataaaaactgtaataaaaaataaaacaaaaattttatttctttttgttttgcttgaacttttcaaaaaaaaattgaaaaattcaagcaaaacaaaaagaaacaaataaaaaatttatgaattttctactttttcaggagttgaaatttctcctttacttaaaacatattttgctaaaaaaagcgcttgtgttgctttttttgctactttttgtttccaagcattttttcgaatatttttttttgattttgatgtgcgtttttgttaacctaaaatcttgaaaagatttactcttttcaaatttttatgtttttattttttttattcataaaaaaaaacaatacataaaaataaagtatttcggcttcaaaa

SEQ ID NO: 31 - DNA, 1284 bp, Artificial Sequence
  codon optimized cytosine deaminase from E. coli
atgtctaacaacgctttacaaactattatcaacgcaagattaccaggtaaagaaggtttatggcaaatccaccttcaagatggtaaaatttctgctattgacgcacaatcaggtgtaatgccaataacagaaaattcattagatgctgaacaaggcttagttattcctcctttcgttgagccacatatacaccttgacacaactcaaacagctggtcaaccaaattggaatcaatcaggcacactttttgaaggtattgaacgttgggctgagcgtaaagcattattaacacatgacgatgttaaacaacgtgcttggcaaacattaaaatggcaaattgcaaatggtatacaacatgttcgtactcatgttgacgtttcagacgcaactttaacagcattaaaagctatgttagaagttaaacaagaagtagctccatggatagacttacaaattgtagcttttccacaagagggtattttatcatatccaaatggcgaagcattattagaagaggctttacgtcttggtgctgacgtagtaggtgctattcctcacttcgaatttactcgtgaatatggtgtagaaagtttacataaaactttcgcacttgctcaaaaatatgatcgtcttattgatgttcactgtgatgaaattgatgacgaacaatcacgtttcgttgaaacagttgctgcattagctcaccgtgaaggtatgggtgctcgtgttacagctagtcatactacagctatgcattcttataatggagcatacacttctcgtctttttagattacttaaaatgtcaggtattaacttcgttgcaaatcctttagtaaacattcacttacaaggtagatttgatacatatcctaaacgtagaggtattacacgtgttaaagaaatgttagaatctggtatcaatgtatgttttggtcacgatgatgtattcgatccatggtatccattaggcacagctaatatgttacaagttttacacatgggtttacatgtttgtcaacttatgggttacggtcaaatcaacgatggtcttaacttaattacacatcattctgcacgtactttaaatcttcaagattatggtattgcagctggtaattcagctaaccttattattcttccagcagaaaatggttttgatgctcttcgtcgtcaagtacctgttcgttattctgttcgtggcggcaaagttattgctagtacacaacctgcacaaacaactgtatatttagaacagccagaagctattgattacaaacgttaa

SEQ ID NO: 32 - DNA, 1653 bp, Artificial Sequence
  codon optimized betaine aldehyde dehydrogenase from S. oleracea
atgatggctttcccaattccagctcgtcaattattcattgacggtgaatggcgtgaaccattattaaaaaaccgtattccaattattaacccatcaacagaagaaattattggtgacattccagctgctacagctgaagacgtagaagtagctgtagtagctgctcgtaaagctttcaaacgtaacaaaggtcgtgactgggctgctttatggtcacaccgtgctaaatacttacgtgctattgctgctaaaattacagaaaaaaaagaccacttcgtaaaattagaaacattagactcaggtaaaccacgtgacgaagctgtattagacattgacgacgtagctacatgtttcgaatacttcgaatacttcgctggtcaagctgaagctttagacgctaaacaaaaagctccagtaacattaccaatggaacgtttcaaatcacacgtattacgtcaaccaattggtgtagtaggtttaatttcaccatggaactacccattattaatggacacatggaaaattgctccagctttagctgctggttgtacaacagtattaaaaccatcagaattagcttcagtaacatgtttagaattcggtgaagtatgtaacgaagtaggtttaccaccaggtgtattaaacattttaacaggtttaggtccagacgctggtgctccaattgtatcacacccagacattgacaaagtagctttcacaggttcatcagctacaggttcaaaaattatggcttcagctgctcaattagtaaaaccagtaacattagaattaggtggtaaatcaccagtaattatgttcgaagacattgacattgaaacagctgtagaatggacattattcggtgtattctggacaaacggtcaaatttgttcagctacatcacgtttattagtacacgaatcaattgctgctgaattcgtagaccgtatggtaaaatggacaaaaaacattaaaatttcagacccattcgaagaaggttgtcgtttaggtccagtaatttcaaaaggtcaatacgacaaaattatgaaattcatttcaacagctaaatcagaaggtgctacaattttatgtggtggttcacgtccagaacacttaaaaaaaggttactacattgaaccaacaattattacagacattacaacatcaatgcaaatttggaaagaagaagtattcggtccagtaatttgtgtaaaaacattcaaaacagaagacgaagctattgaattagctaacgacacagaatacggtttagctggtgctgtattctcaaaagacttagaacgttgtgaacgtgtaacaaaagctttagaagtaggtgctgtatgggtaaactgttcacaaccatgtttcgtacacgctccatggggtggtgtaaaacgttcaggtttcggtcgtgaattaggtgaatggggtattgaaaactacttaaacattaaacaagtaacatcagacatttcagacgaaccatggggttggtacaaatcaccaaccggttacccatacgacgtacctgactatgcttacccttacgacgtaccagactatgcttatccatacgacgtaccagactacgctgaaaacttatacttccaaggtcaccaccaccaccaccatcaccacccaccaggttaa

SEQ ID NO: 33 - DNA, 141 bp, Artificial Sequence
  3xHA tag linked to a 6xHIS tag by a TEV protease site
accggttacccatacgacgtacctgactatgcttacccttacgacgtaccagactatgcttatccatacgacgtaccagactacgctgaaaacttatacttccaaggtcaccaccaccaccaccatcaccacccaccaggt

SEQ ID NO: 34 - DNA, 1647 bp, Artificial Sequence
  codon optimized betaine aldehyde dehydrogenase from B. vulgaris
atgatgtcaatgccaattccatcacgtcaattattcattgacggtgaatggcgtgaaccaattaaaaaaaaccgtattccaattattaacccatcaaacgaagaaattattggtgacattccagctggttcatcagaagacattgaagtagctgtagctgctgctcgtcgtgctttaaaacgtaacaaaggtcgtgaatgggctgctacatcaggtgctcaccgtgctcgttacttacgtgctattgctgctaaagtaacagaacgtaaagaccacttcgtaaaattagaaacaattgactcaggtaaaccattcgacgaagctgtattagacattgacgacgtagctacatgtttcgaatacttcgctggtcaagctgaagctatggacgctaaacaaaaagctccagtaacattaccaatggaacgtttcaaatcacacgtattacgtcaaccaattggtgtagtaggtttaattacaccatggaactacccattattaatggctacatggaaaattgctccagctttagctgctggttgtacagctgtattaaaaccatcagaattagcttcaattacatgtttagaattcggtgaagtatgtaacgaagtaggtttaccaccaggtgtattaaacattgtaacaggtttaggtccagacgctggtgctccattagctgctcacccagacgtagacaaagtagctttcacaggttcatcagctacaggttcaaaagtaatggcttcagctgctcaattagtaaaaccagtaacattagaattaggtggtaaatcaccaattattgtattcgaagacgtagacattgaccaagtagtagaatggacaatgttcggttgtttctggacaaacggtcaaatttgttcagctacatcacgtttattagtacacgaatcaattgctgctgaattcattgaccgtttagtaaaatggacaaaaaacattaaaatttcagacccattcgaagaaggttgtcgtttaggtccagtaatttcaaaaggtcaatacgacaaaattatgaaattcatttcaacagctaaatcagaaggtgctacaattttatgtggtggttcacgtccagaacacttaaaaaaaggttacttcattgaaccaacaattatttcagacatttcaacatcaatgcaaatttggcgtgaagaagtattcggtccagtattatgtgtaaaaacattctcatcagaagacgaagctttagacttagctaacgacacagaatacggtttagcttcagctgtattctcaaaagacttagaacgttgtgaacgtgtatcaaaattattagaatcaggtgctgtatgggtaaactgttcacaaccatgtttcgtacacgctccatggggtggtattaaacgttcaggtttcggtcgtgaattaggtgaatggggtattgaaaactacttaaacattaaacaagtaacatcagacatttcaaacgaaccatggggttggtacaaatcaccaaccggttacccatacgacgtacctgactatgcttacccttacgacgtaccagactatgcttatccatacgacgtaccagactacgctgaaaacttatacttccaaggtcaccaccaccaccaccatcaccacccaccaggttaa

SEQ ID NO: 35 - DNA, 2541 bp, Artificial Sequence
  codon optimized E-alpha-biabolene synthase from A. grandis
atggtaccagcaggtgtatcagctgtgtcaaaagtttcttcattagtatgtgacttaagtagtactagcggcttaattcgtagaactgcaaatcctcaccctaatgtatggggttatgacttagttcattctttaaaatctccatatattgatagtagctatcgtgaacgtgctgaagtgcttgtaagtgaaataaaagctatgttaaatccagcaattactggagatggtgaatcaatgattacaccttcagcttatgacactgcttgggttgcacgtgtaccagcaattgatggtagcgcacgtccacaatttccacaaacagtagattggattttaaagaatcaattaaaagatggttcttggggtattcaatcacactttttactttcagaccgtttattagctactcttagctgtgttttagttttacttaaatggaatgttggtgatttacaggttgagcaaggtattgagtttattaagtcaaaccttgaattagtaaaagatgaaactgatcaagattctttagtgactgattttgagattattttccctagcttacttcgtgaggcccaaagtttacgtttaggtcttccatacgatttaccttacatccacttattacaaacaaaacgtcaggaacgtttagcaaaattaagccgtgaagaaatatatgcagttccaagtccacttttatattctttagagggtattcaagatattgttgagtgggaacgtattatggaagtacaatctcaggatggatcatttttaagttctccagcatcaaccgcatgtgtttttatgcatacaggtgacgctaagtgtttagaatttcttaacagtgtaatgattaagtttggtaattttgtaccatgcctttatcctgtagatttattagaacgtttacttatagtagataatatagttcgtcttggtatttaccgtcacttcgaaaaagaaattaaagaagcattagattatgtatatcgccattggaatgaacgtggtattggttggggtcgtttaaatccaattgctgacttagaaacaactgctttaggttttcgtttattacgtttacaccgttataatgtatctccagcaatctttgataatttcaaagatgccaatggcaaattcatttgtagcactggtcagtttaataaggatgtggcttcaatgttaaacttataccgtgcatcacaattagcattcccaggcgaaaacattttagatgaagctaaatcttttgccaccaaatacttacgtgaagcccttgaaaaatctgaaacttcatcagcttggaacaataaacagaatttaagtcaagaaatcaagtatgcattaaaaacttcatggcacgcttctgtaccacgtgttgaagcaaaacgttattgtcaagtttatcgtcctgattacgctcgtattgctaagtgtgtatacaaattaccatacgttaacaacgaaaaattcttagaattaggtaaattagattttaacatcattcaatcaattcatcaagaagaaatgaaaaatgtgacaagttggtttcgtgattctggcttaccattatttactttcgctcgcgaacgtcctttagaattttacttcttagttgctgctggtacttatgaacctcaatatgctaaatgtcgtttcttattcacaaaagtagcttgtcttcaaacagtattagacgatatgtacgatacttacggtactttagacgaattaaaactttttaccgaggctgtgcgtcgttgggatttatcttttacagaaaatttacctgactatatgaaattatgttatcaaatctattatgacatcgttcatgaagtggcttgggaagctgaaaaagaacaaggtagagaattagtgtcattcttccgtaaaggctgggaagactacttattaggttactatgaagaagcagaatggttagcagcagaatacgttccaacattagatgaatacattaaaaacggtattacatcaatcggccaacgtatcttattactttcaggtgtgttaattatggatggccaacttttatcacaagaagcattagaaaaagttgattaccctggtcgtcgtgttttaactgagttaaactcacttattagccgtttagctgacgacactaaaacttataaagcagaaaaagctcgtggagaattagcctcatcaattgaatgctacatgaaagatcatcctgaatgtacagaagaagaagccttagaccacatttattctattcttgaaccagccgtaaaagaattaactcgtgaatttcttaaaccagacgacgttccatttgcttgtaaaaagatgttattcgaagaaactcgtgttacaatggtgatctttaaagatggtgatggttttggtgtatctaagttagaagttaaagatcacatcaaagaatgcttaattgaaccattaccattaggtaccggtgaaaacttatactttcaaggctcaggtggcggtggaagtgattacaaagatgatgatgataaaggaaccggttaa

SEQ ID NO: 36 - DNA, 542 bp, Artificial Sequence
  modified endogenous promoter from the psbA gene of S. dimorphus
tcgttgagtagtttttcagattaattgctatgcaacccatgtgaattaaaaatataacataaatttcaaaaatgtcaatttttagtctaaaaaatattattcgatgtttttttatgacaattttttttaaatttttcaataaaaacaaaaatatattattaaagaatacataaaaaaatcaaaaattcataaataaatcaccaaaaaatttattttttaatatttgatttcaatatttttatttgaattaaaaatttaattatttaaaatttttatttatatatttgaatttatacttcagtttttattaaacttaagttttcaaatcataaatttaatagttaatatttttttaaactctaaattattaatctttaaaatttcaaatctttaacacttgaaattataaacttcattgtttttgtttgaatttttttttaagtttgaaaactttataaaattaaataaactaaatagaattttgaattgtataaaaattaaaatgaaaagtttgtgtttttcagattaaatgtagcaaccaa

SEQ ID NO: 37 - DNA, 559 bp, Scenedesmus dimorphus
atttaatctgaaaaacacaaacttttcattttaatttttatacaattcaaaattctatttagtttatttaattttataaagttttcaaacttaaaaaaaaattcaaacaaaaacaatgaagtttataatttcaagtgttaaagatttgaaattttaaagattaataatttagagtttaaaaaaatattaactattaaatttatgatttgaaaacttaagtttaataaaaactgaagtataaattcaaatatataaataaaaattttaaataattaaatttttaattcaaataaaaatattgaaatcaaatattaaaaaataaattttttggtgatttatttatgaatttttgatttttttatgtattctttaataatatatttttgtttttattgaaaaatttaaaaaaaattgtcataaaaaaacatcgaataatattttttagactaaaaattgacatttttgaaatttatgttatatttttaattaacaagagttcaattgaattcatgaaaaatcttttaaagattcggagaattttaaatttttatttttttat

SEQ ID NO: 38 - DNA, 716 bp, Artificial Sequence
  modified endogenous promoter for the psbB gene of S. dimorphus
ttaaattactttatttatggaaaaatgtattttttcgatttctagagtttttttacactttttgaattgtgtgtttttttcttttctaaaagaaaaaagacaattgaaagtcttgtttcttcatttttattattttatcataaatctagaatttttaaaatattttttattttttaccgcggagcggactaaattttttttaaacaatattttttaattgaaaacattttttttcttcaaaatataataataaaatttgaaaaaaaagaaaaacaaaaaataaaagctttcctctgcttaagttgtatttttttatgtttttattttttttgaaagttttgaaaaaagtacaaaaaagtttaaattttttttcatttttttcacattttctttatcatattatcgtcttaagtttttttcttttttttcagttttttttaagaaactgaaaaaaaagagaaaaacttaagacaataaaaaagatttttttaacagaaaaaagtttcttttttaaataaaatgaaaacaaaagttgtaggcaaccaaacatttatttaattcataaagagagtacaacttaagcagaggaaagcttttattttttgtttttcttttttttcaatttttattaatatattttgaagaaaaaaaatgtttttcaattaaaaaatattgtttaaaaaaaaatttaatcccgctccgcggtaaacagtaaa

SEQ ID NO: 39 - DNA, 550 bp, Scenedesmus dimorphus
aacttttgttttcattttatttaaaaaagaaacttttttctgttaaaaaaatcttttttattgtcttaagtttttctcttttttttcagtttcttaaaaaaaactgaaaaaaaagaaaaaaacttaagacgataatatgataaagaaaatgtgaaaaaaatgaaaaaaaatttaaacttttttgtacttttttcaaaactttcaaaaaaaataaaaacataaaaaaatacaacttaagcagaggaaagcttttattttttgtttttcttttttttcaaattttattattatattttgaagaaaaaaaatgttttcaattaaaaaatattgtttaaaaaaaatttagaaaaaaataaaaaatattttaaaaattctagatttatgataaaataataaaaatgaagaaacaagactttcaattgtcttttttcttttagaaaagaaaaaaacacacaattcaaaaagtgtaaaaaaactctagaaatcgaaaaaatacatttttccataaataaagtaattaagtaaacaaaaattcttttttcattaattt

SEQ ID NO: 40 - DNA, 597 bp, Scenedesmus dimorphus
tttttgaattagataaatgagtgttctcaatttttttttctttgcattttttgtttgtgttgatttacaaaaacaatagaaaaaagaaaacaatattttctttctaaaaaaaaacaaaattgatgaaaaatagacatgaacaaaaaattttgaaagttgacttttttaaaaaatttttggtataatacaaaaaaagaatttttggaaaggtggcagagtggttgaatgctctggttttgaaaaccagcgtggctttacggtcaccgggggttcgaatccctccctttccgataatatatacaaaaatttttaaagttttttgtttattttgtatagataaaaaatctgcaataaaaatttcgttttttatttattcaaaaattctgtttttttgaaaagaaaataaaaaaaatgccaaaagtgagttttttattcaaatattagaaaaagtttttgaaaaatttaaaaaaatagaaaaaatttttttatttttttcataatttaaaaaattatgttataatttaaattacaaataggttttattaaaaaatttttacgtacagatgaattctataaaattattttggagatcaccat

SEQ ID NO: 41 - DNA, 537 bp, Artificial Sequence
  modified endogenous promoter for the tufA gene of S. dimorphus
ttaaaccaattttccaagtaactttactttatcaaaaattaaaaaattaaaaaacttttattgaacttaaaataaaatttttaacaaaatttattttaaaaaaaagaaaaaatttttttattttggttttatttatttctttttttttacaaacaaaaatttttttaaacagaataataaaaaaaattttatttaaagaatggttttttaatattttgctcatgacaaatgattttttactacttttatgctttttttcaaaaaaagcagcaaagcaaaaaagttataaaaagtgtatggagcaaacagttaaattgacactttttaaaagtatttataggcccaaccggacttgaaccgatgacctattgcttgtaaggcaatcactctaccaactgagttatgggcctaaaaaatattatttatattttataatagaatataaaatctaacaacttctttatcctcctcttttctcacctatcttttttggttgggggtaagtgaaaaaggaaatggttgctcgcaacgaaaccc

SEQ ID NO: 42 - DNA, 492 bp, Scenedesmus dimorphus
taaagaagttgttagattttatattctattataaaatataaataatattttttaggcccataactcagttggtagagtgattgccttacaagcaataggtcatcggttcaagtccggttgggcctataaatacttttaaaaagtgtcaatttaaccgcttgctccatacactttttataacttttttgctttgctgctttttttaaaaaaaagcataaaagtagtaaaaaatcatttgtcatgagcaaaatattaaaaaaccattctttaaataaaattttttttattattctgtttaaaaaaatttttgtttgtaaaaaaaaagaaataaataaaaccaaaataaaaaaattttttcttttttttaaaataaattttgttaaaaattttattttaagttcaataaaagttttttaattttttaatttttgataaagtaaagttacttggaaaattggtttatacagaaaaaattataaattatttattcat

SEQ ID NO: 43 - DNA, 557 bp, Artificial Sequence
  modified endogenous promoter of the rpoA of S. dimorphus
cttgtttttttttctaaatttacctttaacttgaaagatctaaaatgacaaaaattttttttagagagcaagttttacttttgttgtctaaaaaactcaaattacactttgtcttaagaacctttttttattaaaaaacaaaatttttttattttgtttctttttattcatttgaaaaaaaagttttctaaacttaactgataattcaagaaaaaaaatatgcctgctactagattcgaactagtgactttccgcgtatgaaacggatgctctggccaactgagctaagcaggcttaatatcatatattatacaaaatttttttcaacaaaaacaagtttttttttcgaattttagaattttttgattatttttgtttgtttgaatattccaaacaaaaataaagtttttctctttttttttacttttttcctgcttccaaatactaaaaaagtttaatagaaaaactctttttttgaaagttgtcaaatttttcctttcaaaaaattatttttttgttgggctatttttgaaaataaatgtgagctcggaaaaaaa

SEQ ID NO: 44 - DNA, 532 bp, Scenedesmus dimorphus
cgagctcacatttattttcaaaaatagcccaacaaaaaataattttttgaaaggaaaaatttgacaactttcaaaaaaagagtttttctattaaacttttttagtatttggaagcaggaaaaaagtaaaaaaaaagagaaaaactttatttttgtttggaatattcaaacaaacaaaaataatcaaaaaattctaaaattcgaaaaaaaaacttgtttttgttgaaaaaaattttgtataatatatgatattaagcctgcttagctcagttggccagagcatccgtttcatacgcggaaagtcactagttcgaatctagtagcaggcatatttttttcttgaattatcagttaagtttagaacaaatgaataaaaagaaacaaaataaaaaaattttgttttttaataaaaaaaggttcttaagacaaagtgtaatttgagttttttagacaacaaaagtaaaacttgttctctaaaaaaaatttttgtcattttagatctttcaagttaaaggtaaatttagaaaaaaaaa

SEQ ID NO: 45 - DNA, 483 bp, Scenedesmus dimorphus
aaaagaaatttttttttattcctttatttttttgtattccaaaaaatattttgtttttcttcaatacagtttttttgctattgctttttctcacttttttgctttgctgcttttttttttaagcgtaaaagtgaaaaaaaaagtttgtaaaacaaaaaaattgaaaaaaaaagattgattttttatacagtaaaaattttttgttttttaaatatttatttaaattcaactcaatttcaaaaaaaaatgtctttgttgaatgcaaatttttttgaacaaacaacaattcaaaataaaacaatttttggtttaaattcaaaatttttaaattcatctacagaaaattcttccatgagtatttttaaaactttaaagggagtttgttctggaaaaagaaatatactttgcaaaagtacacctcgtcctatgaattctgattttttaaaaaataatagttctattccttctcaaaaacaaaaagga

SEQ ID NO: 46 - DNA, 669 bp, Artificial Sequence
  modified endogenous promoter of the ftsH gene in S. dimorphus
agtagaaaaaatgatgtacttgtataaataaaaaataattatttatacaaaaaatttttttttattttgtttttactttttttcatttttttgctttgctgttttttatgaattttttactttactgctttttttcttctttacttttacgctttttatcacttttttgatttgctgctttttttcttcttcattttttgctttgctgctttttttcttcttcattttttgctttgcaaaaaatgataaaaagcgtaaaatgataaaaagcgtaaaatgataaaaagcgtaaaatgataaaaagcgtaaaagtaaaaaagaaaaaaagcagcaaagtaaaaaagtgataaaaagtttaaaagtgctaaaaagcgtaagtaaacaaaaaaagcaaaagacaaaaaaaagttttcaaaaaaaaatttttatttttgtttttttataaaaattttaacaagttttattttctaaataaaacaaaaaattgtgagttttaaaaaaacgaaaaagttcaaaaatttttttttgatggacaaaaagaaagaaattttaaatttattaatttttcaaataactaataaattttgttcaacaggagagaactattgaaaaaaagcctactatcggagttgaaccgataaccttcgatttagctagcaaccaacaaac

SEQ ID NO: 47 - DNA, 557 bp, Scenedesmus dimorphus
ctgtttcacaacgtttgcatccaacgcaatcttcagtacgtggtgctgaagccatttgatttgctttacaaccattccatggaaccatttctaaaacatctaatggacaagcacgtacacattgtgtacaaccaatacaagtatcataaatttttacaatatgtgacattttttatttttaatagaactattcaaaagaagattgagaatttcaaaaaagaatttttttacatgaaaaacgattttttagaaattttactataaaaaagtaaaaaaaaatattaaattttgtagttttttgcaggagcaggatttgaacctgcgacattgggattatgagccccacgagctaccagactgctctatcctgcgttttttgttttaaaaagttttattcgtaaaaaaaatttcttttatatatattactttattgatttttatttgtcaaatttttttttgtttgaatttttatttcaaattttttaaatatagaaaaaatttttttttatgataatataaaactacagtttgaattataaataaaaaaaaaaaatgaa

SEQ ID NO: 48 - DNA, 580 bp, Artificial Sequence
  modified endogenous promoter of the rbcL gene in S. dimorphus
tttttttggggttggttacggttttttttacttttgctttttttgcttttgttcaaagaaaaaaaaatacaaaataaaaaaaactaaaatgaaaaaacaaagaattctaaaattcataaaaaaaattaaaacccaattttttttttggaaacttttccaaataataaaaaaatcaaaaaaaaatttttctagtatttttttcatattttgaaactttttttgagtttataaaaaaatagaaaaaacaaatagatgaaaatttagaaaaattataaaccaataaaaatgaagttttgcgtagaaaaaaaatttagtttacttgttccccaagagcaagtggtaactttgaaaaaaatatttaaacttaaaaatttgctaaagttttgaatttatgttaaaattttaaaaaaataaaaatttttaaactatttttttatgttaaaaaaatagtttttattattttctataatatagtttagttttttatttttttcaatttcttttttttttttcaaagaaaaaagttttccacggatagatttttataggatcgacaaaatgttctatgaactttaaaaaa

SEQ ID NO: 49 - DNA, 555 bp, Scenedesmus dimorphus
ggttttttttacttttgctttttttgcttttgttcaaagaaaaaaaaatacaaaataaaaaaaactaaaatgaaaaaacaaagaattctaaaattcataaaaaaaattaaaacccaattttttttttggaaacttttccaaataataaaaaaatcaaaaaaaaatttttctagtatttttttcatattttgaaactttttttgagtttataaaaaaatagaaaaaacaaatagatgaaaatttagaaaaattataaaccaataaaaatgaagttttgcgtagaaaaaaaatttagtttacttgttccccaagagcaagtggtaactttgaaaaaaatatttaaacttaaaaatttgctaaagttttgaatttatgttaaaatttaaaaaaaataaaaatttttaaactatttttttatgttaaaaaaatagtttttattattttctataatatagtttagttttttatttttttcaatttctttttttttttcaaagaaaaaagttttccacggatagatttttataggatcgacaaaatgttctatgaactttt

SEQ ID NO: 50 - DNA, 532 bp, Artificial Sequence
  modified endogenous promoter for the chlB gene from S. dimorphus
cccagtcttcagtacgtggtgctgaagccatttgatttgctttacaaccattccatggaaccatttctaaaacatctaatggacaagcacgtacacattgtgtacaaccaatacaagtatcataaatttttacaatatgtgacattttttatttttaatagaactattcaaaagaagattgagaatttcaaaaaagaatttttttttacatgaaaaacgattttttagaaattttactataaaaaagtaaaaaaaaatattaaattttgtagttttttgcaggagcaggatttgaacctgcgacattgggattatgagccccacgagctaccagactgctctatcctgcgttttttgttttaaaaagttttattcgtaaaaaaaatttcttttatatatattactttattgatttttatttgtcaaatttttttttgtttgaatttttatttcaaattttttaaatatagagaaaatttttttttatgataatataaaactacagtttgaattataaataaaaaaaaaaaat

SEQ ID NO: 51 - DNA, 615 bp, Scenedesmus dimorphus
taaaccgaaggttatcgggtcaactccgatagtaggctttttttcaatagttctctcctgtgaacaaaatttattagttatttgacaaaattaataaatttaaaattctttccttttgtccatcaaaaaaaaaatttttgaactttttcgtttttttaaaactcacaattttttgttttattagaaaataaaacttgttaaaatttttataaaaaaacaaaaataaaaatttttttttgaaaacttttttttgtcttttgctttttttgtttacttacgctttttagcacttttaaattttttatcacttttttactttgctgctttttttcttctttacttttacgctttttatcattttacgctttttatcattttacgctttgcaaaaaatgaagaagaaaaaaagcagcaaagcaaaaaatgaagaagaaaaaaagcagcaaatcaaaaaagtgataaaaagcgtaaaagtaaagaagaaaaaaagcagtaaagtaaaaaattcataaaaaacagcaaagcaaaaaaatgaaaaaaagtaaaaacaaaataaaaaaaaattttttgtataaataattattttttatttatacaagtacatcattttttcta

SEQ ID NO: 52 - DNA, 533 bp, Artificial Sequence
  modified endogenous promoter of the petA gene in S. dimorphus
tgcacaaaagaaatcaaatgttttcaaatcgtgcaaacatcaaattgcacaaaataatttttaaaattcattttatgaaagttcgttcttcagttaaaaaaatttgtactaaatgtcgtttaattcgtcgaaaaggtacagtaatggttatttgtacaaatcctaaacataaacaacgtcaaggataattttttttaatggaagaaatgttttttacttcttccattaaaaaagaaaaaagaaaagtgcagaactttttttttgatcaaaaaaaagatacaaaaaatttttttgttttaattttatgatataattctatttcagagaagaaaaaaaaatttaaacaaaacaaaaaaatacagagaagtttaaatttagacctaaaggtagatttccataaattcatttttctcatttgtttttttcttttttttgcattgcgaaaaaaaaaagaaaaaagcaaaaagtcattttaaagagaraaatggagaaagataaaagtttttaactttttttgaacaattccatggggg

SEQ ID NO: 53 - DNA, 521 bp, Scenedesmus dimorphus
acaaaagaaatcaaatgttttcaaatcgtgcaaacatcaaattgcacaaaataatttttaaaattcattttatgaaagttcgttcttcagttaaaaaaatttgtactaaatgtcgtttaattcgtcgaaaaggtacagtaatggttatttgtacaaatcctaaacataaacaacgtcaaggataattttttttaatggaagaaatgttttttacttcttccattaaaaaagaaaaaagaaaagtgcagaactttttttttgatcaaaaaaaagatacaaaaaatttttttgttttaattttatgatagaattctatttcagagaagaaaaaaaaatttaaacaaaacaaaaaaatacagagaagtttaaatttagacctaaaggtagatttccataaattcatttttctcatttgtttttttcttttttttgcattgcaaaaaaaaaagaaaaaagcaaaaagtcattttaaagagaaaaatggagaaagataaaagtttttaactttttttgaacaattc

SEQ ID NO: 54 - DNA, 503 bp, Artificial Sequence
  modified endogenous promoter for the petB gene from S. dimorphus
aagaaagtttaaaaaaatttacataagaagaagatacaaaaacaaattattttcaatttttgttttgtaaaaaaaaaatttcgttttttagaatttctatgtttttttttattttttgttttctgaaaaaaaaagttttttcagaaaacaagaagaaaattaaaggtctacaaaataaaaattattttgttacaaacgaccaatgtttattattttttgtttttttcattttctatttctcacttatctttttaaagtcaaaagaaaatttagaatgaaagtaaaaaaaagaaaaaacaataaaaaacaaaaaagcaaaaaaaattctttgcaatttttgtttaaaaatttttggtttcaattttaaaatttatgaaaaatgtacaccataagtctaaaatttacaaaaattatctaaaaaaacttctttcttttgtaacaaaaggcaataagcccataattcgagcacttttaattgtttttgtaaacaaaccaaaaaaaaa

SEQ ID NO: 55 - DNA, 513 bp, Scenedesmus dimorphus
aaaaacaattaaaagtgctcgaattatgggcttattgccttttgttacaaaagaaagaagtttttttagataatttttgtaaattttagacttatggtgtacatttttcataaattttaaaattgaaaccaaaaatttttaaacaaaaattgcaaagaattttttttgcttttttgtttttttattgttttttcttttttttactttcattctaaattttcttttgactttaaaaagataagtgagaaatagaaaatgaaaaaaacaaaaaataataaacattggtcgtttgtaacaaaataatttttattttgtagacctttaattttcttcttgttttctgaaaaaactttttttttcagaaaacaaaaaataaaaaaaaacatagaaattctaaaaaacaaattttttttttacaaaacaaaattgaaaaaatttgtttttgaatcttattcctatgaatttttttaactttttcgtttaatggtattataaaaaattttctttttctat

SEQ ID NO: 56 - DNA, 537 bp, Artificial Sequence
  modified endogenous terminator region for the rbcL gene from S. dimorphus
tttttaaaatacttcctctttaaaggggaagtatttttttcttgattttagaactctaaaaacacaaattgtttatttttgtttttcattttcatttatttattgataaaaacaaaagaagcagcaaagcaaaaaaacaaaaaaaactaaaaaaatttgttttgttcatttcaatagaataaaaaaaacaaaatttttcaaaaaaatttataaaattattgtaagttttcaatacaaaaaattttttaaaaatattttttaatatgttttttttttaaattgaaaaaaaaaattcaaaacaaagaattttaaaaaaatagaagagtttttaattaaaaaaaattgaaatttcaaaaattttttgttaatataatctttagtataattcatgcaaaataaatgacaaatgagtcaaaaaaaagcatgggccgatgcccgagtggttaatgggggcggattgtaaatccgctggttacgcctacgttggttcgaatccgactcggcccaaaaatacaaaatacattgtttgaaagacta

SEQ ID NO: 57 - DNA, 538 bp, Scenedesmus dimorphus
tttttttttaaaatacttcctctttaaaggggaagtatttttttcttgattttagaactctaaaaacacaaattgtttatttttgtttttcattttcatttatttattgataaaaacaaaagaagcagcaaagcaaaaaaacaaaaaaaactaaaaaaatttgttttgttcatttcaatagaataaaaaaaacaaaatttttcaaaaaaatttataaaattattgtaagttttcaatacaaaaaattttttaaaaatattttttaatatgttttttttttaaattgaaaaaaaaaattcaaaacaaagaattttaaaaaaatagaagagtttttaattaaaaaaaattgaaatttcaaaaattttttgttaatataatctttagtataattcatgcaaaataaatgatcaaatgagtcaaaaaaaagcatgggccgatgcccgagtggttaatgggggcggattgtaaatccgctggttacgcctacgttggttcgaatccgactcggcccaaaaatacaaaatacattgtttgaaag

SEQ ID NO: 58 - DNA, 669 bp, Scenedesmus dimorphus
ctagattttattttttatgaaaaactcaggcttaatttaggcttgagtttttcattctttttgaagctctgaaattttaaaatttctagtcttctttaatgtttttaaattttaaaaaataaatttcttctctgctgtgtttttctttttttttgaaaaaacaaagaaaaaaaatttttttgttttcttctttgtttttttatttctttttgttttgtttattttttagtttcagaatctttgattcaaaaaaaaatttagtccgattactccataggagcaagcagtaaaaaataaaaactgtaataaaaaataaaacaaaaattttatttctttttgttttgcttgaacttttcaaaaaaaaattgaaaaattcaagcaaaacaaaaagaaacaaataaaaaatttatgaattttctactttttcaggagttgaaatttctcctttacttaaaacatattttgctaaaaaaagcgcttgtgttgctttttttgctactttttgtttccaagcattttttcgaatatttttttttgattttgatgtgcgtttttgttaacctaaaatcttgaaaagatttactcttttcaaatttttatgtttttattttttttattcataaaaaaaaacaatacataaaaataaagtatttcggcttcaaaaactag

SEQ ID NO: 59 - DNA, 597 bp, Scenedesmus dimorphus
aatttccatttttttcatttttttttagaaaagttgtatttttcttgatgaaatgaaaatttcaaaaaagaaaaatacaatttttctctacttttttttgattctttacttttttttgaattttttttgtgcttcgtttttgaaaaaaactttttatttgaaaaattttgttgaattaaaaaaaatcaactcaatacattttttttgaacttctttttacttttttgctttgttgcttttcttttcatttttatcattttttgcttcgctgctttttatcattttttgctttgcaaaaaatgaagaagaaaaaaagcgtaaaatgaaaaagaaaaaaagtttcaaagaaaaaaaagcgaaaaagcaagagaagataaaaaacaagaacaaaaaaattgttcaaaaacacaatagaaaattctttaaaaattttttgattttttatagaatttgtgagaaatagtaaaaaaagtcaaaaaatcacaacattataaatagaaaaaatcttgttttgtaaaaatttataattttttatattatttcaattttaaataagatcttttggagctgaaaaaatatgagaaatagtttggacaa

SEQ ID NO: 60 - DNA, 12 bp, Artificial Sequence
  nucleic acid linker
gaggcagatcaa

SEQ ID NO: 61 - DNA, 12 bp, Artificial Sequence
  nucleic acid linker
gaggtaatactt

SEQ ID NO: 62 - DNA, 514 bp, Dunaliella tertiolecta
gtatacgaaacctttaaggttaaagagatatatgttaaattaaacataaacgaaaagactttaaatttttcaaataaaaaaaaagatacagagggtactaatatttaatattatgaccttctgtatcctatacttaataagtataaattataatatagattaataaatctattcaagttaataaactgtgtttttattttatttaatgattttctctactaaatattaaatatgttattatttatacatagtgttttttcttttttttttttaagcctgtttaactcaatcggtagagtattggttttgtaaaccaaaggttgcgggttcgattcctgtagcaggctactaattttttaagatattttatattttaaaaatatctttttaaaataaaaaaaaaattttttaaatcgattttaaaaataaaaaaagctatacttataaatgcaataaaggttaaaaaaaaaattaaacgatatgatgaattataaaaattattatggagatgcac

SEQ ID NO: 63 - DNA, 491 bp, Dunaliella tertiolecta
ctctagctaggacatctccctttcacggaggaaacagggattcgaattcccttgggggtaaaaaaaaaatagtatatatataggtatttatacatttttcaataaatatttgattgagagttactaaataatctaatttgaattaaaataaattgaatgtataattctatgtttcgctctcccacaggtttataataactattattttatttgaattatttttttgaagtatatttccttatttaaagtgcttagaaataattaattattaataacatctgttttttttttacttatttttgaaagttcagtttcaattataaaaatattatttatataatattcagaataaaaattatgagtctaattatgaattaataaaaaaaaaaaaaataaattcatcaaaagtatattaaaaaataaaaaaggctaaaattaaatatactcgaaaaattggcttatatataaaaatatattaataatgataaaca

SEQ ID NO: 64 - DNA, 495 bp, Dunaliella tertiolecta
ttttttcttttaggcgggtccgaagtccttaggcttattcgaaggaaaaacgagaaaaatttacgtagtaaattttctttgctggccctgccaaaaacaacaccattaacctataagtagtaataattctttagtattacttttaggttatttataaatttgagaagtatagaagaatctatagattttgcttatgtgtttatctatagattcttctatacttctcatttttaacaaatttttattaagatttttttaaacaaaaaaaaagttttcaacttatataattaaacctaaacaacgttgtatattttttattttaagttttggtaaagtatgtataccagtaaacctttagtaaatttttttaccgcttaggctaggacctataaaatttagcgcggcgccctaagagctaggataaagatccgttgtctgtccgctgcgctaaattttctttagacgaagcgaagcgtcgagtctcacgcacccacc

SEQ ID NO: 65 - DNA, 311 bp, Dunaliella
ttctaatctcttaacgagattagattaatcccttaatgtctaagggattaatacttatcccatcctgttaaacccaggatgggataaaaaaatttacaagacaaggtattaaaaatgaataaaaatagattatatgtatttaaacgtatataataaaattatgagtaaaataacagagtaaaccaaaacaataagatttttttttttttttttcaattctaaatatcaatatagaaggttaggaaatttccccggaaaaaatcctttggattttttcctaagggcgaaaaaatattctctgaatatttttt

SEQ ID NO: 66 - DNA, 40 bp, Artificial Sequence
  PCR primer
ttggttgggcggccgccgtttaggtgtaacacaatcttgg

SEQ ID NO: 67 - DNA, 40 bp, Artificial Sequence
  PCR primer
ttggttgggcggccgctagccgtggtatttacttcactca

SEQ ID NO: 68 - DNA, 35 bp, Artificial Sequence
  PCR primer
ttggttggctcgaggccctttcgtcttcaagaaat

SEQ ID NO: 69 - DNA, 34 bp, Artificial Sequence
  PCR primer
ttggttggctcgagaagtcttgcgccttaaacca

SEQ ID NO: 70 - DNA, 77 bp, Artificial Sequence
  PCR primer
ttggttggctgcagttaattaaggatccactagtatttaaattcctgatgcggtattttctccttacgcatctgtgc

SEQ ID NO: 71 - DNA, 116 bp, Artificial Sequence
  PCR primer
ttggttgggcggccgctcacagatgagaagatatttgctcgataatcaatactctaggcatctaacttttcccattgtcttaaaccgacttaccttaggaatcatagtttcatgat

SEQ ID NO: 72 - DNA, 116 bp, Artificial Sequence
  PCR primer
ttggttgggcggccgctgctggttgtcattgcctctggataatttttctcgaactatgcctgcgcgttgataccaatccaatggatctacaggcagaacggcctctagcggttttt

SEQ ID NO: 73 - DNA, 62 bp, Artificial Sequence
  PCR primer
ttggttggatttaaatactagtggatccttaattaactgcagggccggcctctagttttatt

SEQ ID NO: 74 - DNA, 100 bp, Artificial Sequence
  PCR primer
ttctgcctgtagatccattggattggtatcaacgcgcaggcatagttcgagaaaaattatccagaggcaatgacaaccagcacgggctcgagactagtgg

SEQ ID NO: 75 - DNA, 100 bp, Artificial Sequence
  PCR primer
gaatttttcaaaaaattcatactttgtttttttattttttctgagtttttaatcaaaaaactttttgtataaaattcgtaagatctcctaggaaaatgaa

SEQ ID NO: 76 - DNA, 100 bp, Artificial Sequence
  PCR primer
tttttttgaaaatagtttttctcaattttttattttttttgttttttctctaaaaatcaaaaattcaattttgagaaaagggctcgagactagtttgtcc

SEQ ID NO: 77 - DNA, 100 bp, Artificial Sequence
  PCR primer
ggtaagtcggtttaagacaatgggaaaagttagatgcctagagtattgattatcgagcaaatatcttctcatctgtgactagtgctagctaaagaagttg

SEQ ID NO: 78 - DNA, 80 bp, Artificial Sequence
  PCR primer
gattggtatcaacgcgcaggcatagttcgagaaaaattatccagaggcaatgacaaccagcacgggctcgagactagtgg

SEQ ID NO: 79 - DNA, 83 bp, Artificial Sequence
  PCR primer
cattaggaattatctttttcgcaattttctttagagaaccacctcgtatggtaaaataacgtaagatctcctaggaaaatgaa

SEQ ID NO: 80 - DNA, 80 bp, Artificial Sequence
  PCR primer
aaaagttagatgcctagagtattgattatcgagcaaatatcttctcatctgtgatctcctagtgctagctaaagaagttg

SEQ ID NO: 81 - DNA, 80 bp, Artificial Sequence
  PCR primer
tcaacgcctatggcaactctgtagaatattcatcagcgtaacgccttagaatatcatacgggctcgagactagtttgtcc

SEQ ID NO: 82 - DNA, 80 bp, Artificial Sequence
  PCR primer
acgctgatgaatattctacagagttgccataggcgttgaacgctacacggacgatacgaatttttgaattagataaatga

SEQ ID NO: 83 - DNA, 80 bp, Artificial Sequence
  PCR primer
tcatactttgtttttttattttttctgagtttttaatcaaaaaactttttgtataaaattttttgaagccgaaatacttt

SEQ ID NO: 84 - DNA, 80 bp, Artificial Sequence
  PCR primer
cattaggaattatctttttcgcaattttctttagagaaccacctcgtatggtaaaataagggctcgagactagtttgtcc

SEQ ID NO: 85 - DNA, 80 bp, Artificial Sequence
  PCR primer
cattaccagagtgctccgcagacgagtatggcacatggctccgcgaggctattttctcctagtgctagctaaagaagttg

SEQ ID NO: 86 - DNA, 80 bp, Artificial Sequence
  PCR primer
actctggtaatgcatatggtccacaggacattcgtcgcttccgggtatgcgctctatgaattcccgttttaagagcttgg

SEQ ID NO: 87 - DNA, 80 bp, Artificial Sequence
  PCR primer
actgattgacacggtttagcagaacgtttgaggactaggtcaaattgagtggtagttcaagagaaaaaaaaagaaaaagc

SEQ ID NO: 88 - DNA, 98 bp, Artificial Sequence
  PCR primer
accactcaatttgacctagtcctcaaacgttctgctaaaccgtgtcaatcagtgtctgcttcctgagtgaaacccgtaagatctcctaggaaaatgaa

SEQ ID NO: 89 - DNA, 80 bp, Artificial Sequence
  PCR primer
acgctgatgaatattctacagagttgccataggcgttgaacgctacacggacgatacgaaagcagttgctttctcctatg

SEQ ID NO: 90 - DNA, 80 bp, Artificial Sequence
  PCR primer
tctttggccgttgtgccgggagcgctcatgtcaatacttttctcctctagaagttgaaagtacaataaaccaagatgaag

SEQ ID NO: 91 - DNA, 80 bp, Artificial Sequence
  PCR primer
aaagtattgacatgagcgctcccggcacaacggccaaagaagtctccaatttcttatttctttttgaattagataaatga

SEQ ID NO: 92 - DNA, 102 bp, Artificial Sequence
  PCR primer
ccatttttttgaaaatagtttttctcaattttttattttttttgttttttctctaaaaatcaaaaattcaattttgagaaaagggtaataactgatataatt

SEQ ID NO: 93 - DNA, 102 bp, Artificial Sequence
  PCR primer
aggtatgaatttttcaaaaaattcatactttgtttttttattttttctgagtttttaatcaaaaaactttttgtataaaattgatcctcgagagatcttatg

SEQ ID NO: 94 - DNA, 80 bp, Artificial Sequence; identical to SEQ ID NO: 82
  PCR primer

SEQ ID NO: 95 - DNA, 34 bp, Artificial Sequence
  PCR primer
ttggttggactagtgggtaataactgatataatt

SEQ ID NO: 96 - DNA, 34 bp, Artificial Sequence
  PCR primer
ttggttggactagtgatcctcgagagatcttatg

SEQ ID NO: 97 - DNA, 83 bp, Artificial Sequence
  PCR primer
ttgtatttttaaatttttagtttgaactacaactaattattttctacgtaagatctactagtgggctcgagactagtttgtcc

SEQ ID NO: 98 - DNA, 83 bp, Artificial Sequence; identical to SEQ ID NO: 97
  PCR primer

SEQ ID NO: 99 - DNA, 43 bp, Artificial Sequence
  PCR primer
ggttggttgcggccgccggtccggtagcagttaataatgtagg

SEQ ID NO: 100 - DNA, 59 bp, Artificial Sequence
  PCR primer
ccggcctctagaggccggcccctaggagatcttacgtagaaaataattagttgtagttc

SEQ ID NO: 101 - DNA, 57 bp, Artificial Sequence
  PCR primer
ccggcctctagaggccggccactagtctcgagcccgggttgaactatcaagtttagg

SEQ ID NO: 102 - DNA, 44 bp, Artificial Sequence
  PCR primer
ccaaccaagcggccgcggcgcgcctttattatgggcgaacgacg

SEQ ID NO: 103 - DNA, 1291 bp, Artificial Sequence
  codon optimized sequence comprising URA3
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccc

SEQ ID NO: 104 - DNA, 2309 bp, Artificial Sequence
  codon optimized sequence comprising ADE2
aagcttgcatgcctgcaggtcgatcgactctagaaatcgatagatctgaattaattcttgaataatacataacttttcttaaaagaatcaaagacagataaaatttaagagatattaaatattagtgagaagccgagaattttgtaacaccaacataacactgacatctttaacaacttttaattatgatacatttcttacgtcatgattgattattacagctatgctgacaaatgactcttgttgcatggctacgaaccgggtaatactaagtgattgactcttgctgaccttttattaagaactaaatggacaatattatggagcatttcatgtataaattggtgcgtaaaatcgttggatctctcttctaagtacatcctactataacaatcaagaaaaacaagaaaatcggacaaaacaatcaagtatggattctagaacagttggtatattaggagggggacaattgggacgtatgattgttgaggcagcaaacaggctcaacattaagacggtaatactagatgctgaaaattctcctgccaaacaaataagcaactccaatgaccacgttaatggctccttttccaatcctcttgatatcgaaaaactagctgaaaaatgtgatgtgctaacgattgagattgagcatgttgatgttcctacactaaagaatcttcaagtaaaacatcccaaattaaaaatttacccttctccagaaacaatcagattgatacaagacaaatatattcaaaaagagcatttaatcaaaaatggtatagcagttacccaaagtgttcctgtggaacaagccagtgagacgtccctattgaatgttggaagagatttgggttttccattcgtcttgaagtcgaggactttggcatacgatggaagaggtaacttcgttgtaaagaataaggaaatgattccggaagctttggaagtactgaaggatcgtcctttgtacgccgaaaaatgggcaccatttactaaagaattagcagtcatgattgtgaggtctgttaacggtttagtgttttcttacccaattgtagagactatccacaaggacaatatttgtgacttatgttatgcgcctgctagagttccggactccgttcaacttaaggcgaagttgttggcagaaaatgcaatcaaatcttttcccggttgtggtatatttggtgtggaaatgttctatttagaaacaggggaattgcttattaacgaaattgccccaaggcctcacaactctggacattataccattgatgcttgcgtcacttctcaatttgaagctcatttgagatcaatattggatttgccaatgccaaagaatttcacatctttctccaccattacaacgaacgccattatgctaaatgttcttggagacaaacatacaaaagataaagagctagaaacttgcgaaagagcattggcgactccaggttcctcagtgtacttatatggaaaagagtctagacctaacagaaaagtaggtcacataaatattattgcctccagtatggcggaatgtgaacaaaggctgaactacattacaggtagaactgatattccaatcaaaatctctgtcgctcaaaagttggacttggaagcaatggtcaaaccattggttggaatcatcatgggatcagactctgacttgccggtaatgtctgccgcatgtgcggttttaaaagattttggcgttccatttgaagtgacaatagtctctgctcatagaactccacataggatgtcagcatatgctatttccgcaagcaagcgtggaattaaaacaattatcgctggagctggtggggctgctcacttgccaggtatggtggctgcaatgacaccacttcctgtcatcggtgtgcccgtaaaaggttcttgtctagatggagtagattctttacattcaattgtgcaaatgcctagaggtgttccagtagctaccgtcgctattaataatagtacgaacgctgcgctgttggctgtcagactgcttggcgcttatgattcaagttatacaacgaaaatggaacagtttttattaaagcaagaagaagaagttcttgtcaaagcacaaaagttagaaactgtcggttacgaagcttatctagaaaacaagtaatatataagtttattgatatacttgtacagcaaataattataaaatgatatacctattttttaggctttgttatgattacatcaaatgtggacttcatacatagaaatcaacgcttacaggtgtccttttttaagaatttcatacataagatctctcgaggatc

SEQ ID NO: 105 - DNA, 3688 bp, Artificial Sequence
  codon optimized sequence comprising URA3-ADE2
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaaggtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgacgtaactataacggtcctaaggtagcgaaggccggccttaattaaatttaaatcgccgagttacgctagggataacagggtaatatagaagcttgcatgcctgcaggtcgatcgactctagaaatcgatagatctgaattaattcttgaataatacataacttttcttaaaagaatcaaagacagataaaatttaagagatattaaatattagtgagaagccgagaattttgtaacaccaacataacactgacatctttaacaacttttaattatgatacatttcttacgtcatgattgattattacagctatgctgacaaatgactcttgttgcatggctacgaaccgggtaatactaagtgattgactcttgctgaccttttattaagaactaaatggacaatattatggagcatttcatgtataaattggtgcgtaaaatcgttggatctctcttctaagtacatcctactataacaatcaagaaaaacaagaaaatcggacaaaacaatcaagtatggattctagaacagttggtatattaggagggggacaattgggacgtatgattgttgaggcagcaaacaggctcaacattaagacggtaatactagatgctgaaaattctcctgccaaacaaataagcaactccaatgaccacgttaatggctccttttccaatcctcttgatatcgaaaaactagctgaaaaatgtgatgtgctaacgattgagattgagcatgttgatgttcctacactaaagaatcttcaagtaaaacatcccaaattaaaaatttacccttctccagaaacaatcagattgatacaagacaaatatattcaaaaagagcatttaatcaaaaatggtatagcagttacccaaagtgttcctgtggaacaagccagtgagacgtccctattgaatgttggaagagatttgggttttccattcgtcttgaagtcgaggactttggcatacgatggaagaggtaacttcgttgtaaagaataaggaaatgattccggaagctttggaagtactgaaggatcgtcctttgtacgccgaaaaatgggcaccatttactaaagaattagcagtcatgattgtgaggtctgttaacggtttagtgttttcttacccaattgtagagactatccacaaggacaatatttgtgacttatgttatgcgcctgctagagttccggactccgttcaacttaaggcgaagttgttggcagaaaatgcaatcaaatcttttcccggttgtggtatatttggtgtggaaatgttctatttagaaacaggggaattgcttattaacgaaattgccccaaggcctcacaactctggacattataccattgatgcttgcgtcacttctcaatttgaagctcatttgagatcaatattggatttgccaatgccaaagaatttcacatctttctccaccattacaacgaacgccattatgctaaatgttcttggagacaaacatacaaaagataaagagctagaaacttgcgaaagagcattggcgactccaggttcctcagtgtacttatatggaaaagagtctagacctaacagaaaagtaggtcacataaatattattgcctccagtatggcggaatgtgaacaaaggctgaactacattacaggtagaactgatattccaatcaaaatctctgtcgctcaaaagttggacttggaagcaatggtcaaaccattggttggaatcatcatgggatcagactctgacttgccggtaatgtctgccgcatgtgcggttttaaaagattttggcgttccatttgaagtgacaatagtctctgctcatagaactccacataggatgtcagcatatgctatttccgcaagcaagcgtggaattaaaacaattatcgctggagctggtggggctgctcacttgccaggtatggtggctgcaatgacaccacttcctgtcatcggtgtgcccgtaaaaggttcttgtctagatggagtagattctttacattcaattgtgcaaatgcctagaggtgttccagtagctaccgtcgctattaataatagtacgaacgctgcgctgttggctgtcagactgcttggcgcttatgattcaagttatacaacgaaaatggaacagtttttattaaagcaagaagaagaagttcttgtcaaagcacaaaagttagaaactgtcggttacgaagcttatctagaaaacaagtaatatataagtttattgatatacttgtacagcaaataattataaaatgatatacctattttttaggctttgttatgattacatcaaatgtggacttcatacatagaaatcaacgcttacaggtgtccttttttaagaatttcatacataagatctctcgaggatc

SEQ ID NO: 106 - DNA, 88 bp, Artificial Sequence
  nucleic acid linker
cgtaactataacggtcctaaggtagcgaaggccggccttaattaaatttaaatcgccgagttacgctagggataacagggtaatatag

SEQ ID NO: 107 - DNA, 3011 bp, Artificial Sequence
  codon optimized comprising TRP1-ARS1-CEN4
gccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctaaatcctctttatatgatctgccgatagatagttctaagtcattgaggttcatcaacaattggattttctgtttactcgacttcaggtaatgaaatgagatgatacttgcttatctcatagttaactggcataaattttagtataggttaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgatttaggcaaaagttactatttctggctcgtgtaatatatgtatgctaatgtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatattaggacttccacaccaactagtaatatgacaataaattcaagatattcttcatgagaatggcccagctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcaattctttagtataaatttcactctgaaccatcttggaaggaccggataattatttgaaatctctttttcaattgtatatgtgttatgtagtatactctttcttcaacaattaaatactctcggtagccaagttggtttaaggcgcaagactt

SEQ ID NO: 108 - DNA, 2103 bp, Artificial Sequence
  codon optimized sequence comprising LEU2
cctgatgcggtattttctccttacgcatctgtgcggtatttcacaccgcatatcgaccggtcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcct

SEQ ID NO: 109 - DNA, 1833 bp, Artificial Sequence
  codon optimized sequence comprising CC-93
atggcaaaaatgcgtgctgttgatgcagcaatgtatgtattagagaaagaaggtattactacagctttcggtgtaccaggtgcagcaatcaatccattttattctgctatgcgtaaacatggtggcattcgtcacattttagctcgtcatgtagaaggtgctagtcacatggcagaaggttatacacgtgctacagcaggtaatattggtgtttgtttaggtacatctggcccagctggtacagatatgattactgcattatatagtgcttcagctgatagtattcctatcttatgtattactggtcaagcacctcgtgctcgtcttcataaagaagattttcaagcagttgatattgaagctattgctaaacctgtaagtaaaatggctgtaactgttcgtgaggctgctttagtaccacgtgtattacaacaggcttttcacttaatgcgttcaggtcgtcctggtcctgtattagttgatttacctttcgatgttcaagtagctgaaattgaattcgatccagacatgtatgaacctttacctgtatataaacctgctgcttctcgtatgcaaattgaaaaagcagtagaaatgttaattcaagctgaacgtcctgtaatcgttgctggtggtggtgttattaatgctgatgcagctgctcttttacagcaatttgcagaattaacttcagtacctgtaattcctactttaatgggctggggttgtattccagatgatcacgaattaatggcaggcatggtaggtttacaaacagctcaccgttacggtaatgctacattattagcatctgatatggtattcggtattggaaatcgtttcgctaatcgtcatactggttcagtagagaaatatactgaaggacgtaaaattgttcacattgacatcgaacctacacaaattggtcgtgtattatgccctgatttaggcattgtaagtgatgcaaaagctgctttaactttacttgttgaggttgctcaagaaatgcaaaaagcaggtcgtttaccttgtagaaaagaatgggtagctgactgccaacaacgtaaacgtactttattaagaaaaactcattttgataatgttcctgttaaacctcaacgtgtatacgaagaaatgaataaagcatttggtcgtgatgtttgctatgttactacaattggtttaagtcaaatagctgctgcacaaatgcttcatgtatttaaagaccgtcattggattaactgtggccaagctggccctttaggttggacaattcctgcagctttaggtgtatgtgcagctgatccaaaacgtaatgtagttgctatttcaggtgattttgactttcaatttcttattgaggaattagctgttggtgcacaatttaacattccatatatccatgttttagtaaataacgcttatttaggtttaattcgtcaatcacaacgtgcttttgatatggattactgtgttcaattagcatttgaaaatataaacagttcagaagttaacggttatggtgtagatcacgtaaaagttgctgaaggcttaggatgtaaagctattcgtgtttttaaaccagaagatatagctccagcttttgaacaggctaaagcattaatggctcaatatcgtgttcctgtagtagttgaagttattttagaaagagttacaaacatttcaatgggttcagaattagataatgttatggagtttgaagatattgcagataatgctgctgacgcacctactgaaacttgctttatgcattatgaagattataaagatgatgatgataaaggacacaaccaccgtcacaaacattaatct

SEQ ID NO: 110 - DNA, 934 bp, Artificial Sequence
  codon optimized sequence comprising CC-94
atgaaagtaggatttataggattaggtattatgggcaaaccaatgtctaaaaacttacttaaagctggttactcattagttgttgctgatcgtaacccagaagcaatcgctgacgtaattgcagctggagctgaaacagcttcaactgctaaagctattgcagaacaatgtgatgttattattactatgttacctaattcacctcacgtaaaagaagtagctttaggcgaaaatggaattattgaaggtgctaaaccaggcacagtattaatagatatgtcatcaattgctccacttgcatcacgtgaaatttctgaagcattaaaagcaaaaggtattgatatgttagatgctccagttagtggtggtgaaccaaaagctatcgatggtacacttagtgttatggtaggcggtgataaagcaatttttgacaaatactacgatttaatgaaagctatggctggttctgtagtacacactggtgaaatcggtgcaggtaacgtaactaaattagctaaccaggttattgttgcattaaatatagctgcaatgtcagaagctcttactttagctacaaaagcaggtgtaaatcctgatttagtatatcaggcaattcgtggcggtttagcaggcagtactgtattagacgcaaaagctccaatggttatggatcgtaatttcaaaccaggttttagaattgatttacatattaaagaccttgctaatgctttagatacatcacacggtgtaggagctcaattaccattaactgcagctgttatggaaatgatgcaagcattacgtgctgatggtttaggtacagcagatcactcagctttagcttgttattatgagaaattagcaaaagttgaagttacacgtgattataaagatgatgatgataaaggacacaaccaccgtcacaaacattaat

SEQ ID NO: 111 - DNA, 8765 bp, Artificial Sequence
  codon optimized sequence comprising CC93-CC94
tccaaactatttctcatattttttcagctccaaaagatcttatttaaaattgaaataatataaaaaattataaatttttacaaaacaagattttttctatttataatgttgtgattttttgactttttttactatttctcacaaattctataaaaaatcaaaaaatttttaaagaattttctattgtgtttttgaacaatttttttgttcttgttttttatcttctcttgctttttcgcttttttttctttgaaactttttttctttttcattttacgctttttttcttcttcattttttgcaaagcaaaaaatgataaaaagcagcgaagcaaaaaatgataaaaatgaaaagaaaagcaacaaagcaaaaaagtaaaaagaagttcaaaaaaaatgtattgagttgattttttttaattcaacaaaatttttcaaataaaaagtttttttcaaaaacgaagcacaaaaaaaattcaaaaaaaagtaaagaatcaaaaaaaagtagagaaaaattgtatttttcttttttgaaattttcatttcatcaagaaaaatacaacttttctaaaaaaaaatgaaaaaaatggaaatttctagattaatgtttgtgacggtggttgtgtcctttatcatcatcatctttataatcttcataatgcataaagcaagtttcagtaggtgcgtcagcagcattatctgcaatatcttcaaactccataacattatctaattctgaacccattgaaatgtttgtaactctttctaaaataacttcaactactacaggaacacgatattgagccattaatgctttagcctgttcaaaagctggagctatatcttctggtttaaaaacacgaatagctttacatcctaagccttcagcaacttttacgtgatctacaccataaccgttaacttctgaactgtttatattttcaaatgctaattgaacacagtaatccatatcaaaagcacgttgtgattgacgaattaaacctaaataagcgttatttactaaaacatggatatatggaatgttaaattgtgcaccaacagctaattcctcaataagaaattgaaagtcaaaatcacctgaaatagcaactacattacgttttggatcagctgcacatacacctaaagctgcaggaattgtccaacctaaagggccagcttggccacagttaatccaatgacggtctttaaatacatgaagcatttgtgcagcagctatttgacttaaaccaattgtagtaacatagcaaacatcacgaccaaatgctttattcatttcttcgtatacacgttgaggtttaacaggaacattatcaaaatgagtttttcttaataaagtacgtttacgttgttggcagtcagctacccattcttttctacaaggtaaacgacctgctttttgcatttcttgagcaacctcaacaagtaaagttaaagcagcttttgcatcacttacaatgcctaaatcagggcataatacacgaccaatttgtgtaggttcgatgtcaatgtgaacaattttacgtccttcagtatatttctctactgaaccagtatgacgattagcgaaacgatttccaataccgaataccatatcagatgctaataatgtagcattaccgtaacggtgagctgtttgtaaacctaccatgcctgccattaattcgtgatcatctggaatacaaccccagcccattaaagtaggaattacaggtactgaagttaattctgcaaattgctgtaaaagagcagctgcatcagcattaataacaccaccaccagcaacgattacaggacgttcagcttgaattaacatttctactgctttttcaatttgcatacgagaagcagcaggtttatatacaggtaaaggttcatacatgtctggatcgaattcaatttcagctacttgaacatcgaaaggtaaatcaactaatacaggaccaggacgacctgaacgcattaagtgaaaagcctgttgtaatacacgtggtactaaagcagcctcacgaacagttacagccattttacttacaggtttagcaatagcttcaatatcaactgcttgaaaatcttctttatgaagacgagcacgaggtgcttgaccagtaatacataagataggaatactatcagctgaagcactatataatgcagtaatcatatctgtaccagctgggccagatgtacctaaacaaacaccaatattacctgctgtagcacgtgtataaccttctgccatgtgactagcaccttctacatgacgagctaaaatgtgacgaatgccaccatgtttacgcatagcagaataaaatggattgattgctgcacctggtacaccgaaagctgtagtaataccttctttctctaatacatacattgctgcatcaacagcacgcatttttgccatatgaataaataatttataattttttctgtataaaccaattttccaagtaactttactttatcaaaaattaaaaaattaaaaaacttttattgaacttaaaataaaatttttaacaaaatttattttaaaaaaaagaaaaaatttttttattttggttttatttatttctttttttttacaaacaaaaatttttttaaacagaataataaaaaaaattttatttaaagaatggttttttaatattttgctcatgacaaatgattttttactacttttatgcttttttttaaaaaaagcagcaaagcaaaaaagttataaaaagtgtatggagcaagcggttaaattgacactttttaaaagtatttataggcccaaccggacttgaaccgatgacctattgcttgtaaggcaatcactctaccaactgagttatgggcctaaaaaatattatttatattttataatagaatataaaatctaacaacttctttagctagcactaggagatcacagatgagaagatatttgctcgataatcaatactctaggcatctaacttttcccattgtcttaaaccgacttaccttaggaatcatagtttcatgattttctgttacacctaactttttgtgtggtgccctcctccttgtcaatattaatgttaaagtgcaattctttttccttatcacgttgagccattagtatcaatttgcttacctgtattcctttacatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtagatatactagaagttctcctcgaccggtcgatatgcggtgtgaaataccgcacagatgcgtaaggagaaaataccgcatcaggaatttaaatactagtggatccttaattaactgcagggccggcctctagttttatttgtaaaccaaaaaaaatgaaaagccaaaaatttaagaaataaaaagtcaaagttatttaacaaaaatgaatttccaaaacttgcacgagataaaaaagataactctttaaatgaaaaaagaatgcttttttcaaaaaaagttttaaacaatacgtaaaaactttattttttttaaacttttttttgaaaaaaggcattcttttttttttaagaaattttaagtaatactttcatatttttttagtatttttttattgaataaaaaaaaactttaaagtaaaaaattggtcactttgaaagtcccagcttttttttaattcacttttttctttatttattttcctgtttaaaagaaaataaaatttttaaaaattttaaaaaataaaagaacaaactttcttgagataacactaaatcatctcaagaaagtttaatatttttgaaaagagttcgttcaaaaatttttcagttttaaatacaaattctcaaatatctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggcgctgacgaaaaacatattctcaataaaccctttagggaaataggccaggttttcaccgtaacacgccacatcttgcgaatatatgtgtagaaactgccggaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattatgaaaagttcatagaacattttgtcgatcctataaaaatctatccgtggaaaacttttttctttgaaaaaaaaaaagaaattgaaaaaaataaaaaactaaactatattatagaaaataataaaaactatttttttaacataaaaaaatagtttaaaaatttttattttttttaaattttaacataaattcaaaactttagcaaatttttaagtttaaatatttttttcaaagttaccacttgctcttggggaacaagtaaactaaattttttttctacgcaaaacttcatttttattggtttataatttttctaaattttcatctatttgttttttctatttttttataaactcaaaaaaagtttcaaaatatgaaaaaaatactagaaaaatttttttttgatttttttattatttggaaaagtttccaaaaaaaaaattgggttttaattttttttatgaattttagaattctttgttttttcattttagttttttttattttgtatttttttttctttgaacaaaagcaaaaaaagcaaaagtaaaaaaactagaggccgttctgcctgtagatccattggattggtatcaacgcgcaggcatagttcgagaaaaattatccagaggcaatgacaaccagcacgggctcgagactagtggccggcctctagtgctagcacaaaagaaatcaaatgttttcaaatcgtgcaaacatcaaattgcacaaaataatttttaaaattcattttatgaaagttcgttcttcagttaaaaaaatttgtactaaatgtcgtttaattcgtcgaaaaggtacagtaatggttatttgtacaaatcctaaacataaacaacgtcaaggataattttttttaatggaagaaatgttttttacttcttccattaaaaaagaaaaaagaaaagtgcagagctttttttttgatcaaaaaaaagatacaaaaaatttttttgttttaattttatgatataattctatttcagagaagaaaaaaaaatttaaacaaaacaaaaaaatatagagaagtttaaatttagacctaaaggtagatttccataaattcatttttctcatttgtttttttcttttttttgcattgcaaaaaaaaaagaaaaaagcaaaaagtcattttaaagagaaaaatggagaaagataaaagtttttaactttttttgaacaattccatatgaaagtaggatttataggattaggtattatgggcaaaccaatgtctaaaaacttacttaaagctggttactcattagttgttgctgatcgtaacccagaagcaatcgctgacgtaattgcagctggagctgaaacagcttcaactgctaaagctattgcagaacaatgtgatgttattattactatgttacctaattcacctcacgtaaaagaagtagctttaggcgaaaatggaattattgaaggtgctaaaccaggcacagtattaatagatatgtcatcaattgctccacttgcatcacgtgaaatttctgaagcattaaaagcaaaaggtattgatatgttagatgctccagttagtggtggtgaaccaaaagctatcgatggtacacttagtgttatggtaggcggtgataaagcaatttttgacaaatactacgatttaatgaaagctatggctggttctgtagtacacactggtgaaatcggtgcaggtaacgtaactaaattagctaaccaggttattgttgcattaaatatagctgcaatgtcagaagctcttactttagctacaaaagcaggtgtaaatcctgatttagtatatcaggcaattcgtggcggtttagcaggcagtactgtattagacgcaaaagctccaatggttatggatcgtaatttcaaaccaggttttagaattgatttacatattaaagaccttgctaatgctttagatacatcacacggtgtaggagctcaattaccattaactgcagctgttatggaaatgatgcaagcattacgtgctgatggtttaggtacagcagatcactcagctttagcttgttattatgagaaattagcaaaagttgaagttacacgtgattataaagatgatgatgataaaggacacaaccaccgtcacaaacattaatctagaaaaaaaagcatctttcaaaattaaactttaagtttttttcttttttttattttttccttttctttttattttatttaacaaaaagaaaaggaaaaaataaaaaaaaattttaagcgacttatgtttttaagtttcattttttttatttttatttattttttatttctttttttacaaaacttaaaaaaagtttaaaaataaaaaatttttgacaaaagaaatcaaatgttttcaaatcgtgcaaacatcaaattgcacaaaataatttttaaaattcattttccta

SEQ ID NO: 112 - DNA, 8759 bp, Artificial Sequence
  codon optimized sequence comprising CC93-CC97
tccaaactatttctcatattttttcagctccaaaagatcttatttaaaattgaaataatataaaaaattataaatttttacaaaacaagattttttctatttataatgttgtgattttttgactttttttactatttctcacaaattctataaaaaatcaaaaaatttttaaagaattttctattgtgtttttgaacaatttttttgttcttgttttttatcttctcttgctttttcgcttttttttctttgaaactttttttctttttcattttacgctttttttcttcttcattttttgcaaagcaaaaaatgataaaaagcagcgaagcaaaaaatgataaaaatgaaaagaaaagcaacaaagcaaaaaagtaaaaagaagttcaaaaaaaatgtattgagttgattttttttaattcaacaaaatttttcaaataaaaagtttttttcaaaaacgaagcacaaaaaaaattcaaaaaaaagtaaagaatcaaaaaaaagtagagaaaaattgtatttttcttttttgaaattttcatttcatcaagaaaaatacaacttttctaaaaaaaaatgaaaaaaatggaaatttctagattaatgtttgtgacggtggttgtgtcctttatcatcatcatctttataatcttcataatgcataaagcaagtttcagtaggtgcgtcagcagcattatctgcaatatcttcaaactccataacattatctaattctgaacccattgaaatgtttgtaactctttctaaaataacttcaactactacaggaacacgatattgagccattaatgctttagcctgttcaaaagctggagctatatcttctggtttaaaaacacgaatagctttacatcctaagccttcagcaacttttacgtgatctacaccataaccgttaacttctgaactgtttatattttcaaatgctaattgaacacagtaatccatatcaaaagcacgttgtgattgacgaattaaacctaaataagcgttatttactaaaacatggatatatggaatgttaaattgtgcaccaacagctaattcctcaataagaaattgaaagtcaaaatcacctgaaatagcaactacattacgttttggatcagctgcacatacacctaaagctgcaggaattgtccaacctaaagggccagcttggccacagttaatccaatgacggtctttaaatacatgaagcatttgtgcagcagctatttgacttaaaccaattgtagtaacatagcaaacatcacgaccaaatgctttattcatttcttcgtatacacgttgaggtttaacaggaacattatcaaaatgagtttttcttaataaagtacgtttacgttgttggcagtcagctacccattcttttctacaaggtaaacgacctgctttttgcatttcttgagcaacctcaacaagtaaagttaaagcagcttttgcatcacttacaatgcctaaatcagggcataatacacgaccaatttgtgtaggttcgatgtcaatgtgaacaattttacgtccttcagtatatttctctactgaaccagtatgacgattagcgaaacgatttccaataccgaataccatatcagatgctaataatgtagcattaccgtaacggtgagctgtttgtaaacctaccatgcctgccattaattcgtgatcatctggaatacaaccccagcccattaaagtaggaattacaggtactgaagttaattctgcaaattgctgtaaaagagcagctgcatcagcattaataacaccaccaccagcaacgattacaggacgttcagcttgaattaacatttctactgctttttcaatttgcatacgagaagcagcaggtttatatacaggtaaaggttcatacatgtctggatcgaattcaatttcagctacttgaacatcgaaaggtaaatcaactaatacaggaccaggacgacctgaacgcattaagtgaaaagcctgttgtaatacacgtggtactaaagcagcctcacgaacagttacagccattttacttacaggtttagcaatagcttcaatatcaactgcttgaaaatcttctttatgaagacgagcacgaggtgcttgaccagtaatacataagataggaatactatcagctgaagcactatataatgcagtaatcatatctgtaccagctgggccagatgtacctaaacaaacaccaatattacctgctgtagcacgtgtataaccttctgccatgtgactagcaccttctacatgacgagctaaaatgtgacgaatgccaccatgtttacgcatagcagaataaaatggattgattgctgcacctggtacaccgaaagctgtagtaataccttctttctctaatacatacattgctgcatcaacagcacgcatttttgccatatgaataaataatttataattttttctgtataaaccaattttccaagtaactttactttatcaaaaattaaaaaattaaaaaacttttattgaacttaaaataaaatttttaacaaaatttattttaaaaaaaagaaaaaatttttttattttggttttatttatttctttttttttacaaacaaaaatttttttaaacagaataataaaaaaaattttatttaaagaatggttttttaatattttgctcatgacaaatgattttttactacttttatgcttttttttaaaaaaagcagcaaagcaaaaaagttataaaaagtgtatggagcaagcggttaaattgacactttttaaaagtatttataggcccaaccggacttgaaccgatgacctattgcttgtaaggcaatcactctaccaactgagttatgggcctaaaaaatattatttatattttataatagaatataaaatctaacaacttctttagctagcactaggagatcacagatgagaagatatttgctcgataatcaatactctaggcatctaacttttcccattgtcttaaaccgacttaccttaggaatcatagtttcatgattttctgttacacctaactttttgtgtggtgccctcctccttgtcaatattaatgttaaagtgcaattctttttccttatcacgttgagccattagtatcaatttgcttacctgtattcctttacatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtagatatactagaagttctcctcgaccggtcgatatgcggtgtgaaataccgcacagatgcgtaaggagaaaataccgcatcaggaatttaaatactagtggatccttaattaactgcagggccggcctctagttttatttgtaaaccaaaaaaaatgaaaagccaaaaatttaagaaataaaaagtcaaagttatttaacaaaaatgaatttccaaaacttgcacgagataaaaaagataactctttaaatgaaaaaagaatgcttttttcaaaaaaagttttaaacaatacgtaaaaactttattttttttaaacttttttttgaaaaaaggcattcttttttttttaagaaattttaagtaatactttcatatttttttagtatttttttattgaataaaaaaaaactttaaagtaaaaaattggtcactttgaaagtcccagcttttttttaattcacttttttctttatttattttcctgtttaaaagaaaataaaatttttaaaaattttaaaaaataaaagaacaaactttcttgagataacactaaatcatctcaagaaagtttaatatttttgaaaagagttcgttcaaaaatttttcagttttaaatacaaattctcaaatatctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggcgctgacgaaaaacatattctcaataaaccctttagggaaataggccaggttttcaccgtaacacgccacatcttgcgaatatatgtgtagaaactgccggaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattatgaaaagttcatagaacattttgtcgatcctataaaaatctatccgtggaaaacttttttctttgaaaaaaaaaaagaaattgaaaaaaataaaaaactaaactatattatagaaaataataaaaactatttttttaacataaaaaaatagtttaaaaatttttattttttttaaattttaacataaattcaaaactttagcaaatttttaagtttaaatatttttttcaaagttaccacttgctcttggggaacaagtaaactaaattttttttctacgcaaaacttcatttttattggtttataatttttctaaattttcatctatttgttttttctatttttttataaactcaaaaaaagtttcaaaatatgaaaaaaatactagaaaaatttttttttgatttttttattatttggaaaagtttccaaaaaaaaaattgggttttaattttttttatgaattttagaattctttgttttttcattttagttttttttattttgtatttttttttctttgaacaaaagcaaaaaaagcaaaagtaaaaaaactagaggccgttctgcctgtagatccattggattggtatcaacgcgcaggcatagttcgagaaaaattatccagaggcaatgacaaccagcacgggctcgagactagtggccggcctctagtgctagcacaaaagaaatcaaatgttttcaaatcgtgcaaacatcaaattgcacaaaataatttttaaaattcattttatgaaagttcgttcttcagttaaaaaaatttgtactaaatgtcgtttaattcgtcgaaaaggtacagtaatggttatttgtacaaatcctaaacataaacaacgtcaaggataattttttttaatggaagaaatgttttttacttcttccattaaaaaagaaaaaagaaaagtgcagagctttttttttgatcaaaaaaaagatacaaaaaatttttttgttttaattttatgatataattctatttcagagaagaaaaaaaaatttaaacaaaacaaaaaaatatagagaagtttaaatttagacctaaaggtagatttccataaattcatttttctcatttgtttttttcttttttttgcattgcaaaaaaaaaagaaaaaagcaaaaagtcattttaaagagaaaaatggagaaagataaaagtttttaactttttttgaacaattccatatgaaattaggtttcatcggtttaggtattatgggtactccaatggcaattaatcttgctagagctggtcatcaattacatgttactactattggtcctgttgcagacgaacttttatctttaggtgctgtatcagttgaaacagcacgtcaagttacagaagcatcagacataatatttattatggttccagatactccacaagtagaagaagttttatttggtgaaaatggttgcactaaagcatctttaaaaggtaaaactattgttgatatgtcttctatctcacctattgaaactaaacgttttgcaagacaagtaaatgaattaggtggtgactatcttgacgctccagtttcaggtggtgaaattggtgctcgtgaaggtacattatctatcatggtaggtggagatgaagctgttttcgaacgtgtaaaacctttatttgaattacttggcaaaaatatcacattagttggaggtaatggtgatggacaaacatgtaaagttgcaaatcaaattattgtagcacttaatattgaagcagtttctgaagctcttttatttgcttcaaaagctggtgcagatcctgttcgtgttcgtcaagctcttatgggcggttttgcttcatcaagaattttagaagtacatggtgagcgtatgattaaacgtacattcaatcctggtttcaaaattgctttacatcaaaaagatttaaacttagctttacagtcagctaaagcattagctttaaacttacctaatactgctacttgccaagaacttttcaatacatgcgctgctaatggtggcagtcagttagatcattcagctttagttcaagcattagaacttatggctaaccataaattagcagattataaagatgacgacgataaaggtcacaatcaccgtcataaacactaatctagaaaaaaaagcatctttcaaaattaaactttaagtttttttcttttttttattttttccttttctttttattttatttaacaaaaagaaaaggaaaaaataaaaaaaaattttaagcgacttatgtttttaagtttcattttttttatttttatttattttttatttctttttttacaaaacttaaaaaaagtttaaaaataaaaaatttttgacaaaagaaatcaaatgttttcaaatcgtgcaaacatcaaattgcacaaaataatttttaaaattcattttccta

SEQ ID NO: 113 - DNA, 927 bp, Artificial Sequence
  codon optimized sequence comprising CC-97
atgaaattaggtttcatcggtttaggtattatgggtactccaatggcaattaatcttgctagagctggtcatcaattacatgttactactattggtcctgttgcagacgaacttttatctttaggtgctgtatcagttgaaacagcacgtcaagttacagaagcatcagacataatatttattatggttccagatactccacaagtagaagaagttttatttggtgaaaatggttgcactaaagcatctttaaaaggtaaaactattgttgatatgtcttctatctcacctattgaaactaaacgttttgcaagacaagtaaatgaattaggtggtgactatcttgacgctccagtttcaggtggtgaaattggtgctcgtgaaggtacattatctatcatggtaggtggagatgaagctgttttcgaacgtgtaaaacctttatttgaattacttggcaaaaatatcacattagttggaggtaatggtgatggacaaacatgtaaagttgcaaatcaaattattgtagcacttaatattgaagcagtttctgaagctcttttatttgcttcaaaagctggtgcagatcctgttcgtgttcgtcaagctcttatgggcggttttgcttcatcaagaattttagaagtacatggtgagcgtatgattaaacgtacattcaatcctggtttcaaaattgctttacatcaaaaagatttaaacttagctttacagtcagctaaagcattagctttaaacttacctaatactgctacttgccaagaacttttcaatacatgcgctgctaatggtggcagtcagttagatcattcagctttagttcaagcattagaacttatggctaaccataaattagcagattataaagatgacgacgataaaggtcacaatcaccgtcataaacactaa

SEQ ID NO: 114 - DNA, 16417 bp, Artificial Sequence
  codon optimized sequence comprising CC90-CC91-CC92
cgtttaggtgtaacacaatcttggggtggatggacaattagcggtgaaacagcaacaaatccaggtatttggagttatgaaggtgttgctgcatctcatattattttatctggtttattattcttagcttcggtttggcactgggtttactgggatttagagttattccgtgacccaagaactggaaaaactgcattagatttaccaaaaattttcggaattcacttattcttatcaggtcttttatgttttggttttggtgctttccacgtaacaggtttatttggtcctggtatttgggtttcagatccttatggattaacaggaagtgttcaaccagttgctccttcttggggtgctgatgggtttgatcctttcaaccctggtggtattgcagcgcaccacattgctgctggtattttaggtgttttagcaggattattccacttatgtgtacgtccttctattcgtttatactttggtttatcaatgggtagtatcgaaacagtattatcaagtagtattgctgctgttttctgggctgctttcgttgttgctggaactatgtggtatggttcagcagctactccaattgaattatttggtcctacacgttatcaatgggaccaaggtttcttccaacaagaaattcaaaaacgagttcaaacaagtttagcaggtggttcttcactttctgatgcttgggcgaaaattccagaaaaattagctttctatgattatattggaaacaaccctgcaaaaggtggtcttttccgtacaggagctatgaatagtggagatggtattgctgttggatggttaggtcacgcagtatttaaagatcaagatggtcgtgaattatacgtacgtcgtatgcctactttctttgaaacattcccagttttattaattgataaagatggtgttgtacgtgctgacgttcctttccgtcgtgctgaatcaaaatatagtattgaacaagttggtgtatcagtaactttctacggtggtgaattagatggattaacatttaatgatccagcaactgttaaaaaatatgctcgtaaagcacaattaggtgaaatttttgaatttgatcgttcaacattacaatctgatggtgtattccgtagtagtccacgtggttggtttacttttggtcacgtttgctttgctttattattcttctttggacatatttggcatggtgcacgtacaatcttccgtgatgtatttgctggtattgatgatgatctaaacgaaagtttagaatttggtaaatacaaaaaacttggtgatacaagttctgttcgtgaagctttctaattcgtttttttctcttttttttcttttttctctttggaaaaagaaaaaacatgtttattttgaattttttgtttagaactttactgttctttttttattttaaagtgtttctgtttttttttaatacaaaaacttttttaaaatgaatttaaaaaacacaaaaaaagagttattgctattcaaaataaacaagagtttaaaaacaaagtttttttctttagaaaaaaacttcttcattttttttgaattgtttttgaacttttttcttctcttgcttttatcgtttttttcttcactttttgcaaaaaagtgagaaaaaacagcaaagcaaaaaagtgaaaaaaagttcaaaaacaattcaaaaaagacaaaacctaaaaaaatatcacttgagatgggtctggattttttccaagcaaaagaattttgtattttgttgaaagtttttcataaaaatacaaatttgcaattattattcttaaaatcaaaatatttgttaaccacatttcattctatggaagcattagtttatacttttttattaatcggaacattaggaattatctttttcgcaattttctttagagaaccacctcgtatggtaaaataacgtaagatctcctaggaaaatgaattttaaaaattattttgtgcaatttgatgtttgcacgatttgaaaacatttgatttcttttgtcaaaaattttttatttttaaactttttttaagttttgtaaaaaaagaaataaaaaataaataaaaataaaaaaaatgaaacttaaaaacataagtcgcttaaaatttttttttattttttccttttctttttgttaaataaaataaaaagaaaaggaaaaaataaaaaaaagaaaaaaacttaaagtttaattttgaaagatgcttttttttctagattaatgtttgtgacggtggttgtgtcctttatcatcatcatctttataatcaaaacgttcaagctctggaaaaggtaaatggccgtgatgtacatgcatagcaccaaactctgcacaacgatgtaaagttggaatatttttaccaggattaagtaaaccgtctggatcaaaagcagcttttacagcgtgaaatgtagtaatttcatcactattgaattgagcacacatttgattgattttttcacggccgattccatgctcaccactaatagaaccacctacttctacacataactctaagatttttccaccaagttcttcagcacgagcaaactcaccaggttcgttagcatcgaataagattaatggatgcatattaccatcgccagcatgaaatacattagctacacgtaaatcgtattgttgagaaagacgtgcaataccttctaaaacacctggtaatgcacgacgtggaattgtaccatccatacaataatagtctggtgaaatacgacctactgcaggaaaagcatttttacgacctgcccaaaatcttacacgttcagcttcatcttgtgctaaacgaacatcagtagcaccagctttaagtaatatgtcgtttacacgctcacagtcttcctgaacatcactctctactccatctaattcacaaagtaaaattgcttcagcatctactggataaccagcgtgtataaaatcttcagctgcacgaattgataagttatccatcatctctaatcctcctggaattataccatttgcaatgatatcgcctacagctaatcctgctttttcaactgaatcaaaagatgctaataaaacacgagcaacaggtggttttggaagtaatttaacagttacttctgttgtaacacctaacattccttctgatcctgtgaataaagctaataaatcaaaaccaggagaatctaatgcatcactacctaaagtaagagcctcaccgtctaaagtttgtacttcaatttttaataagttgtgtactgttaaaccatattttaaacagtgtacgccaccagcattctctgctacattaccaccaattgaacaagcaatttgactactaggatctggagcgtaatataagttatgaggagcaactgcttgtgaaattgctaaattacgaacaccaggttgaacacgtgcacgacgaccaactgggttaatatctaaaatttctttaaaacgtgccataactaataatacacctttttctaaaggaagtgcaccgccacttaagcctgtacctgcaccacgtgtaacaactggaacacgtaatctatgacatactgctaatatagcagtaacttgttccatttgttttggtaaaactactaataaaggtcttgttctgtaagctgataatccatcacattcgtatggaataatttcttcatctgtatgtaaaatctctaagccaggtacgtgttcacgaagtgccattaaaactgaagtacgatcaacatctggtaaagcaccatctaaacgttcttcatataatatactcatatggaattgttcaaaaaaagttaaaaacttttatctttctccatttttctctttaaaatgactttttgcttttttcttttttttttgcaatgcaaaaaaaagaaaaaaacaaatgagaaaaatgaatttatggaaatctacctttaggtctaaatttaaacttctctatatttttttgttttgtttaaatttttttttcttctctgaaatagaattatatcataaaattaaaacaaaaaaattttttgtatcttttttttgatcaaaaaaaaagctctgcacttttcttttttcttttttaatggaagaagtaaaaaacatttcttccattaaaaaaaattatccttgacgttgtttatgtttaggatttgtacaaataaccattactgtaccttttcgacgaattaaacgacatttagtacaaatttttttaactgaagaacgaactttcataaaatgaattttaaaaattattttgtgcaatttgatgtttgcacgatttgaaaacatttgatttcttttgtgctagcactagaggccggccactagtctcgagcccgtgctggttgtcattgcctctggataatttttctcgaactatgcctgcgcgttgataccaatccaatggatctacaggcagaacggcctctagcggttttttttacttttgctttttttgcttttgttcaaagaaaaaaaaatacaaaataaaaaaaactaaaatgaaaaaacaaagaattctaaaattcataaaaaaaattaaaacccaattttttttttggaaacttttccaaataataaaaaaatcaaaaaaaaatttttctagtatttttttcatattttgaaactttttttgagtttataaaaaaatagaaaaaacaaatagatgaaaatttagaaaaattataaaccaataaaaatgaagttttgcgtagaaaaaaaatttagtttacttgttccccaagagcaagtggtaactttgaaaaaaatatttaaacttaaaaatttgctaaagttttgaatttatgttaaaatttaaaaaaaataaaaatttttaaactatttttttatgttaaaaaaatagtttttattattttctataatatagtttagttttttatttttttcaatttctttttttttttcaaagaaaaaagttttccacggatagatttttataggatcgacaaaatgttctatgaacttttcataatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgattccggcagtttctacacatatattcgcaagatgtggcgtgttacggtgaaaacctggcctatttccctaaagggtttattgagaatatgtttttcgtcagcgccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaacctagatatttgagaatttgtatttaaaactgaaaaatttttgaacgaactcttttcaaaaatattaaactttcttgagatgatttagtgttatctcaagaaagtttgttcttttattttttaaaatttttaaaaattttattttcttttaaacaggaaaataaataaagaaaaaagtgaattaaaaaaaagctgggactttcaaagtgaccaattttttactttaaagtttttttttattcaataaaaaaatactaaaaaaatatgaaagtattacttaaaatttcttaaaaaaaaaagaatgccttttttcaaaaaaaagtttaaaaaaaataaagtttttacgtattgtttaaaactttttttgaaaaaagcattcttttttcatttaaagagttatcttttttatctcgtgcaagttttggaaattcatttttgttaaataactttgactttttatttcttaaatttttggcttttcattttttttggtttacaaataaaactagaggccggccctgcagttaattaaggatccactagtatttaaattcctgatgcggtattttctccttacgcatctgtgcggtatttcacaccgcatatcgaccggtcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaaggtaagtcggtttaagacaatgggaaaagttagatgcctagagtattgattatcgagcaaatatcttctcatctgtgatctcctagtgctagctaaagaagttgttagattttatattctattataaaatataaataatattttttaggcccataactcagttggtagagtgattgccttacaagcaataggtcatcggttcaagtccggttgggcctataaatacttttaaaaagtgtcaatttaaccgcttgctccatacactttttataacttttttgctttgctgctttttttaaaaaaaagcataaaagtagtaaaaaatcatttgtcatgagcaaaatattaaaaaaccattctttaaataaaattttttttattattctgtttaaaaaaatttttgtttgtaaaaaaaaagaaataaataaaaccaaaataaaaaaattttttcttttttttaaaataaattttgttaaaaattttattttaagttcaataaaagttttttaattttttaatttttgataaagtaaagttacttggaaaattggtttatacagaaaaaattataaattatttattcatatgttacgtgaatgtgattatagtcaagctcttttagaacaagttaatcaagctatctctgacaaaactccattagttattcaaggctcaaactcaaaagcattcttaggacgtccagtaacaggccaaactttagacgttcgttgtcatcgtggtattgtaaattatgatccaactgagcttgtaattactgcacgtgttggtacacctttagtaacaattgaagctgctttagaaagtgctggtcaaatgttaccatgtgaaccacctcattacggtgaagaagctacttggggtggtatggtagcttgtggtttagctggtccacgtagaccttggagtggttctgttcgtgattttgtattaggtactcgtataattacaggtgctggtaaacatttacgtttcggtggtgaagttatgaaaaatgtagctggttatgatttatcacgtttaatggtaggtagttacggttgcttaggcgttttaacagaaatttcaatgaaagttttaccacgtccaagagcttctttatcattacgtcgtgaaatatcattacaagaagcaatgtcagaaattgcagaatggcaattacaacctttaccaataagtggtttatgttattttgataatgctttatggatcagattagaaggaggcgaaggtagtgttaaagctgctcgtgaattattaggtggtgaggaagtagcaggtcagttttggcaacaattacgtgaacaacagcttccatttttctcattaccaggtacattatggcgtattagtttaccatctgatgcaccaatgatggacttaccaggagaacaacttattgattggggaggtgctcttcgttggttaaaatcaactgctgaagataatcaaatccatcgtattgcacgtaatgctggcggtcacgcaactcgtttctcagcaggtgatggtggtttcgcacctttatctgctccattattccgttatcatcaacaacttaaacaacaattagatccttgtggtgtttttaatccaggacgtatgtacgctgagttagattataaagatgatgatgataaaggacacaaccaccgtcacaaacattagtctagaaatttccatttttttcatttttttttagaaaagttgtatttttcttgatgaaatgaaaatttcaaaaaagaaaaatacaatttttctctacttttttttgattctttacttttttttgaattttttttgtgcttcgtttttgaaaaaaactttttatttgaaaaattttgttgaattaaaaaaaatcaactcaatacattttttttgaacttctttttacttttttgctttgttgcttttcttttcatttttatcattttttgcttcgctgctttttatcattttttgctttgcaaaaaatgaagaagaaaaaaagcgtaaaatgaaaaagaaaaaaagtttcaaagaaaaaaaagcgaaaaagcaagagaagataaaaaacaagaacaaaaaaattgttcaaaaacacaatagaaaattctttaaaaattttttgattttttatagaatttgtgagaaatagtaaaaaaagtcaaaaaatcacaacattataaatagaaaaaatcttgttttgtaaaaatttataattttttatattatttcaattttaaataagatcttttggagctgaaaaaatatgagaaatagtttggacaaactagtctcgagcccgtatgatattctaaggcgttacgctgatgaatattctacagagttgccataggcgttgaacgctacacggacgatacgaatttttgaattagataaatgagtgttctcaatttttttttctttgcattttttgtttgtgttgatttacaaaaacaatagaaaaaagaaaacaatattttctttctaaaaaaaaacaaaattgatgaaaaatagacatgaacaaaaaattttgaaagttgacttttttaaaaaatttttggtataatacaaaaaaagaatttttggaaaggtggcagagtggttgaatgctctggttttgaaaaccagcgtggctttacggtcaccgggggttcgaatccctccctttccgataatatatacaaaaatttttaaagttttttgtttattttgtatagataaaaaatctgcaataaaaatttcgttttttatttattcaaaaattctgtttttttgaaaagaaaataaaaaaaatgccaaaagtgagttttttattcaaatattagaaaaagtttttgaaaaatttaaaaaaatagaaaaaatttttttatttttttcataatttaaaaaattatgttataatttaaattacaaataggttttattaaaaaatttttacgtacagatgaattctataaaattattttggagatcaccatatgcaaacacagttaacagaagaaatgcgtcaaaatgctcgtgctttagaagcagacagtatcttacgtgcttgtgtacattgtggcttttgtactgctacatgccctacataccaacttcttggtgatgaattagatggaccaagaggtcgtatttatttaataaaacaagtattagaaggtaatgaagttactttaaaaactcaagaacacttagaccgttgtttaacatgccgtaattgtgaaactacttgtccaagtggagtacgttatcataatttacttgatataggtcgtgatatcgtagaacaaaaagttaaacgtcctttaccagaacgtattttacgtgaaggacttcgtcaagtagttccaagaccagcagttttccgtgctttaactcaagtaggtttagttttacgtccatttttacctgaacaagtacgtgctaaattacctgcagaaactgttaaagcaaaaccacgtcctcctttacgtcataaacgtcgtgttttaatgttagaaggttgcgctcaaccaacattatctccaaatacaaatgcagcaactgctcgtgtattagatcgtttaggtatttcagttatgccagcaaatgaagcaggttgttgtggtgctgttgattatcacttaaatgctcaagaaaaaggtttagctagagctcgtaacaacattgacgcttggtggccagcaatcgaagcaggtgctgaagctattttacaaactgcatcaggttgcggtgcatttgttaaagaatatggccaaatgttaaaaaacgacgctttatatgctgataaagcacgtcaagtaagtgaacttgctgttgacttagtagaattattacgtgaagaacctcttgaaaaacttgctattcgtggtgataaaaaacttgctttccactgtccatgtactttacaacacgctcaaaaacttaatggtgaagtagaaaaagttcttttaagattaggttttactttaacagatgttcctgattcacacttatgttgtggttcagctggtacatacgctcttacacacccagacttagctcgtcaattacgtgacaacaaaatgaatgcacttgaaagtggaaaaccagaaatgattgttacagctaatattggctgccaaactcacttagcttctgctggtcgtacaagtgttcgtcattggattgaaattgtagaacaagcattagaaaaagaagattataaagatgatgatgataaaggacacaaccaccgtcacaaacattaatctagattttattttttatgaaaaactcaggcttaatttaggcttgagtttttcattctttttgaagctctgaaattttaaaatttctagtcttctttaatgtttttaaattttaaaaaataaatttcttctctgctgtgtttttctttttttttgaaaaaacaaagaaaaaaaatttttttgttttcttctttgtttttttatttctttttgttttgtttattttttagtttcagaatctttgattcaaaaaaaaatttagtccgattactccataggagcaagcagtaaaaaataaaaactgtaataaaaaataaaacaaaaattttatttctttttgttttgcttgaacttttcaaaaaaaaattgaaaaattcaagcaaaacaaaaagaaacaaataaaaaatttatgaattttctactttttcaggagttgaaatttctcctttacttaaaacatattttgctaaaaaaagcgcttgtgttgctttttttgctactttttgtttccaagcattttttcgaatatttttttttgattttgatgtgcgtttttgttaacctaaaatcttgaaaagatttactcttttcaaatttttatgtttttattttttttattcataaaaaaaaacaatacataaaaataaagtatttcggcttcaaaaaattttatacaaaaagttttttgattaaaaactcagaaaaaataaaaaaacaaagtatgaattttttgaaaaattcataccttttatttttttgtaatttttagcctttcaaaaaatttttgaaggcattttttttttaatcctcatgttcttcaaaaggatctctcaatttttttgaaggaggtccaaaactcacatagattgaatatcctgttgcacttaataatagaaaccataaaaaaaaggtaaagaaaaaagcaggactgtccataattctttcatgttttttgttcaaatttattctccaataattatattacgacaaaaagtaaaaaaaatcaaaatttattcaaaaaaatggctactggaacaacttcaaaagctaaatcaagcttatctgatgcacttcaagaaccaggtatcgtaactcctttaggaactttattaagaccgttaaactctgaatcaggaaaagtattacctggatggggaacaactgttttaatgggtgttttcattgtactttttgctgtattcttattaattattttagaaatttataacagttctttattattagataatgttactatgagttgggaaactttagcttcttaattcaatagaatagttttattgctttttttattttttattttatcaaaaattttttttgcaaaaataaagaataaataaaattcaaaaaaattatagaattagataaaattagtttcaagttgaactaagttgtcaataaactttcaaatttgttttctttttactgttcattaagagcaataaaaaaaacttttggtcttggcaatcttttaaaaaagtcagaatcaattctattttaagaatcctatggaatctatgtatttaattttagcaaaattaccagaagcttatgcaccttttgatcctattgtagatgttttaccaattattcctattttcttcttattattagcctttgtatggcaagcatctgtaagttttagataaaaaatttaaaagttttttttgatacttttgtaaaaaatatcaaaaaaaacttttaaatttttttcaattttcattagcaactttagctttaatattagctaaagttgctctcaaaaatataatttttttttgactttttatttttttattttgtttcttttttaaaagttacaacataaagaaaatgaaaatagaaaatttgtgaaacataaaaaaaaagaatgaaatttttatgttcgttttttgttttatcttttccaactaaagtcggcctctagctagaggccggccaaatttttttccaaaattctataaaaaatcaaaaaattaaaaaaaaaagaaaaaactttgttttgtgcaaaacaaaaatcttgaattcaaaacaaaataaagaattcaaaaagattttttttaagcaaaaggtaaaatggaaaaaaatgtttttaaaaaaattttttcttttttttaaagctttgcttttttcatgaaaaaaacaaagctttaaaaaaaagaaaaaattttaaagcaaaaaaaagaattaaacacgtcttttttttggaggacgacatccattatgtggaattcctgttttttctcgaataacatttactttaattccagctttaaaaatttcacgaatagctgtttcgcgaccttgtcctggaccagttactaaaatttttgcttcatttaatgcaaattcacgtgatttttttgccacaacttcagcagctttttttgctgcaaatgttgttgcttttctttttccacggaaaccacaagctccagcagaactccaacaaaggacttcaccacgaagatttgctaatgtaataatagtattatgatgtccagcttgaatataaacaattcctcgatatgtacgttttttaatttttgtaggtgatacttttctagtttgtctagccatatgtaaaaatttaactataaatttcttttatatttttaaatcatttgatttactatctaaaaaaaataagattttgaatctttggaaagaaccattatttggaaagatttatttgttcaattcttttgtattttttttcaaaaaaattttttagaattttttatctaattttttaatgttttgttcaaacataaaaaattttcttttcaaaaaaggaaaaaaaatttcttttttttgaaaaagtaaaaataaaaaaagctgtagcttttttgttgaatcaaaaaaaaataagaaattgtcctatttttatgacaaaagattcaaaaaaatgaaataaaaaagaaaaattcaaactttcaaacaatgtattttgtatttttgggccgagtcggattcgaaccaacgtaggcgtaaccagcggatttacaatccgcccccattaaccactcgggcatcggcccatgcttttttttgactcatttgatcatttattttgcatgaattatactaaagattatattaacaaaaaatttttgaaatttcaattttttttaattaaaaactcttctatttttttaaaattctttgttttgaattttttttttcaatttaaaaaaaaaacatattaaaaaatatttttaaaaaattttttgtattgaaaacttacaataattttataaatttttttgaaaaattttgttttttttattctattgaaatgaacaaaacaaatttttttagttttttttgtttttttgctttgctgcttcttttgtttttatcaataaataaatgaaaatgaaaaacaaaaataaacaatttgtgtttttagagttctaaaatcaagaaaaaaatacttcccctttaaagaggaagtattttaaaaaaaaattatagtttgtcaatagtttcaaattcaaatttgatttctttccaaacttcacaagcagcagctaattctggagaccatttacaagctgagcggataacatcaccaccttcacgagctaaatcacgaccttcgttacgagcttgagtacaagcttctaaagcaacacggttagcaacagcaccaggagcgttaccccaagggtgtcctaaagtacctccaccgaattgtaaacaagcgtcatcaccaaagatttcaactaaagctggcatgtgccatacgtgaataccaccagaagcaactggcatagtaccacccatagaacaccagtcttgagtgaagtaaataccacggcta

SEQ ID NO: 115 - DNA, 1553 bp, Artificial Sequence
  codon optimized sequence comprising CC-90
atgagtatattatatgaagaacgtttagatggtgctttaccagatgttgatcgtacttcagttttaatggcacttcgtgaacacgtacctggcttagagattttacatacagatgaagaaattattccatacgaatgtgatggattatcagcttacagaacaagacctttattagtagttttaccaaaacaaatggaacaagttactgctatattagcagtatgtcatagattacgtgttccagttgttacacgtggtgcaggtacaggcttaagtggcggtgcacttcctttagaaaaaggtgtattattagttatggcacgttttaaagaaattttagatattaacccagttggtcgtcgtgcacgtgttcaacctggtgttcgtaatttagcaatttcacaagcagttgctcctcataacttatattacgctccagatcctagtagtcaaattgcttgttcaattggtggtaatgtagcagagaatgctggtggcgtacactgtttaaaatatggtttaacagtacacaacttattaaaaattgaagtacaaactttagacggtgaggctcttactttaggtagtgatgcattagattctcctggttttgatttattagctttattcacaggatcagaaggaatgttaggtgttacaacagaagtaactgttaaattacttccaaaaccacctgttgctcgtgttttattagcatcttttgattcagttgaaaaagcaggattagctgtaggcgatatcattgcaaatggtataattccaggaggattagagatgatggataacttatcaattcgtgcagctgaagattttatacacgctggttatccagtagatgctgaagcaattttactttgtgaattagatggagtagagagtgatgttcaggaagactgtgagcgtgtaaacgacatattacttaaagctggtgctactgatgttcgtttagcacaagatgaagctgaacgtgtaagattttgggcaggtcgtaaaaatgcttttcctgcagtaggtcgtatttcaccagactattattgtatggatggtacaattccacgtcgtgcattaccaggtgttttagaaggtattgcacgtctttctcaacaatacgatttacgtgtagctaatgtatttcatgctggcgatggtaatatgcatccattaatcttattcgatgctaacgaacctggtgagtttgctcgtgctgaagaacttggtggaaaaatcttagagttatgtgtagaagtaggtggttctattagtggtgagcatggaatcggccgtgaaaaaatcaatcaaatgtgtgctcaattcaatagtgatgaaattactacatttcacgctgtaaaagctgcttttgatccagacggtttacttaatcctggtaaaaatattccaactttacatcgttgtgcagagtttggtgctatgcatgtacatcacggccatttaccttttccagagcttgaacgttttgattataaagatgatgatgataaaggacacaaccaccgtcacaaacattaatctag

SEQ ID NO: 116 - DNA, 1102 bp, Artificial Sequence
  codon optimized sequence comprising CC-91
atgttacgtgaatgtgattatagtcaagctcttttagaacaagttaatcaagctatctctgacaaaactccattagttattcaaggctcaaactcaaaagcattcttaggacgtccagtaacaggccaaactttagacgttcgttgtcatcgtggtattgtaaattatgatccaactgagcttgtaattactgcacgtgttggtacacctttagtaacaattgaagctgctttagaaagtgctggtcaaatgttaccatgtgaaccacctcattacggtgaagaagctacttggggtggtatggtagcttgtggtttagctggtccacgtagaccttggagtggttctgttcgtgattttgtattaggtactcgtataattacaggtgctggtaaacatttacgtttcggtggtgaagttatgaaaaatgtagctggttatgatttatcacgtttaatggtaggtagttacggttgcttaggcgttttaacagaaatttcaatgaaagttttaccacgtccaagagcttctttatcattacgtcgtgaaatatcattacaagaagcaatgtcagaaattgcagaatggcaattacaacctttaccaataagtggtttatgttattttgataatgctttatggatcagattagaaggaggcgaaggtagtgttaaagctgctcgtgaattattaggtggtgaggaagtagcaggtcagttttggcaacaattacgtgaacaacagcttccatttttctcattaccaggtacattatggcgtattagtttaccatctgatgcaccaatgatggacttaccaggagaacaacttattgattggggaggtgctcttcgttggttaaaatcaactgctgaagataatcaaatccatcgtattgcacgtaatgctggcggtcacgcaactcgtttctcagcaggtgatggtggtttcgcacctttatctgctccattattccgttatcatcaacaacttaaacaacaattagatccttgtggtgtttttaatccaggacgtatgtacgctgagttagattataaagatgatgatgataaaggacacaaccaccgtcacaaacattagt

SEQ ID NO: 117 - DNA, 1272 bp, Artificial Sequence
  codon optimized sequence comprising CC-92
atgcaaacacagttaacagaagaaatgcgtcaaaatgctcgtgctttagaagcagacagtatcttacgtgcttgtgtacattgtggcttttgtactgctacatgccctacataccaacttcttggtgatgaattagatggaccaagaggtcgtatttatttaataaaacaagtattagaaggtaatgaagttactttaaaaactcaagaacacttagaccgttgtttaacatgccgtaattgtgaaactacttgtccaagtggagtacgttatcataatttacttgatataggtcgtgatatcgtagaacaaaaagttaaacgtcctttaccagaacgtattttacgtgaaggacttcgtcaagtagttccaagaccagcagttttccgtgctttaactcaagtaggtttagttttacgtccatttttacctgaacaagtacgtgctaaattacctgcagaaactgttaaagcaaaaccacgtcctcctttacgtcataaacgtcgtgttttaatgttagaaggttgcgctcaaccaacattatctccaaatacaaatgcagcaactgctcgtgtattagatcgtttaggtatttcagttatgccagcaaatgaagcaggttgttgtggtgctgttgattatcacttaaatgctcaagaaaaaggtttagctagagctcgtaacaacattgacgcttggtggccagcaatcgaagcaggtgctgaagctattttacaaactgcatcaggttgcggtgcatttgttaaagaatatggccaaatgttaaaaaacgacgctttatatgctgataaagcacgtcaagtaagtgaacttgctgttgacttagtagaattattacgtgaagaacctcttgaaaaacttgctattcgtggtgataaaaaacttgctttccactgtccatgtactttacaacacgctcaaaaacttaatggtgaagtagaaaaagttcttttaagattaggttttactttaacagatgttcctgattcacacttatgttgtggttcagctggtacatacgctcttacacacccagacttagctcgtcaattacgtgacaacaaaatgaatgcacttgaaagtggaaaaccagaaatgattgttacagctaatattggctgccaaactcacttagcttctgctggtcgtacaagtgttcgtcattggattgaaattgtagaacaagcattagaaaaagaagattataaagatgatgatgataaaggacacaaccaccgtcacaaacattaa

SEQ ID NO: 118 - DNA, 1179 bp, Artificial Sequence
  codon optimized sequence comprising HIS3
aattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaact

SEQ ID NO: 119 - DNA, 4879 bp, Artificial Sequence
  codon optimized sequence comprising LYS2
agcagttgctttctcctatgggaagagctttctaagtctgaagaagtaaacagttctttgctatttcacacttcctggttgatggtcacttgctgcctgaaatatatatatatgtatgacatatgtacttgttttcttttttgtgcctttgttacgtctatattcattgaaactgattattcgattttcttcttgctgaccgcttctagaggcatcgcacagttttagcgaggaaaactcttcaatagttttgccagcggaattccacttgcaattacataaaaaattccggcggtttttcgcgtgtgactcaatgtcgaaatacctgcctaatgaacatgaacatcgcccaaatgtatttgaagacccgctgggagaagttcaagatatataagtaacaagcagccaatagtataaaaaaaaatctgagtttattacctttcctggaatttcagtgaaaaactgctaattatagagagatatcacagagttactcactaatgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgagctagctgaaaaaattcaaagttgccaagatctggaaaggacccctcagttgttccgtttggcctttttggaaaaccaagatttcaaattagacgagttcaagcatcatttagtggactttgctttgaatttggataccagtaataatgcgcatgttttgaacttaatttataacagcttactgtattcgaatgaaagagtaaccattgttgcggaccaatttactcaatatttgactgctgcgctaagcgatccatccaattgcataactaaaatctctctgatcaccgcatcatccaaggatagtttacctgatccaactaagaacttgggctggtgcgatttcgtggggtgtattcacgacattttccaggacaatgctgaagccttcccagagagaacctgtgttgtggagactccaacactaaattccgacaagtcccgttctttcacttatcgcgacatcaaccgcacttctaacatagttgcccattatttgattaaaacaggtatcaaaagaggtgatgtagtgatgatctattcttctaggggtgtggatttgatggtatgtgtgatgggtgtcttgaaagccggcgcaaccttttcagttatcgaccctgcatatcccccagccagacaaaccatttacttaggtgttgctaaaccacgtgggttgattgttattagagctgctggacaattggatcaactagtagaagattacatcaatgatgaattggagattgtttcaagaatcaattccatcgctattcaagaaaatggtaccattgaaggtggcaaattggacaatggcgaggatgttttggctccatatgatcactacaaagacaccagaacaggtgttgtagttggaccagattccaacccaaccctatctttcacatctggttccgaaggtattcctaagggtgttcttggtagacatttttccttggcttattatttcaattggatgtccaaaaggttcaacttaacagaaaatgataaattcacaatgctgagcggtattgcacatgatccaattcaaagagatatgtttacaccattatttttaggtgcccaattgtatgtccctactcaagatgatattggtacaccgggccgtttagcggaatggatgagtaagtatggttgcacagttacccatttaacacctgccatgggtcaattacttactgcccaagctactacaccattccctaagttacatcatgcgttctttgtgggtgacattttaacaaaacgtgattgtctgaggttacaaaccttggcagaaaattgccgtattgttaatatgtacggtaccactgaaacacagcgtgcagtttcttatttcgaagttaaatcaaaaaatgacgatccaaactttttgaaaaaattgaaagatgtcatgcctgctggtaaaggtatgttgaacgttcagctactagttgttaacaggaacgatcgtactcaaatatgtggtattggcgaaataggtgagatttatgttcgtgcaggtggtttggccgaaggttatagaggattaccagaattgaataaagaaaaatttgtgaacaactggtttgttgaaaaagatcactggaattatttggataaggataatggtgaaccttggagacaattctggttaggtccaagagatagattgtacagaacgggtgatttaggtcgttatctaccaaacggtgactgtgaatgttgcggtagggctgatgatcaagttaaaattcgtgggttcagaatcgaattaggagaaatagatacgcacatttcccaacatccattggtaagagaaaacattactttagttcgcaaaaatgccgacaatgagccaacattgatcacatttatggtcccaagatttgacaagccagatgacttgtctaagttccaaagtgatgttccaaaggaggttgaaactgaccctatagttaagggcttaatcggttaccatcttttatccaaggacatcaggactttcttaaagaaaagattggctagctatgctatgccttccttgattgtggttatggataaactaccattgaatccaaatggtaaagttgataagcctaaacttcaattcccaactcccaagcaattaaatttggtagctgaaaatacagtttctgaaactgacgactctcagtttaccaatgttgagcgcgaggttagagacttatggttaagtatattacctaccaagccagcatctgtatcaccagatgattcgtttttcgatttaggtggtcattctatcttggctaccaaaatgatttttaccttaaagaaaaagctgcaagttgatttaccattgggcacaattttcaagtatccaacgataaaggcctttgccgcggaaattgacagaattaaatcatcgggtggatcatctcaaggtgaggtcgtcgaaaatgtcactgcaaattatgcggaagacgccaagaaattggttgagacgctaccaagttcgtacccctctcgagaatattttgttgaacctaatagtgccgaaggaaaaacaacaattaatgtgtttgttaccggtgtcacaggatttctgggctcctacatccttgcagatttgttaggacgttctccaaagaactacagtttcaaagtgtttgcccacgtcagggccaaggatgaagaagctgcatttgcaagattacaaaaggcaggtatcacctatggtacttggaacgaaaaatttgcctcaaatattaaagttgtattaggcgatttatctaaaagccaatttggtctttcagatgagaagtggatggatttggcaaacacagttgatataattatccataatggtgcgttagttcactgggtttatccatatgccaaattgagggatccaaatgttatttcaactatcaatgttatgagcttagccgccgtcggcaagccaaagttctttgactttgtttcctccacttctactcttgacactgaatactactttaatttgtcagataaacttgttagcgaagggaagccaggcattttagaatcagacgatttaatgaactctgcaagcgggctcactggtggatatggtcagtccaaatgggctgctgagtacatcattagacgtgcaggtgaaaggggcctacgtgggtgtattgtcagaccaggttacgtaacaggtgcctctgccaatggttcttcaaacacagatgatttcttattgagatttttgaaaggttcagtccaattaggtaagattccagatatcgaaaattccgtgaatatggttccagtagatcatgttgctcgtgttgttgttgctacgtctttgaatcctcccaaagaaaatgaattggccgttgctcaagtaacgggtcacccaagaatattattcaaagactacttgtatactttacacgattatggttacgatgtcgaaatcgaaagctattctaaatggaagaaatcattggaggcgtctgttattgacaggaatgaagaaaatgcgttgtatcctttgctacacatggtcttagacaacttacctgaaagtaccaaagctccggaactagacgataggaacgccgtggcatctttaaagaaagacaccgcatggacaggtgttgattggtctaatggaataggtgttactccagaagaggttggtatatatattgcatttttaaacaaggttggatttttacctccaccaactcataatgacaaacttccactgccaagtatagaactaactcaagcgcaaataagtctagttgcttcaggtgctggtgctcgtggaagctccgcagcagcttaaggttgagcattacgtatgatatgtccatgtacaataattaaatatgaattaggagaaagacttagcttcttttcgggtgatgtcacttaaaaactccgagaataatatataataagagaataaaatattagttattgaataagaactgtaaatcagctggcgttagtctgctaatggcagcttcatcttggtttattgta

SEQ ID NO: 120 - DNA, 24607 bp, Artificial Sequence
  codon optimized sequence comprising IS57-IS116-IS62-IS61
cgtttaggtgtaacacaatcttggggtggatggacaattagcggtgaaacagcaacaaatccaggtatttggagttatgaaggtgttgctgcatctcatattattttatctggtttattattcttagcttcggtttggcactgggtttactgggatttagagttattccgtgacccaagaactggaaaaactgcattagatttaccaaaaattttcggaattcacttattcttatcaggtcttttatgttttggttttggtgctttccacgtaacaggtttatttggtcctggtatttgggtttcagatccttatggattaacaggaagtgttcaaccagttgctccttcttggggtgctgatgggtttgatcctttcaaccctggtggtattgcagcgcaccacattgctgctggtattttaggtgttttagcaggattattccacttatgtgtacgtccttctattcgtttatactttggtttatcaatgggtagtatcgaaacagtattatcaagtagtattgctgctgttttctgggctgctttcgttgttgctggaactatgtggtatggttcagcagctactccaattgaattatttggtcctacacgttatcaatgggaccaaggtttcttccaacaagaaattcaaaaacgagttcaaacaagtttagcaggtggttcttcactttctgatgcttgggcgaaaattccagaaaaattagctttctatgattatattggaaacaaccctgcaaaaggtggtcttttccgtacaggagctatgaatagtggagatggtattgctgttggatggttaggtcacgcagtatttaaagatcaagatggtcgtgaattatacgtacgtcgtatgcctactttctttgaaacattcccagttttattaattgataaagatggtgttgtacgtgctgacgttcctttccgtcgtgctgaatcaaaatatagtattgaacaagttggtgtatcagtaactttctacggtggtgaattagatggattaacatttaatgatccagcaactgttaaaaaatatgctcgtaaagcacaattaggtgaaatttttgaatttgatcgttcaacattacaatctgatggtgtattccgtagtagtccacgtggttggtttacttttggtcacgtttgctttgctttattattcttctttggacatatttggcatggtgcacgtacaatcttccgtgatgtatttgctggtattgatgatgatctaaacgaaagtttagaatttggtaaatacaaaaaacttggtgatacaagttctgttcgtgaagctttctaattcgtttttttctcttttttttcttttttctctttggaaaaagaaaaaacatgtttattttgaattttttgtttagaactttactgttctttttttattttaaagtgtttctgtttttttttaatacaaaaacttttttaaaatgaatttaaaaaacacaaaaaaagagttattgctattcaaaataaacaagagtttaaaaacaaagtttttttctttagaaaaaaacttcttcattttttttgaattgtttttgaacttttttcttctcttgcttttatcgtttttttcttcactttttgcaaaaaagtgagaaaaaacagcaaagcaaaaaagtgaaaaaaagttcaaaaacaattcaaaaaagacaaaacctaaaaaaatatcacttgagatgggtctggattttttccaagcaaaagaattttgtattttgttgaaagtttttcataaaaatacaaatttgcaattattattcttaaaatcaaaatatttgttaaccacatttcattctatggaagcattagtttatacttttttattaatcggaacattaggaattatctttttcgcaattttctttagagaaccacctcgtatggtaaaataagggctcgagactagtttgtccaaactatttctcatattttttcagctccaaaagatcttatttaaaattgaaataatataaaaaattataaatttttacaaaacaagattttttctatttataatgttgtgattttttgactttttttactatttctcacaaattctataaaaaatcaaaaaatttttaaagaattttctattgtgtttttgaacaatttttttgttcttgttttttatcttctcttgctttttcgcttttttttctttgaaactttttttctttttcattttacgctttttttcttcttcattttttgcaaagcaaaaaatgataaaaagcagcgaagcaaaaaatgataaaaatgaaaagaaaagcaacaaagcaaaaaagtaaaaagaagttcaaaaaaaatgtattgagttgattttttttaattcaacaaaatttttcaaataaaaagtttttttcaaaaacgaagcacaaaaaaaattcaaaaaaaagtaaagaatcaaaaaaaagtagagaaaaattgtatttttcttttttgaaattttcatttcatcaagaaaaatacaacttttctaaaaaaaaatgaaaaaaatggaaatttctagattaaccggtgtgtttatgacgatgattgtgaccttgaaagtataagttctcaccacttttatcatcatcatctttgtaatcaccggtaccagcatgaactggacgagctccacttgataattgaacatttgcagcatactcacgagcccataagtcataatgtacaatttcttcaagacttggagaagttactaattcattacggtgtttatcacatgttaattcaactactttgaagatgtctaagtatgaaattttctcgtcaatgaacatttctacagctttctcattagcagcagataaaacaccagtcatagtaccacctgcacgaccagctgcataagcaagatccattgatggatattttacgttgtcaggttttttgaatgtaagagatcctaatttacataaatctaaacgtggccaagtaacttcactgcaaggaacacggtctggccatgacattgtatataaaataggtaaacgcatgtcaggccagcctaattgagctaatactgaagaatcttgtgtttcaatcatactatgaataattgattgtggatgaataacaatttcaatatcgtcgtattcagcaccgaaaagataatgtgcttcaataacttctaaacctttgttgaataaagttgcactatctacagtaatttttttgcccatattccagtttgggtgttttaatgcatcagctacttttacctctttaagtttttctacaggccaatcacgaaatgcaccacctgaagctgttaatatgatttttcttaatgcaccttcaggtaagccttgaatacattgaaaaattgctgaatgttcactgtctgctggaagaatttttacattgtgtttgtttgcaagtggtaatacgaatggaccacctgcgattaatgtttctttatttgctaaagcaatatctttaccagcctcaatagctgcaactgtaggttttaatccagcacaaccaacaatacctgtaactactgtaactgcttctggatgacgagcaacttcaattacaccttgttctcctggaataatctctaatttgtagtctaaatctgctaatgcttcttttaattcattaattaaagattcattacgaactgcaactaaagcaggtttaaaacgacgtacctgatcagctaataaagtaacgttagaaccagcagctaatgcaactacacgaaatttatctggattttctgcaacaatgtctaaagtttgagtaccgattgaaccagttgatcctacaatactaataggtttaggaccatcccatgattgtctaggagcttctggaacagcacgacctggccaagcaggtggtggttgttgctgttgctgtacttttacagagcattttactcctttaccaaaaccacgaccttgattacgacgacgtaaagaaaaaccaccagataatttaggaattggattgaaacgagaagtatctaaaaatgaaattgcttttgattctgctggagataatgaatttaatgttggtaccatatgaataaataatttataattttttctgtataaaccaattttccaagtaactttactttatcaaaaattaaaaaattaaaaaacttttattgaacttaaaataaaatttttaacaaaatttattttaaaaaaaagaaaaaatttttttattttggttttatttatttctttttttttacaaacaaaaatttttttaaacagaataataaaaaaaattttatttaaagaatggttttttaatattttgctcatgacaaatgattttttactacttttatgcttttttttaaaaaaagcagcaaagcaaaaaagttataaaaagtgtatggagcaagcggttaaattgacactttttaaaagtatttataggcccaaccggacttgaaccgatgacctattgcttgtaaggcaatcactctaccaactgagttatgggcctaaaaaatattatttatattttataatagaatataaaatctaacaacttctttagctagcactaggagaaaatagcctcgcggagccatgtgccatactcgtctgcggagcactctggtaatgcatatggtccacaggacattcgtcgcttccgggtatgcgctctatgaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactaccactcaatttgacctagtcctcaaacgttctgctaaaccgtgtcaatcagtgtctgcttcctgagtgaaacccgtaagatctcctaggaaaatgaattttaaaaattattttgtgcaatttgatgtttgcacgatttgaaaacatttgatttcttttgtcaaaaattttttatttttaaactttttttaagttttgtaaaaaaagaaataaaaaataaataaaaataaaaaaaatgaaacttaaaaacataagtcgcttaaaatttttttttattttttccttttctttttgttaaataaaataaaaagaaaaggaaaaaataaaaaaaagaaaaaaacttaaagtttaattttgaaagatgcttttttttctagattaaccggttgtattttcttgatgaattgtacgtgttaaataaaattcagctaaagctaaatcttctggacgtgtaactttaatgttatcagcacgaccttcaactaattgtggatgaaaaccacagtattctaaagctgatgcttcatctgtaattgtagcaccttcatttaaagcacgtgttaaacaatcatgtaataattcacgtggaaaaaattgtggtgttaaagcgtgccataaaccattacgatcaactgtatgagcaatagcatttttacctggttcagcacgtttcattgtatcacgaactggagcagctaaaataccacctgtacgtgatgtttctgataaagctaataaacgagctaaatcatcttgatgtaaacatggacgagcagcatcatgaactaaaacccattgagcatcaccagcagcttttaaaccagctaaaactgaatcagcacgttcatcaccaccatcaacaactgtaatttgtggatgattagctaatggtaattgagcaaaacgtgaatcacctggtgaaatagcaataacaacacgtttaacacgtggatgagctaataaagcatgaactgaatgttctaaaattgtttgattaccaattgataagtattgttttggacattctgtttgcatacgacgaccaaaaccagcagcaggaacaacagcacaaacatctaaatgtgttgtagcacctttgtcgtcatcgtctttataatccatatggaattgttcaaaaaaagttaaaaacttttatctttctccatttttctctttaaaatgactttttgcttttttcttttttttttgcaatgcaaaaaaaagaaaaaaacaaatgagaaaaatgaatttatggaaatctacctttaggtctaaatttaaacttctctatatttttttgttttgtttaaatttttttttcttctctgaaatagaattatatcataaaattaaaacaaaaaaattttttgtatcttttttttgatcaaaaaaaaagctctgcacttttcttttttcttttttaatggaagaagtaaaaaacatttcttccattaaaaaaaattatccttgacgttgtttatgtttaggatttgtacaaataaccattactgtaccttttcgacgaattaaacgacatttagtacaaatttttttaactgaagaacgaactttcataaaatgaattttaaaaattattttgtgcaatttgatgtttgcacgatttgaaaacatttgatttcttttgtgctagcactagaggccggccactagtctcgagcccgtgctggttgtcattgcctctggataatttttctcgaactatgcctgcgcgttgataccaatccaatggatctacaggcagaacggcctctagcggttttttttacttttgctttttttgcttttgttcaaagaaaaaaaaatacaaaataaaaaaaactaaaatgaaaaaacaaagaattctaaaattcataaaaaaaattaaaacccaattttttttttggaaacttttccaaataataaaaaaatcaaaaaaaaatttttctagtatttttttcatattttgaaactttttttgagtttataaaaaaatagaaaaaacaaatagatgaaaatttagaaaaattataaaccaataaaaatgaagttttgcgtagaaaaaaaatttagtttacttgttccccaagagcaagtggtaactttgaaaaaaatatttaaacttaaaaatttgctaaagttttgaatttatgttaaaatttaaaaaaaataaaaatttttaaactatttttttatgttaaaaaaatagtttttattattttctataatatagtttagttttttatttttttcaatttctttttttttttcaaagaaaaaagttttccacggatagatttttataggatcgacaaaatgttctatgaacttttcataatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgattccggcagtttctacacatatattcgcaagatgtggcgtgttacggtgaaaacctggcctatttccctaaagggtttattgagaatatgtttttcgtcagcgccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaacctagatatttgagaatttgtatttaaaactgaaaaatttttgaacgaactcttttcaaaaatattaaactttcttgagatgatttagtgttatctcaagaaagtttgttcttttattttttaaaatttttaaaaattttattttcttttaaacaggaaaataaataaagaaaaaagtgaattaaaaaaaagctgggactttcaaagtgaccaattttttactttaaagtttttttttattcaataaaaaaatactaaaaaaatatgaaagtattacttaaaatttcttaaaaaaaaaagaatgccttttttcaaaaaaaagtttaaaaaaaataaagtttttacgtattgtttaaaactttttttgaaaaaagcattcttttttcatttaaagagttatcttttttatctcgtgcaagttttggaaattcatttttgttaaataactttgactttttatttcttaaatttttggcttttcattttttttggtttacaaataaaactagaggccggccctgcagttaattaaggatccactagtatttaaattcctgatgcggtattttctccttacgcatctgtgcggtatttcacaccgcatatcgaccggtcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaaggtaagtcggtttaagacaatgggaaaagttagatgcctagagtattgattatcgagcaaatatcttctcatctgtgatctcctagtgctagctaaagaagttgttagattttatattctattataaaatataaataatattttttaggcccataactcagttggtagagtgattgccttacaagcaataggtcatcggttcaagtccggttgggcctataaatacttttaaaaagtgtcaatttaaccgcttgctccatacactttttataacttttttgctttgctgctttttttaaaaaaaagcataaaagtagtaaaaaatcatttgtcatgagcaaaatattaaaaaaccattctttaaataaaattttttttattattctgtttaaaaaaatttttgtttgtaaaaaaaaagaaataaataaaaccaaaataaaaaaattttttcttttttttaaaataaattttgttaaaaattttattttaagttcaataaaagttttttaattttttaatttttgataaagtaaagttacttggaaaattggtttatacagaaaaaattataaattatttattcatatggattataaagatgatgacgacaaaggtatgcacaagttcacaggtgttaacgctaaattccagcaaccagcattaagaaatttatctccagtggtagttgagcgcgaacgtgaggaatttgtaggattctttccacaaattgttcgtgacttaactgaagatggtattggtcatccagaagtaggtgacgctgtagctcgtcttaaagaagtattacaatacaacgcacctggtggtaaatgcaatagaggtttaacagttgttgcagcttaccgtgaactttctggaccaggtcaaaaagacgctgaaagtcttcgttgtgctttagcagtaggatggtgtattgaattattccaagcctttttcttagttgctgacgatataatggaccagtcattaactagacgtggtcaattatgttggtacaagaaagaaggtgttggtttagatgcaataaatgattcttttcttttagaaagctctgtgtatcgcgttcttaaaaagtattgccgtcaacgtccatattatgtacatttattagagctttttcttcaaacagcttaccaaacagaattaggacaaatgttagatttaatcactgctcctgtatctaaggtagatttaagccatttctcagaagaacgttacaaagctattgttaagtataaaactgctttctattcattctatttaccagttgcagcagctatgtatatggttggtatagattctaaagaagaacatgaaaacgcaaaagctattttacttgagatgggtgaatacttccaaattcaagatgattatttagattgttttggcgatcctgctttaacaggtaaagtaggtactgatattcaagataacaaatgttcatggttagttgtgcaatgcttacaaagagtaacaccagaacaacgtcaacttttagaagataattacggtcgtaaagaaccagaaaaagttgctaaagttaaagaattatatgaggctgtaggtatgagagccgcctttcaacaatacgaagaaagtagttaccgtcgtcttcaagagttaattgagaaacattctaatcgtttaccaaaagaaattttcttaggtttagctcagaaaatatacaaacgtcaaaaatcaggtccaagatcttaatctagaaatttccatttttttcatttttttttagaaaagttgtatttttcttgatgaaatgaaaatttcaaaaaagaaaaatacaatttttctctacttttttttgattctttacttttttttgaattttttttgtgcttcgtttttgaaaaaaactttttatttgaaaaattttgttgaattaaaaaaaatcaactcaatacattttttttgaacttctttttacttttttgctttgttgcttttcttttcatttttatcattttttgcttcgctgctttttatcattttttgctttgcaaaaaatgaagaagaaaaaaagcgtaaaatgaaaaagaaaaaaagtttcaaagaaaaaaaagcgaaaaagcaagagaagataaaaaacaagaacaaaaaaattgttcaaaaacacaatagaaaattctttaaaaattttttgattttttatagaatttgtgagaaatagtaaaaaaagtcaaaaaatcacaacattataaatagaaaaaatcttgttttgtaaaaatttataattttttatattatttcaattttaaataagatcttttggagctgaaaaaatatgagaaatagtttggacaaactagtctcgagcccgtatgatattctaaggcgttacgctgatgaatattctacagagttgccataggcgttgaacgctacacggacgatacgaaagcagttgctttctcctatgggaagagctttctaagtctgaagaagtaaacagttctttgctatttcacacttcctggttgatggtcacttgctgcctgaaatatatatatatgtatgacatatgtacttgttttcttttttgtgcctttgttacgtctatattcattgaaactgattattcgattttcttcttgctgaccgcttctagaggcatcgcacagttttagcgaggaaaactcttcaatagttttgccagcggaattccacttgcaattacataaaaaattccggcggtttttcgcgtgtgactcaatgtcgaaatacctgcctaatgaacatgaacatcgcccaaatgtatttgaagacccgctgggagaagttcaagatatataagtaacaagcagccaatagtataaaaaaaaatctgagtttattacctttcctggaatttcagtgaaaaactgctaattatagagagatatcacagagttactcactaatgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgagctagctgaaaaaattcaaagttgccaagatctggaaaggacccctcagttgttccgtttggcctttttggaaaaccaagatttcaaattagacgagttcaagcatcatttagtggactttgctttgaatttggataccagtaataatgcgcatgttttgaacttaatttataacagcttactgtattcgaatgaaagagtaaccattgttgcggaccaatttactcaatatttgactgctgcgctaagcgatccatccaattgcataactaaaatctctctgatcaccgcatcatccaaggatagtttacctgatccaactaagaacttgggctggtgcgatttcgtggggtgtattcacgacattttccaggacaatgctgaagccttcccagagagaacctgtgttgtggagactccaacactaaattccgacaagtcccgttctttcacttatcgcgacatcaaccgcacttctaacatagttgcccattatttgattaaaacaggtatcaaaagaggtgatgtagtgatgatctattcttctaggggtgtggatttgatggtatgtgtgatgggtgtcttgaaagccggcgcaaccttttcagttatcgaccctgcatatcccccagccagacaaaccatttacttaggtgttgctaaaccacgtgggttgattgttattagagctgctggacaattggatcaactagtagaagattacatcaatgatgaattggagattgtttcaagaatcaattccatcgctattcaagaaaatggtaccattgaaggtggcaaattggacaatggcgaggatgttttggctccatatgatcactacaaagacaccagaacaggtgttgtagttggaccagattccaacccaaccctatctttcacatctggttccgaaggtattcctaagggtgttcttggtagacatttttccttggcttattatttcaattggatgtccaaaaggttcaacttaacagaaaatgataaattcacaatgctgagcggtattgcacatgatccaattcaaagagatatgtttacaccattatttttaggtgcccaattgtatgtccctactcaagatgatattggtacaccgggccgtttagcggaatggatgagtaagtatggttgcacagttacccatttaacacctgccatgggtcaattacttactgcccaagctactacaccattccctaagttacatcatgcgttctttgtgggtgacattttaacaaaacgtgattgtctgaggttacaaaccttggcagaaaattgccgtattgttaatatgtacggtaccactgaaacacagcgtgcagtttcttatttcgaagttaaatcaaaaaatgacgatccaaactttttgaaaaaattgaaagatgtcatgcctgctggtaaaggtatgttgaacgttcagctactagttgttaacaggaacgatcgtactcaaatatgtggtattggcgaaataggtgagatttatgttcgtgcaggtggtttggccgaaggttatagaggattaccagaattgaataaagaaaaatttgtgaacaactggtttgttgaaaaagatcactggaattatttggataaggataatggtgaaccttggagacaattctggttaggtccaagagatagattgtacagaacgggtgatttaggtcgttatctaccaaacggtgactgtgaatgttgcggtagggctgatgatcaagttaaaattcgtgggttcagaatcgaattaggagaaatagatacgcacatttcccaacatccattggtaagagaaaacattactttagttcgcaaaaatgccgacaatgagccaacattgatcacatttatggtcccaagatttgacaagccagatgacttgtctaagttccaaagtgatgttccaaaggaggttgaaactgaccctatagttaagggcttaatcggttaccatcttttatccaaggacatcaggactttcttaaagaaaagattggctagctatgctatgccttccttgattgtggttatggataaactaccattgaatccaaatggtaaagttgataagcctaaacttcaattcccaactcccaagcaattaaatttggtagctgaaaatacagtttctgaaactgacgactctcagtttaccaatgttgagcgcgaggttagagacttatggttaagtatattacctaccaagccagcatctgtatcaccagatgattcgtttttcgatttaggtggtcattctatcttggctaccaaaatgatttttaccttaaagaaaaagctgcaagttgatttaccattgggcacaattttcaagtatccaacgataaaggcctttgccgcggaaattgacagaattaaatcatcgggtggatcatctcaaggtgaggtcgtcgaaaatgtcactgcaaattatgcggaagacgccaagaaattggttgagacgctaccaagttcgtacccctctcgagaatattttgttgaacctaatagtgccgaaggaaaaacaacaattaatgtgtttgttaccggtgtcacaggatttctgggctcctacatccttgcagatttgttaggacgttctccaaagaactacagtttcaaagtgtttgcccacgtcagggccaaggatgaagaagctgcatttgcaagattacaaaaggcaggtatcacctatggtacttggaacgaaaaatttgcctcaaatattaaagttgtattaggcgatttatctaaaagccaatttggtctttcagatgagaagtggatggatttggcaaacacagttgatataattatccataatggtgcgttagttcactgggtttatccatatgccaaattgagggatccaaatgttatttcaactatcaatgttatgagcttagccgccgtcggcaagccaaagttctttgactttgtttcctccacttctactcttgacactgaatactactttaatttgtcagataaacttgttagcgaagggaagccaggcattttagaatcagacgatttaatgaactctgcaagcgggctcactggtggatatggtcagtccaaatgggctgctgagtacatcattagacgtgcaggtgaaaggggcctacgtgggtgtattgtcagaccaggttacgtaacaggtgcctctgccaatggttcttcaaacacagatgatttcttattgagatttttgaaaggttcagtccaattaggtaagattccagatatcgaaaattccgtgaatatggttccagtagatcatgttgctcgtgttgttgttgctacgtctttgaatcctcccaaagaaaatgaattggccgttgctcaagtaacgggtcacccaagaatattattcaaagactacttgtatactttacacgattatggttacgatgtcgaaatcgaaagctattctaaatggaagaaatcattggaggcgtctgttattgacaggaatgaagaaaatgcgttgtatcctttgctacacatggtcttagacaacttacctgaaagtaccaaagctccggaactagacgataggaacgccgtggcatctttaaagaaagacaccgcatggacaggtgttgattggtctaatggaataggtgttactccagaagaggttggtatatatattgcatttttaaacaaggttggatttttacctccaccaactcataatgacaaacttccactgccaagtatagaactaactcaagcgcaaataagtctagttgcttcaggtgctggtgctcgtggaagctccgcagcagcttaaggttgagcattacgtatgatatgtccatgtacaataattaaatatgaattaggagaaagacttagcttcttttcgggtgatgtcacttaaaaactccgagaataatatataataagagaataaaatattagttattgaataagaactgtaaatcagctggcgttagtctgctaatggcagcttcatcttggtttattgtactttcaacttctagaggagaaaagtattgacatgagcgctcccggcacaacggccaaagaagtctccaatttcttatttctttttgaattagataaatgagtgttctcaatttttttttctttgcattttttgtttgtgttgatttacaaaaacaatagaaaaaagaaaacaatattttctttctaaaaaaaaacaaaattgatgaaaaatagacatgaacaaaaaattttgaaagttgacttttttaaaaaatttttggtataatacaaaaaaagaatttttggaaaggtggcagagtggttgaatgctctggttttgaaaaccagcgtggctttacggtcaccgggggttcgaatccctccctttccgataatatatacaaaaatttttaaagttttttgtttattttgtatagataaaaaatctgcaataaaaatttcgttttttatttattcaaaaattctgtttttttgaaaagaaaataaaaaaaatgccaaaagtgagttttttattcaaatattagaaaaagtttttgaaaaatttaaaaaaatagaaaaaatttttttatttttttcataatttaaaaaattatgttataatttaaattacaaataggttttattaaaaaatttttacgtacagatgaattctataaaattattttggagatcaccatatggtaccaacatcaattcttaatactgtatcaactattcacagttctcgtgtaacttctgttgatcgtgttggtgttttaagtttaagaaattctgattcagttgaatttacacgtcgtcgtagtggatttagtactcttatttacgaatcacctggtagacgttttgtagtacgtgcagctgaaactgatacagataaagtaaaatctcaaactcctgataaagctcctgcaggtggttcttcaattaaccaacttttaggaataaaaggtgctagtcaagaaacaaacaaatggaaaatacgtttacaacttactaaaccagtaacatggccacctttagtatggggtgtagtttgtggtgctgctgcatcaggcaacttccattggacaccagaagatgtagctaaaagtattttatgtatgatgatgtctggtccttgtttaacaggttatacacaaactattaatgattggtatgacagagacattgatgcaatcaatgaaccttaccgtcctataccaagtggtgctatttcagaaccagaagttattacacaagtttgggttcttttacttggtggtcttggaattgctggtatcttagatgtatgggctggtcatacaacacctacagtattctatcttgctttaggaggtagtttactttcttacatttactcagctccacctttaaaacttaaacaaaatggttgggttggtaatttcgctttaggtgcttcatatatatcattaccttggtgggcaggtcaagctctttttggcacattaacaccagacgttgttgtacttactcttctttacagtatagctggcttaggtatcgcaattgtaaatgactttaaatcagttgaaggtgatagagcacttggtttacagagtttaccagttgcttttggaacagaaactgctaaatggatatgtgttggcgctattgacataacacaattaagtgtagcaggttacttacttgctagtggtaaaccatattacgctttagctttagtagctttaatcattcctcaaattgtttttcagttcaaatactttcttaaagatcctgtaaaatacgacgtaaaatatcaagcatctgctcaaccatttttagttttaggtattttcgtaactgctcttgcaagtcaacacggtaccggtgattataaagacgatgatgacaaatcaggtgaaaacttatactttcaaggtcacaatcatcgtcacaaacacaccggttaatctagattttattttttatgaaaaactcaggcttaatttaggcttgagtttttcattctttttgaagctctgaaattttaaaatttctagtcttctttaatgtttttaaattttaaaaaataaatttcttctctgctgtgtttttctttttttttgaaaaaacaaagaaaaaaaatttttttgttttcttctttgtttttttatttctttttgttttgtttattttttagtttcagaatctttgattcaaaaaaaaatttagtccgattactccataggagcaagcagtaaaaaataaaaactgtaataaaaaataaaacaaaaattttatttctttttgttttgcttgaacttttcaaaaaaaaattgaaaaattcaagcaaaacaaaaagaaacaaataaaaaatttatgaattttctactttttcaggagttgaaatttctcctttacttaaaacatattttgctaaaaaaagcgcttgtgttgctttttttgctactttttgtttccaagcattttttcgaatatttttttttgattttgatgtgcgtttttgttaacctaaaatcttgaaaagatttactcttttcaaatttttatgtttttattttttttattcataaaaaaaaacaatacataaaaataaagtatttcggcttcaaaaaattttatacaaaaagttttttgattaaaaactcagaaaaaataaaaaaacaaagtatgaattttttgaaaaattcataccttttatttttttgtaatttttagcctttcaaaaaatttttgaaggcattttttttttaatcctcatgttcttcaaaaggatctctcaatttttttgaaggaggtccaaaactcacatagattgaatatcctgttgcacttaataatagaaaccataaaaaaaaggtaaagaaaaaagcaggactgtccataattctttcatgttttttgttcaaatttattctccaataattatattacgacaaaaagtaaaaaaaatcaaaatttattcaaaaaaatggctactggaacaacttcaaaagctaaatcaagcttatctgatgcacttcaagaaccaggtatcgtaactcctttaggaactttattaagaccgttaaactctgaatcaggaaaagtattacctggatggggaacaactgttttaatgggtgttttcattgtactttttgctgtattcttattaattattttagaaatttataacagttctttattattagataatgttactatgagttgggaaactttagcttcttaattcaatagaatagttttattgctttttttattttttattttatcaaaaattttttttgcaaaaataaagaataaataaaattcaaaaaaattatagaattagataaaattagtttcaagttgaactaagttgtcaataaactttcaaatttgttttctttttactgttcattaagagcaataaaaaaaacttttggtcttggcaatcttttaaaaaagtcagaatcaattctattttaagaatcctatggaatctatgtatttaattttagcaaaattaccagaagcttatgcaccttttgatcctattgtagatgttttaccaattattcctattttcttcttattattagcctttgtatggcaagcatctgtaagttttagataaaaaatttaaaagttttttttgatacttttgtaaaaaatatcaaaaaaaacttttaaatttttttcaattttcattagcaactttagctttaatattagctaaagttgctctcaaaaatataatttttttttgactttttatttttttattttgtttcttttttaaaagttacaacataaagaaaatgaaaatagaaaatttgtgaaacataaaaaaaaagaatgaaatttttatgttcgttttttgttttatcttttccaactaaagtcggcctctagctagaggccggccaaatttttttccaaaattctataaaaaatcaaaaaattaaaaaaaaaagaaaaaactttgttttgtgcaaaacaaaaatcttgaattcaaaacaaaataaagaattcaaaaagattttttttaagcaaaaggtaaaatggaaaaaaatgtttttaaaaaaattttttcttttttttaaagctttgcttttttcatgaaaaaaacaaagctttaaaaaaaagaaaaaattttaaagcaaaaaaaagaattaaacacgtcttttttttggaggacgacatccattatgtggaattcctgttttttctcgaataacatttactttaattccagctttaaaaatttcacgaatagctgtttcgcgaccttgtcctggaccagttactaaaatttttgcttcatttaatgcaaattcacgtgatttttttgccacaacttcagcagctttttttgctgcaaatgttgttgcttttctttttccacggaaaccacaagctccagcagaactccaacaaaggacttcaccacgaagatttgctaatgtaataatagtattatgatgtccagcttgaatataaacaattcctcgatatgtacgttttttaatttttgtaggtgatacttttctagtttgtctagccatatgtaaaaatttaactataaatttcttttatatttttaaatcatttgatttactatctaaaaaaaataagattttgaatctttggaaagaaccattatttggaaagatttatttgttcaattcttttgtattttttttcaaaaaaattttttagaattttttatctaattttttaatgttttgttcaaacataaaaaattttcttttcaaaaaaggaaaaaaaatttcttttttttgaaaaagtaaaaataaaaaaagctgtagcttttttgttgaatcaaaaaaaaataagaaattgtcctatttttatgacaaaagattcaaaaaaatgaaataaaaaagaaaaattcaaactttcaaacaatgtattttgtatttttgggccgagtcggattcgaaccaacgtaggcgtaaccagcggatttacaatccgcccccattaaccactcgggcatcggcccatgcttttttttgactcatttgatcatttattttgcatgaattatactaaagattatattaacaaaaaatttttgaaatttcaattttttttaattaaaaactcttctatttttttaaaattctttgttttgaattttttttttcaatttaaaaaaaaaacatattaaaaaatatttttaaaaaattttttgtattgaaaacttacaataattttataaatttttttgaaaaattttgttttttttattctattgaaatgaacaaaacaaatttttttagttttttttgtttttttgctttgctgcttcttttgtttttatcaataaataaatgaaaatgaaaaacaaaaataaacaatttgtgtttttagagttctaaaatcaagaaaaaaatacttcccctttaaagaggaagtattttaaaaaaaaattatagtttgtcaatagtttcaaattcaaatttgatttctttccaaacttcacaagcagcagctaattctggagaccatttacaagctgagcggataacatcaccaccttcacgagctaaatcacgaccttcgttacgagcttgagtacaagcttctaaagcaacacggttagcaacagcaccaggagcgttaccccaagggtgtcctaaagtacctccaccgaattgtaaacaagcgtcatcaccaaagatttcaactaaagctggcatgtgccatacgtgaataccaccagaagcaactggcatagtaccacccatagaacaccagtcttgagtgaagtaaataccacggcta

SEQ ID NO: 121 - DNA, 1529 bp, Artificial Sequence
  codon optimized sequence comprising IS57
atggtaccaacattaaattcattatctccagcagaatcaaaagcaatttcatttttagatacttctcgtttcaatccaattcctaaattatctggtggtttttctttacgtcgtcgtaatcaaggtcgtggttttggtaaaggagtaaaatgctctgtaaaagtacagcaacagcaacaaccaccacctgcttggccaggtcgtgctgttccagaagctcctagacaatcatgggatggtcctaaacctattagtattgtaggatcaactggttcaatcggtactcaaactttagacattgttgcagaaaatccagataaatttcgtgtagttgcattagctgctggttctaacgttactttattagctgatcaggtacgtcgttttaaacctgctttagttgcagttcgtaatgaatctttaattaatgaattaaaagaagcattagcagatttagactacaaattagagattattccaggagaacaaggtgtaattgaagttgctcgtcatccagaagcagttacagtagttacaggtattgttggttgtgctggattaaaacctacagttgcagctattgaggctggtaaagatattgctttagcaaataaagaaacattaatcgcaggtggtccattcgtattaccacttgcaaacaaacacaatgtaaaaattcttccagcagacagtgaacattcagcaatttttcaatgtattcaaggcttacctgaaggtgcattaagaaaaatcatattaacagcttcaggtggtgcatttcgtgattggcctgtagaaaaacttaaagaggtaaaagtagctgatgcattaaaacacccaaactggaatatgggcaaaaaaattactgtagatagtgcaactttattcaacaaaggtttagaagttattgaagcacattatcttttcggtgctgaatacgacgatattgaaattgttattcatccacaatcaattattcatagtatgattgaaacacaagattcttcagtattagctcaattaggctggcctgacatgcgtttacctattttatatacaatgtcatggccagaccgtgttccttgcagtgaagttacttggccacgtttagatttatgtaaattaggatctcttacattcaaaaaacctgacaacgtaaaatatccatcaatggatcttgcttatgcagctggtcgtgcaggtggtactatgactggtgttttatctgctgctaatgagaaagctgtagaaatgttcattgacgagaaaatttcatacttagacatcttcaaagtagttgaattaacatgtgataaacaccgtaatgaattagtaacttctccaagtcttgaagaaattgtacattatgacttatgggctcgtgagtatgctgcaaatgttcaattatcaagtggagctcgtccagttcatgctggtaccggtgattacaaagatgatgatgataaaagtggtgagaacttatactttcaaggtcacaatcatcgtcataaacacaccggttaatctag

SEQ ID NO: 122 - DNA, 749 bp, Artificial Sequence
  codon optimized sequence comprising IS116
atggattataaagacgatgacgacaaaggtgctacaacacatttagatgtttgtgctgttgttcctgctgctggttttggtcgtcgtatgcaaacagaatgtccaaaacaatacttatcaattggtaatcaaacaattttagaacattcagttcatgctttattagctcatccacgtgttaaacgtgttgttattgctatttcaccaggtgattcacgttttgctcaattaccattagctaatcatccacaaattacagttgttgatggtggtgatgaacgtgctgattcagttttagctggtttaaaagctgctggtgatgctcaatgggttttagttcatgatgctgctcgtccatgtttacatcaagatgatttagctcgtttattagctttatcagaaacatcacgtacaggtggtattttagctgctccagttcgtgatacaatgaaacgtgctgaaccaggtaaaaatgctattgctcatacagttgatcgtaatggtttatggcacgctttaacaccacaattttttccacgtgaattattacatgattgtttaacacgtgctttaaatgaaggtgctacaattacagatgaagcatcagctttagaatactgtggttttcatccacaattagttgaaggtcgtgctgataacattaaagttacacgtccagaagatttagctttagctgaattttatttaacacgtacaattcatcaagaaaatacaaccggttaatctag

SEQ ID NO: 123 - DNA, 1151 bp, Artificial Sequence
  codon optimized sequence comprising IS62
atatggattataaagatgatgacgacaaaggtatgcacaagttcacaggtgttaacgctaaattccagcaaccagcattaagaaatttatctccagtggtagttgagcgcgaacgtgaggaatttgtaggattctttccacaaattgttcgtgacttaactgaagatggtattggtcatccagaagtaggtgacgctgtagctcgtcttaaagaagtattacaatacaacgcacctggtggtaaatgcaatagaggtttaacagttgttgcagcttaccgtgaactttctggaccaggtcaaaaagacgctgaaagtcttcgttgtgctttagcagtaggatggtgtattgaattattccaagcctttttcttagttgctgacgatataatggaccagtcattaactagacgtggtcaattatgttggtacaagaaagaaggtgttggtttagatgcaataaatgattcttttcttttagaaagctctgtgtatcgcgttcttaaaaagtattgccgtcaacgtccatattatgtacatttattagagctttttcttcaaacagcttaccaaacagaattaggacaaatgttagatttaatcactgctcctgtatctaaggtagatttaagccatttctcagaagaacgttacaaagctattgttaagtataaaactgctttctattcattctatttaccagttgcagcagctatgtatatggttggtatagattctaaagaagaacatgaaaacgcaaaagctattttacttgagatgggtgaatacttccaaattcaagatgattatttagattgttttggcgatcctgctttaacaggtaaagtaggtactgatattcaagataacaaatgttcatggttagttgtgcaatgcttacaaagagtaacaccagaacaacgtcaacttttagaagataattacggtcgtaaagaaccagaaaaagttgctaaagttaaagaattatatgaggctgtaggtatgagagccgcctttcaacaatacgaagaaagtagttaccgtcgtcttcaagagttaattgagaaacattctaatcgtttaccaaaagaaattttcttaggtttagctcagaaaatatacaaacgtcaaaaatcaggtccaagatcttaa

SEQ ID NO: 124 - DNA, 1257 bp, Artificial Sequence
  codon optimized sequence comprising IS61
atggtaccaacatcaattcttaatactgtatcaactattcacagttctcgtgtaacttctgttgatcgtgttggtgttttaagtttaagaaattctgattcagttgaatttacacgtcgtcgtagtggatttagtactcttatttacgaatcacctggtagacgttttgtagtacgtgcagctgaaactgatacagataaagtaaaatctcaaactcctgataaagctcctgcaggtggttcttcaattaaccaacttttaggaataaaaggtgctagtcaagaaacaaacaaatggaaaatacgtttacaacttactaaaccagtaacatggccacctttagtatggggtgtagtttgtggtgctgctgcatcaggcaacttccattggacaccagaagatgtagctaaaagtattttatgtatgatgatgtctggtccttgtttaacaggttatacacaaactattaatgattggtatgacagagacattgatgcaatcaatgaaccttaccgtcctataccaagtggtgctatttcagaaccagaagttattacacaagtttgggttcttttacttggtggtcttggaattgctggtatcttagatgtatgggctggtcatacaacacctacagtattctatcttgctttaggaggtagtttactttcttacatttactcagctccacctttaaaacttaaacaaaatggttgggttggtaatttcgctttaggtgcttcatatatatcattaccttggtgggcaggtcaagctctttttggcacattaacaccagacgttgttgtacttactcttctttacagtatagctggcttaggtatcgcaattgtaaatgactttaaatcagttgaaggtgatagagcacttggtttacagagtttaccagttgcttttggaacagaaactgctaaatggatatgtgttggcgctattgacataacacaattaagtgtagcaggttacttacttgctagtggtaaaccatattacgctttagctttagtagctttaatcattcctcaaattgtttttcagttcaaatactttcttaaagatcctgtaaaatacgacgtaaaatatcaagcatctgctcaaccatttttagttttaggtattttcgtaactgctcttgcaagtcaacacggtaccggtgattataaagacgatgatgacaaatcaggtgaaaacttatactttcaaggtcacaatcatcgtcacaaacacaccggttaa

SEQ ID NO: 125 - DNA, 5240 bp, Scenedesmus dimorphus
cgtttaggtgtaacacaatcttggggtggatggacaattagcggtgaaacagcaacaaatccaggtatttggagttatgaaggtgttgctgcatctcatattattttatctggtttattattcttagcttcggtttggcactgggtttactgggatttagagttattccgtgacccaagaactggaaaaactgcattagatttaccaaaaattttcggaattcacttattcttatcaggtcttttatgttttggttttggtgctttccacgtaacaggtttatttggtcctggtatttgggtttcagatccttatggattaacaggaagtgttcaaccagttgctccttcttggggtgctgatgggtttgatcctttcaaccctggtggtattgcagcgcaccacattgctgctggtattttaggtgttttagcaggattattccacttatgtgtacgtccttctattcgtttatactttggtttatcaatgggtagtatcgaaacagtattatcaagtagtattgctgctgttttctgggctgctttcgttgttgctggaactatgtggtatggttcagcagctactccaattgaattatttggtcctacacgttatcaatgggaccaaggtttcttccaacaagaaattcaaaaacgagttcaaacaagtttagcaggtggttcttcactttctgatgcttggtcgaaaattccagaaaaattagctttctatgattatattggaaacaaccctgcaaaaggtggtcttttccgtacaggagctatgaatagtggagatggtattgctgttggatggttaggtcacgcagtatttaaagatcaagatggtcgtgaattatacgtacgtcgtatgcctactttctttgaaacattcccagttttattaattgataaagatggtgttgtacgtgctgacgttcctttccgtcgtgctgaatcaaaatatagtattgaacaagttggtgtatcagtaactttctacggtggtgaattagatggattaacatttaatgatccagcaactgttaaaaaatatgctcgtaaagcacaattaggtgaaatttttgaatttgatcgttcaacattacaatctgatggtgtattccgtagtagtccacgtggttggtttacttttggtcacgtttgctttgctttattattcttctttggacatatttggcatggtgcacgtacaatcttccgtgatgtatttgctggtattgatgatgatctaaacgaaagtttagaatttggtaaatacaaaaaacttggtgatacaagttctgttcgtgaagctttctaattcgtttttttctcttttttttcttttttctctttggaaaaagaaaaaacatgtttattttgaattttttgtttagaactttactgttctttttttattttaaagtgtttttgtttttttttaatacaaaaacttttttaaaatgaatttaaaaaacacaaaaaaaagagttattgctattcaaaataaacaagagtttaaaaacaaagtttttttctaaagaaaaaaacttcttcattttttttgaattgtttttaaacttttttcttctcttgcttttagagtttttttcttcactttttgcaaaaaagtgagaaaaaacagcaaagcaaaaaagtgaaaaaaagttcaaaaacaattcaaaaaagacaaaacctaaaaaaatatcacttgagatgggtctggattttttccaagcaaaagaattttgtattttgttgaaagtttttcataaaaatacaaatttgcaattattattcttaaaatcaaaatatttgttaaccacatttcattctatggaagcattagtttatacttttttattaatcggaacattaggaattatctttttcgcaattttctttagagaaccacctcgtatggtaaaataattgaaattttgctttttttttatggataaaagaaatgatttcaattcatttcttttatccatttttttgaaaatagtttttctcaattttttattttttttgttttttctctaaaaatcaaaaattcaattttgagaaaaaattttatacaaaaagttttttgattaaaaactcagaaaaaagaaaaaaacaaagtatgaattttttgaaaaattcataccttttattttttttgtaatttttagcctttcaaaaaatttttgaaggcattttttttttaatcctcatgttcttcaaaaggatctctcaatttttttgaaggaggtccaaaactcacatagattgaatatcctgttgcacttaataatagaaaccataaaaaaaaggtaaagaaaaaagcaggactgtccataattctttcatgttttttgttcaaatttattctccaataattatattacgacagaaagtaaaaaaaatcaaaatttattcaaaaaaatggctactggaacaacttcaaaagctaaatcaagcttatctgatgcacttcaagaaccaggtatcgtaactcctttaggaactttattaagaccgttaaactctgaatcaggaaaagtattacctggatggggaacaactgttttaatgggtgttttcattgtactttttgctgtattcttattaattattttagaaatttataacagttctttattattagatgatgttactatgagttgggaaactttagcttcttaattcaatagaatagttttattgctttttttattttttattttatcaaaaattttttttgcaaaaataaagaataaataaaattcaaaaaaattatagaattagataaaattagtttcaagttgaactaagttgtcaataaactttcaaatttgttttctttttactgttcattaagagcaataaaaaaaacttttggtcttggcaatcttttaaaaaagtcagaatcaattctattttaagaatcctatggaatctatgtatttaattttagcaaaattaccagaagcttatgcaccttttgatcctattgtagatgttttaccaattattcctattttcttcttattattagcctttgtatggcaagcatctgtaagttttagataaaaaattgaaaagttttttttgatacttttgtaaaaaatatcaaaaaaaacttttcaatttttttcaattttcattcgcaactttagctttaatattagctaaagttgctctcaaaaatataatttttttttgactttttatttttttattttgtttcttttttaaaagttacaacataaagaaaatgaaaatagaaaatttgtgaaacataaaaaaaaagaatgaaatttttatgttcgttttttgttttatcttttccaactaaagtaaattttttttccaaaattctataaaaaatcaaaaaattaaaaaaaaagaaaaaactttgttttgtgcaaaacaaaaatcttgaattcaaaacaaaataaagaattcaaaaagattttttttaagcaaaaggtaaaatggaaaaaaatgttttttgctttaaaattttttcttttttttaaagctttgcttttttcatgaaaaaaacaaagctttaaaaaaaagaaaaaattttaaagcaaaaaaaagaattaaacacgtcttttttttggaggacgacatccattatgtggaattcctgttttttctcgaataacatttactttaattccagctttaaaaatttcacgaatagctgtttcgcgcccttgtcctggaccagttactaaaatttttgcttcatttaatgcaaattcacgtgatttttttgccacaacttcagcagctttttttgctgcaaatgttgttgcttttctttttccacggaaaccacaagctccagcagaactccaacaaaggacttcaccacgaagatttgctaatgtaataatagtattatgatgtccagcttgaatataaacaattcctcgatatgtacgttttttaatttttgtaggtgatacttttctagtttgtctagccatatgtaaaaatttaactataaatttcttttatatttttaaatcatttgatttactatctaaaaaaaataaaattttgaatctttggaaagaaccattatttggaaagatttatttgttcaattcttttgtattttttttcaaaaaaattttttagaattttttatctaattttttaatgttttgttcaaacataaaaaattttcttttcaaaaaagaaaaaaaaatttcttttttttgaaaaaggaaaaataaaaaaagctgtagcttttttgttgaatcaaaaaaaaataagaaattgtcctatttttatgacaaaagattcaaaaaaatgaaataaaaaagaaaaattcaaactttcaaacaatgtattttgtatttttgggccgagtcggattcgaaccaacgtaggcgtaaccagcggatttacaatccgcccccattaaccactcgggcatcggcccatgcttttttttgactcatttaatcatttattttgcatgaattatactaaagattatattaacaaaaaatttttgaaatttcaattttttttaattaaaaactcttctattttttaaaattctttgttttgaatttttttttcaatttaaaaaaaaaaaacatattaaaaaatatttttaaaaaattttttgtattgaaaacttacaataattttataaatttttttgaaaaattttgttttttttattctattgaaatgaacaaaacaaatttttttagttttttttgtttttttgttttgctgcttcttttgtttttatcaataaataaatgaaaatgaaaaacaaaaataaacaatttatgtttttagagttctaaaatcaagaaaaaaatacttcccctttaaagaggaagtattttaaaaaaaaattatagtttgtcaatagtttcaaattcaaatttgatttctttccaaacttcacaagcagcagctaattctggagaccatttacaagctgagcggataacatcaccaccttcacgagctaaatcacgaccttcgttacgagcttgagtacaagcttctaaagcaacacggttagcaacagcaccaggagcgttaccccaagggtgtcctaaagtacctccaccgaattgtaaacaagcgtcatcaccaaagatttcaactaaagctggcatgtgccatacgtgaataccaccagaagcaactggcatagtaccacccatagaacaccagtcttgagtgaagtaaataccacggcta

SEQ ID NO: 126 - DNA, 5240 bp, Scenedesmus dimorphus; identical to SEQ ID NO: 125

SEQ ID NO: 127 - DNA, 876 bp, Scenedesmus dimorphus
ttgaactatcaagtttaggttttaaaatctttatttatttactttattttttaatttgaaaactctgcgagctttgcgagcactgatttcaaaatcttagtttcaagtaaaacttattttcaatctttatttatttgtattttcaaactaaaagtttgaattatctaatttgaaactttatgagctttacaaagactggtttcaaattttttctttgtttagtttgttttttcaaactattagtttaaactatctaatttgaattataagtttgtattttcaaactctttatttgttaactttgttttttagtttgaaattctatgattttggacattagaaggctttgccttaatgattttccccgagcccctcttgggattctttttttttattttcctttcaggacttattataatatatcaaaaataatttttttgtcaattttttttaatattttaaaaattttttaaattaaaaataaatttttttttatttttagattttaatttttatttgtaagtttaatatttctaaaaaatttgaatttaagaattttttaatttgatgaaaaaaattgtttttgaattttttttttttactttaattaacttttttaaaaaatgattaaaaatttgaagtttttaaaaaactatctttttttttgtaaaaatggtattattttgtgtaaaatacaacaaaaataacaattttcaccttattttaagtttaatttttcaatgcaaaatttattttcaacaaaatgaaaaatatttttttagaatatattttatggcgggcgtagccaagtggtaaggcaatggattgtgactccatcattcgcgggttcgaaccccgtcgttcgcccataataaa

SEQ ID NO: 128 - DNA, 1724 bp, Artificial Sequence
  codon optimized sequence comprising rb1L-CAT-psbE
cggttttttttacttttgctttttttgcttttgttcaaagaaaaaaaaatacaaaataaaaaaaactaaaatgaaaaaacaaagaattctaaaattcataaaaaaaattaaaacccaattttttttttggaaacttttccaaataataaaaaaatcaaaaaaaaatttttctagtatttttttcatattttgaaactttttttgagtttataaaaaaatagaaaaaacaaatagatgaaaatttagaaaaattataaaccaataaaaatgaagttttgcgtagaaaaaaaatttagtttacttgttccccaagagcaagtggtaactttgaaaaaaatatttaaacttaaaaatttgctaaagttttgaatttatgttaaaatttaaaaaaaataaaaatttttaaactatttttttatgttaaaaaaatagtttttattattttctataatatagtttagttttttatttttttcaatttctttttttttttcaaagaaaaaagttttccacggatagatttttataggatcgacaaaatgttctatgaacttttcataatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgattccggcagtttctacacatatattcgcaagatgtggcgtgttacggtgaaaacctggcctatttccctaaagggtttattgagaatatgtttttcgtcagcgccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaacctagatatttgagaatttgtatttaaaactgaaaaatttttgaacgaactcttttcaaaaatattaaactttcttgagatgatttagtgttatctcaagaaagtttgttcttttattttttaaaatttttaaaaattttattttcttttaaacaggaaaataaataaagaaaaaagtgaattaaaaaaaagctgggactttcaaagtgaccaattttttactttaaagtttttttttattcaataaaaaaatactaaaaaaatatgaaagtattacttaaaatttcttaaaaaaaaaagaatgccttttttcaaaaaaaagtttaaaaaaaataaagtttttacgtattgtttaaaactttttttgaaaaaagcattcttttttcatttaaagagttatcttttttatctcgtgcaagttttggaaattcatttttgttaaataactttgactttttatttcttaaatttttggcttttcattttttttggtttacaaataaaa

SEQ ID NO: 129 - DNA, 25 bp, Artificial Sequence
  PCR primer
atggghytmccwtggtaycgtgthc

SEQ ID NO: 130 - DNA, 25 bp, Artificial Sequence
  PCR primer
ccratgtggcgrcaaggdatgttyg

SEQ ID NO: 131 - DNA, 25 bp, Artificial Sequence
  PCR primer
tttgwarratrathavdarraawrg

SEQ ID NO: 132 - DNA, 24 bp, Artificial Sequence
  PCR primer
kccwggdrmhactttwccdkmttc

SEQ ID NO: 133 - DNA, 2805 bp, Dunaliella aertiolecta
tctgtgcatttaatgcacacagctctagtagctggttgggctggtgctatgacattatttgaaattgcagtttttgatccatcagatccagtattaaaccctatgtggcgtcaagggatgttcgttcttcctttccttacacgtttaggtgtaacacaatcatggggtggttggacaattagtggtgaaacatcttcaaacccaggtatctggagttatgaaggtgctgcagcttcgcacattgttctttcaggtttattattccttgcttcagtttggcactgggtttactgggatttagaattattccgtgatccacgtacaggtaaaactgcattagatttaccaaaaatttttggtattcatcttttcttagcaggacttctttgttttggttttggtgctttccacgtaacaggtgtttttggacctggtatttgggtatcagatccatatggattaacaggtagtgtacaaccagtagctccttcttggggtgctgaaggttttgacccttacaacccaggtggtgtaccagctcaccatattgctgctggtattttaggtgtattagcaggtttattccacctttgtgttcgtccatcaattcgtttatattttggtttatcaatgggttctattgaatcagttttatcaagtagtattgcagccgtattctgggcagctttcgtagtagcaggtactatgtggtatggttctgcagcaactccaattgaattatttggtccaacacgttaccaatgggatcaaggtttcttccaacaagaaattcaaaaacgagtagcacaaagtacatctgaaaggtttatctgtttcgtagcacaaagtacatctgaaggtttatctgtttcagaagcttgggcaaaaattcctgaaaaattagctttctatgattacattggtaataacccagctaaaggtggattattccgtacaggtgctatgaacagtggtgatggtatcgctgtaggttggttaggacacgctagttttaaagatcaagaaggtcgtgaactttttgttcgtcgtatgcctactttctttgaaactttccctgttgttttaattgataaagacggtgttgttcgtgctgacgtaccattccgtaaagctgaatcaaaatactcaattgaacaagttggtgtttcagttacattctatggtggtgaattaaatggtttaacatttactgacccttcaactgttaaaaaatatgcacgtaaagctcaattaggtgaaatctttgaatttgaccgttcgactttacaatctgacggtgtattccgtagtagcccacgtggttggttcacttttggacacttatcttttgccttattattcttctttggtcatatttggcatggttcaagaactattttccgtgacgttttcgctggtattgatgaagacattaatgatcaattagaattcggtaaatataagaaacttggtgatacttcatctgttcgtgaagctttctaatcactatattaagttttattccaatattctaccaagaatatttagaaattcttgtttttaaattcatatagaaaaaatctattagaatttccgtcgagaaattctaatagattttttctctttttggcggactaaacacttttctttgtttttttgttttccgactagaaaatagaagattcatttaacaaagttcacatattttgtaatgtgatttttttgtaatgtgatttttcttatctttattagattttctatcttaccgtagattttaatacggtataattgtttttttttttttagatgggcttagattttctctgagcaaaagaatttgagttagtgtaaacaaatttgatcaaagtttatttcataaaaagaatttttttttataaatacggaagaaaatatacgagctaaattttatgttcttccgttttatttttataaaatgttaatattttatttttgttaaaaaaatctaagataatattttttttaactccttattggaattaaattttatcttttataactttaggtaagtcgttagatattttaaatttatcttctgaccgcttcgtttattttcttttcgtaattcggaagaaaattctttttgttttacactaatgtaaaatttggtattatgttcctatttgaaaagactagtcttttcaataaatttattaaacacctttcatggaagctttagtttacactttcttattaattggaacgttaggtattatctttttctctatcttttttagagagcctccacgtattgcaaaatagtaatagaataattttttattaaaattacttgtcggaccaaacaataaagttgttttttccgatacgaaaatctacgagaaattctatatttatcgtaaatacgtttcaaatgaagtattataagggttggatttcattaacattaattagtgaagtccaacccttagaatacttaaaatttttacttaactaagattattaatttaatcttcatgttcttcaaaaggatctctcaattttcttgaaggaggtccaaaactaacataaactgaataacctgtagcacttaataaaagaaaccataaaaagaaggtaaagaaaaaagccggactttccataaatttaaaatttttgacaagacaaaattattctccatatatattatacaatattacagaaaggaaaaaaacaaaaagattttattaactatcaattatggctacaggaaaaaatagtacacaaacttcaacatcacaagaaccaggaattgttacaccattagaa

SEQ ID NO: 134 - DNA, 3561 bp, Dunaliella
catttaatgcacactgctctagtagcaggttgggctggttcaatgacattattcgaaatcgccgtatttgacccttcagaccctgtattaaaccctatgtggcgtcaagggatgttcgtacttcctttcttaacacgtctaggtgtaacacaatcatggggtggttggacaattactggtgaatcagcatcaaacccaggtatctggagttatgaaggtgctgcagcttctcacattgttctttcaggtttattattccttgcatcagtttggcactgggtttactgggatttagaattattccgtgacccacgtacaggtaaaacagcattagatttaccaaaaatttttggtattcacctattcttatcaggacttctttgttttggttttggtgctttccatactacaggtgtttttggacctggtatttgggtatcagatccatatggtttaacaggtagtgtacaaccagtagctccttcttggggtgcagaaggttttgatccttacaacccaggtggtgtacctgctcaccatattgcagcaggtattttaggtgtattagctggtttattccacctatgtgttcgtccatctattcgtttatactttggtttatcaatgggttctattgaatcagttttatcaagtagtattgcagcagtattctgggcagcattcgttgtttcaggtactatgtggtacggttcagcaacaactccaattgaattatttggtccaactcgttaccaatgggatcaaggtttcttccaacaagaaattcaaaaacgagtagcacaaagtacatctgacggtttatctctttctgaagcttggtcaaaaattcctgaaaaattagctttctatgattacattggtaacaaccctgctaaaggtggtttattccgtacaggtgctatgaatagtggtgatggtattgccgtaggttggttaggccacgctagtttcaaagatcaagaaggacgtgaactttttgttcgtcgtatgcctactttcttcgaaactttccctgttgttttaattgataaagatggtattgttcgtgctgacgttccattccgtaaagcggaatctaaatactctattgaacaagtaggtgtttcagttacattctacggtggtgaattaaatggtttaacatttactgatccttctacagttaaaaaatacgctcgtaaagctcaattaggtgaaatcttcgaatttgaccgttctactttacaatctgatggtgtattccgtagtagtccacgtggttggtttactttcggacacttatcttttgctttattattcttctttggtcacatttggcatggttcaagaactattttccgtgatgttttcgctggtattgacgaagatattaatgaccaattagaattcggtaaatacaagaaacttggtgatacttcatctgttcgtgaagctttctaatcaattttttgatttaatcatttcgccacagagaacttttacaaaaataattttataaaactctctgaaattatttctccacactttgaccttggtctctagttcgtctggagaccaaggtcaaagtaaagttcttgtatgatatttctagaagaaaaaatttgattttcttcgataaatactcatataaaaataaaaaatcgaagggaagaaaacttcttcgaagttttctttttttaagataccgccagtttaaaatctaacaattttattcctaaggagaaaattcctataggaattttctctgccgcgtttcgaagaaacgcgtaggacttcttagaagtccgcctattaaatttttaaaagaaaatttttattgaatgtttttcttagacaaaagaaaaaacattttttcgtgtaggatttaaaagaaattcaagatttctatcctagtcttaatttaagaaaaagaatattttttctaaggatttacaaaattattattcaattgttgatttaaaatcttaaaattcgaaaatatttttgatttttaaagaaagagattaaaaaaaaaaaaaacgattttctaaatttaactttagattctatttttacttacctaactaataaagttttttagaaatttaaataaaaaattttatgtggtttttttgacgaaaaccctaaatcttgaacactttgtaaaatatcttttaaagattttttaaacttctatatgtaaaattttattttatttataaaagttttttctccttaaaagttttatttatacttttaaagtattatttttcgataaaaaaaaacaaatatttcaataaaatataattatattttataaaataaataaaagatgggcttagaatttctctgagcaaaagaatttaaactaatgtgaaaaacgtaagttaattttataaaaaaaatctaattttgtaaattatatgcgttttttacattaagtttaaatttggttttatgttcctatttgaaaagcaacagcttttcaataaatttattaaacatttcgcatggaagctttagtttacactttcttattagttggaacattaggtattattttcttctctattttctttagagaacctccacgtattgctaaataataaaaaaacatttctcaacgtaaattacaaataatttaataattactttgggatttgtgtaaaactcgcctaagaaatctgttgtatctttaatcttcaggctttgcacggtttaaattttcgtaggtcgcctcactgaggcgacctcgtgcctctcaaaaggtgctcaaagaaaaaaaaaaaaaaaaaagaaagacaaaaaacctttggttttttgtaccttagaaaaaaacttttaattttttttcctactaaaaattattttaggcggtgtcgcgaagcaacataggaaaaaaaattccgctagggaagaaaaatttccttaggaaattttctatttttttaattctagttttttttgtcggaaaaaaaccttaggtttttttcctaagggtctttttgccgaaggcgaaaagcccgccttattataaacagtttattaataaggttgaatttaactataaattttttaaaaaatgattatttttgttaaatcctaataattttatgtatgaaattttcattgttagtaaatcaattatttattaacatgaaaaatttgaagtttaaaaaattctaaaggattttttaaatcttctatatttctcgaagaaaatcaaagattttctccgagaaatacagatatatcttgaaaatcaaagattttcaagatactttatcgtgaataaagattattttttaatcttcatgttcttcaaaaggatctctcaattttcttgaaggtggtccaaaactaatataaactgaataacctgtagcacttaataaaagaaaccataaaaagaaggtaaagaaaaaagccggactttccataaatttaaaatttttgacaagacaaaattattctccataaattattatacaatattacagaaaggaaaaaatataaagatttaatttattttttattatggctacaggtaaaaataataca

SEQ ID NO: 135 - DNA, 2730 bp, N. abundans
  misc_feature (1477)..(1488): N is A, C, T or G
gctgtacatttaatgcatacttcattagtttctggttgggccggttcaatggctttttatgagcttgctgtttttgatccttctgatccagttttaaatccaatgtggcgtcaaggtatgtttgttttaccttttatgacacgtttaggtatcactcaatcctggggtggttggactatcagtggtgaaacggcttcaaatccgggtatctggagttatgagggtgtagccgcagctcacatcgttttatcaggtttactctttgctgcgtctatctggcactgggtttattgggatcttgaactttttcgtgatccaagaacttcaaatccagctttagatcttccaaaaatttttggtatccatttatttttatctggtgttctttgttttggttttggagctttccacgtaacaggtatttttggtcctggtatttgggtttctgatccttatggaattacaggaacagttcaagcagttgcgccttcttgggatgctacagggtttgatccctataatccgggtggaatttcagcacatcatattgctgccggcattttaggtgtattagctggtttattccacctttgtgttcgtccgccacaacgattatacaatggtctccgtatggggaatattgaaacagtactttctagcagtattgcagcagttttttgggcagcttttgttgtttctggtactatgtggtatggttcggcggcaacaccaattgaactttttggtcctactcgttatcaatgggatttaggcttcttccaacaagaaattgaacgtcgtgtacaaacaagtctttctgagggcaaatctgcttcgcaagcgtgggcagaaattccagaaaaattagctttttacgattacattggaaataatccagcaaaaggtggtcttttccgtgcgggtgctatgaacagtggagatggtattgcagtgggctggttaggtcatgctgttttcaaagataaacaaggtaacgaactttttgtacgtcgtatgccaactttctttgaaaccttccctgtcgttcttgtagataaagatggtgttgttcgtgcagatgttcctttccgtcgttctgaatcaaagtacagtatcgaacaagttggtgtttctgtaactttctatggtggagagttagatagtgtaacttttaatgatccagcaactgtgaaaaaatatgctcgacgtgctcaattaggagaaattttcgaatttgatcgtgcaactcttcaatcagacggtgttttccgtacaagtcctcgtggttggtttacatttgctcatttatgttttgctcttcttttcttctttggtcatatttggcatggtgctcgcacaatcttccgagatgtatttgctggtatcgatgcagacttagatgaacaagtagagtttggtgcattcttaaaacttggtgatacttctactcgtcgtcaatcggtttaaaggttcgggnnnnnnnnnnnnagttagtacaaaactactatttttggaaaaggaaatacctttcacaaactagactccaaggtttctattccagaagcggagaaaagaaatagaaccagtgagttttaaaactcactggttcttaaaggctcgtgagatagattttttgaaaattttatctaatctacaaaaattttttgaaactaactaagccagtttcatttttttagaaaaaatgaagaaagaagtatccactcaaattatccaacagaacaaaaaaagaagcttaatcctcatgttcttcaaatggatcacgaagttgttgtgatggtggaccaaaacctacgtaaatagaataaccggtaatacttaataataaacaccataaaaaaatggtaaagaaaaaagctgggctttccatactgatttaatttttagttacttaatactgagacaaaacgtttttaaaaacagagttgttccacaaaaagcccttttgggcctaaatggaattggtaccaattttcttttcggaaggaaatacattgaccgcttatttgtatattttcccgaaattttataaaaagtttagtcttcggcccaaaatagactcaaaacctaattttttagtcgaacacgaaaataagatctttacatttcccaaaaataaaatttttgggaacaacaagtttttgggacctaaaatcttttagtttaattttagcaaaaaccttgttttctttctttaagtaaaacacattttgtaattaaatgagaattagatgaattgaaatcattaaaaagtcaattaggttcgagtttattttttttgtagaaaaatttagctaaaagttttttctaatttaaaagttcctatataatagatttagaaaaaagtcttgaaatttttgaaagccaattacaaaaaaaatttggggtctttctgttttttgttaaacaaacaaatggaacggttactcagtcctaaaaatcttagttttgagaacaaaaacattgtttttaatttttatttttgcctctttaatgtcaaatagaagttaattcatactagtttggttcatctcggccctttcctttagacaagatgttgaggcccaaactgtgtttttgagcgcactaaaaattttgttaataaattttttgtttttcggttttctttttattacagaaagtaaaaaaaaaattatatcattgtaaaaactatggcaactggaactacatcaaaagtaaaatctgacgaca

SEQ ID NO: 136 - DNA, 2996 bp, Chlamydomonas vulgaris
tacgatcgggtcgtttattgctgtgcatttaatgcatacttctttagtttctggttgggctggttcaatggctttttatgaacttgctgtttttgatccttctgatccagttttaaacccaatgtggcgtcaagggatgtttgttcttccttttatgacacgtttaggcatcactcaatcttggggtggttggactatcagtggtgaaacagcatcaaatccaggtatttggagttatgagggggttgctgctgctcacatcattttatctggattactttttgctgcttctatttggcactgggtttattgggaccttgagcttttccgtgacccacgtacgtcaaatccagcattagaccttccaaaaatttttggtattcacttatttttatcaggtcttctttgctttggttttggagctttccatgtaactggattatttggtcctggtatttgggtttcagatccttatggtattacaggaactgttcaagcagttgctccttcttgggatgctacaggatttgatccttacaacccaggtggaatttcagcacatcacattgctgcaggtattttaggcgtcttagctggtttattccacctttgtgttcgtccaccacaaagattatacaatggacttcgcatgggtaacattgaaacagttctttctagcagtattgcagcagttttctgggcagcatttgttgtatcgggaactatgtggtatggttctgctgcaacaccaattgaactttttggtccaactcgttaccaatgggatttaggtttcttccaacaagaaattgaacgtcgtgtacaaacaagtcttgctgaaggaaaatcagcttcacaagcttgggcagaaattccagaaaaattagctttttacgattatattggaaacaacccagcaaaaggtggtcttttccgtgccggtgctatgaatagtggcgatggtattgcagtaggttggttaggccatgctgttttcaaagataaacaaggtaatgaactttttgttcgtcgtatgccaactttctttgaaacattccctgtagttcttcttgacaaagacggtgttgttcgtgcagacgtgcctttccgtcgttctgaatctaaatacagtattgaacaagtgggtgtttctgttacattctacggtggtgaattagatggtgtaacatttagtgatccagcaacagtgaaaaaatatgctcgacgcgcgcaattaggagaaattttcgaatttgatcgtgccactctacaatctgatggggttttccgtacaagccctcgtggttggtttacttttgctcatttatgttttgcactccttttcttctttggtcatatttggcacggtgctcgtacaatcttccgagatgtatttgcaggtattgatgcagatttagacgaacaagtagaatttggtgcattcttaaaacttggtgatacttcaactcgtcgtcaatcagtataattcattttttcttttacttcctctctcaaatttttcaaatttgggagaatttaccaaaactgaaataatttgcaagcctctatcatcttaaaaatattgtttgtaagaaaaaaaagaggtcggttatttgttctccaaactttttttcgaagtttttagagaaaaaagtttggtcccaaaaacgtagtttttggggaaaggaagtattttttcgttttcttccctttttctgtattttttttacctttgctttccaacaaaacagtattgttggagaaatcgaattagtcccaaaatttaaattttgggactaggtattagcatgaaaaaaaatgatagaaaaaggtaaacagtgcacttttctcccaaactaaatgatttttcatctagttggttactaacaaacagaacttttttctatggaagcgttagtttatacttttttattagttggaacattaggtattatcttttttgccattttctttagagaaccaccacgtattgtaaaataaaagtaccatttttggttttcgttgaaaaaaacttttgatatttttcatttatttcaaaagttataaaatttggaataaagggttaattttcagaccagattttttcccaaaaactttgtttttgggattgggagaattattactgttttagaaacaccaaagcttattctaaaacaataatagttttagcaccaaggaaaattcacattcccgaaaattcgaatggtcttaatatttactgactttgtgagttttaaaactcacaaagttatttaaacgaattaaaaagggttaatatatttccttcttatttctatttagtagtttaatgtaaaacagtagtgaagaactagtttgggaatttattttaatctttttcttattaaaggacttttgtgaatagttctaaatttagttggtcacttttgaaaaaaagtaataataattaaagaaaactaatgaaatttgatttactagagcattaatcttcatgttcttcaaaaggatcacgaagtttttgtgacggtggcccaaagccaacatagattgaataaccagtaatacttaataaaagacaccataaaaaaatggtaaagaaaaaagctggactttccataggtaaaaattacaaaactaagaaatttctttagaagataaaactttttttaactgtttagtttttatgaaaacagaaaaaaattatcttcgatctattcttgttttcattttaccaaaaaataagcaaaaagttatgttaaagtattctactttatagaaaagaacttttttacttttgtaaaatgaaaatatcttttttgtattctaaaacttttgagaaacttaaacttactatataataaagataagagggagaaaaaagcttaaaagctttttctcttttttacagaaagtaaaaaaaattcttttatttcaaaaattatggcaactggaactacatcaaaagtaaaatcagaagatactggaattcaactcca

SEQ ID NO: 137 - DNA, 1982 bp, Tetraselmis suecica
  misc_feature (1050)..(1052): N is A, C, T or G
gtcgtttattgcggtgcatttaatgcacacatcattagtttctggttgggctggttcaatggccttttatgaacttgctgtttttgatccatcagatccagttttaaacccaatgtggcgtcaaggtatgtttgttttacctttcatgactcgtttaggtatcacccaatcttggggtggttggacaattagtggtgaaacggcttcaaacccaggtatctggagttatgaaggtgttgctgcggctcacatcgttttatctggagctctttttggggcagctatttggcattgggttttttgggatttagaattattccgtgacccaagaacaggtaaccctgcattagatttaccaaaaatttttggtattcacttattcttatcagggttattatgttttggttttggagcattccatgtaacaggtttatttggacctgggatttgggtttctgatccttatggattaacaggaagtgttcaaccagtatctccatcatggggagccgatgggtttgatccatacaatccgggtggtattgcatctcaccatattgccgcaggtattttaggtattattgctggtttattccatttatgtgttcgtcctccacaacgtttatataatggtcttcgtatggggaacattgaaacagttctttctagtagtattgcggctgttttctgggcagcttttgttgttgcaggaacaatgtggtatggttgtgcggcaacaccaattgaattatttggcccaactcgttaccaatgggatcaagggtatttccaagaggaaattacaaaacgtgtagaaaaatctttatctgaagggcaatctttatcagaagcttggtctcaaattcctgaaaaattagctttctatgattacattggaaacaacccagctaagggtgggtttattccgtactggggctatgaacagtggcgatggtattgctgttgggttggttagtcatgcagttttcccagatttagatggtattgagtttatcagtttcgtcgtatgcccacgttcttgaacttttnnnagttaaatttacggatcccagcaacccgttaagaaatatgctcgtcgtgctcaatttaggagaaaattttgaaatttgaccgtgccacattacaaatcagatggtgttttccgaagcagtccacgtggttggtttactttgggcatttatcatttgctttattatttttctttggtcatatttggcacggagctcgtacaatcttccgtgatgtttttgcagggattgatccagatttagatgagcaagtagaatttggggcattccaaaaattaggagatacaacgactcgtcgtcaatctgtttagtttttcattttgaattcattcctcggattcaattatatccgcttaaatcaattattcttttaaaaatttattatggaagctcttgtttacacatttttacttgtaggaactttaggtatcatcttttttgcaatcttttttagagaaccacctcgtattgcaaaataaatagtttaacttcaaacttattatcaaaattgttgtgaattggggattaacccaattcacaacaattcaaatattaatcaaaaatagtttggttaatcttcatgctcttcaaacggatcacgaagatcctttgaaggtggaccaaacccaacataaactgaataccctgtaatacttaaaagtaaacaccataagaaaacagtaaaaaaaaaagcaggactttccataagtaaatttttttaatcttttaagttaaattcaatagcaataactaacttattgaatttataaaactctattattgttatatcataagtgaaagaacttttgactgaaaaattcaagaaagtaaaaaataattcctacgtttatatattatggcaacaggaacatcaaaagcatctaaaaatacacctgtaaatacagcaatggcgacacaattagaacgctataa

SEQ ID NO: 138 - DNA, 24 bp, Artificial Sequence
  PCR primer
ccacctcgtatggtaaaataattg

SEQ ID NO: 139 - DNA, 24 bp, Artificial Sequence
  PCR primer
gaaagaattatggacagtcctgct

SEQ ID NO: 140 - DNA, 21 bp, Artificial Sequence
  PCR primer
gaaggaggtccaaaactcaca

SEQ ID NO: 141 - DNA, 20 bp, Artificial Sequence
  PCR primer
cctggttcttgaagtgcatc

SEQ ID NO: 142 - DNA, 24 bp, Artificial Sequence
  PCr primer
tgagttgggaaactttagcttctt

SEQ ID NO: 143 - DNA, 20 bp, Artificial Sequence
  PCR primer
aaaagattgccaagaccaaa

SEQ ID NO: 144 - DNA, 25 bp, Artificial Sequence
  PCR primer
aaaaagaatgaaatttttatgttcg

SEQ ID NO: 145 - DNA, 20 bp, Artificial Sequence
  PCR primer
atggatgtcgtcctccaaaa

SEQ ID NO: 146 - DNA, 762 bp, Artificial Sequence
  codon optimized sequence comprising BD11
atggtaccagtatctttcacaagtcttttagcagcatctccaccttcacgtgcaagttgccgtccagctgctgaagtggaatcagttgcagtagaaaaacgtcaaacaattcaaccaggtacaggttacaataacggttacttttattcttactggaatgatggacacggtggtgttacatatactaatggacctggtggtcaatttagtgtaaattggagtaactcaggcaattttgttggaggaaaaggttggcaacctggtacaaagaataaggtaatcaatttctctggtagttacaaccctaatggtaattcttatttaagtgtatacggttggagccgtaacccattaattgaatattatattgtagagaactttggtacatacaacccttcaacaggtgctactaaattaggtgaagttacttcagatggatcagtttatgatatttatcgtactcaacgcgtaaatcaaccatctataattggaactgccactttctaccaatactggagtgtaagacgtaatcatcgttcaagtggtagtgttaatacagcaaaccactttaatgcatgggctcaacaaggtttaacattaggtacaatggactatcaaattgtagctgttgaaggttatttttcatcaggtagtgcttctatcactgttagcggtaccggtgattacaaagatgatgacgataaaagtggtgaaaacctttattttcaaggccataatcaccgtcacaaacacactggttaa

SEQ ID NO: 147 - DNA, 1332 bp, Artificial Sequence
  codon optimized sequence comprising IS99
atgacagtatacacagcttcagtaacagctccagtaaacattgctacattaaaatactggggtaaacgtgacacaaaattaaacttaccaacaaactcatcaatttcagtaacattatcacaagacgacttacgtacattaacatcagctgctacagctccagaattcgaacgtgacacattatggttaaacggtgaaccacactcaattgacaacgaacgtacacaaaactgtttacgtgacttacgtcaattacgtaaagaaatggaatcaaaagacgcttcattaccaacattatcacaatggaaattacacattgtatcagaaaacaacttcccaacagctgctggtttagcttcatcagctgctggtttcgctgctttagtatcagctattgctaaattataccaattaccacaatcaacatcagaaatttcacgtattgctcgtaaaggttcaggttcagcttgtcgttcattattcggtggttacgtagcttgggaaatgggtaaagctgaagacggtcacgactcaatggctgtacaaattgctgactcatcagactggccacaaatgaaagcttgtgtattagtagtatcagacattaaaaaagacgtatcatcaacacaaggtatgcaattaacagtagctacatcagaattattcaaagaacgtattgaacacgtagtaccaaaacgtttcgaagtaatgcgtaaagctattgtagaaaaagacttcgctacattcgctaaagaaacaatgatggactcaaactcattccacgctacatgtttagactcattcccaccaattttctacatgaacgacacatcaaaacgtattatttcatggtgtcacacaattaaccaattctacggtgaaacaattgtagcttacacattcgacgctggtccaaacgctgtattatactacttagctgaaaacgaatcaaaattattcgctttcatttacaaattattcggttcagtaccaggttgggacaaaaaattcacaacagaacaattagaagctttcaaccaccaattcgaatcatcaaacttcacagctcgtgaattagacttagaattacaaaaagacgtagctcgtgtaattttaacacaagtaggttcaggtccacaagaaacaaacgaatcattaattgacgctaaaacaggtttaccaaaagaaaccggttacccatacgacgtacctgactatgcttacccttacgacgtaccagactatgcttatccatacgacgtaccagactacgctgaaaacttatacttccaaggtcaccaccaccaccaccatcaccacccaccaggttaa

SEQ ID NO: 148 - DNA, 660 bp, Artificial Sequence
  codon optimized sequence comprising CAT
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 149 - DNA, 90 bp, Artificial Sequence
  PCR primer
agtttttctcaattttttattttttttgttttttctctaaaaatcaaaaattcaattttgagaaaacgtaagatctcctaggaaaatgaa

SEQ ID NO: 150 - DNA, 90 bp, Artificial Sequence
  PCR primer
tcggtttaagacaatgggaaaagttagatgcctagagtattgattatcgagcaaatatcttctcatctgtgacgggctcgagactagtgg

SEQ ID NO: 151 - DNA, 90 bp, Artificial Sequence
  PCR primer
tggattggtatcaacgcgcaggcatagttcgagaaaaattatccagaggcaatgacaaccagcatctcctagtgctagctaaagaagttg

SEQ ID NO: 152 - DNA, 90 bp, Artificial Sequence
  PCR primer
tcaaaaaattcatactttgtttttttattttttctgagtttttaatcaaaaaactttttgtataaaattgggctcgagactagtttgtcc

SEQ ID NO: 153 - DNA, 22 bp, Artificial Sequence
  PCR primer
tcttactggaatgatggacacg

SEQ ID NO: 154 - DNA, 22 bp, Artificial Sequence
  PCR primer
gtgtttgtgacggtgattatgg

SEQ ID NO: 155 - DNA, 22 bp, Artificial Sequence
  PCR primer
tgtggacctgaacctacttgtg

SEQ ID NO: 156 - DNA, 22 bp, Artificial Sequence
  PCR primer
gaaatgggtaaagctgaagacg

SEQ ID NO: 157 - DNA, 20 bp, Artificial Sequence
  PCR primer
cttccaaaaccacctgttgc

SEQ ID NO: 158 - DNA, 20 bp, Artificial Sequence
  PCR primer
accgtctggatcaaaagcag

SEQ ID NO: 159 - DNA, 20 bp, Artificial Sequence
  PCR primer
ttggagtggttctgttcgtg

SEQ ID NO: 160 - DNA, 21 bp, Artificial Sequence
  PCR primer
cagcgtacatacgtcctggat

SEQ ID NO: 161 - DNA, 20 bp, Artificial Sequence
  PCR primer
gttgcgctcaaccaacatta

SEQ ID NO: 162 - DNA, 20 bp, Artificial Sequence
  PCR primer
gtgacggtggttgtgtcctt

SEQ ID NO: 163 - DNA, 20 bp, Artificial Sequence
  PCR primer
cctgcaggtggttcttcaat

SEQ ID NO: 164 - DNA, 20 bp, Artificial Sequence
  PCR primer
atgtcaatagcgccaacaca

SEQ ID NO: 165 - DNA, 25 bp, Artificial Sequence
  PCR primer
tggattataaagatgatgacgacaa

SEQ ID NO: 166 - DNA, 22 bp, Artificial Sequence
  PCR primer
gctgctgcaactggtaaataga

SEQ ID NO: 167 - DNA, 20 bp, Artificial Sequence
  PCR primer
tccagcagaatcaaaagcaa

SEQ ID NO: 168 - DNA, 20 bp, Artificial Sequence
  PCR primer
gcaccttcaggtaagccttg

SEQ ID NO: 169 - DNA, 21 bp, Artificial Sequence
  PCR primer
aagacgatgacgacaaaggtg

SEQ ID NO: 170 - DNA, 21 bp, Artificial Sequence
  PCR primer
tgttatcagcacgaccttcaa